(19) United States
(12) Patent Application Publication
(10) Pub. No.: US 2022/0235379 A1
(43) Pub. Date: Jul. 28, 2022

(54) TARGETED GENE EDITING CONSTRUCTS AND METHODS OF USING THE SAME

(71) Applicant: UNIVERSITAT POMPEU FABRA, Barcelona (ES)

(72) Inventors: Avencia SÁNCHEZ-MEJÍAS GARCÍA, Barcelona (ES); Marc GÜELL CARGOL, Barcelona (ES); Dimitrie IVANCIC DJERMANOVIC, Barcelona (ES); Maria PALLARÈS MASMITJA, Barcelona (ES)

(73) Assignee: UNIVERSITAT POMPEU FABRA, Barcelona (ES)

(21) Appl. No.: 17/617,252

(22) PCT Filed: Jun. 11, 2020
(86) PCT No.: PCT/IB2020/055507

§ 371 Date: Dec. 7, 2021

Related U.S. Application Data
(60) Provisional application No. 62/860,186, filed on Jun. 11, 2019.

(51) Int. Cl. C12N 15/90; C12N 9/22; C12N 9/12; C12N 15/11; C12N 15/62; C12N 15/85; C12N 15/86; C12N 7/00

(52) U.S. Cl. CPC C12N 15/907; C07K 2319/81; C12N 9/1241; C12N 15/11; C12N 15/62; C12N 15/85; C12N 15/86; C12N 7/00; C12N 2310/20; C12N 2800/80; C12N 2800/107; C12N 2740/16022; C12N 2800/90; C12N 2740/16043; C12N 9/22

(57) ABSTRACT

A nucleic acid constructs are used in improving site-specific insertion of an exogenous nucleic acid into a genome. In some aspects the nucleic acid construct having a first polynucleotide sequence encoding a DNA binding protein engineered to bind to a specific genomic DNA sequence, a second polynucleotide having a modified integrase or a modified transposase that enables insertion of exogenous nucleic acid into the genome, and a nucleic acid sequence encoding a linker between the two nucleotides. In some aspects, the nucleic acid construct encodes a fusion protein, for example, a fusion protein for delivery to a cell by a lentiviral particle.

### REFERENCE TO SEQUENCE LISTING SUBMITTED ELECTRONICALLY

The content of the electronically submitted sequence listing in ASCII text file (Name: 4349.001PC01_Seqlisting_ST25; Size: 389,120 bytes; and Date of Creation: Jun. 11, 2020) filed with the application is incorporated herein by reference in its entirety.

### BACKGROUND

Many diseases such as cancer, developmental disorders, and some infections have genetic and epigenetic aberrations in common. Gene therapy is designed to introduce genetic material into cells to target and edit the genome directly in order to correct genetically dysfunctional cells and thereby cure the associated diseases. Zinc finger nucleases (ZFNs), Talen and Crispr-cas9 gene editing technologies represent some of the recently developed tools for editing DNA. Methods such as electroporation, cationic lipids, microinjections, or viruses have been used for delivery of genetic material into a genome. Current strategies for gene delivery are commonly based on adenoviruses, retroviruses, or naked DNA plasmids.

Lentiviruses, which include HIV, are a powerful tool when used as a vector for nucleic acid delivery. Lentiviruses are capable of stably infecting dividing and non-dividing cells. Lentiviral vectors are prone to random integration in the host genome, and can often integrate at the site of highly transcribed genes which raises the risk of insertional mutagenesis.

HIV-1 integrase catalyzes the insertion of viral DNA in the host genome. In general, HIV-1 integrase consists of a N-terminal domain (NTD), a Catalytic core domain (CCD) and a C-terminal domain (CTD). The NTD is used to bind and coordinate a Zn2+ cation as an important co-factor, while the CTD is used for DNA binding. The CCD forms the catalytic core in which the integration process is catalyzed. Challenges with the insertion mechanisms used by viral vectors include low efficiency and a lack of specificity, which can result in unintended insertion mutagenesis and genotoxicity.

### BRIEF SUMMARY

Some aspects of this disclosure provide constructs, plasmids, vectors, particles, fusion proteins, compositions, methods, and kits that are useful for the targeted editing of nucleic acids, including editing a single site or region within a subject's genome, e.g., the human genome.

Working examples herein provide detailed experimental data plausibly demonstrating the successful generation of constructs of fusion proteins of programmable transposases and integrases with Cas9/Zinc Finger proteins. Furthermore, such constructs were able to cause site-specific integration of an exogenous nucleic acid sequence into the genome of transfected cells. Without being bound to theory, the present inventors believe that this is the first time that fusion proteins of such type, with the ability of site-specific integration of an exogenous nucleic acid in a genome and suitable for gene therapy especially involving large genes, have been generated. The inventors have also identified modified hyperactive PiggyBac transposases which perform specific targeted transpositions.

Accordingly, an aspect of this disclosure relates to a nucleic acid construct comprising:

a) a first polynucleotide sequence comprising a nucleic acid encoding a first DNA binding protein engineered to bind to a specific genomic DNA sequence in a genome; wherein the first DNA binding protein is a zinc finger protein or a Cas9 protein;

b) a second polynucleotide sequence comprising a nucleic acid encoding a second DNA binding protein which enables insertion of an exogenous nucleic acid into a genome, wherein the second DNA binding protein is
i. a hyperactive PiggyBac transposase, or a modified hyperactive PiggyBac with improved specificity of inserting the exogenous nucleic acid into the genome compared to the hyperactive PiggyBac, or
ii. a human immunodeficiency virus (HIV) integrase, or a modified HIV integrase with improved specificity of inserting the exogenous nucleic acid into the genome compared to the HIV integrase; and

c) an optional polynucleotide sequence comprising a nucleic acid encoding a linker;

wherein the nucleic acid construct encodes a fusion protein comprising the first DNA binding protein, the second DNA binding protein, and the optional linker between the first DNA binding protein and the second DNA binding protein; and

wherein the fusion protein enables insertion of the exogenous nucleic acid into a specific site of the genome.

Also provided is a composition comprising a nucleic acid construct, a vector or a fusion protein as described herein, and a polynucleotide sequence encoding an exogenous nucleic acid for insertion in a genome, the composition contained in or bound to a packaging vector.

The present disclosure also provides a method for controlled, site-specific integration of a single copy or multiple copies of an exogenous nucleic acid sequence into a cell, the method comprising: (a) delivering the nucleic acid construct, the vector or the fusion protein described herein to the cell, and (b) delivering the exogenous nucleic acid to the cell; wherein binding of the fusion protein to the specific genomic DNA sequence in the genome of the cell, results in cleavage of the genome and integration of one or more copies of the exogenous nucleic acid into the genome of the cell.

Another aspect relates to the provision of modified hyperactive PiggyBac transposases comprising the amino acid sequence SEQ ID NO: 9, wherein: amino acid at position 245 is A, amino acid at position 275 is R or A, amino acid at position 277 is R or A, amino acid at position 325 is A or G, amino acid at position 347 is N or A, amino acid at position 351 is E, P or A, amino acid at position 372 is R, amino acid at position 375 is A, amino acid at position 450 is D or N, amino acid at position 465 is W or A, amino acid at position 560 is T or A, amino acid at position 564 is P or S, amino acid at position 573 is S or A, amino acid at position 592 is G or S, and amino acid at position 594 is L or F.

In some embodiments, fusion proteins of (i) an integrase, a modified integrase, a transposase or a modified transposase linked to a (ii) Cas9 or a Zinc Finger protein; and nucleic acid constructs encoding the same, are provided.

Certain aspects of the application are directed to a nucleic acid construct comprising: (a) a first polynucleotide sequence encoding a first DNA binding protein engineered to bind to a specific genomic DNA sequence in a genome; (b) a second polynucleotide sequence encoding a second DNA binding protein which enables insertion of an exogenous nucleic acid into the genome, wherein the second DNA binding protein is (i) an integrase or a modified integrase which is modified relative to a wildtype integrase or (ii) a transposase or a modified transposase which is modified relative to a wildtype transposase; and (c) a third polynucleotide sequence comprising a nucleic acid encoding a linker; wherein the nucleic acid construct encodes a fusion protein comprising the first DNA binding protein, the second DNA binding protein, and the linker between the first DNA binding protein and the second DNA binding protein.

In some embodiments, the nucleic acid construct comprises: (a) a first polynucleotide sequence encoding a Cas 9 protein; and (b) a second polynucleotide sequence encoding a transposase or a modified hyperactive PiggyBac of the disclosure or a functional fragment thereof.

In some embodiments, the nucleic acid construct comprises: (a) a first polynucleotide sequence encoding a zinc finger protein; and (b) a second polynucleotide sequence encoding an integrase or a modified integrase of the disclosure or a functional fragment thereof.

In some embodiments, the application is directed to a plasmid, vector, or host cell comprising a nucleic acid construct of the disclosure.

Some aspects of the application are directed to a fusion protein comprising: a first DNA binding protein engineered to bind to a specific genomic DNA sequence in a genome; a second DNA binding protein which enables insertion of an exogenous nucleic acid into the genome, wherein the second DNA binding protein is an integrase, a transposase or a modified integrase or transposase; and a linker connecting the first protein and the second protein.

In some embodiments, the fusion protein comprises: (a) a Cas 9 protein; and (b) a hyperactive PiggyBac or a modified hyperactive PiggyBac of the disclosure or a functional fragment thereof.

In some embodiments, the fusion protein comprises: (a) a zinc finger protein; and (b) an integrase or a modified integrase of the disclosure or a functional fragment thereof.

Some aspects of the application are directed to a lentiviral particle comprising a fusion protein of the disclosure.

Some aspects of the application are directed to a method of inserting an exogenous nucleic acid sequence into genomic DNA of an organism, comprising: administering a lentiviral particle comprising a nucleic acid construct or a fusion protein of the disclosure to the organism such that the first and second DNA binding proteins bind to a specific genomic DNA sequence and insert the exogenous nucleic acid into the genomic DNA; wherein the exogenous nucleic acid becomes integrated at the specific genomic DNA sequence.

Some aspects of the disclosure are directed to a method for controlled, site-specific integration of a single copy or multiple copies of an exogenous nucleic acid sequence into a cell, the method comprising: (a) delivering the fusion protein of the disclosure to the cell, and (b) delivering the exogenous nucleic acid to the cell; wherein binding of the fusion protein to the specific genomic DNA sequence in the genome of the cell, results in cleavage of the genome and integration of one or more copies of the exogenous nucleic acid into the genome of the cell; and wherein the fusion protein is delivered to the cell by a lentiviral particle.

Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. Furthermore, the present invention covers all possible combinations of particular and preferred embodiments described herein. The following examples and drawings are provided herein for illustrative purposes, and without intending to be limiting to the present invention.

### BRIEF DESCRIPTION OF THE DRAWINGS

FIGS. 1A and 1B show the percent of cells that have the exogenous nucleic acid sequence integrated into their genome after transfection with (FIG. 1A) Cas9-PiggyBac fusion proteins (human Cas9 (hCas9), nickase Cas9 (nCas9), or dead Cas9 (dCas9) and hyperactive PiggyBac (PB) transposase) and (FIG. 1B) Cas9-SB100 fusion proteins (human Cas9 (hCas9), nickase Cas9 (nCas9), or dead Cas9 (dCas9) and hyperactive Sleeping Beauty (SB100) transposase). Vectors were created in which the 3′ end of the Cas9 was connected to the 5′ end of each of the transposases by a GGS linker (SEQ ID NOS: 48, 49) (hCas9PB, nCas9PB, dCas9PB, hCas9SB, nCas9SB, and dCas9SB). Other vectors were created in which the 3′ end of each transposase was connected to the 5′ end of the Cas9 by a GGS linker (SEQ ID NOS: 48, 49) (PBhCas9, PBnCas9, PBdCas9, SBhCas9, SbnCas9, and SBdCas9). “PiggyBac” (FIG. 1A) and “SB100” (FIG. 1B) were used as positive control and the transposon alone encoding a RFP (denoted as “Episomal RFP” in FIG. 1A) and GFP (denoted as “Episomal GFP” FIG. 1B) were used as negative controls. FIG. 1C is a different representation of FIG. 1A showing transposition activity with PB and Cas9 in different configurations.

FIG. 2A shows a plasmid construct encoding a Cas9/PB fusion protein.

FIG. 2B shows the percent of cells that have the exogenous nucleic acid sequence integrated into their genome by the fusion constructs formed by a human Cas9-PiggyBac (“Targeted HCas9”) or a nickase Cas9-PiggyBac (“Targeted NCas9”). The 3′ end of the Cas9 was connected to the 5′ end of the transposase by a linker. “Non-targeted” is the control for overall insertion (PiggyBac alone) and “Episomal” is the negative control of no-integration (transposon alone).

FIG. 3 shows an exemplary ZFP-integrase fusion protein. The ZFP and the integrase are linked by a GGS sequence. NLS refers to Nuclear Localization Sequence.

FIG. 4 shows the lentivirus titer of wild-type integrase lentivirus (LV), empty viral particles (LVO), non-integrative lentivirus (NILV), non-integrative lentivirus with wild-type integrase (NILV+IN), non-integrative lentivirus with ZFP-integrase fusion protein (NILV+ZP-IN (AAVS1)), non-integrative lentivirus with Cas9-integrase fusion protein (NILV+Cas-IN), and wild-type integrase lentivirus with wild-type integrase (LV+IN). (′) denotes a technical replicate.

FIG. 5 shows the percent of cells that integrated (overall integration) the exogenous nucleic acid sequence into their genome after infection with wild-type integrase lentivirus (LV), empty viral particles (LVO), non-integrative lentivirus (NILV), non-integrative lentivirus with wild-type integrase (NILV+IN), non-integrative lentivirus with ZFP-integrase fusion protein (NILV+ZP-IN(AAVS1)), non-integrative lentivirus with Cas9-integrase fusion protein (NILV+Cas-IN), and wild-type integrase lentivirus with wild-type integrase (LV+IN). For each condition, from left to right, the first column refers to Day 3, the second column to Day 5, the third column to Day 7, the fourth column to Day 10 and the fifth column to Day 12.

FIG. 6 shows an image of chromosomes with representative AAVS1 integration and non-integration sites. A star symbol represents the site for AAVS1 in chromosome 19, a triangle symbol means non-targeted integration sites; and a diamond symbol means targeted integration.

FIG. 7A shows the virus titer generated by wild-type integrase lentivirus (LV), empty viral particles (LVO), non-integrative lentivirus (NILV), non-integrative lentivirus with wild-type integrase (NILV+IN), non-integrative lentivirus with ZFP-IN fusion protein targeted to the AAVS1 site (NILV+ZP-IN(AAVS1)), and non-integrative lentivirus with ZFP-IN fusion protein targeted to the CCR5 site (NILV+ZP-IN(CCR5)).

FIG. 7B shows percent of cells that integrated (overall integration) the exogenous nucleic acid sequence into their genome after infection with wild-type integrase lentivirus (LV), non-integrative lentivirus (NILV), non-integrative lentivirus with wild-type integrase (NILV+IN), non-integrative lentivirus with ZFP-IN fusion protein targeted to the AAVS1 site (NILV+ZP-IN(AAVS1)), and non-integrative lentivirus with ZFP-IN fusion protein targeted to the CCR5 site (NILV+ZP-IN(CCR5)).

FIG. 7C shows percent of cells that integrated the exogenous nucleic acid sequence into their genome after infection with wild-type integrase lentivirus (LV), empty viral particles (LVO), non-integrative lentivirus (NILV), non-integrative lentivirus with wild-type integrase (NILV+IN), non-integrative lentivirus with ZFP-IN fusion protein targeted to the AAVS1 site (NILV+ZP-IN(AAVS1)), and non-integrative lentivirus with ZFP-IN fusion protein targeted to the CCR5 site (NILV+ZP-IN(CCR5)).

FIG. 7D shows percent of cells that integrated the exogenous nucleic acid sequence into their genome after infection with wild-type integrase lentivirus (LV), non-integrative lentivirus (NILV), non-integrative lentivirus with wild-type integrase (NILV+IN), non-integrative lentivirus with ZFP-IN fusion protein targeted to the AAVS1 site (NILV+ZP-IN(AAVS1)), and non-integrative lentivirus with ZFP-IN fusion protein targeted to the CCR5 site (NILV+ZP-IN(CCR5)).

FIG. 8A-8C show the lentivirus titer (FIG. 8A) and the % of CAR expressing cells at day 3 and day 14 (FIG. 8B), and the % of CD3 expression cells is shown in FIG. 8C. Jurkat cells were infected with several conditions of lentivirus: Wild-type integrase lentivirus (LV), empty viral particles (LVO), non-integrative lentivirus (NILV), non-integrative lentivirus with wild-type integrase (NILV+IN), non-integrative lentivirus with ZFP-integrase fusion protein (NILV+ZFP-IN(TRCa-1), non-integrative lentivirus with Cas9-integrase fusion protein (NILV+Cas-IN). NILV showed a drastic decrease in the titer; and transcomplementation with the expression of IN WT or fusion ZNF-IN in the virus producing cells did not have a rescue effect on titter, nor on integration capacity. Additionally, cells did not lose the expression of CD3 when integration is targeted towards the TCR locus (CD3 protein expression). This denotes the need to use additional factors for transcomplementation such as VPR protein; especially in the context of this cell line.

FIG. 9A-9B show titer for WT lentivirus and two different integrase deficient virus systems (NILV and TAA, the latter indicating that a stop codon has been introduced at the beginning of the IN-coding region in the lentiviral packaging plasmid) alone or transcomplemented with IN or VPR IN fusion. Titers were detected by Fluorescent cytometry analysis at day 3 after infection (FIG. 9A). FIG. 9B shows the relative integration efficiencies of transcomplemented integration machineries showing the advantage of VPR protein fusion to IN for transcomplementation. WT: Lentivirus produced with WT IN; NILV: Lentivirus produced with non-integrative IN, harboring two mutations on its catalytic center; TAA: Lentivirus produced with a IN defective IN, where the protein is not expressed; +IN: Lentivirus transcomplemented with IN; +VPR-IN: Lentivirus transcomplemented with IN fused to VPR in the C-terminal end.

FIG. 10A shows a scheme of the nucleic acid construct formed by an insertion domain with a DNA binding domain and a programmable DNA recognition domain fused by means of a linker. FIG. 10B is a scheme showing the fusion of Cas9 and a transposase joined by a linker in different configurations.

FIG. 11 shows results of Cas9 activity in Cas9 linked to hyPB using different linkers size and compositions. Cas9 activity was measured by sequencing the gRNA target site and using CRISPR-GA to analyze indel frequency. 2 different gRNAs were used targeting AAVS1 site. Linkers used are SEQ ID NOS 50 to 63.

FIG. 12 shows results of programmable transposase genetrap transposition efficiency. RFP fluorescence was measured by Flow Cytometry 10 days after transfection. Different linkers were used to determine linkers' length and composition importance in targeted insertion. Average of 2 independent experiments. Linkers used are SEQ ID NOS 50 to 63.

FIG. 13 shows results of hcas9_PB linkers targeted transposition. Targeted transposition efficiencies of different cas9-PB linkers constructs using the split GFP cell line using 2 different gRNAs. GFP expression was measured by flow cytometry 72 h post-transfection.

FIG. 14 shows a scheme of the split GFP reporter cell line generated for the screening of high throughput analysis of the library of the different hyPB mutations as well as the validation of individual mutants. A Splice acceptor (SA) followed by half of the coding sequence of GFP (Ct-GFP), downstream of a target region site was introduced into the genome of Hek293T cells using the Sleeping Beauty 100× system. The PiggyBac transposon flanked by the Inverted Terminal Repeats (ITRs) for this screening was either a full RPF expressing cassette followed by a promoter and the other half of GFP (Nt-GFP) and a splice donor (SD); of just the half GFP fragment; as shown in the figure.

FIG. 15 shows results of hcas9_PB selected mutants targeted transposition. Targeted transposition efficiencies of hcas9_PB D450N and hcas9_PB R372A K375A D450. GFP expression was measured by flow cytometry 72 h post-transfection. Average of 4 independent experiments.

FIG. 16 shows results of hcas9_PB selected mutants random and targeted transposition. Targeted and random transposition efficiencies of hcas9_PB D450N and hcas9_PB R372A K375A D450. GFP expression was measured by flow cytometry 72 h post-transfection and RFP expression was measured by flow cytometry at 15 days post-transfection and normalized by RFP fluorescence 48 h after transfection assumed as transfection efficiency.

FIG. 17 is a scheme showing the fusion of ZFP and a transposase joined by a linker in different configurations.

FIG. 18 shows results of ZFP-PB fusion proteins targeted transposition. Targeted transposition efficiencies of ZFP_hyPB or ZFP_hyPBD450N in N and C-terminal conformations. GFP expression was measured by flow cytometry 5 days post-transfection. More than 1 independent repeat. ZFP_PB: Fusion ZFP and hyPB in C-terminal configuration using XTEN linker; PB_ZFP: Fusion ZFP and hyPB in N-terminal configuration using XTEN linker, ZFP_450: Fusion ZFP and hyPB (D450N) in C-terminal configuration using XTEN linker; 450_ZFP: Fusion ZFP and hyPB (D450N) in N-terminal configuration using XTEN linker; hyPB: hyPB without modifications; ½ GFP: Control transposon alone.

FIG. 19 shows a scheme of the analysis method used in the screening of a library of PiggyBac mutations.

In FIG. 20, PiggyBac 1116 bp region with all library variants were sequenced with Illumina NGS technology. 17 Index primer was replaced by a custom primer to allow the full sequencing of the different variants, except for variants 450 and 465.

FIG. 21A-21B show the results of the hyPB library diversity generation. FIG. 21A is an example of sorting plot. Positive targeted integration hits (GFP fluorescence) were selected in gate P4 while negative targeted integration hits (no GFP fluorescence) were selected in gate P5. Non viable cells and debris were negative selective in previous gates with DAPI staining. FIG. 21B shows the results of double plasmid transfection efficiency. Transfection efficiency was measured by transfecting a GFP and an RFP plasmid equimolar to ½ GFP and gRNA transfection on the same day and with same conditions. Gate P8 selects for double plasmid transfection. Non viable cells and debris were negative selective in previous gates with DAPI staining.

FIG. 22A-22K show the results of the analysis of library screening comparing positive hits to negative. FIG. 22A-22B: Sequencing of the bulk library as quality control is shown; were the vast majority of variants were shown only once. Logo of the bulk representative Piggyback library is shown were positions correspond to amino acid positions: 1-R245; 2-R275; 3-R277; 4-G325; 5-N347; 6-5351; 7-R372; 8-K375; 9-R388; 10-T560; 11-5564; 12-5573; 13-M589; 14-5592; 15-F594. In addition, the logo for the negative selected cells is shown with a similar patter to bulk library. FIG. 22C-22K correspond to 3 independent repeats of positive hits; variant calling for the positive logos (bottom) as well as Top1 variant after selection (top). Logos for the top 5 and top 10 variants are also shown. In the left panels of B, C the relative enrichment of Piggyback variants in the positive versus negative sorted populations is shown in log 2 scale.

FIG. 23A shows Top 1 and Top 3 positive variants of independent repeat 3. There is a difference of only 1 amino acid at position 254. FIG. 23B shows the 3 top1 variants identified in 3 independent repeats. WT hyPB is also shown for reference.

FIG. 24A shows the most overrepresented variants in GFP positive versus RFP positive cells. Clustering of the GPF, targeted insertion; RPF, random insertion and negative population is shown. In FIGS. 24B and 24C variants found among the positive hit in more than 1 independent repeat are shown. Rep: Independent Experimental Repeat; Pos: Positive cells with targeted integration; Neg: Negative cells where targeted integration did not occur.

FIG. 25 shows a histogram of variants covariation. It shows the percentage of a variant seen together with another in the positive sample divided by the negative sample. In addition to variants included in the library design, variants that were randomly introduced by the lentiviral retrotranscriptase during viral library generation were analyzed. Some of these new variants are associated in the positive hits and perform the targeted integration on combination. Example of D450N and W465A.

FIG. 26 shows that modified hyPB showed a greater increase on the target integration compared to WT hyPB when fused with Cas9. Cas9 was fused to hyPB or different mutant combinations of hyPB (Unilarge-A: D450N; Unilarge-B: R245A/D450N; Unilarge-C: R245A/G325A/D450N/S573P; Unilarge-D: R245A/G325A/S573P) using a 4GGS linker and the reporter cell line system.

FIG. 27 shows results of integrase deficient transcomplementation. Viral production efficiency measured at day 2 and integration capacity measured at day 7, were assessed for different systems in Hek293T cells. Western blots showed the presence of IN in trans in the viral particles. Viral production efficiency and its integration capacity were assessed by infecting the different condition of integration deficient virus and transcomplemented virus into Hek293T. Cells were passed for 7 days until no episomal signal was detected and GFP signal was analyzed by Flow Cytometry at day 2, 5 and 7. Different production efficiencies could be detected for different systems, being NILV the closed to WT upon production. In all cases a clear rescue of the integration activity was apparent when transcomplementation was done with WT-HIV_IN. Proof of IN being loaded in the transcomplementation system was obtained by western blot. WT: Lentivirus produced with WT IN; NILV: Lentivirus produced with non-integrative IN, harboring two mutations on its catalytic center; TAA: Lentivirus produced with a IN defective IN, where the protein is not expressed due to the presence of a stop codon at the beginning of the IN coding sequence, TAAx3: Lentivirus produced with a IN defective IN, where the protein is not expressed due to the presence of 3 consecutive stop codons at the beginning of the IN coding sequence; Delta-IN: Lentivirus produced with a IN defective IN, where the coding sequence of IN has been removed; Delta-IN cPPT: Lentivirus produced with a IN defective IN, where the coding sequence of IN has been substituted by the central polypyrimidine trac (cPPT) sequence; +VPR-IN: Lentivirus trans complemented with IN fused to VPR in the C-terminal end.

### DETAILED DESCRIPTION OF THE INVENTION

### I. Definitions

As used herein, the singular forms “a,” “an,” and “the” include the singular and the plural reference unless the context clearly indicates otherwise. Thus, for example, a reference to “an agent” includes a single agent and a plurality of such agents.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide,” and “protein” are used interchangeably to refer to a polymer of amino acid residues. The term also applies to amino acid polymers in which one or more amino acids are chemical analogues or modified derivatives of corresponding naturally-occurring amino acids.

The term “binding protein,” as used herein, refers to a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein binding protein, it can bind to itself (to form homodimers, homotrimers, etc.) and/or it can bind to one or more molecules of a different protein or proteins. A binding protein can have more than one type of binding activity. For example, Zinc finger proteins have DNA-binding, RNA-binding and protein-binding activity.

The term “Zinc finger protein,” as used herein, is a protein, or a domain within a larger protein, that binds DNA in a sequence-specific manner through one or more zinc fingers, which are regions of amino acid sequence within a binding domain of the zinc finger protein whose structure is stabilized through coordination of a zinc ion. The term zinc finger protein is often abbreviated as ZFP.

The term “Zinc-finger nucleases” refer to artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. Zinc finger nuclease is often abbreviated as ZFN or ZNP.

The term “nucleic acid sequence” or “polynucleotide sequence” or “gene sequence,” as used herein, refers to a nucleotide sequence of any length, which can be DNA or RNA; can be linear, circular or branched and can be either single-stranded or double stranded.

The term “amino acid sequence” or “polypeptide” or “protein” as used herein, refers a polymer of amino acid residues. Unless specified, a polymer of amino acid residues can be any length.

The term “exogenous,” as used herein, refers to a molecule that is not normally present in a cell, but can be introduced into a cell by one or more genetic, biochemical or other methods. Normal presence in the cell is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally functioning endogenous molecule.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally occurring episomal nucleic acid. Additional endogenous molecules can include proteins, for example, transcription factors and enzymes.

A “target site” or “target sequence” is a sequence that defines a portion of a nucleic acid or a polypeptide to which a binding molecule will bind, provided sufficient conditions for binding exist. For example, the sequence 5′-GAATTC-3′ is a target site for the EcoRI restriction endonuclease.

The term “fusion,” as used herein, refers to a molecule in which two or more subunit molecules are linked, preferably covalently. The subunit molecules can be the same chemical type of molecule, or can be different chemical types of molecules.

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively.

The terms “gene” or “genome” as used herein, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

The term “eukaryotic,” cells include, but are not limited to, fungal cells (such as yeast), plant cells, animal cells, mammalian cells and human cells (e.g., T-cells).

The term “linked,” as used herein, refers to the juxtaposition of two or more components (such as sequence elements), in which the components are arranged such that both components function normally and allow the possibility that at least one of the components can mediate a function that is exerted upon at least one of the other components.

A “functional fragment” of a protein, polypeptide or nucleic acid is a protein, polypeptide or nucleic acid, respectively, whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions.

The term “transfect,” as used herein, refers to the introduction of nucleic acids (either DNA or RNA) into eukaryotic or prokaryotic cells or organisms.

The term “cleavage,” as used herein, refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

The term “integrase,” as used herein, refers to an enzyme produced by a virus that enables genetic material to be integrated into the DNA, e.g., genomic DNA, of an infected cell.

The term “specificity,” as used herein, refers to the ability to selectively bind a sequence which shares a degree of sequence identity to a selected sequence.

The terms “insertion,” and “integration,” as used herein, refer to the addition of a nucleic acid sequence into a second nucleic acid sequence or genome.

The terms “specific”, “site-specific”, “targeted” and “on-targeted” in relation to insertion or integration, are used herein interchangeably to refer to the insertion of a nucleic acid into a specific site of a second nucleic acid or genome. The terms “random”, “non-targeted” and “off-targeted” refer to non-specific and unintended genetic insertion. The terms “total” or “overall” refer to the total number of insertions.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The term “transposase,” as used herein, refers to an enzyme that binds to the end of a transposon and catalyzes its movement to another part of the genome by a cut and paste mechanism or a replicative transposition mechanism.

The term “modified,” as used herein, refers to a protein or nucleic acid sequence that is different than a corresponding unmodified protein or nucleic acid sequence.

The term “linker,” as used herein, refers to a chemical group or a molecule linking two adjacent molecules or moieties.

The terms “vector” and “plasmid” as used herein, refer to any polynucleotide that can carry, e.g., a second polynucleotide of interest, and e.g., which can transfer gene sequences to target cells. Thus, the term includes cloning, and expression vehicles, as well as integrating vectors. Particularly, the term “expression vector,” as used herein, refers to any polynucleotide capable of directing the expression of a nucleic acid. In some aspects, the terms “vector” and “plasmid” are used interchangeably with the term “nucleic acid construct.”

The term “percent identity” as used herein, refers to the percent identity of two sequences, whether nucleic acid or amino acid sequences, and is the number of exact matches between two aligned sequences divided by the length of the shorter sequences and multiplied by 100.

The terms “recombinant” or “engineered,” as used herein, refer to a protein or nucleic acid sequence that has been artificially created.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal.

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent, reduce the likelihood of developing, or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### II. Nucleic Acid Construct

Targeted editing of nucleic acid sequences, e.g., the introduction of a specific modification (e.g., insertion of an exogenous nucleic acid) into genomic DNA, is a promising approach for treating human genetic diseases. To this end, the inventors aim to provide improved nucleic acid constructs for use in genomic editing that are highly efficient at installing a desired modification; minimal off-target activity; and the ability to be programmed to edit precisely a site within the human genome.

Certain aspects of the present application are directed to a nucleic acid construct for use in improving site-specific insertion of an exogenous nucleic acid, e.g., a gene of interest (GOI), into a genome. In some embodiments, the GOI is a therapeutic gene, e.g., a gene that encodes a therapeutic protein. Examples of a therapeutic genes of interest include CFTR gene (Cystic fibrosis transmembrane conductance regulator) to treat Cystic Fibrosis disease; SMN1 gene (Survival motor neuron 1) to treat Spinal muscular atrophy (SMA); LRP5 gene (LDL receptor related protein 5) variant G171V to prevent osteoporosis and bone fractures; and APP gene (amyloid beta precursor protein) variant A673T to reduce Alzheimer's predisposition.

In some embodiments, the exogenous nucleic acid for insertion (e.g., the GOI) can be up to about 10 kb, up to about 15 kb, up to about 20 kb in length, up to about 25 kb in length, up to about 30 kb in length, up to about 35 kb in length, or up to about 40 kb in length.

In some embodiments, the polynucleotide sequence encoding a DNA binding protein which enables insertion of an exogenous nucleic acid into the genome comprises an integrase or an integrase which is modified relative to a wildtype integrase, and the exogenous nucleic acid for insertion can be up to 10 kb, up to 15 kb, or up to 20 kb in length, e.g., about 1 kb to about 20 kb, about 1 kb to about 19 kb, about 1 to about 18 kb, about 1 kb to about 17 kb, about 1 kb to about 16 kb, or about 1 kb to about 15 kb.

In some embodiments, the polynucleotide sequence encoding a second DNA binding protein which enables insertion of an exogenous nucleic acid into the genome comprises a transposase or a transposase which is modified relative to a wildtype transposase, and the exogenous nucleic acid for insertion can be up to 10 kb, up to 15 kb, up to 20 kb in length, up to 25 kb in length, up to 30 kb in length, up to 35 kb in length, or up to 40 kb in length, e.g., about 1 kb to about 40 kb, about 1 kb to about 39 kb, about 1 to about 38 kb, about 1 kb to about 37 kb, about 1 kb to about 36 kb, or about 1 kb to about 35 kb.

In some embodiments, the nucleic acid construct comprises a polynucleotide sequence that encodes a first DNA binding protein, e.g., a gene editing polypeptide, and a polynucleotide sequence that encodes a second DNA binding protein, e.g., an integrase or a transposase, wherein the nucleic acid construct encodes the first and second binding proteins as a fusion protein. In some embodiments, the nucleic acid construct further comprises a nucleic acid sequence encoding a linker between the first and the second binding protein. In some embodiments, the nucleic acid construct encodes a fusion protein that enables and/or promotes site specific insertion of the exogenous nucleic acid into a genome. In some embodiments, the first or second binding protein is an integrase which is modified relative to wild-type. In some embodiments, the first or second binding protein is a transposase which is modified relative to wild-type. In some embodiments are directed to a vector or plasmid comprising a nucleic acid construct of the disclosure. In certain aspects, the nucleic acid construct of the disclosure encodes a fusion protein which improves specificity of the insertion of a nucleic acid, e.g., a GOI, into the genome. In some embodiments, the fusion protein and exogenous nucleic acid are delivered to a cell using a lentivirus particle.

In some embodiments, first and second binding proteins are on separate nucleic acid constructs, e.g., the transposase or integrase (e.g., a transposase and/or integrase modified with respect to the wild type) is on a separate nucleic acid construct from the Cas9 or ZFP.

Certain aspects are directed to a plasmid or vector comprising a nucleic acid construct disclosed herein. In some embodiments, the plasmid comprising the nucleic acid construct is a packaging plasmid. In some embodiments, the plasmid comprising the nucleic acid construct further comprises a polynucleotide encoding capsid proteins, e.g., gag and pol. In some embodiments, (i) the plasmid comprising the nucleic acid construct is combined with (ii) a plasmid comprising a polynucleotide that encode proteins for a viral envelope (envelope plasmid); and (iii) a plasmid comprising an exogenous nucleic acid sequence (e.g., a GOI), wherein when the combination is introduced into a production cell line (e.g., eukaryotic cells, prokaryotic cells and/or cell lines), a virus particle comprising the exogenous nucleic acid, e.g., GOI, and the fusion protein comprising the first and the second binding protein is produced.

In some embodiments, (i) the plasmid comprising the nucleic acid construct is combined with (ii) a plasmid comprising the nucleic acid construct further comprises a polynucleotide encoding capsid proteins, e.g., gag and pol (a packaging plasmid, wherein the packaging plasmid lacks a functional integrase); (iii) a plasmid comprising a polynucleotide that encode proteins for a viral envelope (envelope plasmid) and (iv) a plasmid comprising an exogenous nucleic acid sequence (e.g., a GOI), wherein when the combination is introduced into a production cell line (e.g., eukaryotic and prokaryotic cells and/or cell lines), a virus particle comprising the exogenous nucleic acid, e.g., GOI, and the fusion protein comprising the first and the second binding protein is produced.

The nucleic acid construct comprises a first polynucleotide sequence encoding a first DNA binding protein engineered to bind a specific DNA sequence, a second polynucleotide sequence encoding a second DNA binding protein which enables insertion of exogenous nucleic acid into the genome wherein the second DNA binding protein is an integrase or a transposase (e.g., a transposase and/or integrase which is modified relative to the wild type), and third polynucleotide sequence comprising a nucleic acid sequence encoding a linker between the first and second polynucleotides. In some embodiments, the first DNA binding protein is a zinc finger protein or a Cas 9 protein.

In some embodiments, the nucleic acid construct comprises a linker selected from the group consisting of a (GGS)n, a (GGGGS)n (SEQ ID NO:133), a (G)n, an (EAAAK)n (SEQ ID NO:134), a XTEN-based linker, or an (XP)n motif, or a combination of any of these, wherein n is independently an integer between 1 and 50. In some embodiments the nucleic acid encodes a linker comprising a XTEN sequence or a GGS sequence. In some embodiments, the linker nucleic acid sequence is between 3 to 150 nucleotides in length. In some embodiments, the linker is 12 to 24 amino acids, or 36 to 72 nucleic acids in length. In some embodiments, the nucleic acid construct comprises a linker nucleic acid sequence which is 6 to 120, 6 to 90, 6 to 78, 6 to 72, 9 to 120, 9 to 90, 9 to 78, 9 to 72, 12 to 120, 12 to 90, 12 to 78, 12 to 72, 15 to 120, 15 to 90, 15 to 78, 15 to 72, 18 to 120, 18 to 90, 18 to 78, 18 to 72, 21 to 120, 21 to 90, 21 to 78, 21 to 72, 24 to 120, 24 to 90, 24 to 78, 24 to 72, 27 to 120, 27 to 90, 27 to 78, 27 to 72, 30 to 120, 30 to 90, 30 to 78, 30 to 72, 33 to 120, 33 to 90, 33 to 78, 33 to 72, 36 to 120, 36 to 90, 36 to 78, or 36 to 72 nucleotides in length. In some embodiments, the nucleic acid encoding the linker is between 9 to 150 nucleic acids in length. In some embodiments, a zinc finger protein is linked to a modified integrase of the disclosure with a linker comprising a GGS sequence. In some embodiments, the linker is between 1 to 50 amino acids in length. In some embodiments, the linker is 3 to 40, 3 to 30, 3 to 29, 3 to 24, 4 to 40, 4 to 30, 4 to 29, 4 to 24, 5 to 40, 5 to 30, 5 to 29, 5 to 24, 6 to 40, 6 to 30, 6 to 29, 6 to 24, 7 to 40, 7 to 30, 7 to 29, 7 to 24, 8 to 40, 8 to 30, 8 to 29, 8 to 24, 9 to 40, 9 to 30, 9 to 29, 9 to 24, 10 to 40, 10 to 30, 10 to 29, 10 to 24, 11 to 40, 11 to 30, 11 to 29, 11 to 24, 12 to 40, 12 to 30, 12 to 29, or 12 to 24 amino acids in length.

In some embodiments the 3′ end of the first polynucleotide sequence is connected to the 5′ end of the second polynucleotide sequence by the nucleic acid encoding a linker. In some embodiments the 5′ end of the first polynucleotide sequence is connected to the 3′ end of the second polynucleotide sequence by the nucleic acid encoding a linker. In some embodiments the 3′ end of the Cas 9 protein is connected to the 5′ end of the transposase by a linker. In some embodiments the 5′ end of the Cas 9 protein is connected to the 3′ end of the transposase by a linker. In some embodiments the 3′ zinc finger protein is connected to the 5′ end of the integrase by a linker. In some embodiments the 5′ zinc finger protein is connected to the 3′ end of the integrase by a linker.

In some embodiments, a linker is not needed because the modified integrase or modified transposase expressed from a separate plasmid from the Cas9 or ZFP.

Certain aspects of the disclosure are directed to a vector or a plasmid (e.g., an expression vector or a packaging vector) comprising a nucleic acid construct of the disclosure suitable for expression in a host cell, e.g., mammalian cells, yeast cells, insect cells, plant cells, fungal cells, or algal cells.

In some embodiments, the nucleic acid construct comprises: (a) a first polynucleotide sequence comprising a nucleic acid encoding a first DNA binding protein engineered to bind to a specific genomic DNA sequence in a genome; wherein the first DNA binding protein is a zinc finger protein or a Cas9 protein; (b) a second polynucleotide sequence comprising a nucleic acid encoding a second DNA binding protein which enables insertion of an exogenous nucleic acid into a genome, wherein the second DNA binding protein is (i) a hyperactive PiggyBac transposase, or a modified hyperactive PiggyBac with improved specificity of inserting the exogenous nucleic acid into the genome compared to the hyperactive PiggyBac, or (ii) a human immunodeficiency virus (HIV) integrase, or a modified HIV integrase with improved specificity of inserting the exogenous nucleic acid into the genome compared to the HIV integrase; and (c) an optional polynucleotide sequence comprising a nucleic acid encoding a linker; wherein the nucleic acid construct encodes a fusion protein comprising the first DNA binding protein, the second DNA binding protein, and the optional linker between the first DNA binding protein and the second DNA binding protein; and wherein the fusion protein enables insertion of the exogenous nucleic acid into a specific site of the genome.

In an embodiment, (a) the first DNA binding protein is a Cas 9 protein or a zinc finger protein; and (b) the second DNA binding protein is a hyperactive PiggyBac transposase, or a modified hyperactive PiggyBac transposase with improved specificity of inserting the exogenous nucleic acid into the genome compared to the hyperactive PiggyBac transposase.

In another embodiment, (a) the first DNA binding protein is a Cas 9 protein or a and zinc finger protein; and (b) the second DNA binding protein is a HIV integrase, or a modified HIV integrase with improved specificity of inserting the exogenous nucleic acid into the genome compared to the HIV integrase.

In some embodiments, the Cas9 protein is one described in this disclosure and particularly selected from the group consisting of a human Cas9, a nickase Cas9 and a dead Cas 9, and more particularly is human Cas9 or nickase Cas9.

In one embodiment, when dCas9 is used, the second DNA binding protein is not a Gin, Hin or Tn3 recombinase catalytic domain or a FokI DNA cleavage domain. Such recombinases and FoKI need a known site (an acceptor sequence in the genome) to be able to integrate; therefore the possibilities of targeting sites are much more limited; and they also need the formation of dimers of e.g. Gin to be functional.

In another embodiment, the zinc finger protein is one described in this disclosure and particularly is a C2H2 zinc finger protein comprising 6 binding domains.

In another embodiment, the linker is one described in this disclosure and particularly the linker comprises a XTEN sequence (e.g., SEQ ID NO: 61, encoded by SEQ ID NO:60) or a GGS sequence, more particularly a GGS×3 (SEQ ID NO: 49, encoded by SEQ ID NO:48), GGS×4 (SEQ ID NO: 51, encoded by SEQ ID NO:50), GGS×5 (SEQ ID NO: 53, encoded by SEQ ID NO:52), GGS×6 (SEQ ID NO: 55, encoded by SEQ ID NO:54), GGS×7 (SEQ ID NO: 57, encoded by SEQ ID NO:56) or GGS×8 (SEQ ID NO: 59, encoded by SEQ ID NO:58).

In another embodiment, the 3′ end of the first polynucleotide sequence is connected to the 5′ end of the second polynucleotide.

In some embodiments, the modified hyperactive PiggyBac transposase is one described in this disclosure. In other embodiments, the modified HIV integrase is one described in disclosure.

In other embodiments, a linker is not used. Instead, e.g., the first and/or the second polynucleotide sequences comprise nucleic acids encoding a first and second DNA binding protein and further comprise additional nucleic acids in at least one of their ends that make the function of linker.

In an embodiment, (a) the first DNA binding protein is a Cas 9 protein or a zinc finger protein, and (b) the second DNA binding protein is a hyperactive PiggyBac transposase, or a modified hyperactive PiggyBac with improved specificity of inserting the exogenous nucleic acid into the genome compared to the hyperactive PiggyBac, wherein the nucleic acid construct comprises the (c) polynucleotide sequence comprising a nucleic acid encoding a linker comprising a XTEN sequence or a GGS sequence, and wherein the 3′ end of the first polynucleotide sequence is connected to the 5′ end of the second polynucleotide.

In one embodiment, (a) the first DNA binding protein is a Cas 9 protein, and (b) the second DNA binding protein is a hyperactive PiggyBac transposase, or a modified hyperactive PiggyBac with the proviso that when Cas9 is an inactive Cas9 (dcas9) the linker is not KLAGGAPAVGGGPK (SEQ ID NO: 130).

In one embodiment, a) the first DNA binding protein is a zinc finger protein, and (b) the second DNA binding protein is a hyperactive PiggyBac transposase, or a modified hyperactive PiggyBac, wherein the zinc finger protein is able to recognize multiple recognition sites, since as explained in this disclosure the binding domain of the zin finger protein can be engineered to bind to a sequence of choice.

In one embodiment, a) the first DNA binding protein is a zinc finger protein, and (b) the second DNA binding protein is a hyperactive PiggyBac transposase, or a modified hyperactive PiggyBac, and the linker is XTEN.

In one embodiment, (a) the first DNA binding protein is a zinc finger protein, and (b) the second DNA binding protein is a hyperactive PiggyBac transposase, or a modified hyperactive PiggyBac, wherein the zinc binding protein does not have a Gal4 DNA binding domain. Gal4 binds to CGG-N₁₁-CCG, where N can be any base. This protein is a positive regulator for the gene expression of the galactose-induced genes such as GAL1, GAL2, GALT, GAL10, and MEL1 which code for the enzymes used to convert galactose to glucose. It recognizes a 17 base pair sequence in (5′-CGGRNNRCYNYNCNCCG-3′) (SEQ ID NO:135) the upstream activating sequence (UAS-G) of these genes. Therefore, Gal4 recognizes a short and very frequent sequence in the genome, thus not being site specific. In a particular embodiment, the zinc binding protein has a Gal4 DNA binding domain engineered to be site-specific.

In one embodiment, (a) the first DNA binding protein is a zinc finger protein, and (b) the second DNA binding protein is a hyperactive PiggyBac transposase, or a modified hyperactive PiggyBac transposase with the proviso that the linker is not EFGGGGSGGGGSGGGGSQF (SEQ ID NO: 131).

In another embodiment, (a) the first DNA binding protein is a Cas 9 protein or a and zinc finger protein, and (b) the second DNA binding protein is a HIV integrase, or a modified HIV integrase with improved specificity of inserting the exogenous nucleic acid into the genome compared to the HIV integrase, wherein the nucleic acid construct comprises the (c) polynucleotide sequence comprising a nucleic acid encoding a linker comprising a XTEN sequence or a GGS sequence, and wherein the 3′ end of the first polynucleotide sequence is connected to the 5′ end of the second polynucleotide.

In some embodiments, the nucleic acid construct is in DNA or RNA form.

Also provided herein, are vectors comprising any of the nucleic acid constructs provided in this disclosure. Particularly, the vectors are suitable for expression in mammalian cells, yeast cells, insect cells, plant cells, fungal cells, or algal cells. Also provided herein, are host cells comprising any of the nucleic acid constructs or vectors provided in this disclosure.

### III. Integrase and Modified Integrase

Integrase is a key enzyme for stable integration of the viral genome into a host cell, but integrase is also associated with insertional mutagenesis since the site of integration by wild-type integrase is unpredictable. Integration has been shown to be preferred for highly transcribed genes, which increases risk of mutation of important genes and regulators. In general, the HIV-1 Integrase consists of a N-terminal-domain (NTD), a catalytic core-(CCD) and a C-terminal-domain (CTD). The NTD is used to bind and coordinate a Zn²⁺ cation as an important co-factor, while the CTD is used for DNA binding. The CCD-domain forms the catalytic core in which the integration process is catalyzed. After entering the host cell and reverse transcription of the viral-RNA genome, four integrase molecules form a tetramer and attach to the ends of the viral DNA, which is then called intasome. The pre-integration complex (PIC) digests the 3′OH end of the DNA forming a 5′OH-overhang, which is later needed for a nucleophilic attack on the host DNA. During the formation of this PIC, the complex is transported into the nucleus. After transportation into the nucleus the PIC forms a complex with the host DNA, called a strand transfer complex (STC). Here, both 3′OH overhangs of the viral DNA attacks both sites of the host DNA backbone with space of about 5 nucleotides. This leads to a target duplication of the 5 nucleotides. After the nucleophilic attack, the viral DNA is integrated and single stranded DNA-parts get repaired by the host-cell DNA repair machinery.

The present disclosure provides nucleic acid constructs comprising polynucleotides encoding integrases and modified integrases for insertion of exogenous nucleic acid into a specific site of a genome. In some embodiments, the exogenous nucleic acid for insertion can be up to 10 kb, up to 15 kb, or up to 20 kb in length, e.g., about 1 kb to about 20 kb, about 1 kb to about 19 kb, about 1 to about 18 kb, about 1 kb to about 17 kb, about 1 kb to about 16 kb, or about 1 kb to about 15 kb. In some embodiments, the polynucleotide sequence encoding a DNA binding protein which enables insertion of an exogenous nucleic acid into the genome comprises an integrase which can be modified relative to a wildtype integrase, and the exogenous nucleic acid for insertion can be up to 10 kb or up to 15 kb in length.

Some aspects of this disclosure provide integrase fusion proteins that are designed using the methods and strategies described herein. Some embodiments of this disclosure provide nucleic acids encoding integrases or modified integrases and/or fusion proteins comprising the same. Some embodiments of this disclosure provide plasmids or expression vectors comprising such nucleic acid constructs encoding integrases or modified integrases and/or fusion proteins comprising the same.

The integrase or modified integrase of the disclosure can be any integrase that can insert an exogenous nucleic acid into a specific site of a genome. Non-limiting examples of integrases include HIV integrase, lentiviral integrase, adenoviral integrase, retroviral integrase, and mammary mouse tumor virus integrase. In some embodiments, the integrase (e.g., a modified integrase comprising one or more modification relative to the wild-type) is an HIV integrase, particularly the HIV integrase sequence corresponding to NC 001802.1 (SEQ ID NOs: 1 and 2, amino acid and nucleic acid sequences, respectively). In some embodiments, the modified integrase comprises one or more modifications relative to the wild-type HIV integrase (SEQ ID NOS: 1 and 2).

In some embodiments, the integrase is a modified HIV integrase. The modified HIV integrase can comprise a mutation of one or more of amino acids selected from amino acid: 10, 13, 64, 94, 116, 117, 119, 120, 122, 124, 128, 152, 168, 170, 185, 231, 264, 266, or 273 corresponding to the amino acid numbering of SEQ ID NO: 1. The modified HIV integrase mutation can comprise one or more of the amino acid modifications listed in Table 8. The modified HIV integrase mutation can comprise one or more of the amino acid modifications selected from D10K, E13K, D64A, D64E, G94D, G94E, G94R, G94K, D116A, D116E, N117D, N117E, N117R, N117K, S119A, S119P, S119T, S119G, S119D, S119E, S119R, S119K, N120D, N120E, N120R, N120K, T122K, T122I, T122V, T122A, T122R, A124D, A124E, A124R, A124K, A128T, E152A, E152D, Q168L, Q168A, E170G, F185K, R231G, R231K, R231D, R231E, R231S, K264R, K266R, or K273R corresponding to the amino acid numbering of SEQ ID NO: 1 or SEQ ID NO: 3.

In some embodiments, the modified integrase can comprise one or more mutations relative to wild-type that impair DNA binding, e.g., at amino acid 94, 117, 119, 120, 124, and/or 231 (e.g., G94D, G94E, G94R, G94K, N117D, N117E, N117R, N117K, S119A, S119P, S119T, S119G, S119D, S119E, S119R, S119K, N120D, N120E, N120R, N120K, A124D, A124E, A124R, A124K, R231G, R231K, R231D, R231E, and/or R231K) corresponding to the amino acid numbering of SEQ ID NO: 1 or SEQ ID NO: 4.

In some embodiments, the modified integrase can comprise one or more mutations relative to wild-type that enhance DNA binding, e.g., at amino acid 94, 117, 119, 120, 122, 124, and/or 231 (e.g., G94D, G94E, G94R, G94K, N117D, N117E, N117R, N117K, S119A, S119P, S119T, S119G, S119D, S119E, S119R, S119K, N120D, N120E, N120R, N120K, T122K, T122I, T122V, T122A, T122R, A124D, A124E, A124R, A124K, R231G, R231K, R231D, R231E, and/or R231S) corresponding to the amino acid numbering of SEQ ID NO: 1 or SEQ ID NO: 5.

In some embodiments, the modified integrase can comprise one or more mutations relative to wild-type that are involved in integrase acetylation by p300, e.g., at amino acid 264, 266, and/or 273 (e.g., K264R, K266R, and/or K273R) corresponding to the amino acid numbering of SEQ ID NO: 1 or SEQ ID NO: 6.

In some embodiments, the modified integrase can comprise one or more mutations in highly conserved amino acids that are critical for retroviral integrative recombination, e.g., at amino acid 10, 13, 64, 116, 128, 152, 168, and/or 170 (e.g., D10K, E13K, D64A, D64E, D116A, D116E, A128T, E152A, E152D, Q168L, Q168A, and/or E170G) corresponding to the amino acid numbering of SEQ ID NO: 1 or SEQ ID NO: 7.

In some embodiments, the modified integrase can comprise one or more mutations that interfere with interaction with LEDGF/p75 and impair chromosome tethering and HIV-1 replication, e.g., amino acid 168 (e.g., Q168L or Q168A) corresponding to the amino acid numbering of SEQ ID NO: 1 or SEQ ID NO: 8.

In some embodiments, the modified HIV integrase comprises an amino acid sequence at least 80%, at least 85%, at least 90%, at least 95%, at least 96%, at least 97%, at least 98%, or at least 99% identical to the sequence set forth in SEQ ID NO: 1. In some embodiments, the modified HIV integrase comprises an amino acid sequence having one or more of the modifications disclosed herein relative to SEQ ID NO: 1, 3, 4, 5, 6, 7, or 8, and retains at least 80%, at least 85%, at least 90%, at least 95%, at least 96%, at least 97%, at least 98%, or at least 99% identical to the sequence set forth in SEQ ID NO: 1, 3, 4, 5, 6, 7, or 8, respectively. In some embodiments, the modified HIV integrase is selected for its high specificity of DNA integration into a genome compared to wildtype HIV integrase.

Certain aspects of the disclosure are directed to a vector or a plasmid (e.g., an expression vector or a packaging vector) comprising a nucleic acid construct comprising an integrase or a modified integrase of the disclosure suitable for expression in a host cell, e.g., mammalian cells, yeast cells, insect cells, plant cells, fungal cells, or algal cells. In some embodiments, the integrase or modified integrase is expressed as a fusion protein with a Cas9 or a Zinc Finger protein. In some embodiments, the integrase or modified integrase is co-expressed with a Cas9 or a Zinc Finger protein from separate vectors, but delivered to the same cell. In some embodiments, the integrase or modified integrase or the fusion protein comprising the same is packaged in a lentivirus particle for delivery to a cell.

### IV. Transposase and Modified Transposase

Transposons are chromosomal segments that can undergo transposition, e.g., DNA that can be translocated as a whole in the absence of a complementary sequence in the host DNA. Transposons can be used to perform long range DNA engineering in human cells. Common transposon systems used in mammalian cells include Sleeping Beauty (SB), which was reconstructed from inactive transposons, and PiggyBac (PB), isolated from the moth Trichoplusia. PiggyBac has higher transposition activity than SB and it can be excised scarlessly.

Native DNA transposons typically contain a single gene coding for the transposase protein, which is flanked by Terminal Inverted Repeats (ITRs) that carry transposase binding sites. During their transposition, the transposase protein recognizes these ITRs to catalyze excision and subsequent reintegration of the element elsewhere in a random manner. Moreover, some of these transposons can be adapted for use in gene therapy protocols, employing them as bi-component systems, in which a plasmid contains an expression cassette where a DNA sequence, placed between the transposon ITRs, can be introduced into a host genome directed by the co-transfected plasmid containing the sequence encoding the transposase enzyme or its mRNA synthesized in vitro. In certain aspects of the disclosure, a transposon-based is used to efficiently mediate stable integration and persistent expression of transgenes, such as therapeutic genes.

The present disclosure provides nucleic acid constructs comprising polynucleotides encoding transposases or modified transposases for insertion of exogenous nucleic acid into a specific site of a genome. In some embodiments, the exogenous nucleic acid for insertion can be up to 20 kb in length, up to 25 kb in length, up to 30 kb in length, or up to 40 kb in length, e.g., about 1 kb to about 40 kb, about 1 kb to about 39 kb, about 1 to about 38 kb, about 1 kb to about 37 kb, about 1 kb to about 36 kb, about 1 kb to about 35 kb, about 1 kb to about 30 kb, about 1 kb to about 30 kb, or about 1 kb to about 25 kb. In some embodiments, the polynucleotide sequence encoding a DNA binding protein which enables insertion of an exogenous nucleic acid into the genome comprises a transposase or a transposase which is modified relative to a wildtype transposase, and the exogenous nucleic acid for insertion can be up to 35 kb or up to 40 kb in length.

A transposase or modified transposase of the disclosure can be any transposase that can insert an exogenous nucleic acid into a specific site of a genome. Some aspects of this disclosure provide transposase fusion proteins that are designed using the methods and strategies described herein. Some embodiments of this disclosure provide nucleic acids encoding such transposases or modified transposases and/or fusion proteins comprising the same. Some embodiments of this disclosure provide plasmids or expression vectors comprising such nucleic acid constructs encoding transposases or modified transposases and/or fusion proteins comprising the same.

Non-limiting examples of transposases include Frog Prince, Sleeping Beauty, hyperactive Sleeping Beauty, PiggyBac, and hyperactive PiggyBac. In some embodiments, the transposase is the hyperactive PiggyBac transposase corresponding to SEQ ID NO: 9 and 67 (referred in this disclosure also as hyPB or simply as PB). In some embodiments, the modified transposase comprises one or more modifications relative to the to the hyperactive PiggyBac transposase (SEQ ID NO: 9).

In some embodiments, the transposase is a modified hyperactive PiggyBac transposase. The modified hyperactive PiggyBac transposase can comprise a mutation of one or more of amino acids selected from amino acid: 245, 268, 275, 277, 287, 290, 315, 325, 341, 346, 347, 350, 351, 356, 357, 372, 375, 388, 409, 412, 432, 447, 450, 460, 461, 465, 517, 560, 564, 571, 573, 576, 586, 587, 589, 592, and 594 corresponding to the amino acid numbering of SEQ ID NO: 9. The modified hyperactive PiggyBac mutation can comprise one or more of the amino acid modifications listed in Table 3. The modified hyperactive PiggyBac transposase mutation can comprise one or more of the amino acid modifications selected from: R245A, D268N, R275A/R277A, K287A, K290A, K287A/K290A, R315A, G325A, R341A, D346N, N347A, N347S, T350A, S351E, S351P, S351A, K356E, N357A, R372A, K375A, R372A/K375A, R388A, K409A, K412A, K409A/K412A, K432A, D447A, D447N, D450N, R460A, K461A, R460A/K461A, W465A, S517A, T560A, S564P, S571N, S573A, K576A, H586A, I587A, M589V, S592G, or F594L corresponding to the amino acid numbering of SEQ ID NO: 9 or SEQ ID NO: 10.

In some embodiments, the modified transposase can comprise one or more mutations relative to hyPB that are involved in the conserved catalytic triad, e.g., at amino acid 268 and/or 346 (e.g., D268N and/or D346N) corresponding to the amino acid numbering of SEQ ID NO: 9 or SEQ ID NO: 11.

In some embodiments, the modified transposase can comprise one or more mutations relative to hyPB that are critical for excision, e.g., at amino acid 287, 287/290 and/or 460/461 (e.g., K287A, K287A/K290A, and/or R460A/K461A) corresponding to the amino acid numbering of SEQ ID NO: 9 or SEQ ID NO: 12.

In some embodiments, the modified transposase can comprise one or more mutations relative to hyPB that are involved in target joining, e.g., at amino acid 351, 356, and/or 379 (e.g., S351E, S351P, S351A, and/or K356E) corresponding to the amino acid numbering of SEQ ID NO: 9 or SEQ ID NO: 13.

In some embodiments, the modified transposase can comprise one or more mutations relative to hyPB that are critical for integration, e.g., at amino acid 560, 564, 571, 573, 589, 592, and/or 594 (e.g., T560A, S564P, S571N, S573A, M589V, S592G, and/or F594L) corresponding to the amino acid numbering of SEQ ID NO: 9 or SEQ ID NO: 14.

In some embodiments, the modified transposase can comprise one or more mutations relative to hyPB that are involved in alignment, e.g., at amino acid 325, 347, 350, 357 and/or 465 (e.g., G325A, N347A, N347S, T350A and/or W465A) corresponding to the amino acid numbering of SEQ ID NO: 9 or SEQ ID NO: 15.

In some embodiments, the modified transposase can comprise one or more mutations relative to hyPB that are well conserved, e.g., at amino acid 576 and/or 587 (e.g., K576A and/or I587A) corresponding to the amino acid numbering of SEQ ID NO: 9 or SEQ ID NO: 16.

In some embodiments, the modified transposase can comprise one or more mutations relative to hyPB that are involved in Zn²⁺ binding, e.g., 586 (e.g., H586A) corresponding to the amino acid numbering of SEQ ID NO: 9 or SEQ ID NO: 17.

In some embodiments, the programmable transposase can comprise one or more mutations relative to hyPB that are involved in integration e.g., 315, 341, 372, and/or 375 (e.g., R315A, R341A, R372A, and/or K375A) corresponding to the amino acid numbering of SEQ ID NO: 9 or SEQ ID NO: 18.

In some embodiments, the modified hyperactive PiggyBac comprises an amino acid sequence at least 85%, at least 90%, at least 95%, at least 96%, at least 97%, at least 98%, or at least 99% identical to the sequence set forth in SEQ ID NO: 9. In some embodiments, the modified hyperactive PiggyBac is selected for its high specificity of DNA integration into a genome compared to hyperactive PiggyBac. In some embodiments, the modified hyperactive PiggyBac comprises an amino acid sequence having one or more of the modifications disclosed herein relative to SEQ ID NO: 9, 10, 11, 12, 13, 14, 15, 16, 17, or 18, and retains at least 80%, at least 85%, at least 90%, at least 95%, at least 96%, at least 97%, at least 98%, or at least 99% identical to the sequence set forth in SEQ ID NO: 9, 10, 11, 12, 13, 14, 15, 16, 17, or 18, respectively.

In some embodiments, the hyperactive PiggyBac transposase is encoded by a nucleic acid sequence having at least 85%, 90%, 95%, 96%, 97%, 98%, 99%, or 100% sequence identity to SEQ ID NO: 67. In some embodiments, the SB100 transposase is encoded by a nucleic acid sequence having at least 85%, 90%, 95%, 96%, 97%, 98%, 99%, or 100% sequence identity to SEQ ID NO: 68.

In some embodiments, the PB transposase comprises an amino acid sequence having at least 85%, 90%, 95%, 96%, 97%, 98%, 99%, or 100% sequence identity to SEQ ID NO: 72. In some embodiments, the SB100 transposase comprises an amino acid sequence having at least 85%, 90%, 95%, 96%, 97%, 98%, 99%, or 100% sequence identity to SEQ ID NO: 73.

In some embodiments, the modified transposase is a modified Sleeping Beauty transposase comprising one or more mutations. In some embodiments, the one or more mutations in Hyper Active Sleeping Beauty Transposase or SB100 corresponds to: L25F, R36A, 142K, G59D, 1212K, N245S, K252A and Q271L of SEQ ID NO: 9 or SEQ ID NO: 73.

In certain embodiments, the modified transposase is not a Himar1C9 mutant.

Certain aspects of the disclosure are directed to a vector or a plasmid (e.g., an expression vector or a packaging vector) comprising a nucleic acid construct comprising a transposase or a modified transposase of the disclosure suitable for expression in a host cell, e.g., mammalian cells, yeast cells, insect cells, plant cells, fungal cells, or algal cells. In some embodiments, the transposase or modified transposase is expressed as a fusion protein with a Cas9. In some embodiments, the transposase or modified transposase is co-expressed with a Cas9 from separate vectors, but delivered to the same cell. In some embodiments, the transposase or modified transposase or the fusion protein comprising the same is packaged in a lentivirus particle for delivery to a cell.

As shown in Example 20, a newly developed hyperactive PiggyBac transposase mutations library can be used to identify modified hyperactive PiggyBac which perform specific targeted transpositions. Modified hyperactive PiggyBac with positive targeted transposition were identified using such library.

In some embodiments, the modified hyperactive PiggyBac transposase can comprise a mutation of one or more of amino acids selected from amino acid: 245, 275, 277, 325, 347, 351, 372, 375, 388, 450, 465, 560, 564, 573, 589, 592, 594 corresponding to the amino acid numbering of SEQ ID NO: 9.

In some embodiments, the modified hyperactive PiggyBac mutation can comprise one or more of the amino acid modifications listed in Table 11.

In some embodiments, the modified hyperactive PiggyBac transposase mutation can comprise one or more of the amino acid modifications selected from: R245A, R275A, R277A, R275A/R277A, G325A, N347A, N347S, S351E, S351P, S351A, R372A, K375A, R388A, D450N, W465A, T560A, S564P, S573A, M589V, S592G, or F594L corresponding to the amino acid numbering of SEQ ID NO: 9 or SEQ ID NO:119.

In an embodiment, the modified hyperactive PiggyBac transposase comprises the amino acid modification D450 corresponding to the amino acid numbering of SEQ ID NO: 9 or SEQ ID NO: 119.

In an embodiment, the modified hyperactive PiggyBac transposase comprises the amino acid modifications R372A, K375A and D450, corresponding to the amino acid numbering of SEQ ID NO: 9 or SEQ ID NO: 119.

In an embodiment, the modified hyperactive PiggyBac transposase comprises the amino acid modifications R245A and D450, corresponding to the amino acid numbering of SEQ ID NO: 9 or SEQ ID NO: 119.

In an embodiment, the modified hyperactive PiggyBac transposase comprises the amino acid modifications R245A, G325A, and S573P, corresponding to the amino acid numbering of SEQ ID NO: 9 or SEQ ID NO: 119.

In an embodiment, the modified hyperactive PiggyBac transposase comprises the amino acid modifications R245A, G325A, D450 and S573P, corresponding to the amino acid numbering of SEQ ID NO: 9 or SEQ ID NO: 119.

As said before, herein provided are modified hyperactive PiggyBac transposases which can be fused to the elements disclosed herein but can also be used alone or in combination with different elements. Said transposases have been generated by the inventors. Thus, modified hyperactive PiggyBac transposases are provided which comprises the amino acid sequence SEQ ID NO: 9, wherein:

i. amino acid at position 245 is A,

ii. amino acid at position 275 is R or A,

iii. amino acid at position 277 is R or A,

iv. amino acid at position 325 is A or G,

v. amino acid at position 347 is N or A,

vi. amino acid at position 351 is E, P or A,

vii. amino acid at position 372 is R,

viii. amino acid at position 375 is A,

ix. amino acid at position 450 is D or N,

x. amino acid at position 465 is W or A,

xi. amino acid at position 560 is T or A,

xii. amino acid at position 564 is P or S,

xiii. amino acid at position 573 is S or A,

xiv. amino acid at position 592 is G or S, and

xv. amino acid at position 594 is L or F.

In some embodiments, the modified hyperactive PiggyBac comprises an amino acid sequence selected from the group consisting of SEQ ID NO: 120, 121, 122, 123, 124, 125, 126, 127, 128, and 129.

In some embodiments, the modified hyperactive PiggyBac comprises an amino acid sequence having one or more of the modifications disclosed herein relative to SEQ ID NO: 119, 120, 121, 122, 123, 124, 125, 126, 127, 128 or 129, and retains at least 80%, at least 85%, at least 90%, at least 95%, at least 96%, at least 97%, at least 98%, or at least 99% identical to the sequence set forth in SEQ ID NO: 119, 120, 121, 122, 123, 124, 125, 126, 127, 128 or 129, respectively. In some embodiments, the modified hyperactive PiggyBac is selected for its high specificity of DNA integration into a genome compared to hyperactive PiggyBac.

The present disclosure also relates to the modified hyperactive PiggyBac transposases provided herein for use as medicaments, particularly in gene therapy, ex vivo or in vivo.

### V. Cas9 and Zinc Finger Gene Editing

Current genome engineering tools, including engineered zinc finger proteins (ZFPs), transcription activator like effector nucleases (TALENs), and more recently, the RNA-guided DNA endonuclease Cas9, effect sequence-specific DNA cleavage in a genome. This programmable cleavage can result in mutation of the DNA at the cleavage site via non-homologous end joining (NHEJ) or replacement of the DNA surrounding the cleavage site via homology-directed repair (HDR).

Certain aspects of the disclosure are directed to nucleic acid constructs comprising polynucleotides encoding a DNA binding protein engineered to bind to a specific genomic DNA sequence, e.g., Cas9 and ZFPs. In some embodiments, such DNA binding proteins are fused to the modified integrase or the modified transposase disclosed herein for gene editing.

i. Cas9

The CRISPR-Cas9 system is a highly effective tool for inactivating or modifying genes via sequence-specific double-strand breaks (DSBs). These DSBs are recognized by the cellular DNA damage response machinery and can be repaired by endogenous DSB repair pathways. The predominant repair pathway is non-homologous end joining (NHEJ), which often results in small insertions and/or deletions that can create frameshift mutations and disrupt the function of genes. This pathway can be exploited to generate genetic knockout mutations. Alternatively, in the presence of repair templates, the damage can be repaired seamlessly by homology-directed repair (HDR). However, despite remarkable progress, HDR-mediated genome editing to introduce precise genetic modifications is much less efficient than NHEJ-mediated gene disruption. Furthermore, large multi-kb replacements by the HDR pathways results challenging and requires selection and/or large population cell sorting. Consequently, the major applications for the HDR pathways are the local replacement of key regions within genes.

The term “Cas9” and “Cas9 nuclease” refer to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA,” or simply “gNRA”) can be engineered so as to incorporate aspects of both the crRNA and tracrRNA into a single RNA species.

Cas9 recognizes a short motif in the CRISPR repeat sequences (the PAM or protospacer adjacent motif) to help distinguish self vs non-self. Cas9 nuclease sequences and structures are well known to those of skill in the art. Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, et al., “The tracrRNA and Cas9 families of type II CRISPR-Cas immunity systems” (2013) RNA Biology 10:5, 726-737; the entire contents of which are incorporated herein by reference.

In some embodiments, a Cas9 nuclease has an inactive (e.g., an inactivated) DNA cleavage domain. A nuclease-inactivated Cas9 protein can interchangeably be referred to as a “dCas9” protein (for nuclease-“dead” Cas9). Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each are incorporated herein by reference).

For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H841A completely inactivate the nuclease activity of S. pyogenes Cas9. Cas9 Nickase is a variant of Cas9 nuclease differing by a point mutation (D10A) in the RuvC nuclease domain, which enables it to nick, but not cleave, DNA.

The term “Cas9” also includes variants and functional fragments thereof. In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, the protein comprising Cas9 or fragments thereof is referred to as a “Cas9 variant.” A Cas9 variant shares homology to Cas9, or a fragment thereof. For example, a Cas9 variant can be at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to a wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding fragment of wild type Cas9. In some embodiments, Cas9 refers to Cas9 from: Corynebacterium ulcerans (NCBI Refs: NC 015683.1, NC 017317.1) (SEQ ID NOs: 19); Corynebacterium diphtheria (NCBI Refs: NC 016782.1, NC 016786.1) (SEQ ID NO: 20); Spiroplasma syrphidicola (NCBI Ref: NC 021284.1) (SEQ ID NO: 21); Prevotella intermedia (NCBI Ref: NC 017861.1) (SEQ ID NO: 22); Spiroplasma taiwanense (NCBI Ref: NC 021846.1) (SEQ ID NO: 23); Streptococcus iniae (NCBI Ref: NC 021314.1) (SEQ ID NO: 24); Belliella baltica (NCBI Ref: NC 018010.1) (SEQ ID NO: 25); Psychroflexus torquisi (NCBI Ref: NC 018721.1) (SEQ ID NO:26); Streptococcus thermophilus (NCBI Ref: YP 820832.1) (SEQ ID NO:27); Listeria innocua (NCBI Ref: NP 472073.1) (SEQ ID NO:28); Campylobacter jejuni (NCBI Ref: YP_002344900.1) (SEQ ID NO: 29); or Neisseria. meningitidis (NCBI Ref: YP_002342100.1) (SEQ ID NO: 30). In some embodiments, wild type Cas9 corresponds to Cas9 from Streptococcus pyogenes (NCBI Reference Sequence: NC_017053.1) (SEQ ID NO: 31).

Among the known Cas9 proteins, S. pyogenes Cas9 has been widely used as a tool for genome engineering. This Cas9 protein is a large, multi-domain protein containing two distinct nuclease domains. Point mutations can be introduced into Cas9 to abolish nuclease activity, resulting in a dead Cas9 (dCas9) that still retains its ability to bind DNA in a sgRNA-programmed manner. In principle, when fused to another protein or domain, dCas9 can target that protein to virtually any DNA sequence simply by co-expression with an appropriate sgRNA.

The present disclosure provides nucleic acid constructs comprising polynucleotides encoding Cas9 proteins for insertion of exogenous nucleic acid into a specific site of a genome. Some aspects of this disclosure provide fusion proteins comprising a Cas9 protein and a modified integrase or a modified transposase of the disclosure. Some embodiments of this disclosure provide nucleic acids encoding such Cas9 proteins or fusion proteins. Some embodiments provide a plasmid or expression vector comprising such nucleic acids.

The Cas9 encoded by the nucleic acid construct disclosed herein can be any Cas9 that can bind to a specific genomic DNA sequence in a genome. Non-limiting examples of Cas9 proteins include human Cas9 (hCas9), nickase Cas9 (nCas9), dead Cas9 (dCas9), Streptococcus pyogenes Cas9, Staphylococcus aureus Cas9, Cas12a, Cas12b, dead Cas9 (dCas9), variants and functional fragments thereof. In some embodiments, the Cas9 is a human Cas9 or a variant or functional fragment thereof.

In some embodiments, the hCas9 is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 64. In some embodiments, the nCas9 is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 65. In some embodiments, the dCas9 is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 66.

In some embodiments, the hCas9 comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 69. In some embodiments, the nCas9 comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 70. In some embodiments, the dCas9 comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 71.

Certain aspects of the disclosure are directed to a vector or a plasmid (e.g., an expression vector or a packaging vector) comprising a nucleic acid construct comprising a Cas9 suitable for expression in a host cell, e.g., mammalian cells, yeast cells, insect cells, plant cells, fungal cells, or algal cells. In some embodiments, the nucleic acid construct comprises a polynucleotide sequence encoding a Cas9 that is expressed as a fusion protein with a modified transposase of the disclosure.

ii. Zinc Finger Proteins

The present disclosure also provides nucleic acid constructs comprising polynucleotides encoding a zinc finger protein (ZFP) for insertion of exogenous nucleic acid into a specific site of a genome. Some aspects of this disclosure provide fusion proteins comprising a ZFP and a modified integrase or a modified transposase of the disclosure. Some embodiments of this disclosure provide nucleic acids encoding such ZFP or fusion proteins. Some embodiments of this disclosure provide plasmids or an expression vectors comprising such encoding nucleic acids.

Zinc finger proteins used herein are proteins that can bind to DNA in a sequence-specific manner. ZFP are unevenly distributed in eukaryotes. ZFP have been identified that are involved in DNA recognition, RNA binding, and protein binding. Certain classifications for zinc finger proteins are based on “fold groups” in view of the overall shape of the protein backbone in the folded domain. The most common “fold groups” of zinc fingers are the C₂H₂ or Cys₂His₂-like (the “classic zinc finger”), treble clef, and zinc ribbon. Representative motif characterizing one class of these proteins (C2H2 class) is, -Cys-(X) 2-4-Cys-(X) 12-His-(X) 3-5-His (where in X is a is any amino acid).

The ZFP of the disclosure can be any ZFP, variant or functional fragment thereof, that can bind to a specific genomic DNA sequence in a genome. Non-limiting examples of ZFPs include ZFPs comprising a fold group or zinc finger motif selected from C2H2, gag knuckle, treble clef, zinc ribbon, Zn2/Cys6-like, or TAZ2 domain-like, or any combination thereof. In some embodiments, the ZFP is a C2H2 zinc finger protein. In some embodiments, the ZFP is an engineered ZFP.

Engineered zinc finger arrays can be fused to a DNA cleavage domain (usually the cleavage domain of FokI) to generate zinc finger nucleases. Such zinc finger-FokI fusions have become useful reagents for manipulating genomes.

The ZFP of the disclosure can comprise 2, 3, 4, 5, 6, 7, 8, 9, 10, 11, 12, or more zinc finger domains. The ZFP can comprise 2-12, 2-10, 2-8, 3-8, 4-8, or 5-8 zinc finger domains. In some embodiments, the ZFP comprises 6 zinc finger domains.

A common modular assembly process involves combining separate zinc fingers that can each recognize a 3-basepair DNA sequence to generate 3-finger, 4-, 5-, or 6-finger arrays that recognize target sites ranging from 9 basepairs to 18 basepairs in length. Another method uses 2-finger modules to generate zinc finger arrays with up to six individual zinc fingers.

In some embodiments, the binding domain of the ZFP can be engineered to bind to a sequence of choice. An engineered zinc finger binding domain can have improved binding specificity, compared to a naturally occurring ZFP. In some embodiments, the nucleic acid sequence encoding the ZFP corresponds to SEQ ID NO: 32, SEQ ID NO: 34, SEQ ID NO: 36, or SEQ ID NO: 38. In some embodiments, the amino acid sequence of the ZFP corresponds to SEQ ID NO: 33, SEQ ID NO: 35, SEQ ID NO: 37, or SEQ ID NO: 39. In some embodiments, the ZFP comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to any of SEQ ID NOs: 33, 35, 37 or 39.

Certain aspects of the disclosure are directed to a vector or a plasmid (e.g., an expression vector or a packaging vector) comprising a nucleic acid construct comprising a ZFP suitable for expression in a host cell, e.g., mammalian cells, yeast cells, insect cells, plant cells, fungal cells, or algal cells. In some embodiments, the nucleic acid construct comprises a polynucleotide sequence encoding a ZFP which is expressed as a fusion protein with a modified integrase or a modified transposase of the disclosure.

### VII. Fusion Protein

The present disclosure provides fusion proteins for site-specific insertion of exogenous nucleic acids into a genome. In certain embodiments, the fusion protein comprises a first DNA binding protein engineered to bind to a specific genomic DNA sequence, a second DNA binding protein which enables insertion of an exogenous nucleic acid into the genome wherein the second DNA binding protein is an integrase or a transposase of this disclosure, and a linker connecting the first and second protein. In some embodiments the first DNA binding protein is a Cas9 protein or a zinc finger protein. In some embodiments the first DNA binding protein is a Cas9 and the second binding protein is a modified transposase disclosed herein, wherein the first and second binding protein can be oriented in the construct in either order. In some embodiments the first DNA binding protein is a zinc finger protein and the second binding protein is a modified integrase, wherein the first and second binding protein can be oriented in the construct in either order.

In some embodiments, the fusion protein comprises a linker between the first binding protein and the second binding protein, wherein the linker comprises a (GGS)n, a (GGGGS)n (SEQ ID NO: 133), a (G)n, an (EAAAK)n (SEQ ID NO: 134), a XTEN-based, or an (XP)n motif, or a combination of any of any of these, wherein n is independently an integer between 1 and 50. In some embodiments, the linker is 12 to 24 amino acids, or encoded by a nucleic acid sequence that is 36 to 72 nucleic acids in length. In some embodiments the linker comprises a XTEN sequence or a GGS sequence. In some embodiments, the fusion protein comprises a zinc finger protein linked to a modified integrase of the disclosure, wherein the linker comprises a GGS sequence or an XTEN sequence, and wherein the modified integrase can be 5′ or 3′ to the linker. In some embodiments, the fusion protein comprises a Cas9 protein linked to a modified transposase of the disclosure, wherein the linker comprises a GGS sequence or an XTEN sequence, and wherein the modified transposase can be 5′ or 3′ to the linker. In some embodiments, the linker is a linker shown in Table 1. In some embodiments, the linker is comprises the amino acid sequence of SEQ ID NO: 49. In some embodiments, the linker comprises an amino acid sequence selected from the group consisting of SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, or any combination thereof. In some embodiments, the linker is encoded by a nucleic acid sequence comprising SEQ ID NO: 48. In some embodiments, the linker is encoded by a nucleic acid sequence comprising a sequence selected from the group consisting of SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, or any combination thereof.

TABLE 1
Linkers

| | Nucleic Acid Sequence | Amino Acid Sequence |
|---|---|---|
| Linker | (SEQ ID NO) | (SEQ ID NO) |
| GGSx3 | ggtggatctggcggtggatctggtggcggt | GGSGGGSGGG (SEQ ID NO: |
| | (SEQ ID NO: 48) | 49) |
| GGS4x | ggagggagtggtgggtccggtggtagtggcggatcc | GGSGGSGGSGGS |
| | (SEQ ID NO: 50) | (SEQ ID NO: 51) |
| GGS5x | ggaggctccggtgggtctggtgggagcggtggtagtggcgg | GGSGGSGGSGGSGGS |
| | atcc (SEQ ID NO: 52) | (SEQ ID NO: 53) |
| GGS6x | ggaggcagtggtgggagcggtggttccgggggtagtggtggt | GGSGGSGGSGGSGGSGGS |
| | tccgggggatcc (SEQ ID NO: 54) | (SEQ ID NO: 55) |
| GGS7x | ggaggttctggaggctccggtgggtccgggggaagtggggg | GGSGGSGGSGGSGGSGGSG |
| | gtcaggcggatcaggaggatcc (SEQ ID NO: 56) | GS (SEQ ID NO: 57) |
| GGS8x | ggaggtagcggaggttccggagggagcggcgggagtgggg | GGSGGSGGSGGSGGSGGSG |
| | gaagcgggggaagtggaggatccgggggaggatcc (SEQ | GS (SEQ ID NO: 59) |
| | ID NO: 58) | |
| Linker | tccggtagcgaaacaccggggacttcagaatcggccaccccg | SGSETPGTSESATPES |
| XTEN | gagtct (SEQ ID NO: 60) | (SEQ ID NO: 61) |
| Linker | ggaagcgccggtagtgcggctgggtctggcgagttc | GSAGSAAGSGEF |
| B | (SEQ ID NO: 62) | (SEQ ID NO: 63) |

In some embodiments, the 3′ end of the first DNA binding protein is connected to the 5′ end of the second DNA binding protein by a linker. In some embodiments the 3′ end of the second DNA binding protein is connected to the 5′ end of the first DNA binding protein by a linker. In some embodiments, the 3′ end of the Cas 9 protein is connected to the 5′ end of the transposase by a linker. In some embodiments, the 5′ end of the Cas 9 protein is connected to the 3′ end of the transposase by a linker. In some embodiments, the 3′ zinc finger protein is connected to the 5′ end of the integrase by a linker. In some embodiments, the 5′ zinc finger protein is connected to the 3′ end of the integrase by a linker.

Also provided herein are fusion proteins obtained from the expression of any of the nucleic acid constructs provided in this disclosure.

### VIII. Host Cells/Organism

In some embodiments, the nucleic acid construct of the disclosure is expressed in a host cell. Suitable host cells include but not limited to eukaryotic and prokaryotic cells and/or cell lines. Non-limiting examples of such host cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), and perC6 cells as well as insect cells such as Spodoptera fugiperda (Sf), or fungal cells such as Saccharomyces, Pichia and Schizosaccharomyces.

In some embodiments, the host cell is from a microorganism. Microorganisms which are useful for certain methods disclosed herein include, for example, bacteria (e.g., E coli), yeast (e.g., Saccharomyces cerevisiae), and plants. The host cell can be prokaryotic or eukaryotic. In some embodiments, the host cell is eukaryotic. Suitable eukaryotic host cells include, but are not limited to, yeast cells, insect cells, plant cells, fungal cells, and algal cells.

In some embodiments, the host cell is a competent host cell. In some embodiments, the host cell is naturally competent. In some embodiments, the host cells are made competent, e.g., by a process that uses calcium chloride and heat shock. The cells used can be any cell competent, particularly eukaryotic cells, in particular mammalian, e.g. human or animal. They can be somatic or embryonic stem or differentiated. In some aspects, the cells include 293T cells, fibroblast cells, hepatocytes, muscle cells (skeletal, cardiac, smooth, blood vessel, etc.), nerve cells (neurons, glial cells, astrocytes) of epithelial cells, renal, ocular etc. It may also include, insect, plant cells, yeast, or prokaryotic cells. Additionally, primary cells may be isolated and used ex vivo for reintroduction into the subject to be treated following treatment with the nucleases (e.g. ZFNs or TALENs) or nuclease systems (e.g. CRISPR/Cas). Suitable primary cells include peripheral blood mononuclear cells (PBMC), and other blood cell subsets such as, but not limited to, T-lymphocytes such as CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In some embodiments, the host cell is transfected with a plasmid comprising a nucleic acid construct disclosed herein. In some embodiments, the plasmid comprising the nucleic acid construct is an packaging plasmid. In some embodiments, the plasmid comprising the nucleic acid construct further comprises a polynucleotide encoding capsid proteins, e.g., gag and pol. In some embodiments, the host cell is transfected with (i) the plasmid comprising the nucleic acid construct is combined in the host cell with (ii) a plasmid comprising a polynucleotide that encode proteins for a viral envelope (envelope plasmid); and (iii) a plasmid comprising an exogenous nucleic acid sequence (e.g., a GOI), wherein a virus particle comprising the exogenous nucleic acid, e.g., GOI, and the fusion protein comprising the first and the second binding protein is produced.

In some embodiments, the host cell is transfected with (i) the plasmid comprising the nucleic acid construct is combined with (ii) a plasmid comprising the nucleic acid construct further comprises a polynucleotide encoding capsid proteins, e.g., gag and pol (a packaging plasmid, wherein the packaging plasmid lacks a functional integrase); (iii) a plasmid comprising a polynucleotide that encode proteins for a viral envelope (envelope plasmid) and (iv) a plasmid comprising an exogenous nucleic acid sequence (e.g., a GOI), wherein a virus particle comprising the exogenous nucleic acid, e.g., GOI, and the fusion protein comprising the first and the second binding protein is produced.

In further embodiments, a vector, e.g., a lentiviral vector according to the disclosure, can be used for delivering a fusion protein encoded by a nucleic acid construct of the disclosure and an exogenous nucleic acid to an organism, e.g., a mammal, and more particularly to a mammalian target cell of interest. The lentiviral vectors comprising fusion proteins of the disclosure are able to transduce various cell types such as, for example, liver cells (e.g. hepatocytes), muscle cells, brain cells, kidney cells, retinal cells, and hematopoietic cells. In some embodiments, the target cells of the present disclosure are “non-dividing” cells. These cells include cells such as neuronal cells that do not normally divide. However, it is not intended that the present disclosure be limited to non-dividing cells (including, but not limited to muscle cells, white blood cells, spleen cells, liver cells, eye cells, epithelial cells, etc.).

In certain embodiments, a packaged fusion protein of the disclosure is administered to an organism, e.g., for gene editing of the organism's DNA. In some embodiments, the organism is a human. In some embodiments, the organism is a non-human mammal. In some embodiments, the organism is a non-human primate. In some embodiments, the organism is a rodent. In some embodiments, the organism is a sheep, a goat, a cattle, a cat, or a dog. In some embodiments, the organism is a vertebrate, an amphibian, a reptile, a fish, an insect, a fly, or a nematode. In some embodiments, the organism is a research animal. In some embodiments, the organism is genetically engineered, e.g., a genetically engineered non-human subject. The organism may be of either sex and at any stage of development.

### IX. Method of Inserting into Genome

Methods for inserting exogenous nucleic acids into a genome have been described. See, e.g., Yusa et al. PNAS 4(108):1531-1536 (2011); Feng et al. Nuc. Acid Res. 4(38):1204-1216 (2009); Kettlun et al. Amer. Soc. Gene and Cell Ther. 9(19):1636-1644 (2011); Skipper et al. 20(92):1-23 (2013); Li et al. PNAS 25:E2279-E2287 (2013); Mates et al. Nature Genetics 41(6):753-761 (2009); Mali et al. Nat. Methods 10(10):957-963; Vargas et al. J. Trans. Med. 14(288):1-15 (2016); Gersbach et al. Acc. Chem. Res. 47:2309-2318 (2014); Chandrasegaran et al. Cell Gene Ther. Ins. 3(1):33-41 (2017); Wilson et al. 649:353-363 (2010); Zhao Zhang, et al. Mol Ther Nucleic Acids. 9: 230-241 (2017); Naldini L. EMBO Mol Med. 11(3) (2019); and Naldini L, et al. Hum Gene Ther. 27(10):727-728 (2016), each of which is incorporated herein by reference.

The present disclosure provides a nucleic acid construct encoding a fusion protein for insertion of exogenous nucleic acid into a specific site of a genome. The present invention also provides fusion proteins for insertion of exogenous nucleic acid into a specific site of the genome. In some embodiments the exogenous nucleic acid for insertion can be up to up to 5 kb in length, up to 10 kb in length, up to 15 kb in length, 20 kb in length, up to 25 kb in length, up to 30 kb in length, up to 35 kb in length, or up to 40 kb in length.

In another embodiment, methods for site-specific nucleic acid insertion into the genome are provided. In some embodiments, the methods comprise contacting a target DNA with any of the fusion proteins comprising a Cas9 and a transposase described herein. For example, in some embodiments, the method comprises contacting a DNA with a fusion protein that comprises two linked polypeptides: (i) a Cas9; and (ii) a transposase, wherein the active Cas9 binds a gRNA that hybridizes to a region of the DNA, e.g., a genomic DNA.

In some embodiments, the methods comprise contacting a target DNA with any of the fusion proteins comprising a Cas9 and an integrase described herein. For example, in some embodiments, the method comprises contacting a DNA with a fusion protein that comprises two linked polypeptides: (i) a Cas9; and (ii) an integrase, wherein the active Cas9 binds a gRNA that hybridizes to a region of the DNA, e.g., a genomic DNA.

In some embodiments, the methods comprise contacting a target DNA with any of the fusion proteins comprising a ZFP and an integrase described herein. For example, in some embodiments, the method comprises contacting a DNA with a fusion protein that comprises two linked polypeptides: (i) ZFP; and (ii) an integrase, wherein the active ZFP hybridizes to a region of the DNA, e.g., a genomic DNA.

In some embodiments, the fusion protein is delivered to an organism and/or a cell comprising the target DNA, e.g., genomic DNA, using a viral vector, e.g., a lentiviral particle.

### X. Lentiviral Packaging

Methods for lentiviral packaging have been described. See, Grandchamp et al. 9(6):1-13 (2014); Voelkel et al. 107(17):7805-7810 (2010); Tan et al. 80(4)1939-1948; Li et al. 9(8):1-9 (2014); Mátés et al. Nature Genetics 41(6):753-761 (2009); and Robert H Kutnerl, et al. NATURE PROTOCOLS 4(4):495 (2009), each of which is incorporated herein by reference.

Typically, lentiviral delivery systems use a split system with different lentiviral genes on separate plasmids being used to produce a complete virus that does not contain the genetic components needed to cause the viral disease. For example, one plasmid (an envelope plasmid) can encode the proteins for the viral envelope (env); another plasmid (a packaging plasmid) can encode capsid proteins (e.g., gag and pol) and the enzymes like reverse transcriptase and/or integrase; and a further plasmid comprising the gene of interest (GOI) flanked by long-terminal repeats (for genome integration) and a psi-sequence (which displays a signal to package the gene into the virus) (a transfer plasmid). If these plasmids are simultaneously introduced into a cell, viruses will be produced containing the GOI without the viral genes that are needed to cause disease.

In certain aspects of the disclosure, the lentiviral vector (or particle) of the invention is obtainable by a split system, e.g., a transcomplementation system (vector/packaging system), by transfecting in vitro a permissive cell (such as 293T cells) with a plasmid containing certain components of the lentiviral vector genome, and at least one other plasmid providing, in trans, the gag, pol and env sequences encoding the polypeptides GAG, POL and the envelope protein(s), or for a portion of these polypeptides sufficient to enable formation of retroviral particles.

As an example, host cells are transfected with a) packaging plasmid, comprising a lentiviral gag and pol sequence, b) a second plasmid (envelope expression plasmid or pseudotyping env plasmid) comprising a gene encoding an envelope protein(s) (such as VSV-G), c) a plasmid vector comprising between 5′ and 3′ LTR sequences, a psi encapsidation sequence, and a transgene, and d) a plasmid vector comprising a nucleic acid construct encoding an engineered fusion protein disclosed herein. In some embodiments, the nucleic acid construct encoding the engineered fusion protein disclosed herein is on the packaging plasmid instead of a separate plasmid. Nucleic acids encoding gag, pol and env cDNA can be advantageously prepared according to conventional techniques, from viral gene sequences available in the prior art and databases.

In some embodiments, a lentiviral vector comprises a nucleic acid construct as described herein. In some embodiments, a lentiviral vector comprises a fusion protein as described herein.

The promoters used in the plasmids can be identical or different. In some embodiments, in the plasmid transcomplementation system, the envelope plasmid and the plasmid vector, respectively, to promote the expression of gag and pol of the coat protein, the mRNA of the vector genome and the transgene are promoters which can be identical or different. Such promoters can be chosen advantageously from ubiquitous promoters or specific, for example, from viral promoters CMV, TK, RSV LTR promoter and the RNA polymerase III promoter such as U6 or H1 or promoters of helper viruses encoding env, gag and pol (i.e. adenoviral, baculoviral, herpes viruses).

For the production of the lentiviral vector of the disclosure, the plasmids described herein can be introduced into host cells and the viruses are produced and harvested. Suitable cells include but not limited to eukaryotic and prokaryotic cells and/or cell lines. Non-limiting examples of such cells or cell lines generated from such cells include, e.g., COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), and perC6 cells as well as insect cells such as Spodoptera fugiperda (Sf), or fungal cells such as Saccharomyces, Pichia and Schizosaccharomyces.

Once host cells are transfected with the plasmids and a lentiviral vector (or particles) of the disclosure is produced, the lentiviral vectors (or particles) of the disclosure can be purified from the supernatant of the cells. Purification of the lentiviral vector to enhance the concentration can be accomplished by any suitable method, such as by density gradient purification (e.g., cesium chloride (CsCl)), by chromatography techniques (e.g., column or batch chromatography), or by ultracentrifugation. For example, the vector of the invention can be subjected to two or three CsCl density gradient purification steps. The vector, is desirably purified from infected cells using a method that comprises lysing cells, applying the lysate to a chromatography resin, eluting the virus from the chromatography resin, and collecting a fraction containing the lentiviral vector of the disclosure.

### XI. Method of Delivery

Methods of delivery of lentiviral vectors have been described. See, e.g., Vargas et al. J. Trans. Med. 14(288):1-15 (2016); Mali et al. Nat. Methods 10(10):957-963; Mates et al. Nature Genetics 41(6):753-761 (2009); Skipper et al. 20(92):1-23 (2013).

Lentiviral vectors comprising a fusion protein of encoded by a nucleic acid construct of the disclosure can be administered to a subject by any route. In some embodiments, a lentiviral vector of the disclosure can be delivered to cells of a subject either in vivo or ex vivo.

In some embodiments, the lentiviral vector of the disclosure can be delivered in vivo. In some embodiments, a lentiviral vectors comprising a fusion protein encoded by a nucleic acid construct of the disclosure can be used to deliver a GOI and/or to target a genetic defect in a subject's DNA. In some embodiments, the lentiviral vector is administered to the subject parenterally, preferably intravascularly (including intravenously). When administered parenterally, it is preferred that the vectors be given in a pharmaceutical vehicle suitable for injection such as a sterile aqueous solution or dispersion.

In some embodiments, the lentiviral vector of the disclosure can be used ex vivo.

In some embodiments, a lentiviral vector comprising a fusion protein encoded by a nucleic acid construct of the disclosure can be used to deliver a GOI and/or target a genetic defect in a subject's DNA. In some embodiments, cells are removed from a subject and lentiviral vector comprising a fusion protein encoded by a nucleic acid construct of the disclosure is administered to the cells ex vivo to modify the DNA of the cells. The cells carrying the modified DNA are then expanded and reinfused back into the subject. In certain embodiments, a lentiviral vectors comprising a fusion protein encoded by a nucleic acid construct of the disclosure can be used for Chimeric Antigen Receptor (CAR) T-cell therapy to genetically modify a patient's autologous T-cells to express a CAR specific for a tumor antigen. In a further embodiment, the modified CAR-T cells are expanded ex vivo and re-infusion back to the patient. In some embodiments, the altered T cells more specifically target cancer cells. Unlike antibody therapies, CAR-T cells are able to replicate in vivo resulting in long-term persistence.

Following administration of a lentiviral vector of the disclosure or cells modified ex vivo using a lentiviral vector of the disclosure, the subject can be monitored to detect the expression of the transgene. Dose and duration of treatment is determined individually depending on the condition or disease to be treated. A variety of conditions or diseases can be treated based on the gene expression produced by administration of the gene of interest in the vector of the present invention. The dosage of vector delivered using the method of the invention will vary depending on the desired response by the host and the vector used.

In some gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest.

Certain aspects of the disclosure are directed to a method of inserting an exogenous nucleic acid sequence into genomic DNA of an organism, comprising: identifying the specific genomic DNA sequence in the genome of the organism; administering a lentiviral particle comprising the nucleic acid construct of the disclosure to the organism to bind to the specific genomic DNA sequence and insert the exogenous nucleic acid into the genomic DNA; wherein the exogenous nucleic acid becomes integrated at the specific genomic DNA sequence.

Certain aspects of the disclosure are directed to a method for controlled, site-specific integration of a single copy or multiple copies of an exogenous nucleic acid sequence into a cell, the method comprising: a) delivering the nucleic acid construct, the vector, or the fusion protein of the disclosure to the cell, and b) delivering the exogenous nucleic acid to the cell; wherein binding of the fusion protein to the specific genomic DNA sequence in the genome of the cell, results in cleavage of the genome and integration of one or more copies of the exogenous nucleic acid into the genome of the cell. In some aspects, the delivery to the cell is by means of a lentiviral particle.

### XII. Method of Use/Applications

Several strategies can be used to test for integrations sites, and to screen for the best machinery for directed integration.

For analysis of the modified integrase and transposons disclosed herein, a reporter cell line with a promoter, half of the coding sequence of the GFP and a splice site donor downstream of the targeted insertion site in the genome can be used. For example, the lentiviral payload can have a fusion integrase variant followed by the inverted splice site acceptor and the other half of the GPF. The expression of GFP will occur when direct insertion happens and splicing of the GFP containing mRNA generated from the insertion site and integrated payload originates the full GFP CDS.

VPR transcomplementation systems can also be used for screening and comparing integration mutants. The transcomplementation system can be use for targeted insertion of the lentiviral payload containing a fusion integrase variant that, when expressed and loaded in the particle promote its own integration will be loaded in the viral particle using a VPR fusion. This will complement in trans the integration defective IN coded in the packaging vector used for particle production. Other methods that can be used for integration mapping including IC, or FISH probes. Targeted insertion can also be screened by TCRa or RFP targeted disruption, or GFP activation by targeted splice site integration.

For the FISH approach to co-staining of the insertion and target region in the chromatin, a Fluorescence in situ hybridization to localize the GOI transposon in the Hek293T genome can be performed. Hek293T can be transfected with 1) GOI-transposon 2) Programmable transposase and 3) gRNA to PPP1R12. Probes are designed to target the PPP1R12 gene, CD46 gene (as negative control) and GOI, and can be synthesized with Nick Translation Mix (Sigma) from PCR amplified DNA.

In some embodiments, a fusion protein comprising a modified transposase or a modified integrase as disclosed herein improve the specificity of insertion of the exogenous nucleic acid into the genome compared to a fusion protein containing the corresponding wildtype protein, e.g., as determined by a Genetrap assay. In some embodiments, HEK293T cells, or any other permissible cells, are transfected or transduced with lentiviral particles with the following plasmids or payloads: (i) a plasmid comprising a gRNA that targets a specific region of DNA, (ii) a plasmid comprising the nucleic acid construct of the disclosure encoding a modified transposase fusion protein or modified integrase fusion protein, and (iii) a genetrap plasmid comprising a nucleic acid sequence encoding a reporter protein, e.g., GFP, that lacks a promoter. In some embodiments, the genetrap plasmid further comprises a transposon with inverted repeats.

In some embodiments, the percent of cells containing the GFP insertion can be determined by flow cytometry. In some embodiments, the programmable transposase fusion protein increases the percent of cells containing insertion of GFP by at least 5%, at least 10%, at least 15%, at least 20%, at least 25%, or at least 30% compared to the corresponding wildtype protein. In some embodiments, the programmable transposase fusion protein increases the percent of cells containing insertion of GFP by about 15-30%.

In some embodiments, the percent of insertions at the targeted site and percent of coverage at the target site (number of reads per insertion site) can be determined by genomic DNA extraction and targeted sequencing with oligonucleotides specific for viral LTRs. In some embodiments, the modified transposase fusion protein increases the percent of insertions at the targeted site by at least 10-fold, at least 20-fold, at least 30-fold, at least 40-fold, at least 50-fold, at least 60-fold, at least 70-fold, at least 80-fold, at least 90-fold, or at least 100-fold compared to the corresponding wildtype protein. In some embodiments, the percent of insertions at the targeted site is increased by about 10-100 fold. In some embodiments, the modified transposase fusion protein increases the percent of coverage at the target site (number of reads per insertion site) by at least 10-fold, at least 20-fold, at least 30-fold, at least 40-fold, at least 50-fold, at least 60-fold, at least 70-fold, at least 80-fold, at least 90-fold, at least 100-fold, at least 110-fold, at least 120-fold, at least 130-fold, at least 140-fold, at least 150-fold, at least 160-fold, at least 170-fold, at least 180-fold, at least 190-fold, or at least 200-fold compared to the corresponding wildtype protein. In some embodiments, the percent of coverage at the target site (number of reads per insertion site) by at least 100-fold.

In some embodiments, the modified integrase fusion protein improves the specificity of inserting the exogenous nucleic acid into the genome compared to the corresponding wildtype protein as quantified by GFP integration. In some embodiments, lentivirus containing the modified integrase fusion protein was generated by transfecting HEK293T cells, or any other permissible cells, with (i) a plasmid containing a nucleic acid sequence encoding GFP, (ii) a plasmid containing packaging proteins, (iii) a plasmid containing an envelope protein, and (iv) a plasmid containing the nucleic acid construct encoding the modified integrase fusion protein. The supernatant containing the lentivirus was collected 48 hrs post-transfection.

For targeted insertion, HEK293T cells were infected with the lentivirus containing the modified integrase fusion protein. In some embodiments, the percent of GFP positive cells were quantified by flow cytometry at 3, 5, 7, 10, and 12 days post-infection. In some embodiments the, the modified integrase fusion protein increases the percent of cells containing insertion of GFP by at least 5%, at least 10%, at least 15%, at least 20%, at least 25%, or at least 30% compared to the corresponding wildtype protein.

In some embodiments, the percent of insertions at the targeted site and percent of coverage at the target site (number of reads per insertion site) can be determined by genomic DNA extraction and targeted sequencing with oligonucleotides specific for viral inserted LTR. In some embodiments, the modified integrase fusion protein increases the percent of insertions at the targeted site by at least 10-fold, at least 20-fold, at least 30-fold, at least 40-fold, at least 50-fold, at least 60-fold, at least 70-fold, at least 80-fold, at least 90-fold, or at least 100-fold compared to the corresponding wildtype protein. In some embodiments, the modified integrase fusion protein increases the percent of coverage at the target site (number of reads per insertion site) by at least 10-fold, at least 20-fold, at least 30-fold, at least 40-fold, at least 50-fold, at least 60-fold, at least 70-fold, at least 80-fold, at least 90-fold, at least 100-fold, at least 110-fold, at least 120-fold, at least 130-fold, at least 140-fold, at least 150-fold, at least 160-fold, at least 170-fold, at least 180-fold, at least 190-fold, or at least 200-fold compared to the corresponding wildtype protein.

Possible applications of lentiviral vectors comprising the fusion proteins of the disclosure include gene therapy, i.e., the gene transfer in any mammal cell, in particular in human cells. It may be dividing cells or quiescent cells, cells belonging to the central organs or peripheral organs such as the liver, pancreas, muscle, heart, etc. Gene therapy may allow the expression of proteins, e.g. neurotrophic factors, enzymes, transcription factors, receptors, etc. Lentiviral vectors according to the invention may also particularly suitable for research purposes.

In some embodiments, a nucleic acid constructs, a fusion protein, and/or a lentiviral vector of the disclosure is administered to a subject to treat a disease. In some embodiments, the disease is a genetic disorder that can benefit from gene therapy.

In some embodiments, the lentiviral vectors comprising the fusion proteins according to the disclosure can be used as a medicament. The lentiviral vector according to the disclosure may be particularly suitable for treating a genetic disease in a subject.

### XIII. Compositions and Kits

The present disclosure also provides compositions for practicing the disclosed methods as described herein. In some embodiments, a composition comprises a nucleic acid construct or a vector as defined in this disclosure, and a polynucleotide sequence encoding an exogenous nucleic acid for insertion in a genome, contained in in or bound to a packaging vector.

In some embodiments, the nucleic acid construct is in form of RNA, DNA or protein, and the polynucleotide sequence encoding the exogenous nucleic acid is in form of RNA or DNA, depending on the method of delivery. Particularly, the polynucleotide sequence encoding the exogenous nucleic acid is in form of RNA.

In some embodiments, the composition is viral-free and the packaging vector is a nanoparticle e.g. a polymeric or lipidic nanoparticle. The packaging vector can also be a carrier which is bound to the elements of the composition. In some embodiments, the composition is contained in a viral vector, particularly a lentiviral particle.

In some embodiments, the composition comprises (a) the nucleic acid construct described herein (e.g. comprising Cas9 and a transposase) in form of RNA, (b) a guide RNA if needed (e.g. as separate lineal single strand RNA molecule), and (c) a polynucleotide comprising the exogenous gene for insertion in DNA form (e.g. in a vector), contained in in or bound to a packaging vector.

In some embodiments, the composition comprises (a) the fusion protein described herein (e.g. comprising Cas9 and a transposase) in form of protein, (b) a guide RNA if needed (e.g. as separate lineal single strand RNA molecule), wherein the fusion protein and the guide RNA form a ribonucleic protein complex (RNP), and (c) a polynucleotide comprising the exogenous gene for insertion in DNA form (e.g. in a vector), contained in in or bound to a packaging vector.

In some embodiments, the composition comprises (a) the nucleic acid construct described herein (e.g. comprising Cas9 and a transposase) in form of DNA, (b) a guide RNA if needed (e.g. as separate lineal RNA molecule or as DNA in a vector), and (c) a polynucleotide comprising the exogenous gene for insertion in DNA form (e.g. in a vector), contained in in or bound to a packaging vector.

In some embodiments, the composition comprises (a) the fusion protein described herein (e.g. comprising Cas9 and an integrase) in form of protein, (b) a guide RNA if needed (e.g. as separate RNA molecule complexing with the fusion protein), and (c) a polynucleotide comprising the exogenous gene for insertion, contained in in or bound to a packaging vector. In a particular embodiment, the packaging vector is a lentiviral particle. In some embodiments, the (a) fusion protein is bound to the lentiviral capside by means of gag-pol or VPR (Viral Protein R). In some embodiments, the (c) polynucleotide is in form of RNA as payload of the integrase.

In a particular embodiment, when ZFP is used, (b) the guide RNA can not be needed.

Also provided by the present disclosure are kits for practicing the disclosed methods, as described herein. The kit can contain the nucleic acid constructs or fusion proteins as described herein. In some aspects, the kit can contain the lentiviral particles containing the nucleic acid constructs or fusion proteins as described herein.

The subject kit can further include instructions for using the components of the kit to practice the subject methods. The instructions for practicing the subject methods are generally recorded on a suitable recording medium. For example, the instructions can be printed on a substrate, such as paper or plastic, etc. As such, the instructions can be present in the kit as a package insert, in the labeling of the container of the kit or components thereof (i.e., associated with the packaging or subpackaging), etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### XIV. Embodiments

E1. A nucleic acid construct comprising:

a) a first polynucleotide sequence encoding a first DNA binding protein engineered to bind to a specific genomic DNA sequence in a genome;

b) a second polynucleotide sequence encoding a second DNA binding protein which enables insertion of an exogenous nucleic acid into the genome, wherein the second DNA binding protein is (i) an integrase which is modified relative to a wildtype integrase or (ii) a transposase which is modified relative to a wildtype transposase; and

c) a third polynucleotide sequence comprising a nucleic acid encoding a linker;

wherein the nucleic acid construct encodes a fusion protein comprising the first DNA binding protein, the second DNA binding protein, and the linker between the first DNA binding protein and the second DNA binding protein.

E2. The nucleic acid construct of embodiment E1, wherein the second DNA binding protein is modified to improve specificity of inserting the exogenous nucleic acid into the genome compared to the corresponding wildtype protein.

E3. The nucleic acid construct of embodiment E1 or E2, wherein the exogenous nucleic acid for insertion can be up to about 20 kb in length.

E4. The nucleic acid construct of any one of embodiments E1 or E3, wherein the first polynucleotide sequence encodes a protein selected from the group consisting of a zinc finger protein, a Cas9 protein, and any variant or functional fragment thereof.

E5. The nucleic acid construct of embodiment E4, wherein the Cas9 protein is selected from the group consisting of a human Cas9, a nickase Cas9, Streptococcus pyogenes Cas9, Staphylococcus aureus Cas9, Cas12a, Cas12b, and a dead Cas 9.

E6. The nucleic acid construct of embodiment E4, wherein the zinc finger protein is a C2H2 zinc finger protein.

E7. The nucleic acid construct of any one of embodiments E1-E6, wherein the modified integrase is a modified human immunodeficiency virus (HIV) integrase or functional fragment thereof.

E8. The nucleic acid construct of embodiment E7, wherein the modified HIV integrase comprises a mutation of one or more of amino acids 10, 13, 64, 94, 116, 117, 119, 120, 122, 124, 128, 152, 168, 170, 185, 231, 264, 266, or 273 corresponding to the amino acid number of the wildtype HIV integrase sequence (SEQ ID NO: 1).

E9. The nucleic acid construct of embodiment E8, wherein the modified HIV integrase mutation comprises one or more of D10K, E13K, D64A, D64E, G94D, G94E, G94R, G94K, D116A, D116E, N117D, N117E, N117R, N117K, S119A, S119P, S119T, S119G, S119D, S119E, S119R, S119K, N120D, N120E, N120R, N120K, T122K, T122I, T122V, T122A, T122R, A124D, A124E, A124R, A124K, A128T, E152A, E152D, Q168L, Q168A, E170G, F185K, R231G, R231K, R231D, R231E, R231S, K264R, K266R, or K273R, corresponding to the amino acid number of the wildtype HIV integrase sequence (SEQ ID NO: 1).

E10. The nucleic acid construct of any one of embodiments E7-E9, wherein the modified HIV integrase comprises an amino acid sequence at least 85%, at least 90%, or at least 95% identical to the sequence set forth in SEQ ID NO: 3.

E11. The nucleic acid construct of any one of embodiments E1-E6, wherein the modified transposase is selected from the group consisting of a modified Frog Prince, a modified Sleeping Beauty, a modified hyperactive Sleeping Beauty (SB100X), a modified PiggyBac, a modified hyperactive PiggyBac, and any functional fragment thereof.

E12. The nucleic acid construct of embodiment E11, wherein the modified transposase is a modified hyperactive PiggyBac or functional fragment thereof.

E13. The nucleic acid construct of embodiment E12, wherein the modified hyperactive PiggyBac comprises a mutation of one or more of amino acids 245, 268, 275, 277, 287, 290, 315, 325, 341, 346, 347, 350, 351, 356, 357, 372, 375, 388, 409, 412, 432, 447, 450, 460, 461, 465, 517, 560, 564, 571, 573, 576, 586, 587, 589, 592, and 594 corresponding to the amino acid number of the hyperactive PiggyBac sequence (SEQ ID NO: 9).

E14. The nucleic acid construct of embodiment E13, wherein the modified hyperactive PiggyBac mutation comprises one or more of R245A, D268N, R275A/R277A, K287A, K290A, K287A/K290A, R315A, G325A, R341A, D346N, N347A, N347S, T350A, S351E, S351P, S351A, K356E, N357A, R372A, K375A, R372A/K375A, R388A, K409A, K412A, K409A/K412A, K432A, D447A, D447N, D450N, R460A, K461A, R460A/K461A, W465A, S517A, T560A, S564P, S571N, S573A, K576A, H586A, I587A, M589V, S592G, or F594L corresponding to the amino acid number of the hyperactive PiggyBac sequence (SEQ ID NO: 9).

E15. The nucleic acid construct of any one of embodiments E12-E14, wherein the modified hyperactive PiggyBac comprises an amino acid sequence at least 85%, at least 90%, or at least 95% identical to the sequence set forth in SEQ ID NO: 10.

E16. The nucleic acid construct of any one of embodiments E1-E15, wherein the linker comprises a XTEN sequence or a GGS sequence.

E17. The nucleic acid construct of any one of embodiments E1-E16, wherein the sequence encoding the linker is between about 9 to about 150 nucleic acids in length.

E18. The nucleic acid construct of any one of embodiments E1-E17, wherein the 3′ end of the first polynucleotide sequence is connected to the 5′ end of the second polynucleotide by the nucleic acid linker.

E19. The nucleic acid construct of any one of embodiments E1-E17, wherein the 3′ end of the second polynucleotide sequence is connected to the 5′ end of the first polynucleotide sequence by the nucleic acid linker.

E20. A vector comprising the nucleic acid construct of any one of embodiments E1-E19, wherein the expression vector suitable for expression in mammalian cells, yeast cells, insect cells, plant cells, fungal cells, or algal cells.

E21. The nucleic acid construct of embodiment E1, wherein:

a) the first polynucleotide sequence encodes a Cas 9 protein; and

b) the second polynucleotide sequence encodes a modified transposase which is a modified hyperactive PiggyBac or functional fragment thereof.

E22. The nucleic acid construct of embodiment E21, wherein the Cas 9 protein is selected from the group consisting of a human Cas 9, a nickase Cas 9, Streptococcus pyogenes Cas9, Staphylococcus aureus Cas9, Cas12a, Cas12b, and a dead Cas 9.

E23. The nucleic acid construct of any one of embodiments E21 or E22, wherein the modified hyperactive PiggyBac comprises a mutation of one or more of amino acids 245, 268, 275, 277, 287, 290, 315, 325, 341, 346, 347, 350, 351, 356, 357, 372, 375, 388, 409, 412, 432, 447, 450, 460, 461, 465, 517, 560, 564, 571, 573, 576, 586, 587, 589, 592, and 594 corresponding to the amino acid number of the hyperactive PiggyBac sequence (SEQ ID NO: 9).

E24. The nucleic acid construct of embodiment E23, wherein the modified hyperactive PiggyBac mutation comprises one or more of R245A, D268N, R275A/R277A, K287A, K290A, K287A/K290A, R315A, G325A, R341A, D346N, N347A, N347S, T350A, S351E, S351P, S351A, K356E, N357A, R372A, K375A, R372A/K375A, R388A, K409A, K412A, K409A/K412A, K432A, D447A, D447N, D450N, R460A, K461A, R460A/K461A, W465A, S517A, T560A, S564P, S571N, S573A, K576A, H586A, I587A, M589V, S592G, or F594L corresponding to the amino acid number of the hyperactive PiggyBac sequence (SEQ ID NO: 9).

E25. The nucleic acid construct of any one of embodiments E21 or E22, wherein the modified hyperactive PiggyBac comprises an amino acid sequence at least 85%, at least 90%, or at least 95% identical to the sequence set forth in SEQ ID NO: 10.

E26. The nucleic acid construct of any one of embodiments E21-E25, wherein the nucleic acid encoding the linker comprises a XTEN sequence or a GGS sequence.

E27. The nucleic acid construct of any one of embodiments E21-E26, wherein the sequence encoding the linker is between 9 to 150 nucleic acids in length.

E28. The nucleic acid construct of any one of embodiments E22-E27, wherein the 3′ end of the second polynucleotide sequence is connected to the 5′ end of the first polynucleotide sequence by the linker.

E29. The nucleic acid construct of embodiment E1, wherein:

a) the first polynucleotide sequence encodes a zinc finger protein; and

b) the second polynucleotide sequence encodes a modified integrase or functional fragment thereof.

E30. The nucleic acid construct of embodiment E29, wherein the zinc finger protein is a C2H2 zinc finger protein.

E31. The nucleic acid construct of any one of embodiments E29 or E30, wherein the modified integrase is a modified human immunodeficiency virus (HIV) integrase or functional fragment thereof.

E32. The nucleic acid construct of embodiment E31, wherein the modified HIV integrase comprises a mutation of one or more of amino acids 10, 13, 64, 94, 116, 117, 119, 120, 122, 124, 128, 152, 168, 170, 185, 231, 264, 266, or 273 corresponding to the amino acid number of the wildtype HIV integrase sequence (SEQ ID NO: 1).

E33. The nucleic acid construct of embodiment E32, wherein the modified HIV integrase mutation comprises one or more of D10K, E13K, D64A, D64E, G94D, G94E, G94R, G94K, D116A, D116E, N117D, N117E, N117R, N117K, S119A, S119P, S119T, S119G, S119D, S119E, S119R, S119K, N120D, N120E, N120R, N120K, T122K, T122I, T122V, T122A, T122R, A124D, A124E, A124R, A124K, A128T, E152A, E152D, Q168L, Q168A, E170G, F185K, R231G, R231K, R231D, R231E, R231S, K264R, K266R, or K273R corresponding to the amino acid number of the wildtype HIV integrase sequence (SEQ ID NO: 1).

E34. The nucleic acid construct of any one of embodiments E31-E33, wherein the modified HIV integrase comprises an amino acid sequence at least 85%, at least 90%, or at least 95% identical to the sequence set forth in SEQ ID NO: 3.

E35. The nucleic acid construct of any one of embodiments E29-E34, wherein the linker comprises a XTEN sequence or a GGS sequence.

E36. The nucleic acid construct of any one of embodiments E29-E35, wherein the sequence encoding the linker is 9 to 150 nucleic acids in length.

E37. The nucleic acid construct of any one of embodiments E29-E37, wherein the 3′ end of the second polynucleotide sequence is connected to the 5′ end of the first polynucleotide sequence by the linker.

E38. A vector comprising the nucleic acid construct of any one of embodiments E21-E37, wherein the expression vector suitable for expression in mammalian cells, yeast cells, insect cells, plant cells, fungal cells, or algal cells.

E39. A host cell comprising the nucleic acid construct or vector of any one of embodiments E1-E38.

E40. A fusion protein comprising:

a first DNA binding protein engineered to bind to a specific genomic DNA sequence in a genome;

a second DNA binding protein which enables insertion of an exogenous nucleic acid into the genome, wherein the second DNA binding protein is an integrase or a transposase which is modified relative to wildtype; and

a linker connecting the first protein and the second protein.

E41. The fusion protein of embodiment E40, wherein the second DNA binding protein is modified to improve specificity of inserting the exogenous nucleic acid into the genome compared to the corresponding wildtype protein.

E42. The fusion protein of any one of embodiments E40 or E41, wherein the exogenous nucleic acid can be up to about 20 kb in length.

E43. The fusion protein of any one of embodiments E40-E42, wherein the first DNA binding protein is selected from the group consisting of a zinc finger protein, a Cas 9 protein, and any variant or functional fragment portion thereof.

E44. The fusion protein of embodiment E43, wherein the Cas 9 protein is selected from the group consisting of a human Cas 9, a nickase Cas 9, Streptococcus pyogenes Cas9, Staphylococcus aureus Cas9, Cas12a, Cas12b, and a dead Cas 9.

E45. The fusion protein of embodiment E43, wherein the zinc finger protein is a C2H2 zinc finger protein.

E46. The fusion protein of any one of embodiments E40-E45, wherein the modified integrase is a modified human immunodeficiency virus (HIV) integrase or functional fragment thereof.

E47. The fusion protein of embodiment E46, wherein the modified HIV integrase comprises a mutation of one or more of amino acids 10, 13, 64, 94, 116, 117, 119, 120, 122, 124, 128, 152, 168, 170, 185, 231, 264, 266, or 273 corresponding to the amino acid number of the wildtype HIV integrase sequence (SEQ ID NO: 1).

E48. The fusion protein of embodiment E47, wherein the modified HIV integrase mutation comprises one or more of D10K, E13K, D64A, D64E, G94D, G94E, G94R, G94K, D116A, D116E, N117D, N117E, N117R, N117K, S119A, S119P, S119T, S119G, S119D, S119E, S119R, S119K, N120D, N120E, N120R, N120K, T122K, T122I, T122V, T122A, T122R, A124D, A124E, A124R, A124K, A128T, E152A, E152D, Q168L, Q168A, E170G, F185K, R231G, R231K, R231D, R231E, R231S, K264R, K266R, or K273R corresponding to the amino acid number of the wildtype HIV integrase sequence (SEQ ID NO: 1).

E49. The fusion protein of any one of embodiments E46-E48, wherein the modified HIV integrase comprises an amino acid sequence at least 85%, at least 90%, or at least 95% identical to the sequence set forth in SEQ ID NO: 3.

E50. The fusion protein of any one of embodiments E40-E45, wherein the modified transposase is selected from the group consisting of a modified Frog Prince, a modified Sleeping Beauty, a modified hyperactive Sleeping Beauty (SB100X), a modified PiggyBac, a modified hyperactive PiggyBac, and any functional fragment thereof.

E51. The fusion protein of embodiment E50, wherein the modified transposase is a modified hyperactive PiggyBac or functional fragment thereof.

E52. The fusion protein of embodiment E51, wherein the modified hyperactive PiggyBac comprises a mutation of one or more of amino acids 245, 268, 275, 277, 287, 290, 315, 325, 341, 346, 347, 350, 351, 356, 357, 372, 375, 388, 409, 412, 432, 447, 450, 460, 461, 465, 517, 560, 564, 571, 573, 576, 586, 587, 589, 592, and 594 corresponding to the amino acid number of the hyperactive PiggyBac sequence (SEQ ID NO: 9).

E53. The fusion protein of embodiment E52, wherein the modified hyperactive PiggyBac mutation comprises one or more of R245A, D268N, R275A/R277A, K287A, K290A, K287A/K290A, R315A, G325A, R341A, D346N, N347A, N347S, T350A, S351E, S351P, S351A, K356E, N357A, R372A, K375A, R372A/K375A, R388A, K409A, K412A, K409A/K412A, K432A, D447A, D447N, D450N, R460A, K461A, R460A/K461A, W465A, S517A, T560A, S564P, S571N, S573A, K576A, H586A, I587A, M589V, S592G, or F594L corresponding to the amino acid number of the hyperactive PiggyBac sequence (SEQ ID NO: 9).

E54. The fusion protein of any one of embodiments E50-E53, wherein the modified hyperactive PiggyBac comprises an amino acid sequence at least 85%, at least 90%, or at least 95% identical to the sequence set forth in SEQ ID NO:10.

E55. The fusion protein of any one of embodiments E40-E54, wherein the linker comprises a XTEN sequence or a GGS sequence.

E56. The fusion protein of any one of embodiments E40-E55, wherein the linker is between 3 to 50 amino acids in length.

E57. The fusion protein of embodiment E40, wherein:

a) the first DNA binding protein is a Cas 9 protein; and

b) the second DNA binding protein is a modified hyperactive PiggyBac or functional fragment thereof.

E58. The fusion protein of embodiment E57, wherein the Cas 9 protein is selected from the group consisting of a human Cas 9, a nickase Cas 9, Streptococcus pyogenes Cas9, Staphylococcus aureus Cas9, Cas12a, Cas12b, and a dead Cas 9.

E59. The fusion protein of any one of embodiments E57 or E58, wherein the modified hyperactive PiggyBac comprises a mutation of one or more of amino acids 245, 268, 275, 277, 287, 290, 315, 325, 341, 346, 347, 350, 351, 356, 357, 372, 375, 388, 409, 412, 432, 447, 450, 460, 461, 465, 517, 560, 564, 571, 573, 576, 586, 587, 589, 592, and 594 corresponding to the amino acid number of the hyperactive PiggyBac sequence (SEQ ID NO: 9).

E60. The fusion protein of embodiment E59, wherein the modified hyperactive PiggyBac mutation comprises one or more of R245A, D268N, R275A/R277A, K287A, K290A, K287A/K290A, R315A, G325A, R341A, D346N, N347A, N347S, T350A, S351E, S351P, S351A, K356E, N357A, R372A, K375A, R372A/K375A, R388A, K409A, K412A, K409A/K412A, K432A, D447A, D447N, D450N, R460A, K461A, R460A/K461A, W465A, S517A, T560A, S564P, S571N, S573A, K576A, H586A, I587A, M589V, S592G, or F594L corresponding to the amino acid number of the hyperactive PiggyBac sequence (SEQ ID NO: 9).

E61. The fusion protein of any one of embodiments E57-E60, wherein the modified hyperactive PiggyBac comprises an amino acid sequence at least 85%, at least 90%, or at least 95% identical to the sequence set forth in SEQ ID NO: 10.

E62. The fusion protein of embodiment E40, wherein:

a) the first DNA binding protein is a zinc finger protein; and

b) the second DNA binding protein is a modified integrase or functional fragment thereof.

E63. The fusion protein of embodiment E62, wherein the zinc finger protein is a C2H2 zinc finger protein.

E64. The fusion protein of any one of embodiments E62 or E63, wherein the modified integrase is a modified human immunodeficiency virus (HIV) integrase or functional fragment thereof.

E65. The fusion protein of embodiment E64, wherein the modified HIV integrase comprises a mutation of one or more of amino acids 10, 13, 64, 94, 116, 117, 119, 120, 122, 124, 128, 152, 168, 170, 185, 231, 264, 266, or 273 corresponding to the amino acid number of the wildtype HIV integrase sequence (SEQ ID NO: 1).

E66. The fusion protein of embodiment E65, wherein the modified HIV integrase mutation comprises one or more of D10K, E13K, D64A, D64E, G94D, G94E, G94R, G94K, D116A, D116E, N117D, N117E, N117R, N117K, S119A, S119P, S119T, S119G, S119D, S119E, S119R, S119K, N120D, N120E, N120R, N120K, T122K, T122I, T122V, T122A, T122R, A124D, A124E, A124R, A124K, A128T, E152A, E152D, Q168L, Q168A, E170G, F185K, R231G, R231K, R231D, R231E, R231S, K264R, K266R, or K273R corresponding to the amino acid number of the wildtype HIV integrase sequence (SEQ ID NO: 1).

E67. The fusion protein of embodiment E62, wherein the modified HIV integrase comprises an amino acid sequence at least 85%, at least 90%, or at least 95% identical to the sequence set forth in SEQ ID NO: 3.

E68. The fusion protein of any one of embodiments E57-E67, wherein the linker comprises a XTEN sequence or a GGS sequence.

E69. The fusion protein of any one of embodiments E57-E68, wherein the linker is 3 to 50 amino acids in length.

E70. The fusion protein of any one of embodiments E40-E69, wherein the 3′ end of the second DNA binding protein is connected to the 5′ end of the first DNA binding protein by the linker.

E71. A lentiviral particle comprising the fusion protein of any one of embodiments E40-E69.

E72. A method of producing a lentiviral particle for gene editing comprising expressing in a host cell:

a) a polynucleotide comprising the nucleic acid construct of any one of embodiments E1-E38; and

b) a polynucleotide that encodes proteins for a lentiviral envelope.

E73. The method of embodiment E72, further comprising expressing c) a polynucleotide sequence comprising the exogenous nucleic acid.

E74. The method of any one of embodiments E72 or E73, wherein the polynucleotide comprising the nucleic acid construct further comprises a nucleic acid sequence encoding lentiviral capsid proteins.

E75. The method of any one of embodiments E72-E74, further comprising recovering the lentiviral particle from the host cell.

E76. The method of any one of embodiments E72-E75, further comprising purifying the lentiviral particle.

E77. A method of inserting an exogenous nucleic acid sequence into genomic DNA of an organism, comprising: administering a lentiviral particle comprising the nucleic acid construct of any of embodiments E1-E38 or a fusion protein of any of embodiments E40-E71 to the organism such that the first and second DNA binding proteins bind to a specific genomic DNA sequence and insert the exogenous nucleic acid into the genomic DNA; wherein the exogenous nucleic acid becomes integrated at the specific genomic DNA sequence.

E78. A method for controlled, site-specific integration of a single copy or multiple copies of an exogenous nucleic acid sequence into a cell, the method comprising:

a) delivering the fusion protein of any one of embodiments E40-E71 to the cell, and

b) delivering the exogenous nucleic acid to the cell;

wherein binding of the fusion protein to the specific genomic DNA sequence in the genome of the cell, results in cleavage of the genome and integration of one or more copies of the exogenous nucleic acid into the genome of the cell; and wherein the fusion protein is delivered to the cell by a lentiviral particle.

E79. A nucleic acid construct comprising:

a) a first polynucleotide sequence comprising a nucleic acid encoding a first DNA binding protein engineered to bind to a specific genomic DNA sequence in a genome; wherein the first DNA binding protein is a zinc finger protein or a Cas9 protein;

b) a second polynucleotide sequence comprising a nucleic acid encoding a second DNA binding protein which enables insertion of an exogenous nucleic acid into a genome, wherein the second DNA binding protein is
(i) a hyperactive PiggyBac transposase, or a modified hyperactive PiggyBac with improved specificity of inserting the exogenous nucleic acid into the genome compared to the hyperactive PiggyBac, or
(ii) a human immunodeficiency virus (HIV) integrase, or a modified HIV integrase with improved specificity of inserting the exogenous nucleic acid into the genome compared to the HIV integrase; and

c) an optional polynucleotide sequence comprising a nucleic acid encoding a linker;

wherein the nucleic acid construct encodes a fusion protein comprising the first DNA binding protein, the second DNA binding protein, and the optional linker between the first DNA binding protein and the second DNA binding protein; and

wherein the fusion protein enables insertion of the exogenous nucleic acid into a specific site of the genome.

E80. The nucleic acid construct of embodiment E79, wherein the Cas9 protein is selected from the group consisting of a human Cas9, a nickase Cas9 and a dead Cas 9.

E81. The nucleic acid construct of embodiment E79, wherein the zinc finger protein is a C2H2 zinc finger protein comprising 6 domains.

E82. The nucleic acid construct of any one of embodiments E79-E81, wherein the linker comprises a XTEN sequence or a GGS sequence.

E83. The nucleic acid construct of any one of embodiments E79-E82, wherein the 3′ end of the first polynucleotide sequence is connected to the 5′ end of the second polynucleotide.

E84. The nucleic acid construct of any one of embodiments E79-E83, wherein: (a) the first DNA binding protein is a Cas 9 protein or a zinc finger protein, and (b) the second DNA binding protein is a hyperactive PiggyBac transposase, or a modified hyperactive PiggyBac with improved specificity of inserting the exogenous nucleic acid into the genome compared to the hyperactive PiggyBac, wherein the nucleic acid construct comprises the (c) polynucleotide sequence comprising a nucleic acid encoding a linker comprising a XTEN sequence or a GGS sequence, and wherein the 3′ end of the first polynucleotide sequence is connected to the 5′ end of the second polynucleotide.

E85. The nucleic acid construct of any one of embodiments E79-E83, wherein: (a) the first DNA binding protein is a Cas 9 protein or a and zinc finger protein, and (b) the second DNA binding protein is a HIV integrase, or a modified HIV integrase with improved specificity of inserting the exogenous nucleic acid into the genome compared to the HIV integrase, wherein the nucleic acid construct comprises the (c) polynucleotide sequence comprising a nucleic acid encoding a linker comprising a XTEN sequence or a GGS sequence, and wherein the 3′ end of the first polynucleotide sequence is connected to the 5′ end of the second polynucleotide.

E86. The nucleic acid construct of any one of embodiments E79-E84, wherein the modified hyperactive PiggyBac transposase comprises a mutation of one or more of amino acids 245, 268, 275, 277, 287, 290, 315, 325, 341, 346, 347, 350, 351, 356, 357, 372, 375, 388, 409, 412, 432, 447, 450, 460, 461, 465, 517, 560, 564, 571, 573, 576, 586, 587, 589, 592, and 594 corresponding to the amino acid sequence SEQ ID NO: 9 of the hyperactive PiggyBac.

E87. The nucleic acid construct of embodiment E86, wherein the modified hyperactive PiggyBac transposase mutation comprises one or more of the amino acid modifications selected from: R245A, D268N, R275A/R277A, K287A, K290A, K287A/K290A, R315A, G325A, R341A, D346N, N347A, N347S, T350A, S351E, S351P, S351A, K356E, N357A, R372A, K375A, R372A/K375A, R388A, K409A, K412A, K409A/K412A, K432A, D447A, D447N, D450N, R460A, K461A, R460A/K461A, W465A, S517A, T560A, S564P, S571N, S573A, K576A, H586A, I587A, M589V, S592G, or F594L corresponding to the amino acid sequence SEQ ID NO: 9 of the hyperactive PiggyBac.

E88. The nucleic acid construct of any one of embodiments E79-E84, wherein the modified hyperactive PiggyBac transposase comprises a mutation of one or more of amino acids 245, 275, 277, 325, 347, 351, 372, 375, 388, 450, 465, 560, 564, 573, 589, 592, 594 corresponding to the amino acid sequence SEQ ID NO: 9 of the hyperactive PiggyBac.

E89. The nucleic acid construct of embodiment E88, wherein the modified hyperactive PiggyBac transposase mutation comprises one or more of the amino acid modifications selected from: R245A, R275A, R277A, R275A/R277A, G325A, N347A, N347S, S351E, S351P, S351A, R372A, K375A, R388A, D450N, W465A, T560A, S564P, S573A, M589V, S592G, or F594L corresponding to the amino acid sequence SEQ ID NO: 9 of the hyperactive PiggyBac.

E90. The nucleic acid construct of embodiment E88, wherein the modified hyperactive PiggyBac transposase comprises the amino acid sequence SEQ ID NO: 9, wherein: amino acid at position 245 is A, amino acid at position 275 is R or A, amino acid at position 277 is R or A, amino acid at position 325 is A or G, amino acid at position 347 is N or A, amino acid at position 351 is E, P or A, amino acid at position 372 is R, amino acid at position 375 is A, amino acid at position 450 is D or N, amino acid at position 465 is W or A, amino acid at position 560 is T or A, amino acid at position 564 is P or S, amino acid at position 573 is S or A, amino acid at position 592 is G or S, and amino acid at position 594 is L or F.

E91. The nucleic acid construct of embodiment E88, wherein the modified hyperactive PiggyBac transposase comprises an amino acid sequence selected from the group consisting of SEQ ID NO: 120, 121, 122, 123, 124, 125, 126, 127, 128, and 129.

E92. The nucleic acid construct of embodiment E88, wherein the modified hyperactive PiggyBac transposase comprises an amino acid sequence having at least 80% identical to a sequence selected from the group consisting of SEQ ID NO: 119, 120, 121, 122, 123, 124, 125, 126, 127, 128 and 129, wherein the modified hyperactive PiggyBac shows higher specificity of DNA integration into a genome compared to hyperactive PiggyBac.

E93. The nucleic acid construct of any one of embodiments E79-E83 or E85, wherein the modified HIV integrase comprises a mutation of one or more of amino acids 10, 13, 64, 94, 116, 117, 119, 120, 122, 124, 128, 152, 168, 170, 185, 231, 264, 266, or 273 corresponding to the amino acid sequence SEQ ID NO: 1 of the wildtype HIV integrase.

E94. The nucleic acid construct of embodiment E93, wherein the modified HIV integrase mutation comprises one or more of D10K, E13K, D64A, D64E, G94D, G94E, G94R, G94K, D116A, D116E, N117D, N117E, N117R, N117K, S119A, S119P, S119T, S119G, S119D, S119E, S119R, S119K, N120D, N120E, N120R, N120K, T122K, T122I, T122V, T122A, T122R, A124D, A124E, A124R, A124K, A128T, E152A, E152D, Q168L, Q168A, E170G, F185K, R231G, R231K, R231D, R231E, R231S, K264R, K266R, or K273R, corresponding to the amino acid sequence SEQ ID NO: 1 of the wildtype HIV integrase.

E95. A vector comprising the nucleic acid construct of any one of embodiments E79-E95, wherein the vector is suitable for expression in mammalian cells, yeast cells, insect cells, plant cells, fungal cells, or algal cells.

E96. A host cell comprising the nucleic acid construct or the vector of any one of embodiments E79-E95.

E97. A fusion protein obtained from the expression of the nucleic acid construct of any one of embodiments E79-E94.

E98. A composition comprising a nucleic acid construct, a vector or a fusion protein of any one of embodiments E79-E95 or E97, and a polynucleotide sequence encoding an exogenous nucleic acid for insertion in a genome, the composition contained in or bound to a packaging vector.

E99. The composition of embodiment E98, wherein the nucleic acid construct is in form of RNA, DNA or protein, and the polynucleotide sequence encoding the exogenous nucleic acid is in form of DNA or RNA.

E100. The composition of any one of embodiments E98-E99, wherein the packaging vector is a nanoparticle or a lentiviral particle.

E101. A method for controlled, site-specific integration of a single copy or multiple copies of an exogenous nucleic acid sequence into a cell, the method comprising: (a) delivering the nucleic acid construct, the vector or the fusion protein of any one of embodiments E79-E95 or E97 to the cell, and (b) delivering the exogenous nucleic acid to the cell; wherein binding of the fusion protein to the specific genomic DNA sequence in the genome of the cell, results in cleavage of the genome and integration of one or more copies of the exogenous nucleic acid into the genome of the cell.

E102. A modified hyperactive PiggyBac transposase comprising the amino acid sequence SEQ ID NO: 9, wherein: amino acid at position 245 is A, amino acid at position 275 is R or A, amino acid at position 277 is R or A, amino acid at position 325 is A or G, amino acid at position 347 is N or A, amino acid at position 351 is E, P or A, amino acid at position 372 is R, amino acid at position 375 is A, amino acid at position 450 is D or N, amino acid at position 465 is W or A, amino acid at position 560 is T or A, amino acid at position 564 is P or S, amino acid at position 573 is S or A, amino acid at position 592 is G or S, and amino acid at position 594 is L or F.

E103. The modified hyperactive PiggyBac transposase of embodiment E102, which comprises an amino acid sequence selected from the group consisting of SEQ ID NO: 120, 121, 122, 123, 124, 125, 126, 127, 128, and 129.

E104. The modified hyperactive PiggyBac transposase of claim E012, which comprises an amino acid sequence having at least 80% identical to a sequence selected from the group consisting of SEQ ID NO: 119, 120, 121, 122, 123, 124, 125, 126, 127, 128 and 129, wherein the modified hyperactive PiggyBac shows higher specificity of DNA integration into a genome compared to hyperactive PiggyBac.

The contents of all cited references (including literature references, patents, patent applications, and websites) that may be cited throughout this application are hereby expressly incorporated by reference in their entirety for any purpose, as are the references cited therein. The following examples are offered by way of illustration and not by way of limitation.

### EXAMPLES

“PB” and “hyPB” are used interchangeably to refer to the hyperactive PiggyBac transposase. Examples 1-3 hereinafter, are related to the generation and performance in terms of targeted integration of constructs of fusion proteins of programmable transposases and Cas9. In Example 1 different DNA constructs of the transposases Hyperactive PiggyBac and Sleeping Beauty fused to different versions of Cas9 were successfully generated, causing integration of the transposon into the genome of the transfected cells. Remarkably, constructs of PiggyBac and Cas9 were able to promote targeted integration into the site of interest of the genome (Example 2). Example 3 provides modified transposases generated to increase the specificity of exogenous nucleic acid sequence insertion into the genome.

### Example 1: DNA Vectors for the Expression of Programmable Transposase Fusion Proteins

This experiment aims to test different configurations of the fusion of Hyperactive PiggyBac transposases (referred herein as hyPB or PB) and Sleeping Beauty (referred herein as SB100x) to nuclease (h), nickase (n) and dead (d) Cas9 for the performance of transposon integration. Programmable transposase fusion proteins were created by incorporating into a pcDNA3.3-TOPO expression vector (Invitrogen plasmid backbone, Addgene Plasmid #41815) the DNA sequences encoding wild-type human Cas9 (hCas9), nickase Cas9 (nCas9), or dead Cas9 (dCas9) (SEQ ID NOs: 64-66, respectively) and hyperactive PiggyBac (PB) or hyperactive Sleeping Beauty (SB100) transposase (SEQ ID NOs: 67-68, respectively). Vectors were created in which the 3′ end of the Cas9 was connected to the 5′ end of each of the transposases by a nucleic acid linker sequence (SEQ ID NO: 48) encoding a GGS linker (hCas9PB, nCas9PB, dCas9PB, hCas9SB, nCas9SB, and dCas9SB). Other vectors were created in which the 3′ end of each of the transposases was connected to the 5′ end of the Cas9 by a nucleic acid linker sequence (SEQ ID NO: 48) encoding a GGS linker (PBhCas9, PBnCas9, PBdCas9, SBhCas9, SBnCas9, and SBdCas9). A summary of the fusion constructs is provided in Table 2.

TABLE 2
List of Programmable Transposase Proteins Generated in Example 1
Programmable Transposase Fusion Proteins

| Cas 9 | Transposase | Orientation | Linker |
|---|---|---|---|
| Human Cas9 | Hyperactive PiggyBac | hCas9-PB | GGS linker |
| Nickase Cas9 | Hyperactive PiggyBac | nCas9-PB | GGS linker |
| Dead Cas9 | Hyperactive PiggyBac | dCas9-PB | GGS linker |
| Human Cas9 | Hyperactive PiggyBac | PB-hCas9 | GGS linker |
| Nickase Cas9 | Hyperactive PiggyBac | PB-nCas9 | GGS linker |
| Dead Cas9 | Hyperactive PiggyBac | PB-dCas9 | GGS linker |
| Human Cas9 | Hyperactive Sleeping Beauty | hCas9-SB | GGS linker |
| Nickase Cas9 | Hyperactive Sleeping Beauty | nCas9-SB | GGS linker |
| Dead Cas9 | Hyperactive Sleeping Beauty | dCas9-SB | GGS linker |
| Human Cas9 | Hyperactive Sleeping Beauty | SB-hCas9 | GGS linker |
| Nickase Cas9 | Hyperactive Sleeping Beauty | SB-nCas9 | GGS linker |
| Dead Cas9 | Hyperactive Sleeping Beauty | SB-dCas9 | GGS linker |

Prior to transfection, frozen HEK293T cells were thawed quickly at 37° C., then resuspended in 5 mL pre-warmed media and pelleted by centrifugation at 1,000 rpm for 4 min. The pellet was resuspended in fresh media and −1.6×10⁶ cells were seeded in a new T75 flask. When cells reached a confluency of 95% they were passaged using trypsin and seeded at a confluency of 40%. Cells were passaged twice before using for experiments.

For transfection experiments, 5×10⁵ HEK293T cells per well were seeded on a multi-well plate with complete DMEM medium (Dulbecco's Modified Eagle Medium (DMEM), supplemented with 10% fetal bovine serum, 2 mM glutamine and 100 U penicillin/0.1 mg/mL streptomycin). Prior to transfection the media was replaced with 2.7 mL fresh complete DMEM medium. Opti-MEM I Reduced Serum Medium was mixed with each combination of plasmids as well as with linear polyethylenimine (PEI 25K) solution 1 mg/mL. A 3:1 ratio of PEI 25K (μg):total DNA (μg) was used. The two solutions were mixed and incubated at room temperature for 15 min. After incubation, 300 μL of the mixture was applied dropwise to the cells. 24 h after transfection, the media was replaced with fresh complete media. Cells were harvested after transfection for flow cytometry or cell sorting and DNA extraction.

HEK293T cells were co-transfected with a plasmid encoding a programmable transposase fusion protein from Table 2, a plasmid encoding the nucleic acid to be integrated, being a RFP (Red Fluorescent Protein) or GFP (Green Fluorescent Protein) transposon, and a guide RNA targeted to the AAVS1 site (Adeno-Associated Virus Integration Site 1) in the human genome. Hyperactive PiggyBac and SB100 were used as a positive control and the transposon alone was used as a negative control for episomal expression detection (i.e. expression from the non-inserted plasmid). Fluorescence was analyzed by flow cytometry until day 14, after which episomal fluorescence could not be detected. Cells were then sorted by GFP expression and two days after sorting, integration of the target DNA was quantified by counting the percent of fluorescent cells.

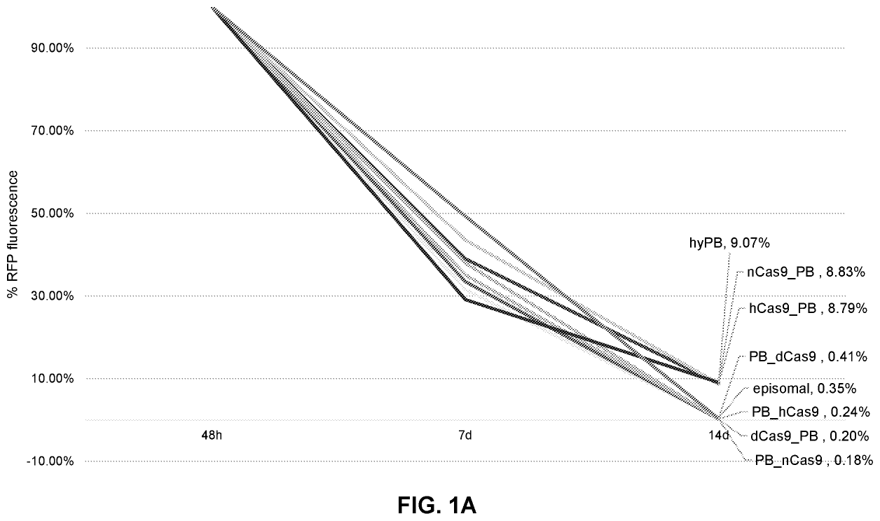

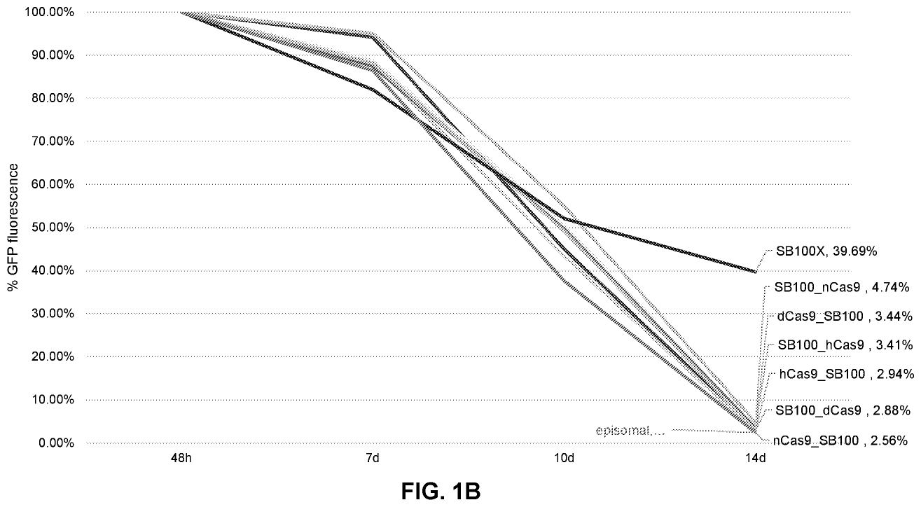

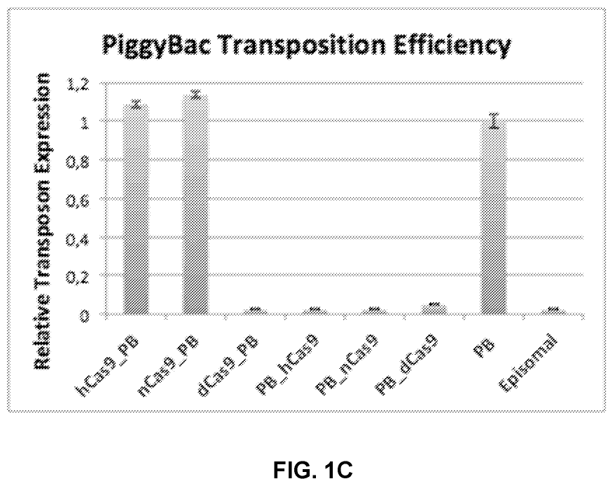

Results and conclusions: The results for the Cas9-PB fusions are shown in FIG. 1A and FIG. 1C; and the results for the Cas9-SB100 fusions are shown in FIG. 1B. Human Cas9 fused to hyperactive PiggyBac (hCas9PB) and nickase Cas9 fused to hyperactive PiggyBac (nCas9PB) increased the percent of fluorescent cells by about 8% compared to the episomal RFP negative control after 14 days (FIG. 1A, 1C). Therefore, said fusion proteins were able to successfully integrate the exogenous DNA into the cell genome. The tested Cas9-Sleeping Beauty fusion proteins were unable to produce more fluorescent cells than the episomal GFP negative control after 14 days (FIG. 1B).

### Example 2: Targeted Transposition Efficiency of Programmable Transposase Fusion Proteins

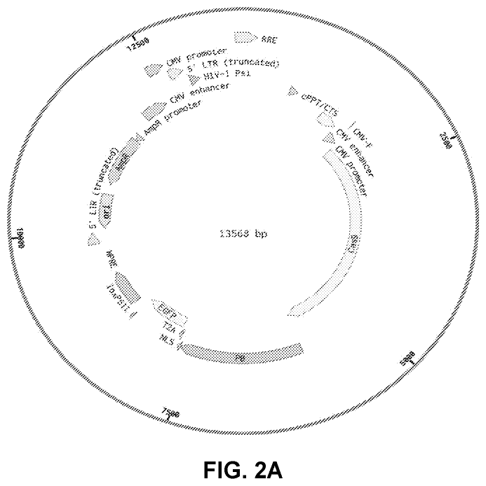

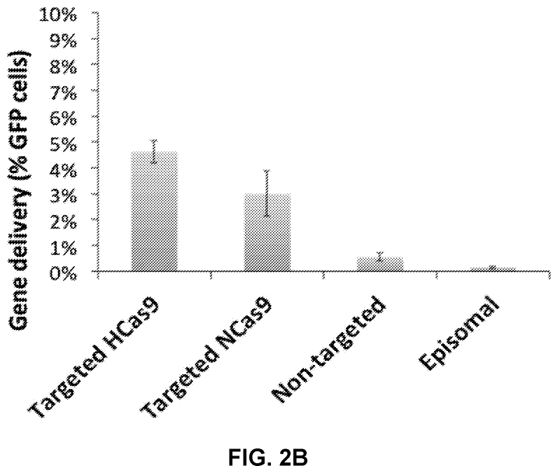

Following the previous example, it was studied whether there was targeted insertion (vs non-targeted) with the configurations that had the best overall insertion in Example 1. To this end, HEK293T were co-transfected using lipofectamine 3000 with a plasmid (pSico) encoding hCas9PB or nCas9PB, a genetrap plasmid encoding a transposon with inverted repeats and a promoter-less GFP, and a guide RNA (gRNA) targeted to the AAVS1 site or a site within the CD46 gene after the promoter on the human genome. The 3′ end of the Cas9 was connected to the 5′ end of the transposase by a linker (SEQ ID NO: 48). An example of the Cas9PB expression vector structure is shown in FIG. 2A. The transposase contained a splicing acceptor and a promoterless GFP in between 3′ and 5′ repeats. The gRNA and Cas9 direct the transposase to integrate the transposon into a promoter region. Using this approach, cells only become fluorescent if the transposon is inserted into the target site.

Results and conclusions: Quantification of the percent of GFP expressing cells showed that the programmable transposase fusion proteins Cas9-PiggyBac (“Targeted HCas9”) and nickase Cas9-PiggyBac (“Targeted NCas9”) had a higher targeted delivery of target DNA compared to controls “Non-targeted” (control for overall insertion (PiggyBac alone)) and “Episomal” (negative control of no-integration (transposon alone)) (FIG. 2B). In this case the increase of 3 times and 4 tines of the signal above background was significant; specially taking into account that not all the cells were efficiently transformed with all the vectors needed for transposon insertion; and the efficiency of random insertion for hyPB in non optimized conditions as the ones used here is 10-15%.

### Example 3: Generation of Modified Hyperactive PiggyBac Transposases

Modified hyperactive PiggyBac transposases were generated to increase the specificity of exogenous nucleic acid sequence insertion into the genome. A list of transposase amino acid mutations is provided in Table 3.

TABLE 3
Mutation Sites for Hyperactive PiggyBac vs Hyperactive PiggyBac SEQ ID NO: 9

| | Wild-type | | |
|---|---|---|---|
| Position | Amino Acid | Mutation | Classifications |
| 245 | R | A | Alanine screening |
| 268 | D | N | Conserved catalytic triad |
| 275 | R | A | Alanine screening |
| 277 | R | A | Alanine screening |
| 275/277 | R/R | A/A | Alanine screening |
| 287 | K | A | Alanine screening: decreased excision |
| 290 | K | A | Alanine screening |
| 287/290 | K/K | A/A | Alanine screening: decreased excision |
| 315 | R | A | Alanine screening: integration competent |
| 325 | G | A | Alignment integrase |
| 341 | R | A | Alanine screening: integration competent |
| 346 | D | N | Conserved catalytic triad |
| 347 | N | A, S | Alignment integrase |
| 350 | T | A | Alignment integrase |
| 351 | S | E, P, A | Mutant comparable with integrase mutations altering |
| | | | target joining --> k351 is integration competent |
| 357 | N | A | Alignment integrase |
| 356 | K | E | Mutant comparable with integrase mutations altering |
| | | | target joining --> k356 is integration competent |
| 372 | R | A | Alanine screening: integration competent |
| 375 | K | A | Alanine screening: integration competent |
| 372/375 | R/K | A/A | Alanine screening |
| 388 | R | A | Alanine screening |
| 409 | K | A | Alanine screening |
| 412 | K | A | Alanine screening |
| 409/412 | K/K | A/A | Alanine screening |
| 432 | K | A | Alanine screening |
| 447 | D | A, N | Conserved Catalytic triad |
| 460 | R | A | Alanine screening: Decreased excision |
| 461 | K | A | Alanine screening: Decreased excision |
| 460/461 | R/K | A/A | Alanine screening: decreased excision |
| 465 | W | A | Alignment integrase |
| 517 | S | N | Int−/Exc+ |
| 560 | T | A | Int−/Exc+ |
| 564 | S | P | Int−/Exc+ |
| 571 | N | S | Int−/Exc+ |
| 573 | S | A | Int−/Exc+ |
| 576 | K | A | Well conserved residues, other important functions not |
| | | | DNA binding as it's a flexible tail. |
| 586 | H | X | Zn²⁺ ligand C-terminus |
| 587 | I | A | Well conserved residues, other important functions not |
| | | | DNA binding as it's a flexible tail. |
| 589 | M | V | Int−/Exc+ |
| 592 | S | G | Int−/Exc+ |
| 594 | F | L | Int−/Exc+ |

In Example 4 hereinafter, several constructs were generated with the aim that Zinc Finger Protein (ZFP) were able to bind to a chromosomal target site for the insertion of the gene of interest. ZFP constitutes an alternative to Cas9 as DNA binding protein. Examples 5-13 are generally related to the generation and performance in terms of targeted integration of constructs of fusion proteins of HIV-1 integrase and Cas9/ZFP. Particularly, in Example 5 fusion proteins of ZFP and Integrase were generated. Examples 6-10 provide different integrase defective packaging systems (i.e. non-integrative vectors) created to serve as a basis for in vitro studies to demonstrate the recovery of the integration function with the integrase fusion proteins created in Example 11. In Example 12 it is observed that the targeted integrase fusion proteins increased the percentage of targeted insertion.

### Example 4: Generation of a Targeted Zinc Finger Protein (ZFP)

The aim was generating several ZFPs that bind to a chromosomal target site for the insertion of the gene of interest. A 6 domain zinc finger protein was generated to target the AAVS1 site (SEQ ID NO: 40) on the human genome. The target DNA sequences and corresponding ZFP helices are shown in Table 4. A construct encoding the target sites and ZFP was prepared (AAVS1-6d-ZFP). The nucleic acid and amino acid sequences encoding the ZFP are SEQ ID NOs: 32 and 33, respectively.

TABLE 4
List of AAVS1 Target Sites and Corresponding
ZFP helices

| Finger | Triplet | Helix | SEQ ID NO |
|---|---|---|---|
| 1 | AGC | ERSHLRE | 41 |
| 2 | CAG | RADNLTE | 42 |
| 3 | CGT | SRRTCRA | 43 |
| 4 | CCG | RNDTLTE | 44 |
| 5 | CGG | RSDKLTE | 45 |
| 6 | AGA | QLAHLRA | 46 |

### Example 5: Generation of a ZFP-Integrase Fusion Protein

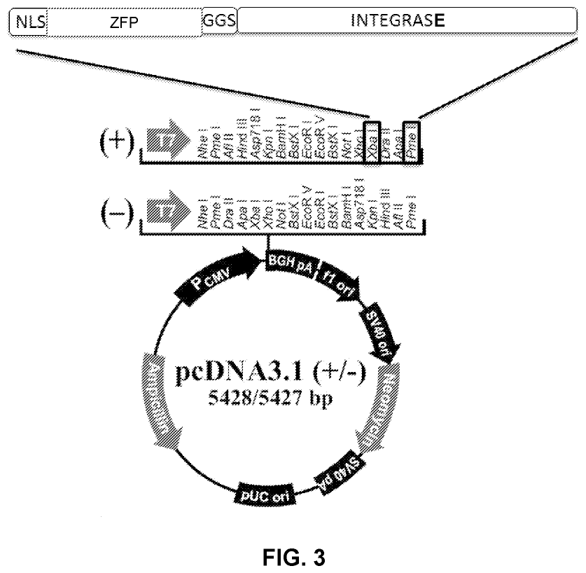

Integrase fusion proteins with ZFPs having 6 domains (effectively sequence specific) were generated. To generate a site specific integrase, the ZFP generated in Example 4 (AAVS1-6d-ZFP) was cloned into a pcDNA3.1 expression vector along with HIV-1 integrase (SEQ ID NO: 1) (pZFP-AAVS1-6d-IN). The sequence encoding the fusion protein contains a N-terminal nuclear localization signal (SEQ ID NO: 47) and a GGS linker sequence (SEQ ID NO: 48) between the ZFP and integrase (FIG. 3).

Additional integrase fusion vectors were generated such as pZFP-TRCa-IN (including SEQ ID NO: 38, targeting TRCa locus) and pZFP-AAVs1-TEX-IN (including a TEX linker (SEQ ID NO: 61)), which were prepared using similar methods.

### Example 6: Generation of DNA Vectors with Defective Integrase

Integrase defective packaging systems were created to serve as a basis for in vitro studies using an engineered integrase. Defective integrase constructs were created from the non-integrative packing plasmid (NILV) psPAX2. The psPAX2 plasmids have a single N64D mutation and double N64D/N116D mutations. A deleted integrase (ΔIN) plasmid was created which lacked the entire integrase coding region. A non-coding plasmid was created which contained a stop codon before the integrase coding sequence (Example 8 hereinafter). Plasmids containing truncated integrases were created, including a construct containing the C-terminal domain and DNA binding domain without the cPPT/CTS (Example 10 hereinafter). General cloning protocols were followed as briefly described below.

### KAPA HiFi HotStart Protocol

For PCR experiments employing KAPA HiFi HotStart, the PCR reaction mixture was prepared according to the KAPA HiFi PCR Kit manufacturer's protocol. KAPA HIFi PCR reactions were performed with the Mastercycler Pro.

### Plasmid DNA Extraction

Plasmid DNA was extracted using the QIAprep Spin Miniprep Kit according to the manufacturer's protocol. Bacterial cultures were harvested by centrifugation at 5,000 rpm for 3 min. The pellet of cells was resuspended in 250 μL of Buffer P1 and mixed by inverting the tube 4-6 times with 250 μL of Buffer P2. 350 μL of Buffer N3 was added and mixed by inverting the tube. The Eppendorf tube was centrifuged for 10 min at 12,000 rpm to remove the cell debris and chromosomal DNA. The supernatant was transferred to the supplied QIAprep spin column and centrifuged for 1 min (12,000 rpm). The sample was washed twice with 0.5 mL of Buffer PB and 0.75 ml of Buffer PE and each time centrifuged for 1 min at 12,000 rpm. An additional centrifugation for 1 min at 12000 rpm removed the residual wash solution buffer. QIAprep spin column was transferred to a new 1.5 ml microcentrifuge tube and 50 μL of water was added to elute the plasmid by letting the tube stand for 1 min and following centrifuging 1 min at 12,000 rpm. Concentration was measured with a NanoDrop One.

### Isolation and Purification of Plasmid DNA

Bacterial strains (DH5a or DH10B) containing the desired plasmid were grown overnight in LB media containing 100 μg/mL carbenicillin. Plasmids were isolated using either the plasmid mini or maxi kits from NZYTech, according to the manufacturer's protocol. Plasmids were eluted in either 30 μL (miniprep) or 500 μL (maxiprep) of 65° C. hot water. Plasmids were stored at −20° C. For PCR purification, the reaction mix was processed using the PCR purification kit. The DNA was eluted in 30 μL, 65° C. hot water.

### DNA Gel Electrophoresis

Agarose was dissolved in 100 mL TAE-Buffer by boiling. The liquid gels were supplemented with 4 μL greensafe per 100 mL agarose solution and poured into a tray. To visualize DNA preparations, the DNA was mixed with 6× loading dye and loaded onto a 1% agarose gel. In addition, one chamber was loaded with 1 μL gene ladder per 1 mm gel lane. Gels were run for 1.5 hr at 100V and visualized using a transilluminator.

### Transformation

For transformation experiments with DH5a, plasmids were transformed into 50 μL DH5α cells according to the manufacturer's protocol. After recovering in s.o.c. media, the bacteria were pelleted at 15,000 g for 30 sec and resuspended in 50 μl LB media. The cells were spread on a LB-Agar plate containing 100 μg/mL carbenicillin and incubated at 37° C. overnight. Cultures were picked and inoculated overnight in LB media containing 100 μg/mL carbenicillin. The liquid culture was either used for plasmid isolation again or for a glycerol stock. For the glycerol stock, 500 μL liquid culture was mixed with 500 μL 50% glycerol and stored at −80° C.

For transformation experiments with XL-10 Gold ultracompetent cells, cells were first thawed on ice and 45 μL of cells were added to a pre-chilled 14 mL Falcon polypropylene round-bottom tube. 2 μL of the β-ME mix provided with the kit was added to the cells. The contents of the tube were swirled gently and the cells were incubated on ice for 10 min (swirling every 2 min). 1.5 μL of the DpnI treated DNA was added to an aliquot of cells, mixed, and incubated on ice for 30 min. The cell/DNA mixture was heat-pulsed in the tube at 42° C. for 30 sec. The tubes were then incubated on ice for 2 min. Then 0.5 mL of preheated (42° C.) NZY+ broth was added to each tube and then incubated at 37° C. for 1 hr with shaking at 225-250 rpm. The mixture was then plated onto agar plates containing the appropriate antibiotic for the plasmid vector. Five colonies were selected for DNA extraction and the sequences were verified. Colony 1 was selected and maintained.

### Example 7: Generation of Non-Integrating Vectors Containing PPT or a ZFP-Modified Integrase Fusion Protein

To create an integrase (IN) defective but otherwise fully functional psPAX2 plasmid, the polypyrimidine tract domain (PPT) (SEQ ID NO: 74, which is crucial for the subsequent double-stranded cDNA formation of all retroviral RNA genomes such as lentivirus), was cloned into a psPAX2 vector that did not contain an integrase (psPAX2-ΔIN). The synthetic zinc finger construct targeting AAVS1 generated in Example 4 (AAVS1-6d-ZFP-IN) was cloned into psPAX2-ΔIN. Two different forward primers and the same reverse primer (SEQ ID NO: 75-77) were designed for PPT with and without a stop codon (IN+PPT and IN+PPT(STOP)). Two different forward primers (SEQ ID NO: 78-80) and the same reverse primer were designed for AVS1-6d-ZFP-IN with and without a nuclear localization signal (AAVS1-6d-ZFP-IN and AAVS1-6d-ZFP-IN(-NLS)). Inserts were amplified by PCR using Kappa standard conditions, an annealing temperature of 62° C., and extension times of 40 sec for PPT and 90 sec for AAVS1-6d-ZFP-IN. PCR products were separated by gel electrophoresis.

The amplified products were purified and an assembly protocol was performed with a ratio of 1:2.5 backbone:insert and 5 cycles. 50 μL of competent cells were transformed with 4 μL ligation product and 60% of competent cells were seeded onto carbenicillin plates. Initial verification of the colonies was determined by restriction digestion and DNA gel electrophoresis. The following colonies were picked: colonies 1 and 2 (IN+PPT F1+R, AAVS1-6d-ZFP-IN F1+R, AAVS1-6d-ZFP-IN(-NLS) F2+R) and colonies 7 and 8 (IN+PPT(STOP) F2+R). To further verify the colonies contained the correct insert, colony PCR was performed with 4 mM Mg, 62-STS, and NEB standard taq.

### Example 8: Generation of Non-Integrating Vectors by Insertion of a Stop Codon

A non-integrating vector was generated by insertion of a stop coding prior to the integrase open reading frame (psPAX2-TAA-IN). psPAX2-TAA-IN was generated by site-directed mutagenesis by adding two stop codons after the protease cut site at the beginning of the integrase. PCR conditions for site-directed mutagenesis were used to create psPAX2-TAA-IN.

After PCR, the reaction tubes were placed on ice for 2 minutes to cool. Then 1 μL DpnI was added directly to each amplification reaction and incubated at 37° C. for 5 min to digest the parental (nonmutated) double stranded DNA.

Plasmid DNA was digested to confirm that site-directed mutagenesis did not produce any unwanted modifications. Digestion of psPAX2 and psPAX2-TAA-IN with SacI and AgeI should result in three bands of 7,500, 1,900, and 1,300 bp. Digestion of psPax2-ΔIN with SacI and AgeI should result in three bands of 7,500, 1,300, and 800 bp. The digestion reaction was performed and digestion resulted in the correct banding pattern.

### Example 9: Reconstitution of Wild-Type Integrase into an Integrase Defective Vector

The aim was to develop the methodology to see whether a non-integrative vector could recover the insertion activity with the expression of different forms of the integrase fusion proteins. To confirm that psPAX2-ΔIN was fully functional, an integrase was added into the vector using Gibson Assembly. Additionally, to test if the assembly sites are good for cloning the fusion “IN”, a wt-IN was cloned with the additional N-term of IN that is in the backbone before the site (with the Leu that should not be there). This was also done with an extra protease target sequence to avoid this fake N-terminal domain. A PCR reaction was performed to amplify IN-1, IN-2, and IN-3 fragments.

PCR amplified products were separated by DNA gel electrophoresis. Amplified bands were purified and assembly was performed with a ratio of 1:2.5 backbone:insert and 5 cycles at 37° C. 50 μL competent cells were transformed with 4 μL of ligation product and seeded on carbenicillin plates.

To generate the construct containing IN-3, Gibson assembly was performed following the standard protocol for Gibson Assembly HiFi 1 step kit (using the CRG MA/I) (SGI-DNA, Inc., www.sgidna.com/products/gibson-assembly-reagents/). Reaction mixtures were created and assembled for 1 hr at 50° C. Competent cells were transformed with 2 μL of the reaction mixture.

50 μL of competent cells were transformed with 2 μL of ligation product and seeded on carbenicillin plates.

### Example 10: Generation of Non-Integrating Vectors Containing a C-Terminal Domain Truncated Integrase

C-terminal domain (CTD) (nucleic acids 83-118 of SEQ ID NO: 74) and CppT+CTD (SEQ ID NO: 74) integrase fragments were cloned into the psPAX2 vector.

PCR amplified products were separated by DNA gel electrophoresis. Ligation of CppT+CTD was performed using conditions as used in Example 9.

Ligation was performed for 5 cycles at 65° C. and the ligation product was transformed. No colonies grew. Ligation and transformation was performed again and three colonies were verified by sequencing with an IN-fw primer (SEQ ID NO: 81).

### Example 11: Generation of Integrase Fusion Proteins

Targeted integrase fusion proteins were created by incorporating into a pcDNA3.3 expression vector, HIV-1 integrase and either the targeted ZFP or human Cas9. One vector was created in which the 3′ end of the ZFP or Cas9 was connected to the 5′ end of the integrase by a nucleic acid linker. A second vector was created in which the 3′ end of the integrase was connected to the 5′ end of the ZFP or Cas9 by a nucleic acid linker. The linkers used were XTEN or GGS in the range of 13, 16, 19, 22, 25, or 28 amino acids in length. The ZFP-integrase fusion protein was engineered to target the AAVS1 site or the T-cell receptor alpha (TCRa) locus in the human genome. The Cas9-integrase fusion protein was used in combination with guide RNAs targeting the AAVS1 site or the TCRa locus in the human genome. A list of modified integrase fusion proteins is shown in Table 5.

TABLE 5
List of Modified Integrase Fusion Proteins Generated in Example 11

| | DNA Binding | | | |
|---|---|---|---|---|
| Integrase | Protein | Target Site | Linker | Orientation |
| HIV-1 integrase | Zinc Finger Protein | AAVS1 | XTEN or GGS | ZFP-Integrase |
| | | | 12, 16, 19, 22, 25, or | |
| | | | 28 amino acids long | |
| HIV-1 integrase | Zinc Finger Protein | AAVS1 | GGS | Integrase-ZFP |
| HIV-1 integrase | Zinc Finger Protein | TCRa | XTEN or GGS | ZFP-Integrase |
| | | | 12, 16, 19, 22, 25, or | |
| | | | 28 amino acids long | |
| HIV-1 integrase | Zinc Finger Protein | TCRa | GGS | Integrase-ZFP |
| HIV-1 integrase | Zinc Finger Protein | CCR5 | GGS | ZFP-Integrase |
| HIV-1 integrase | Cas9 | AAVS1 | XTEN | Cas9-Integrase |
| HIV-1 integrase | Cas9 | AAVS1 | GGS | Integrase-Cas9 |
| HIV-1 integrase | Cas9 | TCRa | XTEN | Cas9-Integrase |
| HIV-1 integrase | Cas9 | TCRa | XTEN | Integrase-Cas9 |

### Example 12: Cys and Trans Complementation of Integrase Defective Lentivirus with Targeted Integrase Fusion Proteins

The targeted integrase fusion proteins of Example 11 were used to complement the lack of integration capacity of the non-integrative lentivirus, expressing an IN with two mutations in the catalytic domain (D64V/D116N). For this experiment, the targeted integrase fusion proteins were cloned into a pcDNA3.1 vector. Lentivirus was produced by co-transfecting cells with pSICO (GFP expression payload), pmd2.g (VSVG for envelope expression), pax2 (containing packaging proteins and integrase) or NILV-pax2 (containing packaging proteins), and the pcDNA3.1 vector containing either wild-type integrase or the targeted integrases (Table 6).

TABLE 6
Conditions for Complementation of Integrase Defective
Lentivirus with Targeted Integrase Fusion Proteins

| Packages/ | | | | | NILV + | NILV + |
|---|---|---|---|---|---|---|
| Plasmids | LV | LVO | NILV | NILV + IN | ZP-IN(AAVS1) | Cas9_IN(AAVS1) |
| pSICO | + | | + | + | + | + |
| psPAX2 | + | + | | | | |
| psPAX2-NILV | | | + | + | + | + |
| pMD2.G | + | + | + | + | + | + |
| pHIV1-IN | | | | + | | |
| pZFP-AAVS1-R | | | | | + | |
| pCas9_IN(AAVS1) | | | | | | + |

6×10⁵ HEK293T cells (passage 8) per well were seeded onto a 6-well plate and incubated overnight. 5 hours before starting virus production, the media was changed to 1.7 mL media containing 1:1000 chloroquine diphosphate (CD; Stock=25 mM). The plasmids were infected in a molar ratio 1.6:1.32:0.72:3.32 (pSICO:pax2:VSVG:wtIN-rescue). PEI (polyethylenimine; stock=1 mg/mL) was used as a transfection reagent, while 3 μL PEI was used for 1 μg total DNA used for transfection. DNA was diluted in 83 μL Opti-MEM and 83 μL PEI, mixed, and incubated for 15-20 min at room temperature. Each transfection mix was added dropwise to the cells with the CD-media. Cells were incubated overnight and media was replaced the next day with 2.5 mL fresh media. The next day, the supernatant of the cells was centrifuged for 5 min at 1,000 rpm and passed through a 4504 filter. The supernatant containing virus was stored at −80° C.

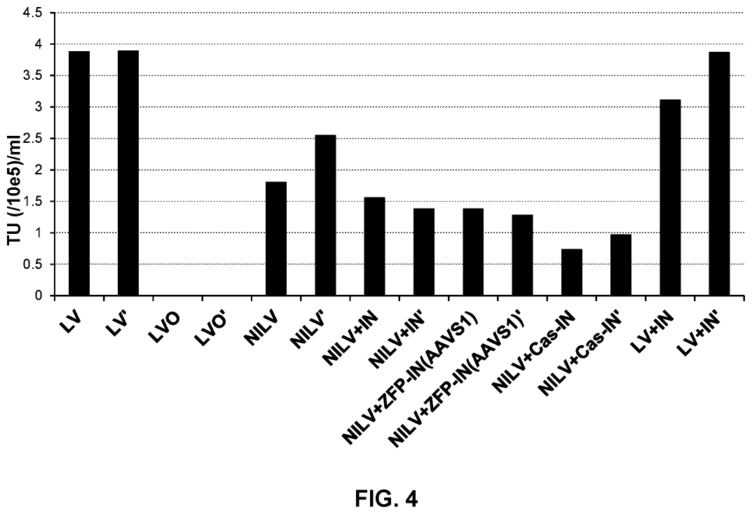

The first step was to confirm that the different lentivirus packages maintained the capacity of infecting cells independently from their content. To determine virus titer, 75,000 HEK293T cells per well were seeded on a 6-well plate. Cells were infected with a mix of 1 mL media containing 1:100 polybrene and 500 μL previously produced virus supernatant (1:3). The media was changed the next day. The following day, the media was aspirated and cells were detached using 200 μL trypsin. The reaction was stopped by added 800 μL normal media and analyzed by flow cytometry. Virus titer was quantified for wild-type integrase lentivirus (LV), empty viral particles (LVO), non-integrative lentivirus (NILV), non-integrative lentivirus with wild-type integrase (NILV+IN), non-integrative lentivirus with ZFP-integrase fusion protein (NILV+ZP-IN(AAVS1)), non-integrative lentivirus with Cas9-integrase fusion protein (NILV+Cas-IN), and wild-type integrase lentivirus with wild-type integrase (LV+IN). LV and LVO were used as positive and negative controls, respectively. HEK293T cells were infected and virus titer was quantified by counting the number of GFP positive cells (FIG. 4). Results: Virus titer was within the same order of magnitude for all conditions.

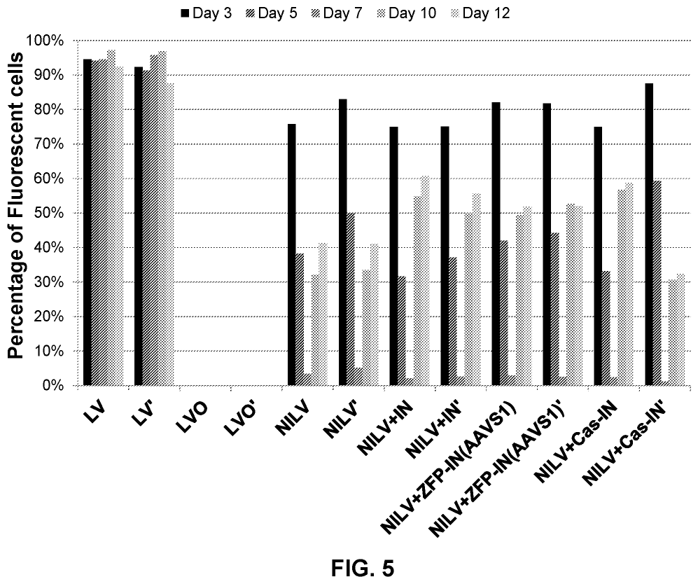

Next, the overall integrative capacity of the targeted integrase fusion proteins was determined by flow cytometry and next-generation sequencing of the target insert. HEK293T cells were infected with the same multiplicity of infection for all conditions and GFP fluorescence was monitored at 3, 5, 7, 10, and 12 days post-infection. Seven days post-infection, cells were sorted by GFP expression. Results: At day 12, cells infected with non-complemented NILV had a smaller percentage of GFP expressing cells (FIG. 5) indicating a reduction on the viral production capacity.

To assess the targeted integration capacity of the integrase fusion proteins tested, genomic DNA was extracted according to the DNeasy Blood and Tissue Kit Protocol (Qiagen) at day 12. Cell cultures were harvested by centrifugation at 190 rpm for 5 min (maximum 5×10⁵). The pellet was dissolved in 200 μL PBS (phosphate buffered saline). 20 μL Proteinase K was added together with 200 μL of Buffer AL. After vortexing, the samples were incubated at 56° C. for 10 min. After the addition of 200 μL ethanol (96-100%) and brief vortexing, the mixture was transferred to a DNeasy Mini spin column, placed into a 3 mL collection tube, and centrifuged at 8,000 rpm for 1 min. The spin column was moved to a new 2 mL collection tube and 500 μL of Buffer AW1 was added. Tubes were centrifuged at 8,000 rpm for 1 min. This washing step was repeated for Buffer AW2 (centrifugation of 3 min). Then, the spin was transferred to a new 1.5 mL microcentrifuge tube and 200 μL of Buffer AE was added to the center of the spin column membrane to elute the DNA by letting the tube stand for 1 min and it was followed by a centrifugation of 1 min at 8,000 rpm. Genomic DNA concentration was quantified with a NanoDrop One.

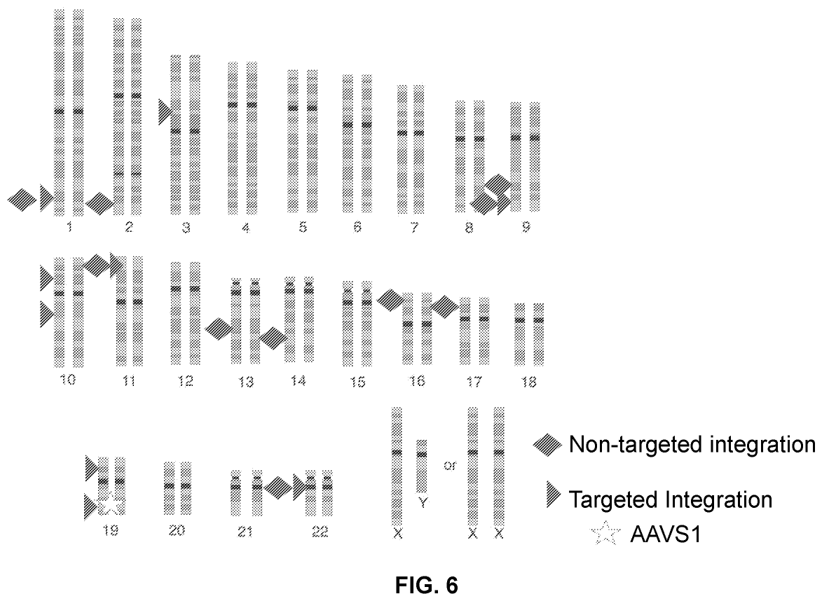

Inverse cloning was performed with oligos specific for viral inserted LTR. Next generation targeted sequencing was analyzed by the following parameters: filter the read such as both R1 and R2 contain the corresponding sequencing primer, restrict the checking to the leftmost bases (as much bp as the primer has), allow for 2 mismatches, trim the primer sequences (SEQ ID NO: 82-89), filter the reads such as both R1 and R2 contain the corresponding LTR bases, restrict the checking to the leftmost 5 bases of the read, use the 5 first LTR bases (following the sequencing primer) with K=3 (means that for the sequence ACTGA will check the presence on the read of one of the following k-mers: ACT, CTG, TGA), allow for 2 mismatches, trim the corresponding LTR basepairs, map reads to the reference genome, retrieve the coverage (number of reads per insertion site), divide by 2 the regions where there is R1 and R2 overlapping, add only one of the insertion sites if there is no R1 and R2 overlapping, apply a coverage threshold, calculate coverage per each 10 mb of the reference genome and perform the coverage plots, calculate the percentage of coverage for each insertion site. Results: The targeted integrase fusion proteins increased the coverage of the AAVS1 site and the percentage of targeted insertion (Table 7 and FIG. 6). As seen in Table 7, there are more numbers of reads on the target site when the insertion is done by the integrase fusion proteins; compared to IN WT, which is indicative of targeted insertion. FIG. 6 is a representation of the most common targeted sites in the genome for IN and ZFP IN (AAVS1); denoting the presence of targeted insertion only in the fusion condition.

TABLE 7
AAVS1 number of reads and Percent of Targeted Insertion
by the Targeted Integrase Fusion Proteins

| | Number of reads | % Targeted |
|---|---|---|
| Sample | on AAVS1 | Insertion |
| Native (LV) | 6 | 0 |
| Non-Integrative + Native (NILV + IN) | 3 | 0 |
| Non-Integrative (NILV) + ZFP-IN(AAVS1) | 216 | 30 |
| Non-Integrative (NILV) + Cas9-IN(AAVS1) | 71 | 10 |

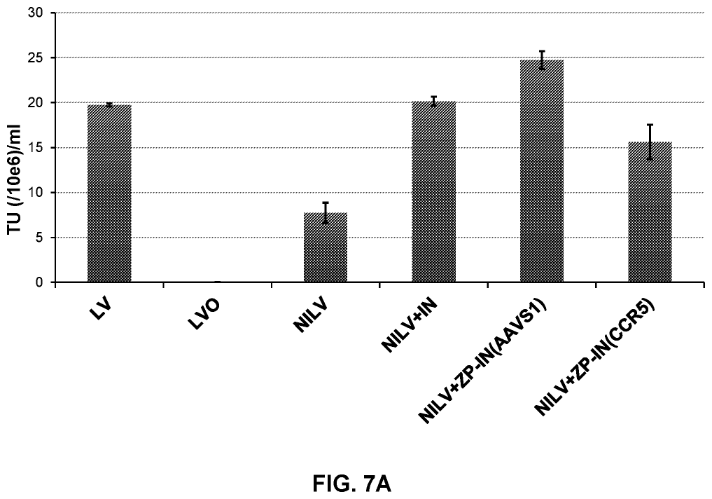

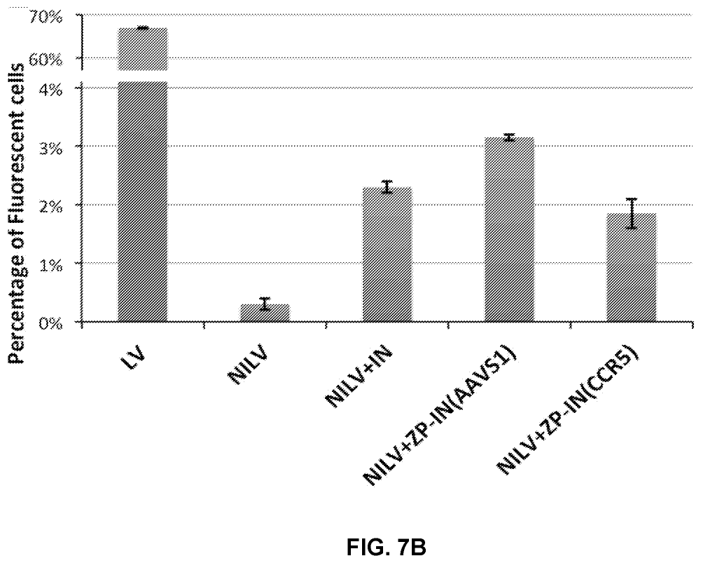

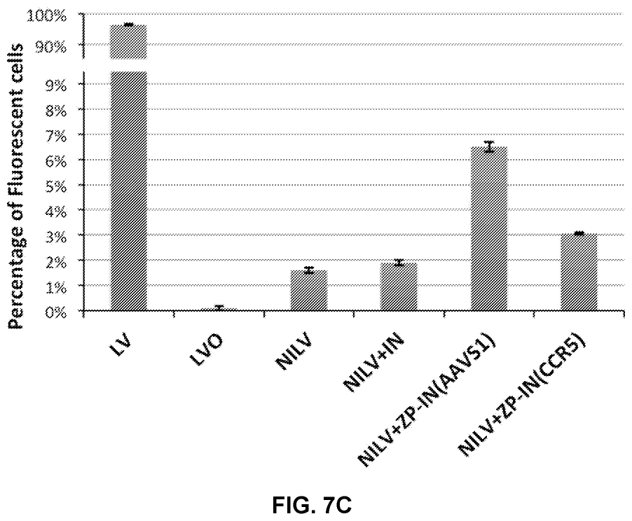

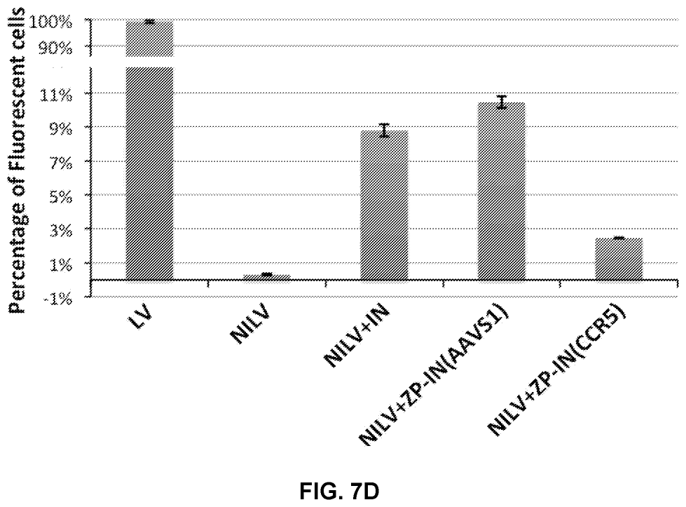

A second ZFP was also generated to target a nucleic acid segment within the CCR5 gene. This zinc-finger protein was fused to HIV-1 integrase to create a CCR5 targeted integrase. Lentivirus containing this ZFP-IN was produced as described above and transduced into HEK293T cells (NILV+ZP-IN(CCR5)) (Table 6). Results: The virus titer of NILV+ZP-IN(CCR5) was similar to LV and NILV+IN (FIG. 7A). This construct was able to produce viral particles with the same efficiency as the other ZFP IN fusion tested (FIGS. 7B and C). Its capacity to integrate DNA in a site specific manner was not tested for CCR5.

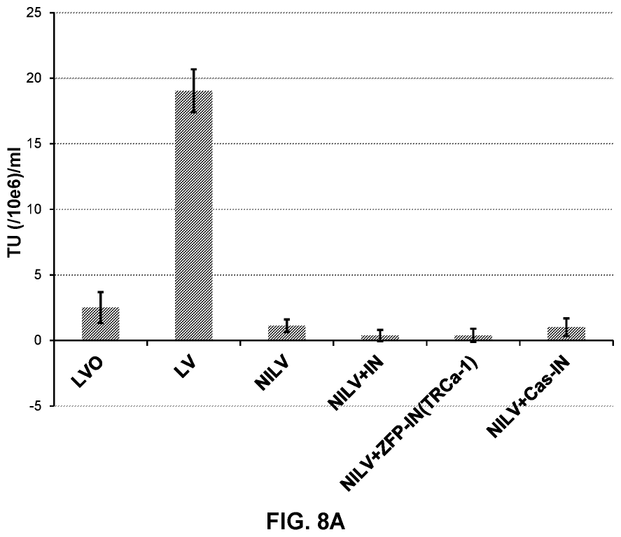

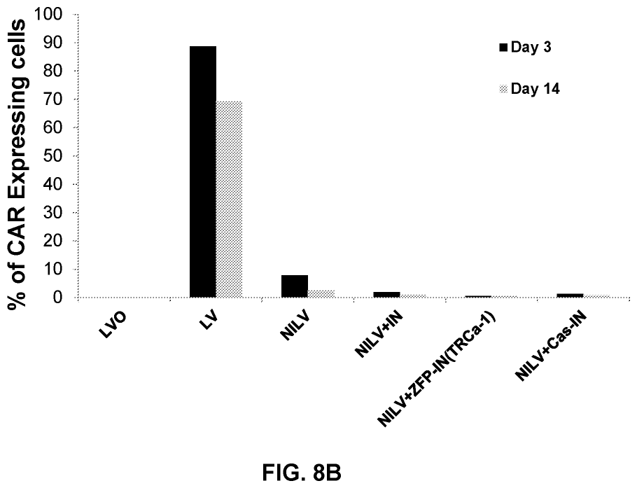

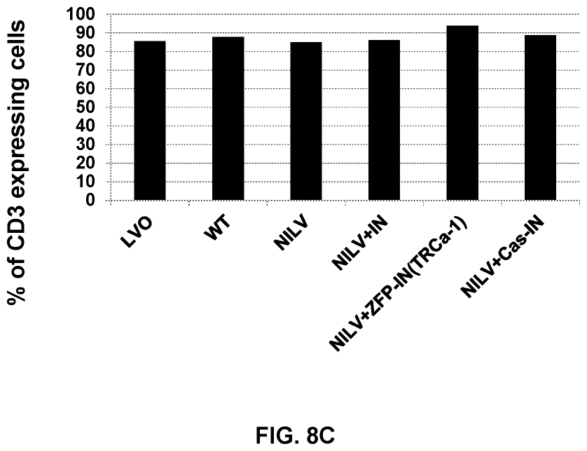

In another experiment, the newly cloned expression vectors for Fusion ZFP-IN with 6d targeting TCRa locus and gRNA targeting the same site (See Example 11). The assay tested whether wild-type integrase and ZFP-integrase fusion can complement the NILV capacity and promote selective integration of a CAR-T cassette. Jurkat cells were infected at the same multiplicity of infection for all TCRa targeted insertion particles. In this experiment, virus particles were loaded with a CD19 CAR-T cassette which would result in the loss of CD3 (encoded by TCRa gene) protein expression after targeted insertion. The percentage of CD19 positive and CD3 negative cells were tracked over time. The lentivirus titer is shown in FIG. 8A and the % of CAR expressing cells at day 3 and day 14 is shown in FIG. 8B. The % of CD3 expression cells is shown in FIG. 8C. This indicates that the transcomplementation did not work in the context of this cell line, in the absence of VPR, an important factor for efficient IN transcomplementation.

### Example 13: Generation of a Modified Integrase by Site-Directed Mutagenesis and Saturation Mutagenesis

Modified HIV-1 integrases were generated by site-directed mutagenesis and saturation mutagenesis. For site-directed mutagenesis, a modified HIV-1 integrase will be created by mutating amino acids by site-directed mutagenesis. The QuikChange Lightning Multi Site-Directed Mutagenesis Kit will be used and primers were designed according to the manufacturer's recommendations (SEQ ID NO: 90-97). The plasmid to be mutated is about 7,000 bp. About 5 colonies per approach will be screened by sequencing. Glycerol stocks of colonies will be prepared containing the desired plasmids.

Saturation mutagenesis of the HIV-1 integrase will be performed to generate a large combinatorial library of different HIV-1 integrase molecules. The protocol was adopted from Cornell et al., (Biochemistry, 57(5)604-613, 2018). Several forward primers containing a degenerated NNS sequence at the mutational site will be used and one reverse primer in one PCR reaction (SEQ ID NO: 90-97). The whole plasmid will be amplified to generate mutated integrase molecules. The primers will be optimized to a melting temperature of 68° C. During the cycles the annealing temperature will be increased by 0.3° C. per cycle. A list of amino acid mutation is provided in Table 8.

TABLE 8
Sites of Mutation of HIV-1 Integrase vs Wildtype HIV-1 integrase
aa sequence NC_001802.1-NP_705928 (SEQ ID NO: 1)

| Amino | | Amino | |
|---|---|---|---|
| Acid | Wildtype | Acid | |
| Position | Amino Acid | Mutation | Classifications |
| 10 | D | K | Residue critical for retroviral integrative |
| | | | recombination in a region that is highly conserved |
| 13 | E | K | Residue critical for retroviral integrative |
| | | | recombination in a region that is highly conserved |
| 64 | D | A, E | Residue critical for retroviral integrative |
| | | | recombination in a region that is highly conserved |
| 94 | G | D, E | Negative amino acids that might impair DNA |
| | | | binding (proven for 231E) |
| 94 | G | R, K | Positive amino acids that might enhance DNA |
| | | | binding |
| 116 | D | A, E | Residue critical for retroviral integrative |
| | | | recombination in a region that is highly conserved |
| 117 | N | D, E | Negative amino acids that might impair DNA |
| | | | binding (proven for 231E) |
| 117 | N | R, K | Positive amino acids that might enhance DNA |
| | | | binding |
| 119 | S | A, P, T, G | Positions that are found in other integrase variants |
| | | | (taken from an alignment from Gijbers et al 2014) |
| 119 | S | D, E | Negative amino acids that might impair DNA |
| | | | binding (proven for 231E) |
| 119 | S | R, K | Positive amino acids that might enhance DNA |
| | | | binding |
| 120 | N | D, E | Negative amino acids that might impair DNA |
| | | | binding (proven for 231E) |
| 120 | N | R, K | Positive amino acids that might enhance DNA |
| | | | binding |
| 122 | T | K, I, V, A | Positions that are found in other integrase variants |
| | | | (taken from an alignment from Gijbers et al 2014) |
| 122 | T | R | Positive amino acids that might enhance DNA |
| | | | binding |
| 124 | A | D, E | Negative amino acids that might impair DNA |
| | | | binding (proven for 231E) |
| 124 | A | R, K | Positive amino acids that might enhance DNA |
| | | | binding |
| 128 | A | T | Residue critical for retroviral integrative |
| | | | recombination in a region that is highly conserved |
| 152 | E | A, D | Residue critical for retroviral integrative |
| | | | recombination in a region that is highly conserved |
| 168 | Q | L, A | Residue critical for retroviral integrative |
| | | | recombination in a region that is highly conserved |
| | | | and integrase mutants defective for interaction |
| | | | with LEDGF/p75 are impaired in chromosome |
| | | | tethering and HIV-1 replication |
| 170 | E | G | Residue critical for retroviral integrative |
| | | | recombination in a region that is highly conserved |
| 185 | F | K | |
| 231 | R | G, K | Positions that are found in other integrase variants |
| | | | (taken from an alignment from Gijbers et al 2014) |
| 231 | R | D, E | Positive amino acids that might enhance DNA |
| | | | binding |
| 231 | R | K | Negative amino acids that might impair DNA |
| | | | binding (proven for 231E) |
| 231 | R | S | Negative amino acids that might impair DNA |
| | | | binding (proven for 231E) |
| 264 | K | R | IN acetylation “Acetylation of HIV-1 integrase by |
| | | | p300 regulations viral integration” |
| 266 | K | R | IN acetylation “Acetylation of HIV-1 integrase by |
| | | | p300 regulations viral integration” |
| 273 | K | R | IN acetylation “Acetylation of HIV-1 integrase by |
| | | | p300 regulations viral integration” |

### Example 14: Generation of pRRLVPR Integrase Constructs and Testing Transcomplementation Efficiency in Hek293T Cells

pRRLIN, pRRLVPRIN and pRRLINGFP vectors were generated for use in VPR trancomplementation (Table 9).

TABLE 9
pRRL Constructs

| | GFP(−) | GFP(+) |
|---|---|---|
| VPR(−) | pRRL_IN | pRRL_IN_GFP |
| VPR(+) | Prrl_VIN | pRRL_VIN_GFP |

The constructs were tested using a GFP expression assay. HEK293T cells were transfected with pSICO MAXI, pSICO MINI and pRRL_INGFP to test pRRLINGFP episomal expression. Expression of VPRINGFP construct in lentivirus producing cells was detected positive. Next, transcomplementation efficiency in HEK293T cells was tested.

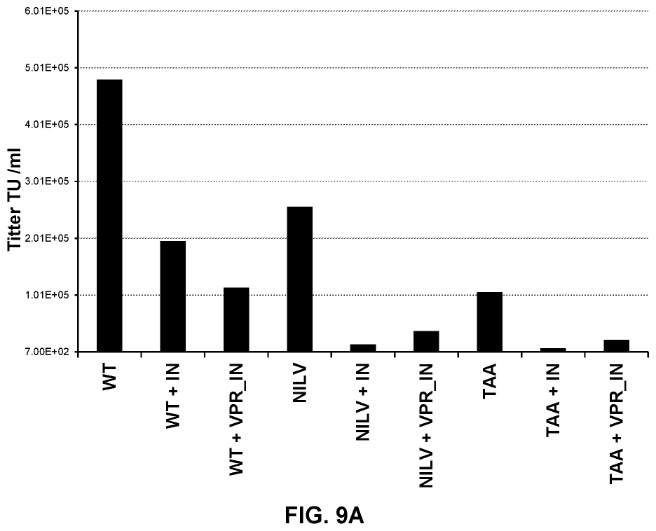

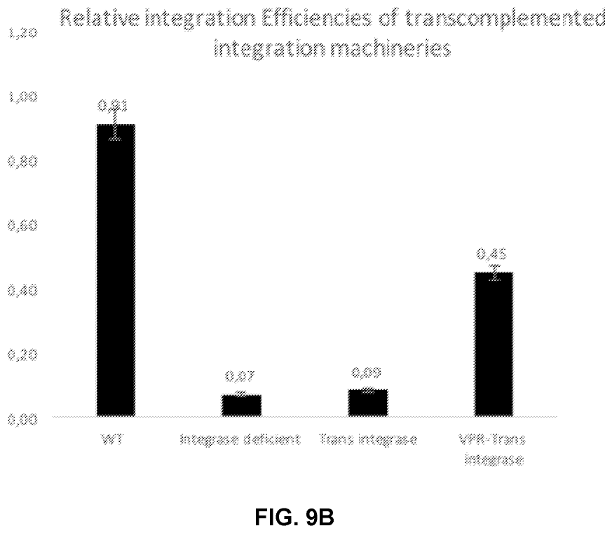

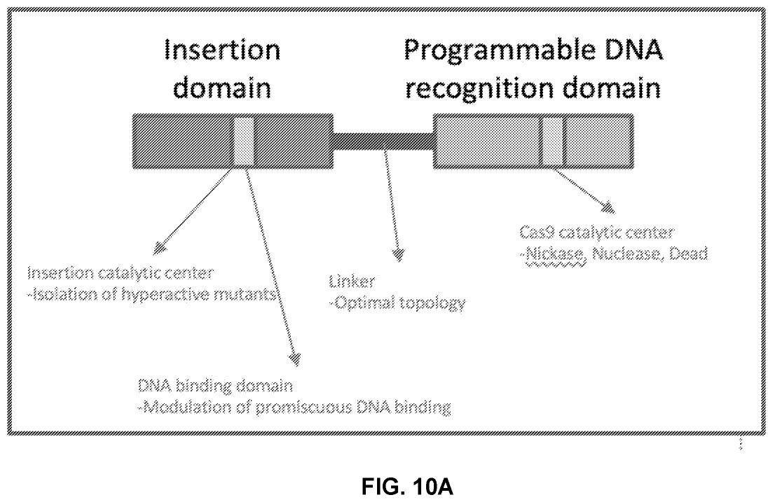

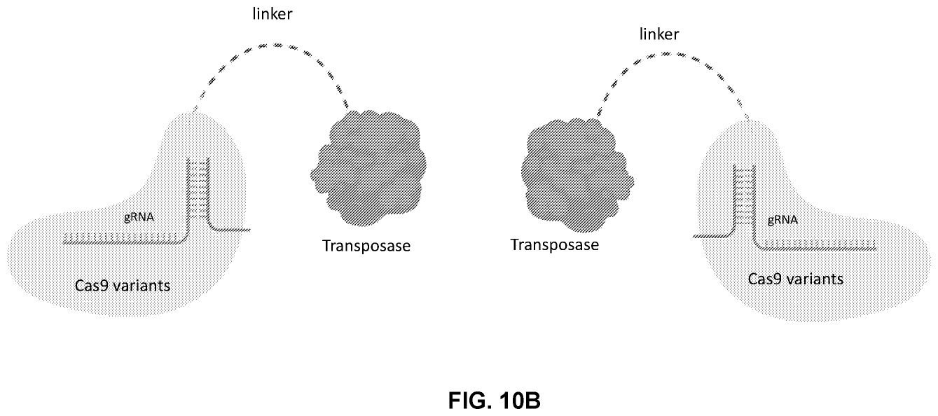

LV media was ultracentrifuged, left to resuspend, and cells where seeded. Infection was done in a volume of 0.6 ml (1.5*0.4). Polybrene was added. Titer was determined by cytometry. Titer (1:100) is shown in FIG. 9.

The VPR transcomplementation system will be used to compare the modified integrase sequences for integration.

In Examples 15-19 hereinafter, different constructs of fusion protein with modified hyperactive PiggyBac transposase were generated. Total and targeted transposition activity of the constructs were determined, resulting in relevant results especially for constructions of hcas9_mutated PB. Evidence is also provided for the generation and targeted transposition activity determination of constructs of fusion protein of mutated PB and ZFP. Different linkers are tested, showing that XTEN had better performance than the rest of linkers tested. 5GGS and 7GGS also worked properly, indicating that the length of the linker and its flexibility plays an important role on its performance.

### Example 15: Methods for Generation of Fusion Proteins with Modified Hyperactive Piggybac Transposases and Determination of Targeted Transposition Efficiency

### Transfections:

Hek293T cells were seeded the day before to achieve 70-80% confluency on transfection day (usually 290.000 cells in p12 well plate). Transfections were performed using lipofectamine 3000 reagent following manufacturer's instructions or PEI at 1:3 DNA-PEI ratio in OptiMem.

Programmable transposase (PT), gRNA and transposon plasmids were transfected together in a 1 PT: 2.5 gRNA: 2.5 transposon ratio.

Cells were passed and maintained until desired end-point depending on the experiment.

### Pb Mutant's Generation:

Different mutations were introduced into hyPB sequence fused to Cas9 (hCas9_PB plasmid) by site directed mutagenesis following Quickchange lightning Agilent mutagenesis kit's instructions. Primers were designed with QuikChange Primer Design to achieve the following mutations: PB R245A, PB R275-277A, PB R388A, PB S351A, PB W465A, PB R372A-K375A, PB D450N (SEQ ID NO: 100-106).

### Cas9 Activity:

Programmable transposase plasmid with nuclease Cas9 and gRNa plasmid were transfected together at 1:2.5 ratio. Cells were harvested after 48 h and genomic DNA was extracted. PCR was performed with primers targeting 150-200 bp around the gRNA target site (NGS-aays fw & NGS-aays rv, SEQ ID NO: 98-99). Illumina adapters and barcodes were introduced in a second PCR and miseq sequencing was performed usually in a 2×250 Nano flow cell. Results were analysed with CRISPR-GA web tool.

### Genetrap Assay:

A promoterless RFP transposon was produced preceded by and splicing acceptor and gRNAs targeting PPR1alpha and CD46 intron 1 were designed and cloned under U6 promoter regulation. RFP fluorescence would only be detected if transposon was inserted in the targeted regions or in other promoter regions by chance. For genetrap assay, Hek293T cells were transfected with genetrap transposon, programmable transposase and gRNA and RFP signal was analysed by Flow Cytometry.

### Split GPF Reporter Cell Line Cell Line:

A 293T reporter cell lines was produced for targeted transposition evidence experiments. Briefly the cell line has a target region (with different gRNAs and ZFP target sequences) and a splicing acceptor sequence followed by a half of a GFP coding sequence. This cell line was generated by random insertion of the reporter cassette using the hyperactive version of Sleeping Beauty transposase, SB100X. The targeted introduction of a transposon with the first half of the GFP sequence with a promoter and splicing donor results on GFP signal detectable by flow cytometry.

A second transposon was generated containing the half GFP sequence and a full RFP sequence preceded by EF1alpha constitutive promoter to assess targeted vs random insertion. Around 15 days after transfection there was a good decay of episomal signal which allows analysis of total insertion (RFP signal) versus targeted insertion (GFP signal).

### Example 16: Generation of Plasmid Constructions of Fusion Proteins with Modified Hyperactive Piggybac Transposases

Different plasmid constructions were cloned to achieve a fusion between a programmable element targeting DNA (cas9, ZNF) and a mammalian transposase (Piggybac, SB100). The linker in between the two modules was variable in the different constructs, chosen from a linker library with SEQ ID NO: 50-63. The constructs are shown in Table 10.

TABLE 10
List of Fusion Proteins Generated

| Fusions cas9 | Fusions cas9 | Fusions ZFN | |
|---|---|---|---|
| and hyPB | and SB100 | and hyPB | Fusions with hyPB mutations |
| hcas9_hyPB | hcas9_SB100 | ZFN_hyPB | hcas9_ hyPB_D450N 4GGS linker, |
| ncas9_hyPB | ncas9_SB100 | hyPB_ZFN | ncas9_hyPB_D450N 4 ggs linker, |
| dcas9_hyPB | dcas9_SB100 | | dcas9_hyPB, D450N 4 GGSlinker |
| hyPB_hcas9 | SB100_hcas9 | | hcas9_hyPB_D450N-R372-375A 4 GGS |
| hyPB_ncas9 | SB100_ncas9 | | linker, ncas9_ hyPB_D450N-R372-375A 4 |
| hyPB_dcas9 | SB100_dcas9 | | GGS linker, dcas9_ hyPB_D450N-R372- |
| | | | 375A 4 GGS linker |
| | | | hcas9_hyPB with the following mutations: |
| | | | R245A, R275-277A, R388A, S351A, |
| | | | W465A |
| | | | ZFP_ hyPB D450N |
| | | | hyPB D450N_ZFP |
| | | | ZFP_hyPB D450N-R372-375A |
| | | | hyPB D450N-R372-375A_ ZFP |

hcas9: cas9 nuclease human codon optimized; ncas9: nickase cas9 human codon optimized; dcas9: dead cas9 human codon optimized.

### Example 17: Transposition Efficiency of Different Linkers

Hek 293T cells were transfected with hcas9_PB constructs with different linkers in length and structure (linker library) and with 2 different gRNAs (AAVS1 1 and AAVS1 2). Genomic DNA was extracted 48 after transfection, the targeted region was PCR amplified and sequenced with an Illumina miseq sequencing.

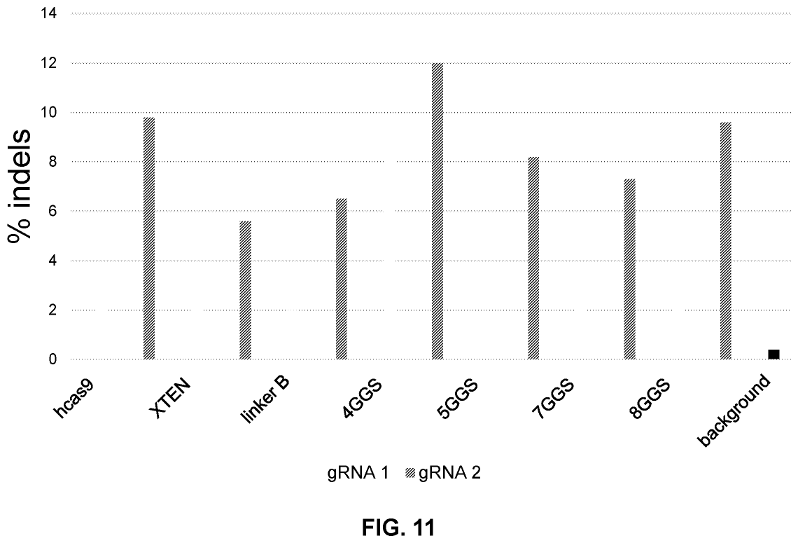

Results: Constructions with different linkers length and structure do not obstruct cas9 nuclease activity. 4GGS linker gives a higher cas9 activity on both gRNAs target sites in comparison to hcas9 activity (FIG. 11).

### Example 18: Targeted Transposition of Fusion Proteins with Modified Hyperactive Piggybac Transposases

### 18.1. GeneTrap:

Targeted transposition activity of hcas9_PB construct (hcas9 linked to hyPB using different linkers described before) was assessed using a genetrap transposon. Genetrap transposon contains a promoterless RFP sequence preceded by a splicing acceptor sequence which can only be expressed if it is inserted in a promoter region after a splicing donor.

Genetrap transposon was contransfected with PPR1 intron 1 gRNA and programmable transposase with different linkers constructions. Results were analysed 10 days after transfection by RFP fluorescence using Flow Cytometry.

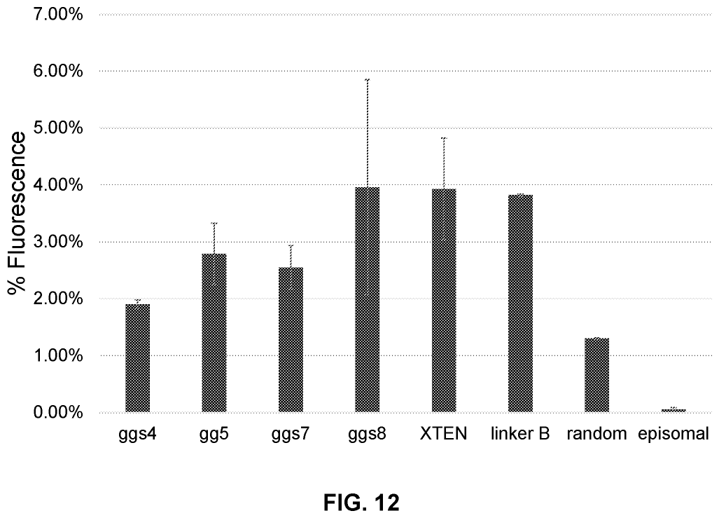

Results: Targeted activity was increased by programmable transposase in comparison to hyPB random insertion having more fluorescence the conditions transfected with programmable transposase than the condition transfected with wild typ hyPB. 8ggs, XTEN linkers increased Genetrap targeted activity in comparison to the other linkers (FIG. 12).

### Split GFP Reporter Cell Line:

18.2 Targeted transposition hcas9_PB with different linkers

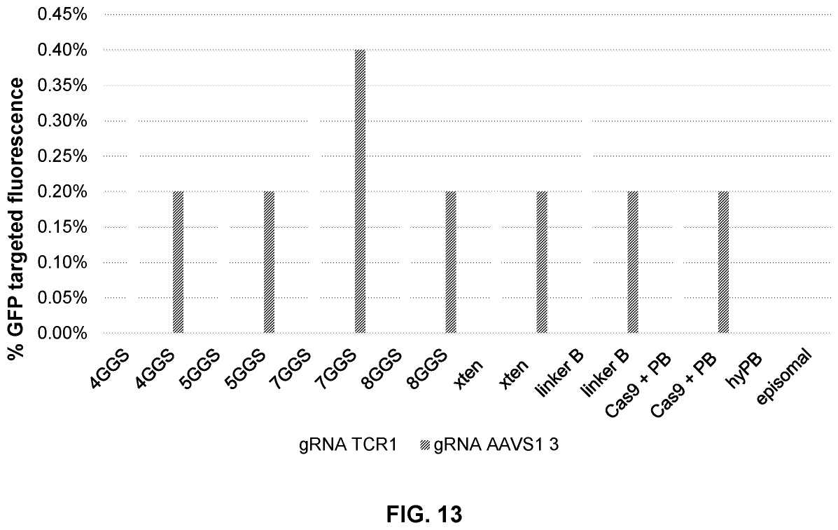

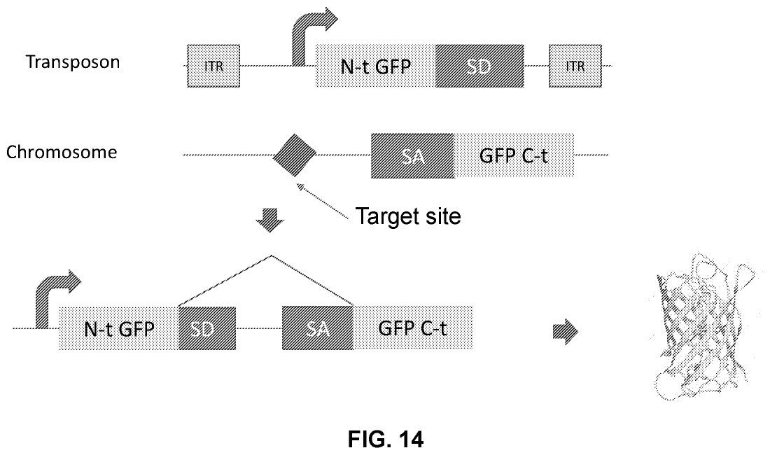

Targeted transposition activity of hcas9_PB construct was assessed using a reporter cell line. hcas9_PB construct with different linkers were transfected with gRNA AAVS1 3 or TCR1alpha and a half GFP transposon. Results: Big differences were not appreciated regarding to different linkers constructs transposition (FIG. 13).

18.3. Targeted Transposition of Selected Mutants:

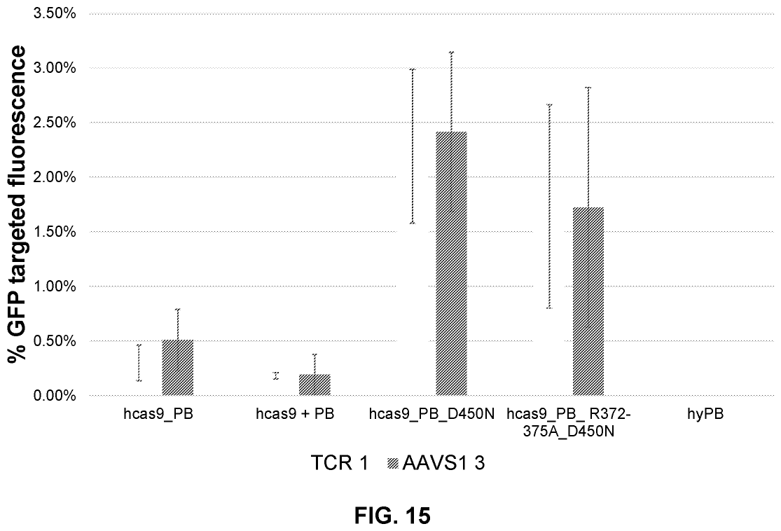

PB 450 and PB 372-375-450 were selected for further targeted transposition experiments due to their good targeted transposition efficiencies. Experiments were performed as mentioned before using gRNA aays1 3 and tcr 1. Results: Targeted transposition of hcas9_PB 450 and hcas9_PB 372-374-450 was 6 to 10-fold higher in comparison to hcas9_PB with hyPB WT sequence. hcas9+hyPB transfected in separated plasmids showed some targeted activity while hyPB with no hCas9 showed 0 activity indicating that the split GFP reporter cell line is a robust method for targeted insertion for the selection of variants that perform this function over the noise of Ther methods that are not specific enough (FIG. 15).

18.4. Targeted and Random Transposition Selected PB Mutants:

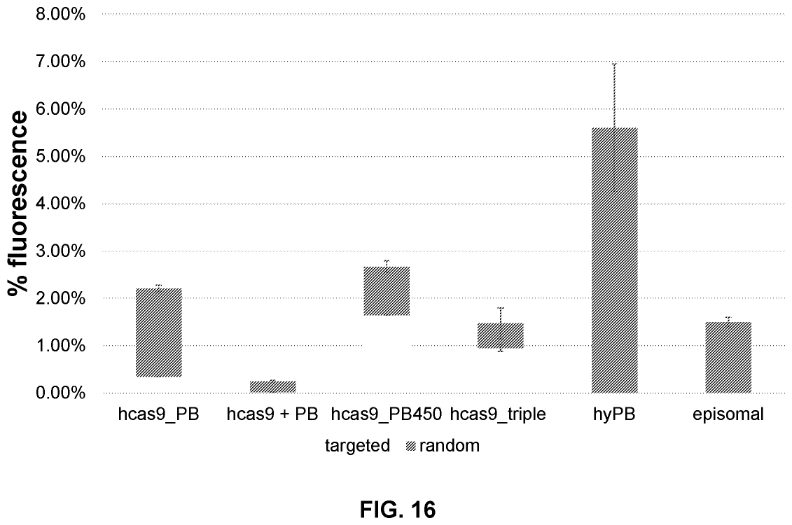

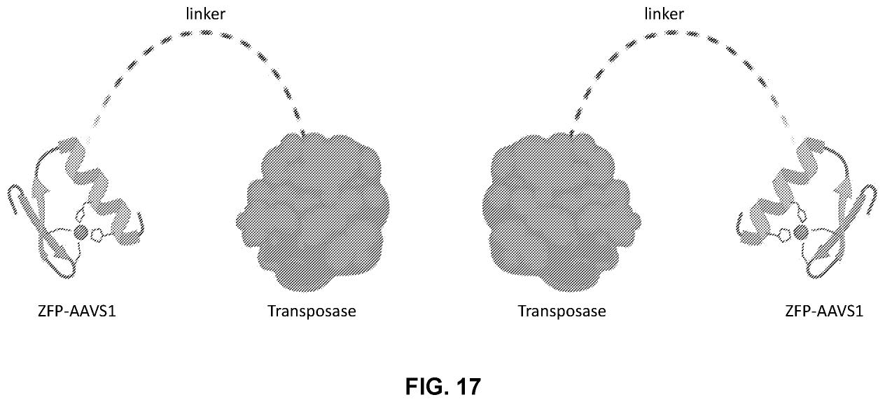

Targeted and random transposition were assessed using an RFP-GFP dual transposon mentioned before for selected mutants on example 19.4. Red fluorescence indicates total insertion (RFP being expressed constitutively) around 15 days after transfection (to ensure non episomal signal) and GFP fluorescence indicates targeted transposition. Results: FIG. 16 shows that higher targeted transposition compared to random transposition was shown on both hcas9_PB D450N and hcas9_PB R372A K375A D450 selected mutants in comparison with hcas9:PB with wt hyPB sequence. Total transposition efficiency is lower in both mutants and targeted results are consistent with FIG. 15.

18.5. Targeted Transposition ZFP-PB Constructs:

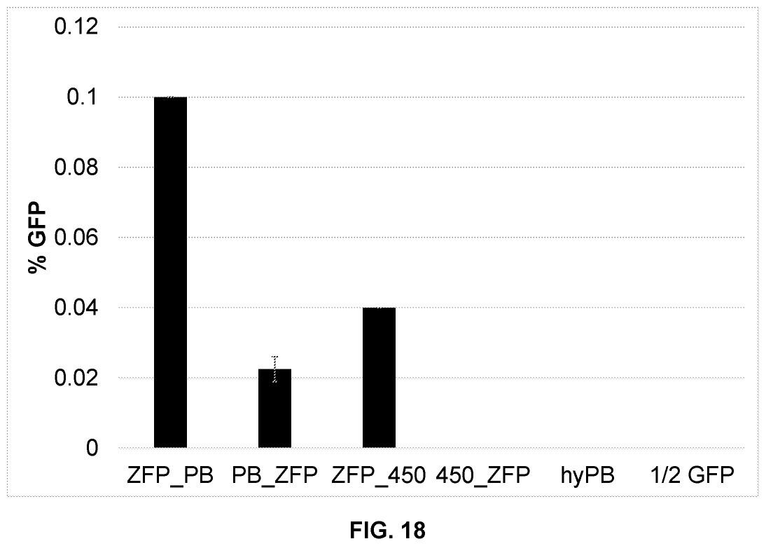

Constructs for Zinc finger-hyperactive PiggyBac fusion proteins were cloned using ZFP targeting tcr4 sequence present on the split GFP reporter cell line and hyPB or hyPB with D450N mutations. Cells were transfected with ZFP-PB combinations and ½ GFP transposon following protocol of Example 15. GFP signal was analysed 5 days after transfection. Results: Targeted transposition was observed above the background (hyPB random insertion) in all the constructions. Results: Targeted transposition is higher in ZFP in N-terminal position for both hyPB and hyPB D450N (FIG. 18). ZFP sequence for these experiments correspond to a protein of 6 finger domains with nucleic acid and amino acid sequences SEQ ID NO: 117 and 118, respectively.

In Example 20 hereinafter, a library of PB mutations was designed and submitted to a screening method to identify modified PB for positive targeted transposition. Some hits for modified PB with positive targeted transposition were identified and validated.

### Example 20: Generation of a Hyperactive Piggybac Mutations Library and Screening for Targeted Transposition

### Methods:

A library of hyPB mutations was designed and purchased from Twist Biosciences.

TABLE 11
Mutation Sites for hyPiggyBac

| | Wild-type | |
|---|---|---|
| Position | Amino Acid | Mutation |
| 245 | R | A |
| 275 | R | A |
| 277 | R | A |
| 325 | G | A |
| 347 | N | A, S |
| 351 | S | E, P, A |
| 372 | R | A |
| 375 | K | A |
| 388 | R | A |
| 450 | D | N |
| 465 | W | A |
| 560 | T | A |
| 564 | S | P |
| 573 | S | A |
| 589 | M | V |
| 592 | S | G |
| 594 | F | L |

### Screening Method:

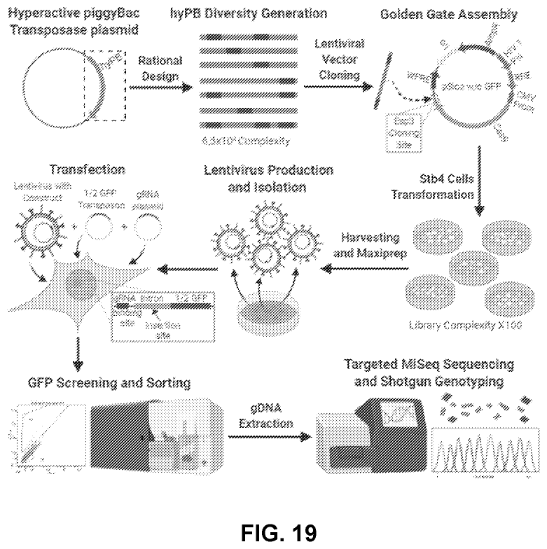

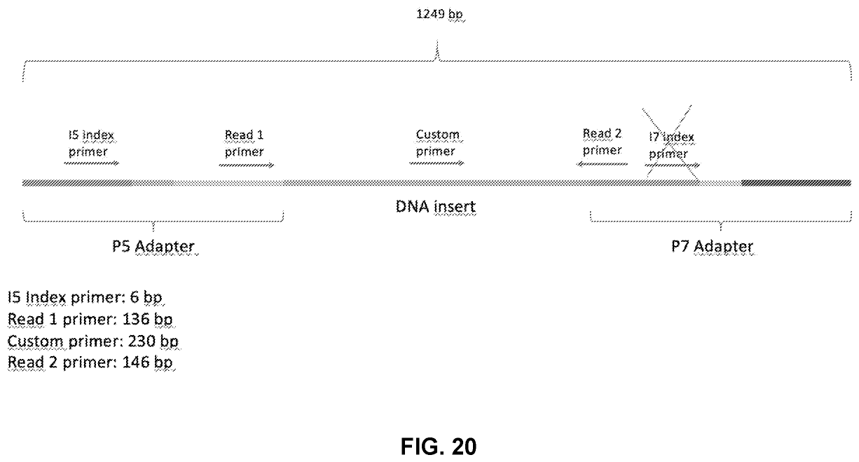

A screening method was designed to identify Piggybac variants from the designed mutant library which linked to a targetable DNA binding protein such as cas9 and performed specific targeted transpositions. A scheme of the screening method is shown in FIG. 19. PB library was cloned by Golden Gate assembly using Esp3I enzyme into a SIN transfer lentiviral plasmid containing hcas9 and XTEN linker followed by Esp3I cloning sites before an NLS to achieve hcas9 XTEN_PB_NLS fusion protein under CMV promoter regulation. Around 6.000.000 colonies were harvested after ElectroMAX™ Stb14™ Competent Cells from Invitrogen electroporation, and plasmid were extracted with maxiprep using HiPure Maxiprep kit, LifeTechnologies. Lentiviruses were produced (using pMD2.G and psPAX2 helper plasmids purchased from Addgene) using lentivirus production protocol from Addgene. Lentiviruses were ultracentrifuged and tittered by copy number analysis qPCR (with the oligonucleotides SEQ ID NO: 107-110). Briefly, 80.000 Hek293T cells were seeded the day before in p12 well plates. Cells were infected with Library lentiviruses and standard GFP lentivirus at dilutions ½, 1/10 for library lentiviruses and 1/50, 1/100, 1/1000 for GFP lentiviruses. GFP signal was analysed 3 days after infection by flow cytometry. Cells were harvested and gDNA was extracted. qPCR assay was designed to assess WPRE gene copy number and normalized by RNAse gene copy number.

Hek293T Reporter cells were infected at MOI 0.8, in 500 cm² square dishes using 1:1000 polybrene, 10M cells were plated the day before. 3-4 days after infection, cells were transfected with 8.1 pmol gRNA AAVS1 plasmid and ½ GFP transposon using PEI 1:3. 9M cells were plated the day before in 15 cm dishes. 3-4 days after transfection cells were sorted using FACSAria cytometer an 0.70m nozzle. A transfection control was performed in 10 cm dish using an RFP and GFP plasmids with the same molarity and analysed in Fortessa cytometer for GFP-RFP positive cells. After sorting, gDNA was directly extracted.

Different sequencing methods were used to analyze PB mutants with positive targeted transposition:

### PiggyBac Library Region Targeted Sequencing:

PiggyBac 1116 bp region with all library variants was PCR amplified with primers NGS cluster 1 fw and NGS cluster 2 ry using KAPA HiFi Hotstart ReadyMix. Illumina adapters and barcodes were added in a second PCR, NEBNext 9 primer and Illumina custom barcodes were used (SEQ ID NO: 111-114). Targeted sequencing was performed in v2 or v3 Illumina miseq flow cells. I7 Index primer was replaced by a custom primer to allow the full sequencing of the different variants.

### Piggybac and Cas9 Sequence Shotgun Library Generation and Sequencing:

A 6000 bp PCR from genomic DNA of GFP positive sorted cells was performed with primers CMV-F and SV40 pA ry (SEQ ID NO: 115 and 132), amplifying cas9 and PB sequence with KAPA HiFi HotStart ReadyMix. DNA was then purified with Qiagen gel extraction kit and fragmented at 500 bp with Covaris S220 and microtube AFA fiber Crimp-Cap. Shotgun library was prepared with KAPA hyperprep kit according to manufacturer's instructions.

### Results:

20.1. hyPB Library Diversity Generation:

½ GFP reporter cell line was infected at MOI 0.8 with lentiviruses containing hcas9_PB with PB library mutations. 3 days after infection, cells were transfected with gRNA AAVS1 3 and ½ GFP Transposon with 75-90% Transfection Efficiency.

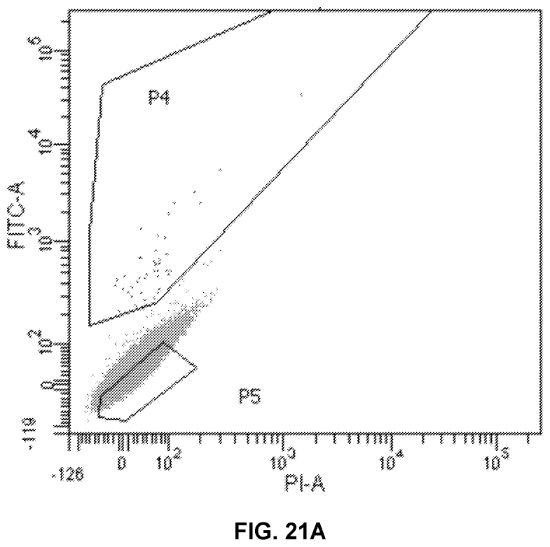

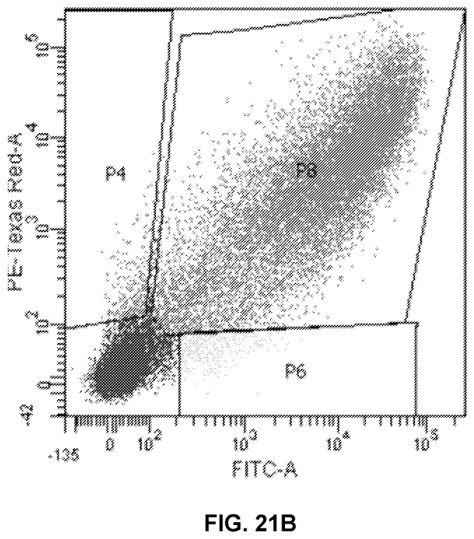

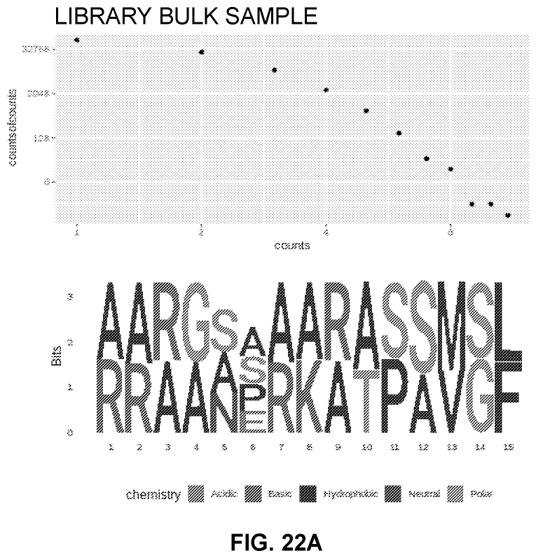

In a first experiment, total of 254M cells were sorted and 185.757 positive cells were obtained showing 0.073% targeted transposition positive variants. In a second experiment 120M cells were sorted and 70.974 positive cells were obtained showing 0.059% targeted transposition positive variants (FIGS. 21A and 21B).

Genomic DNA was directly extracted from positive and negative sorted cells. ⅔ of the DNA obtained was processed for targeted sequencing analysis and V3 was processed as a shotgun library sequencing as specified above in section METHODS of this Example.

20.2. hyPB Library Screening Analysis by Targeted Sequencing of the Variable Region:

Positive and negative cells analysis of Cas9-PB variants were analyzed as follows. Reads from targeted sequencing were mapped against the reference sequence. All library variation positions were retrieved using two different approaches: by position, using the aligned reads, and by sequence, using a pattern match of the surrounding sequence. The logarithmical fold change of all variant counts was calculated between positive (GFP positive cells with targeted integration) and negative samples (non targeted integration samples, regardless of weather or not integration had occurred), and the top variants were retrieved. Additionally, negative selection of those samples with random integration were done with RFP positive selection; where the transposon was inserted randomly in the genome.

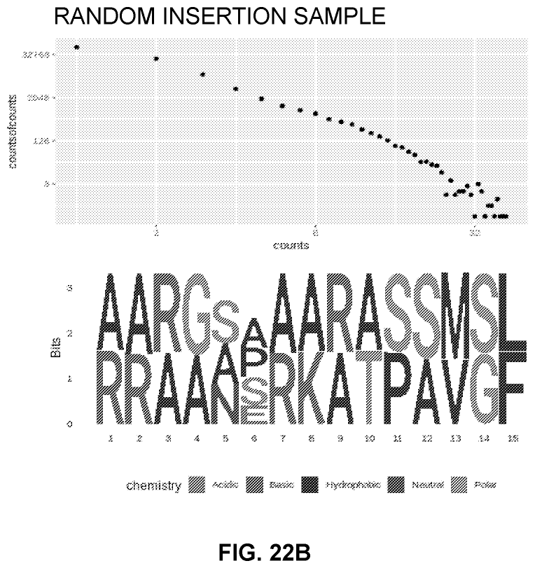

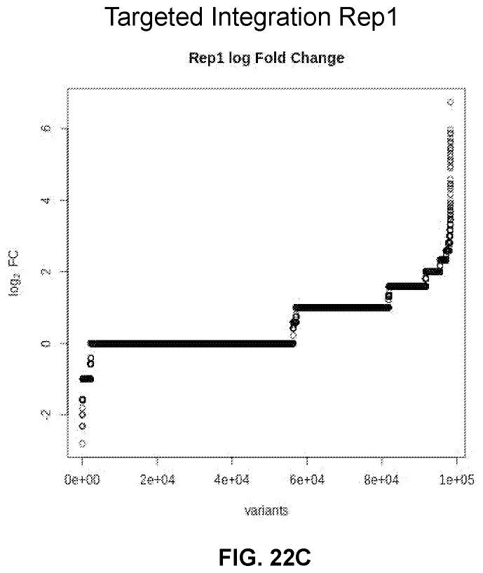

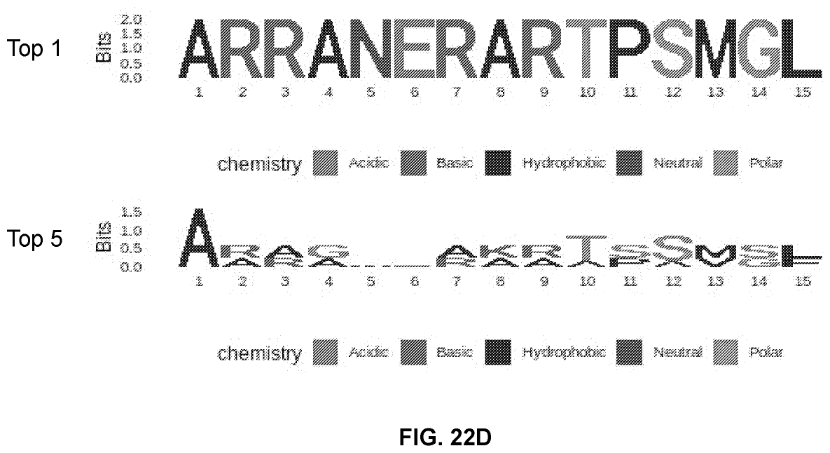

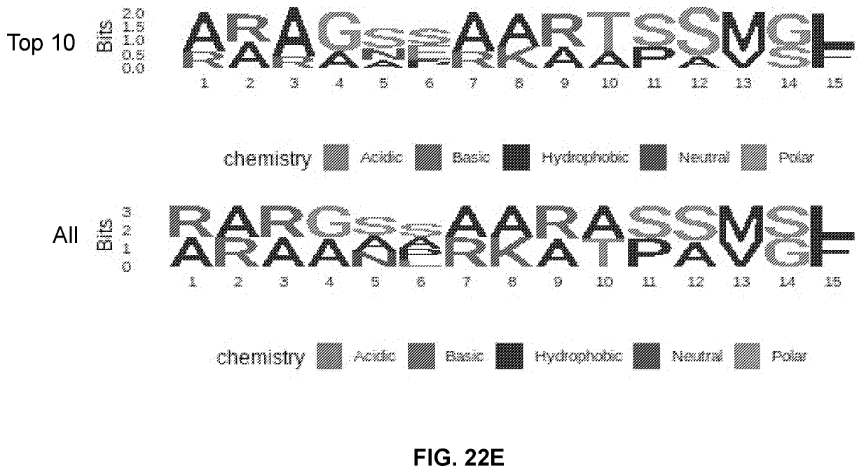

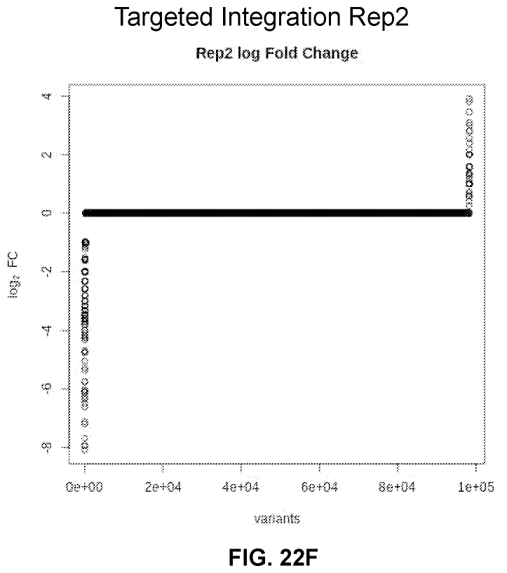

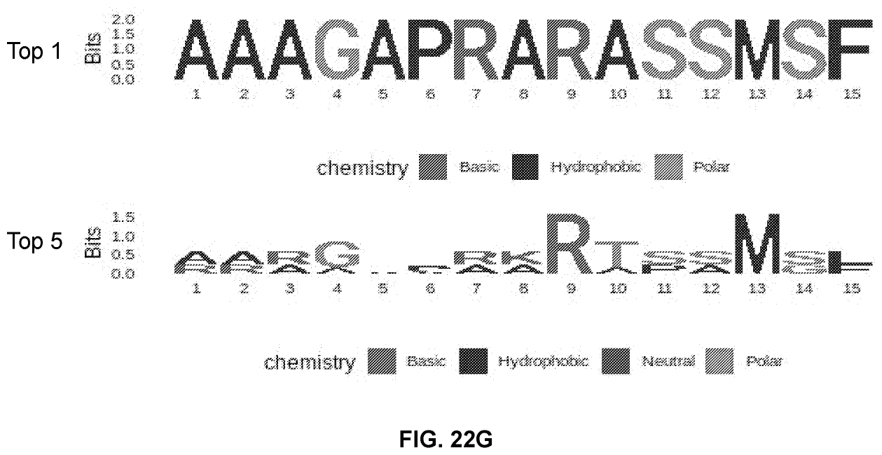

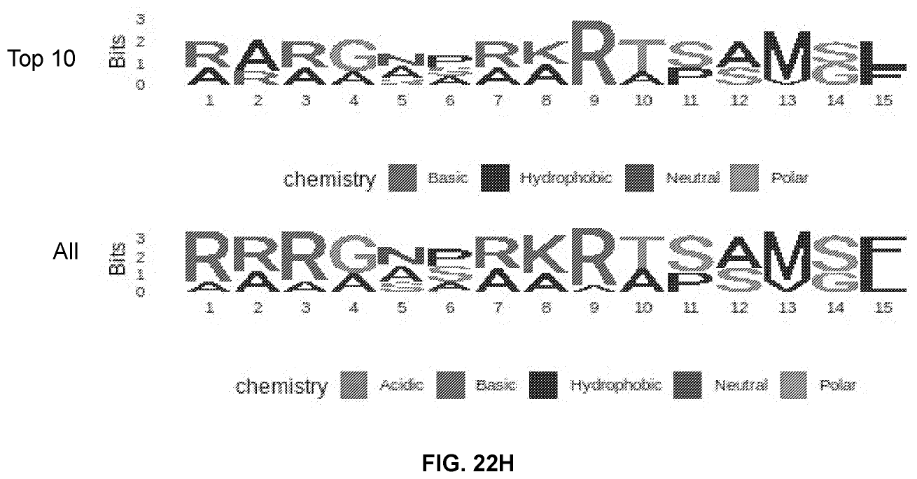

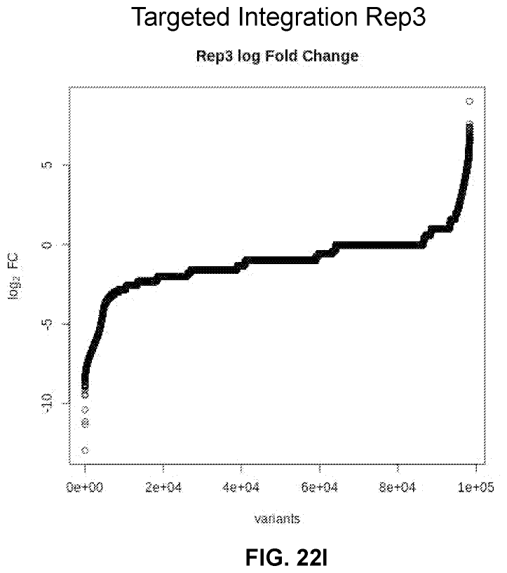

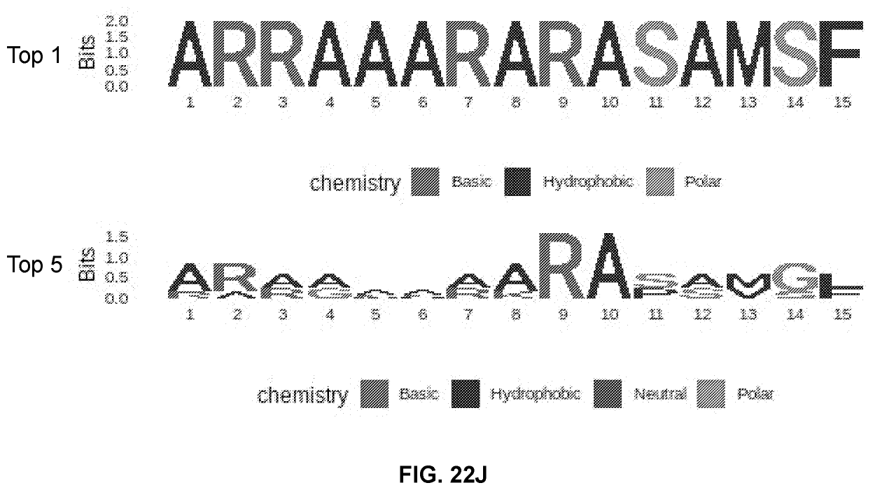

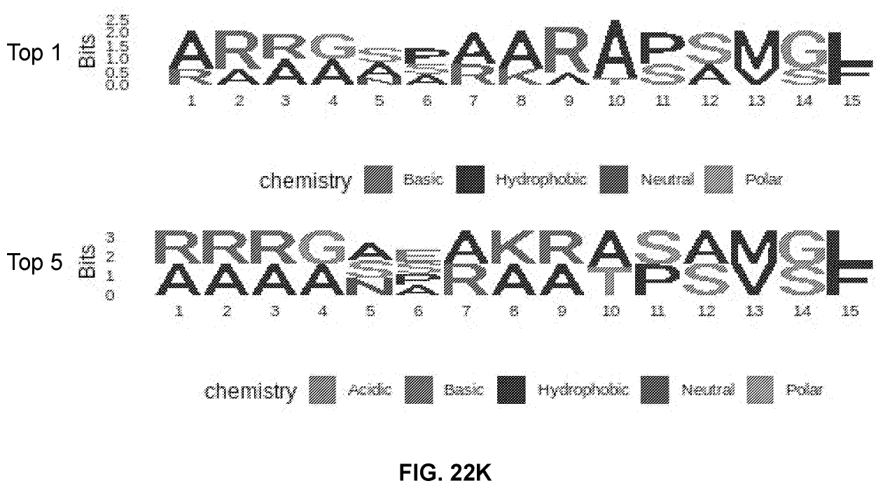

Results are shown in FIG. 22A-22K. Therefore, using an unsupervised high-throughput screening approach of a combinatorial library of variants, a collection of mutants for Piggyback able to perform site directed insertion with a high efficiency were identified, as indicated by the comparison of presence in the positive versus negative cell population.

Next, targeted and random transposition of top positive hit in repeat 1 was assessed using an RFP-GFP dual transposon mentioned before. Red fluorescence indicates total insertion (RFP being expressed constitutively) around 15 days after transfection and GFP fluorescence indicates targeted transposition.

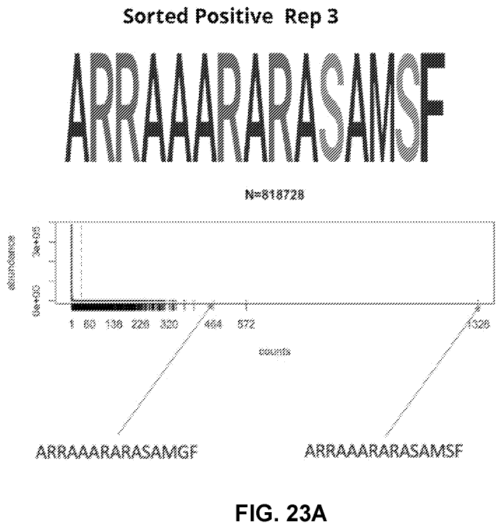

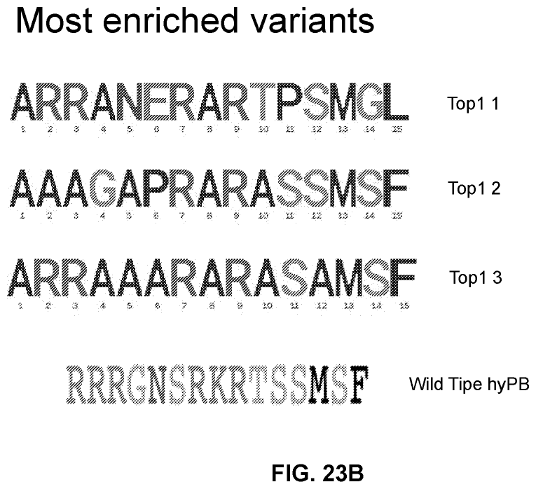

Results: Higher targeted transposition compared to random transposition was shown on Top1 of repeat 1 variant in comparison with hcas9_PB and with wt hyPB (FIG. 23A-23B). An independent validation of on-target insertion using our reporter cell line was performed, and significant on-target activity was observed compared to WT version, and to the D450N mutant.

20.3. Identification of Over-Represented Positive Hits:

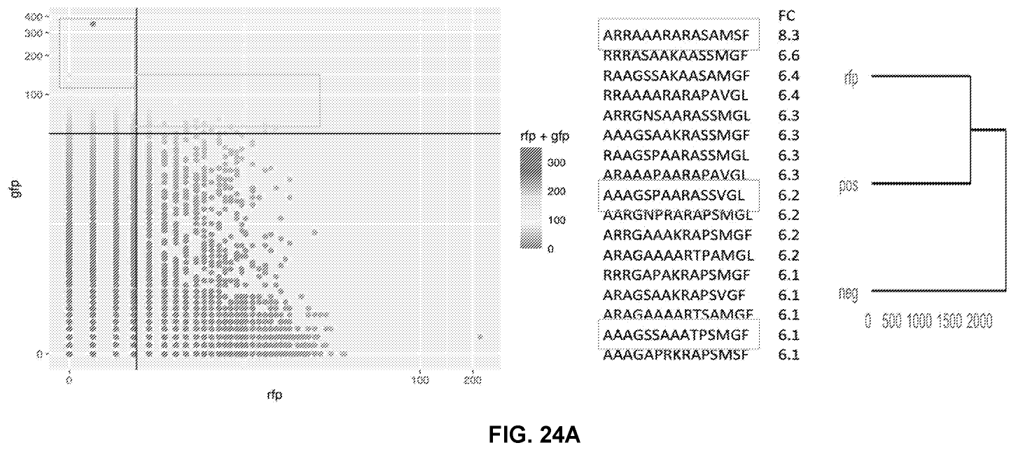

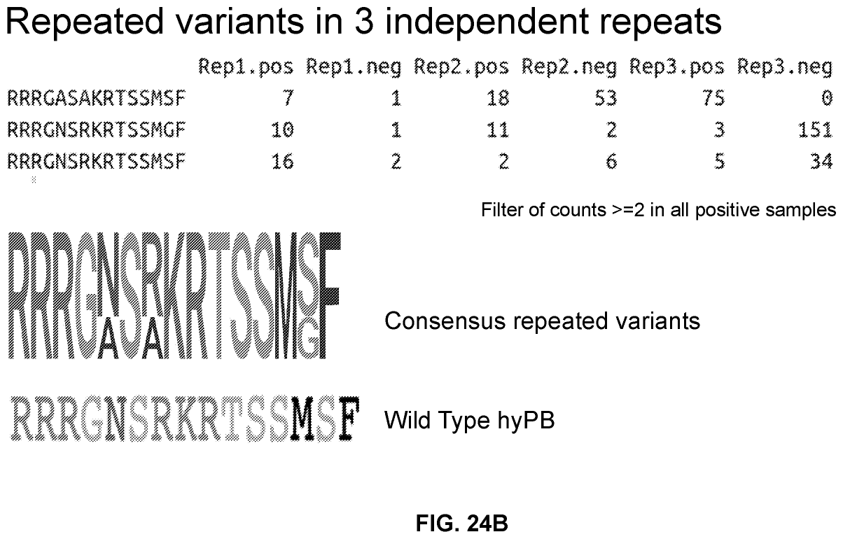

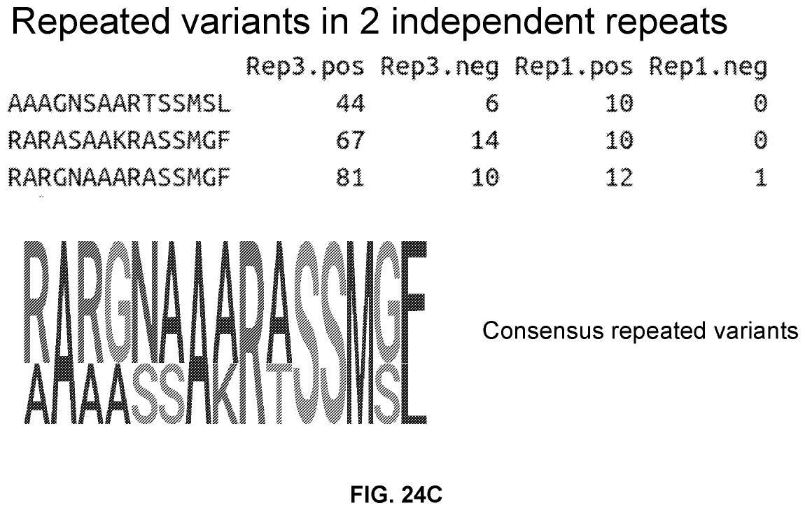

Several positive hits that are over-represented in the GPF population versus negative selected variants were identified in the screening. Some of them were also not found in RFP population that represent overall insertion, which indicates an increase in integration capacity. Moreover, RFP includes random and targeted integration. Thus, a collection of combinatorial mutants for Piggyback able to perform site directed insertion with a high efficiency was identified (FIG. 24A-24C).

20.4. hyPB Library Screening Analysis by Shotgun Sequencing:

For shotgun sequencing, reads were mapped against the reference sequence, a variant calling was performed retrieving all variations from the reference and the Euclidean and correlation distance were calculated between positive and negative allele counts. The most different positions were retrieved as variants; and the association between these variants were calculated.

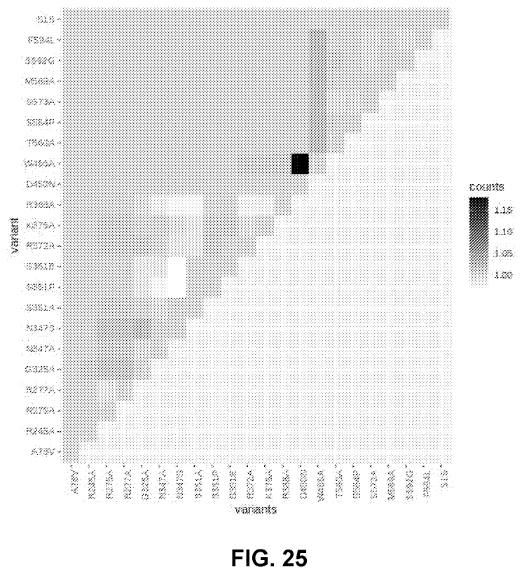

Results: In addition to variants included in the library design, the variants that were randomly introduced by the lentiviral retrotranscriptase during viral library generation were analyzed. Some of these new variants were associated with the positive hits and probably perform the targeted integration on combination, and they maybe need to be present in the mutant form in the variant version of hyPB to perform targeted integration. Example of D450N and W465A is shown in FIG. 25.

The mutated PB sequences identified in Example 20 are listed in Table 12 (SEQ ID NO: 120-129).

20.5. hyPB Library Screening Validation:

Targeted and random transposition of several combinations of single mutations seen in Top1-1 identified in the screening positive hits (Unilarge-A, -B, -C and Unilarge-D) were assessed using an RFP-GFP dual transposon mentioned before. Red fluorescence indicates total insertion (RFP being expressed constitutively) around 15 days after transfection and GFP fluorescence indicates targeted transposition.

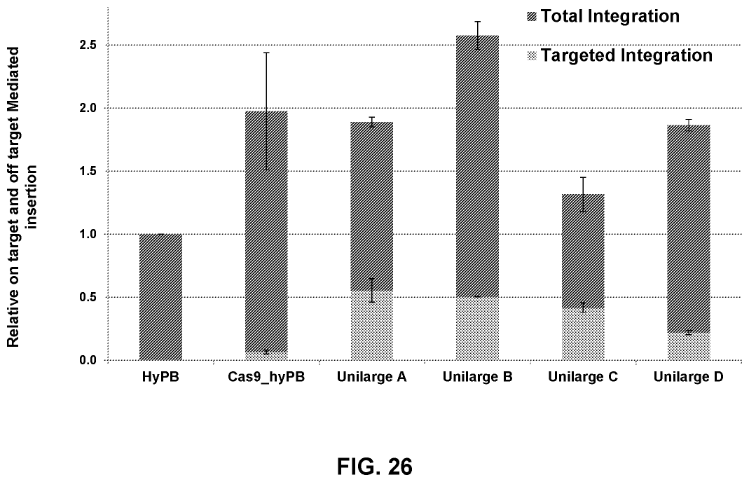

Results: In all cases an increase in the targeted insertion relative to overall integration was observed for Cas9 fused to different mutant combinations of hyPB with 4GGS linker (Unilarge-A: D450N; Unilarge-B: R245A/D450N; Unilarge-C: R245A/G325A/D450N/S573P; Unilarge-D: R245A/G325A/S573P) when compared to fusion of Cas9- to the WT version of hyPB. Some of the mutant combinations tested (R245A/G325A/D450N/S573P) had a great increase of the targeted insertion being up to 30% of total integrative events instead of a 3% percent in the hyPB fusion (Unilarge C) (FIG. 26).

Examples 21 hereinafter provides an overview of the developmental state of the different integration deficient viral vectors, as well as the best transcomplementation system; and data on transcomplementation with IN fusion proteins.

### Example 21: Transcomplementation of Different Integrase Deficient Systems

To generate an efficient transcomplementation system to test IN fusion proteins, viral production efficiency and its integration capacity were assessed by infecting the different condition of integration deficient virus and transcomplemented virus into Hek293T and Jurkats cells. Cells were passed for 7 days until no episomal signal was detected and GFP signal was analyzed by Flow Cytometry at day 2, 5 and 7.

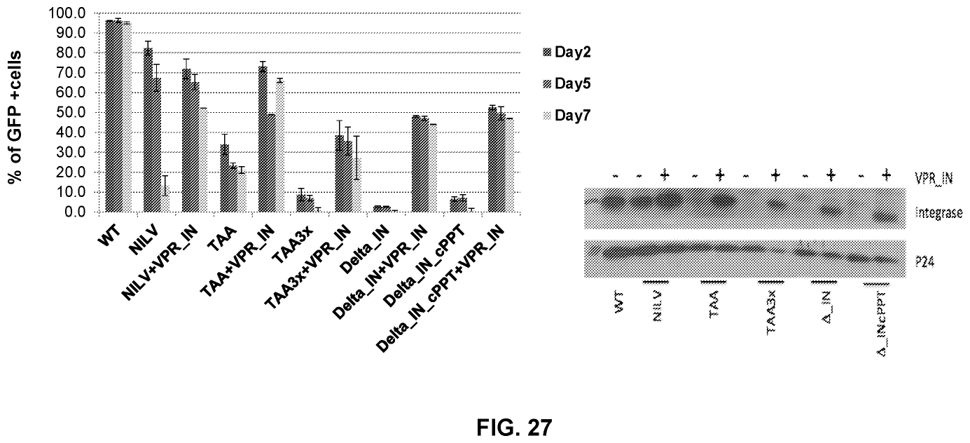

Results: Different production efficiencies could be detected for different systems, being NILV the closed to WT upon production. In all cases a clear rescue of the integration activity was apparent when transcomplementation was done with WT-HIV_IN. (FIG. 27). Proof of IN being loaded in the transcomplementation system was obtained by western blot.

TABLE 12
Sequences. “na sequence” denotes nucleic acid sequence and “aa sequence”
amino acid sequence.

| SEQ ID | | |
|---|---|---|
| NO | SEQUENCE NAME | SEQUENCE |
| 1 | Wildtype HIV-1 integrase | FLDGIDKAQDEHEKYHSNWRAMASDFNLPPVVAKEIVASCDKC |
| | aa sequence NC_001802.1- | QLKGEAMHGQVDCSPGIWQLDCTHLEGKVILVAVHVASGYIEA |
| | NP_705928 | EVIPAETGQETAYFLLKLAGRWPVKTIHTDNGSNFTGATVRAA |
| | | CWWAGIKQEFGIPYNPQSQGVVESMNKELKKIIGQVRDQAEHL |
| | | KTAVQMAVFIHNFKRKGGIGGYSAGERIVDIIATDIQTKELQK |
| | | QITKIQNFRVYYRDSRNPLWKGPAKLLWKGEGAVVIQDNSDIK |
| | | VVPRRKAKIIRDYGKQMAGDDCVASRQDED |
| 2 | Wildtype HIV-1 integrase | tttttagatggaatagataaggcccaagatgaacatgagaaat |
| | na sequence NC_001802.1 | atcacagtaattggagagcaatggctagtgattttaacctgcc |
| | | acctgtagtagcaaaagaaatagtagccagctgtgataaatgt |
| | | cagctaaaaggagaagccatgcatggacaagtagactgtagtc |
| | | caggaatatggcaactagattgtacacatttagaaggaaaagt |
| | | tatcctggtagcagttcatgtagccagtggatatatagaagca |
| | | gaagttattccagcagaaacagggcaggaaacagcatattttc |
| | | ttttaaaattagcaggaagatggccagtaaaaacaatacatac |
| | | tgacaatggcagcaatttcaccggtgctacggttagggccgcc |
| | | tgttggtgggcgggaatcaagcaggaatttggaattccctaca |
| | | atccccaaagtcaaggagtagtagaatctatgaataaagaatt |
| | | aaagaaaattataggacaggtaagagatcaggctgaacatctt |
| | | aagacagcagtacaaatggcagtattcatccacaattttaaaa |
| | | gaaaaggggggattggggggtacagtgcaggggaaagaatagt |
| | | agacataatagcaacagacatacaaactaaagaattacaaaaa |
| | | caaattacaaaaattcaaaattttcgggtttattacagggaca |
| | | gcagaaatccactttggaaaggaccagcaaagctcctctggaa |
| | | aggtgaaggggcagtagtaatacaagataatagtgacataaaa |
| | | gtagtgccaagaagaaaagcaaagatcattagggattatggaa |
| | | aacagatggcaggtgatgattgtgtggcaagtagacaggatga |
| | | ggattag |
| 3 | Modified HIV-1 integrase | SEQ ID NO: 1 |
| | aa sequence | With D10K, E13K, D64A, D64E, G94D, G94E, |
| | | G94R, G94K, D116A, D116E, N117D, N117E, |
| | | N117R, N117K, S119A, S119P, S119T, S119G, |
| | | S119D, S119E, S119R, S119K, N120D, N120E, |
| | | N120R, N120K, T122K, T122I, T122V, T122A, |
| | | T122R, A124D, A124E, A124R, A124K, A128T, |
| | | E152A, E152D, Q168L, Q168A, E170G, F185K, |
| | | R231G, R231K, R231D, R231E, R231S, K264R, |
| | | K266R, K273R, or any combination thereof |
| 4 | Modified integrase aa | SEQ ID NO: 1 |
| | sequence with impaired | With G94D, G94E, G94R, G94K, N117D, N117E, |
| | DNA binding | N117R, N117K, S119A, S119P, S119T, S119G, |
| | | S119D, S119E, S119R, S119K, N120D, N120E, |
| | | N120R, N120K, A124D, A124E, A124R, A124K, |
| | | R231G, R231K, R231D, R231E, R231K, or any |
| | | combination thereof |
| 5 | Modified integrase aa | SEQ ID NO: 1 |
| | sequence with enhanced | With G94D, G94E, G94R, G94K, N117D, N117E, |
| | DNA binding | N117R, N117K, S119A, S119P, S119T, S119G, |
| | | S119D, S119E, S119R, S119K, N120D, N120E, |
| | | N120R, N120K, T122K, T122I, T122V, T122A, |
| | | T122R, A124D, A124E, A124R, A124K, R231G, |
| | | R231K, R231D, R231E, R231S, or any |
| | | combination thereof |
| 6 | Modified integrase aa | SEQ ID NO: 1 |
| | sequence with acetylation | With K264R, K266R, K273R, or any |
| | mutations | combination thereof |
| 7 | Modified integrase aa | SEQ ID NO: 1 |
| | sequence with mutations | With DlOK, E13K, D64A, D64E, D116A, D116E, |
| | in retroviral integrative | A128T, E152A, E152D, Q168L, Q168A, E170G, |
| | recombination | or any combination thereof |
| 8 | Modified integrase with | SEQ ID NO: 1 |
| | mutations in HIV-1 | With Q168L and/or Q168A |
| | replication aa sequence | |
| 9 | Hyperactive PiggyBac aa | MGSSLDDEHILSALLQSDDELVGEDSDSEVSDHVSEDDVQSDT |
| | sequence | EEAFIDEVHEVQPTSSGSEILDEQNVIEQPGSSLASNRILTLP |
| | | QRTIRGKNKHCWSTSKPTRRSRVSALNIVRSQRGPTRMCRNIY |
| | | DPLLCFKLFFTDEIISEIVKWTNAEISLKRRESMTSATFRDTN |
| | | EDEIYAFFGILVMTAVRKDNHMSTDDLFDRSLSMVYVSVMSRD |
| | | RFDFLIRCLRMDDKSIRPTLRENDVFTPVRKIWDLFIHQCIQN |
| | | YTPGAHLTIDEQLLGERGRCPERVYIPNKPSKYGIKILMMCDS |
| | | GTKYMINGMPYLGRGTQTNGVPLGEYYVKELSKPVHGSCRNIT |
| | | CDNWFTSIPLAKNLLQEPYKLTIVGTVRSNKREIPEVLKNSRS |
| | | RPVGTSMFCFDGPLTLVSYKPKPAKMVYLLSSCDEDASINEST |
| | | GKPQMVMYYNQTKGGVDTLDQMCSVMTCSRKTNRWPMALLYGM |
| | | INIACINSFIIYSHNVSSKGEKVQSRKKFMRNLYMGLTSSFMR |
| | | KRLEAPTLKRYLRDNISNILPKEVPGTSDDSTEEPVMKKRTYC |
| | | TYCPSKIRRKASASCKKCKKVICREHNIDMCQSCF |
| 10 | Modified hyperactive | SEQ ID NO: 9 |
| | PiggyBac aa sequence | With R245A, D268N, R275A/R277A, K287A, |
| | | K290A, K287A/K290A, R315A, G325A, R341A, |
| | | D346N, N347A, N347S, T350A, S351E, S351P, |
| | | S351A, K356E, N357A, R372A, K375A, |
| | | R372A/K375A, R388A, K409A, K412A, |
| | | K409A/K412A, K432A, D447A, D447N, D450N, |
| | | R460A, K461A, R460A/K461A, W465A, S517A, |
| | | T560A, S564P, S571N, S573A, K576A, H586A, |
| | | I587A, M589V, S592G, F594L, or any |
| | | combination thereof |
| 11 | Modified hyperactive | SEQ ID NO: 9 |
| | PiggyBac aa sequence with | With D268N and/or D346N |
| | mutations in the | |
| | catalytic triad | |
| 12 | Modified hyperactive | SEQ ID NO: 9 |
| | PiggyBac aa sequence with | With K287A, K287A/K290A, R460A/K461A, or |
| | mutations in amino acids | any combination thereof |
| | that are critical for | |
| | excision | |
| 13 | Modified hyperactive | SEQ ID NO: 9 |
| | PiggyBac aa sequence with | With S351E, S351P, S351A, K356E, or any |
| | mutation that are | combination thereof |
| | involved in target | |
| | joining | |
| 14 | Modified hyperactive | SEQ ID NO: 9 |
| | PiggyBac aa sequence with | With T560A, S564P, S571N, S573A, M589V, |
| | mutations that are | S592G, F594L, or any combination thereof |
| | critical for integration | |
| 15 | Modified hyperactive | SEQ ID NO: 9 |
| | PiggyBac aa sequence with | With G325A, N347A, N347S, T350A, W465A, or |
| | mutations that are | any combination thereof |
| | involved in alignment | |
| 16 | Modified hyperactive | SEQ ID NO: 9 |
| | PiggyBac aa sequence with | With K576A and/or I587A |
| | mutations at well | |
| | conserved amino acids | |
| 17 | Modified hyperactive | SEQ ID NO: 9 |
| | PiggyBac aa sequence with | With H586A |
| | mutations involved in Zn²⁺ | |
| | binding | |
| 18 | Modified hyperactive | SEQ ID NO: 9 |
| | PiggyBac aa sequence with | With R315A, R341A, R372A, K375A, or any |
| | mutations that are | combination thereof |
| | involved in integration | |
| 19 | Cas9 from Corynebacterium | MTNAVANHHVLWAKEDNVSEPYPLLAHLLDTATAATCLENHWL |
| | ulcerans aa sequence | RKGLRDRLSTELGPDAEKILGFVAGIHDLGKANPYFQAQRRNK |
| | | KEEWITLRDAIQKAGFPLSNGTSALFEETKEKRRHENITLSIL |
| | | GWEITKFLQVKDVWPQLAIIGHHGNFSAPGFLSDEDDLEDIED |
| | | IFDDNGWSPTHELLVSSLLQAVGLEKQPEIKHISPASAILISG |
| | | LVVLADRIASQSEMASDGLQALQKEELFFHQPEKWIANRKAFC |
| | | REIIENTVGTYHPWESEAAGIRAVLGDYEPRFTQKAALNAGDG |
| | | LENVMETTGAGKTEAALLRHVKRKERLLFFLPTQATTNAIMDR |
| | | IGKIFDGTPNVASLAHGLAVTEDFYAHPILPVQGSSDDANYKD |
| | | NGGLYPTEFVRSAGTPRLLAPVCVGTIDQALMGALPSKENHLR |
| | | LLALANAHVVVDEVHTMDQYQSELMSGLLEWWSATDTPVTLLT |
| | | ATMPAWQREKFHLSYTGKDPHFKGVFPSLEDWSTPSKNTETSQ |
| | | ENIPTEAFTIPINIDKIAHNEIVDSHVQWVIEQRKLFPQARIG |
| | | IICNTVGRAQSIAEALAHESPIVLHSRMTAGHRKEAATKLEQA |
| | | IGKKGTANATLVIGTQAIEASLDIDLDLLRTELCPAPSLIQRA |
| | | GRLWRRLDPQREVRVPGMVGKKLTIAVVDSPSTGQTLPYLRSQ |
| | | LYRVESWLKQRDRIEFPADIQDFIDATTPGLQELFQKVSLPED |
| | | CGSAEEREALADDYLNEVASWVTKQRQAGTSRIDFAKHGKPRQ |
| | | VLASDCVVEDFLQITSANNLEESATRLIDYPSISAILCDPTGT |
| | | IPGAWTDSVEKLIAISAKDSESLRRALRASISIPHSKKFLPIT |
| | | SREIPLSEAKTLLSGYSAVHIQPDEYDLQSGLKGPQK |
| 20 | Cas9 from Corynebacterium | MNPHEELWAKQKGLAKPYPLLAHLLDSAAVAGALWDHWLRQDL |
| | diphtheria aa sequence | RQMFIEELGSNAREIIQFVVGSHDIGKATPLFQYQKAQKGEVW |
| | | DSIRYAIDRTGRYQKPLPSSYLVKKTSGGPNRHEQWSSFASKN |
| | | EYLKPSAAAKENWIGLAIGGHHGRFEPVGYGRHQRKAAEDLAK |
| | | SGWSAAQQDLLRALEKASGITRASLPSELSPELTLVLSGLTIL |
| | | ADRISSTESFVITGARMIDDGTLHLATPIDWLKTRKLDSEKHV |
| | | AKTVGIYHGWNNHESAIHSILKGYDPRPLQTIALQNQVGLLNL |
| | | MAPTGNGKTEAAILRHSLKENDRLIFLLPTQATSNAIMRRVQG |
| | | IYSDTPNAAALAHSLASVEDFYQTPLSVFDDHYDPSKEQFESS |
| | | MSGGLYPSSFVCSGAARLLAPICIGTVDQALATALPGKWIHLR |
| | | ILALANAHIVIDEVHTLDHYQTALLENILPILAKLKTKITFLT |
| | | ATMPSWQRTKLLTAYGGEDLQIPPTVFPAAETVLPGQFNRTLI |
| | | DSDSTTIDFTMEETSYDHLVESHVKWHQTTRLNAPHARIGLIC |
| | | NTVKRAQEIAAALEKTNDRIVLLHSRMTTEHRRRSAELLESLL |
| | | GPNGNRKTITVVGTQAIEASLDIDLDILRTELCPAPSLVQRAG |
| | | RVWRRNDPYRSSRITADHKPISVVFIAEAKDWQVLPYLRAETS |
| | | RTQRWLEKHNQMFLPQMAQEFIDAATVDLDTATSEMDLDALAL |
| | | MGIHLMKADGAKARIQDVLNSDSKVSDFALLTSKNEIDEAQTR |
| | | LIEEGTHLRIILGDENESIPGGWKHGLSSLLKLKASDRESLRT |
| | | ALLASIPLLVSEKQKQLLYQHNLVPLSSSKTVLAGFYFLPKAQ |
| | | NFYSKNLGFIWPEEKD |
| 21 | Cas9 from Spiroplasma | MNYKKLILGLDLGIASCGWAVTGQMEDGNWVLDDFGVRLFQTP |
| | syrphidicola aa sequence | ENSKDGTTNAAARRLKRGARRLIKRRKNRIKDLKNLFEKINFI |
| | | NKASLDKYINEHSATNLVEDFNRHELYNPYFLRSIGITEKLTR |
| | | EELVWSLIHIANRRGYKNKFAFDIEGDGKKRETKLDEAISNAL |
| | | ISSNLTISQEIVRNKKFRDAKNKKALLVRNKGGKEGENNFQFL |
| | | FARDDYKKEVDLLLAKQAKFYPELTEEIRAKAADIIFRQRDFE |
| | | DGPGPKKQELREIYKKENKQFSKNFTQLEGRCTFLRELSVGYK |
| | | SSILFDLFHIISEVSKISKYIEENDQLAQDIISSFLYNEAGKK |
| | | GKTLLKEILKKHHINDDIFDTNAYKNIDEKTNYLNLLKEVEGN |
| | | DVLKNLSLNRLEDNIYHQLGFIIHTNITPERKEKAINQWLLEN |
| | | NIILAKEKLNILLKPNSSISTTVKTSFKWMSIAISNFLKGIPY |
| | | GKFQAQFIKEDNFKLPESYAKQYQKYLTGEKTFEMFAPIIDPD |
| | | LWRNPIVFRAINQARKVIKKLFEKYTFIDQINIELTREMGLSF |
| | | SDRKKVKERQDDSLKENAKAKEFLMANGIIVNDTNVLKYKLWI |
| | | QQNKKSLYSGKEITIADLGASNVLQIDHIIPYSKLADDSFNNK |
| | | VLVFSKENQEKGNQFADQYVKSLGTENYNNYKKRVNYLLFQNQ |
| | | INQKKAEYLLCSNQNEEILNDFVSRNLNDTRYITRYVTNWLKA |
| | | EFELQSRFGLAKPKIMTLNGAITSRFRRTWLRNSPWGLEKKS |
| 22 | Cas9 from Prevotella | MKRILGLDLGTTSIGWALVNEAENNNEASSIVRLGVRVNPLTV |
| | intermedia aa sequence | DEKSNFEKGKAITTNADRQLRHGARINLQRYKLRRQNLHDCLQ |
| | | KQGWLGTEAMYEEGKASTFETYKLRAKAAEEEISLHEFARVLF |
| | | MLNKKRGYKSNRKANNKEDGQLFDGMTIAKKLYEEHLTPAEYS |
| | | LQLLNKGKKFTQGYYRSDLNAELERIWDEQKKYYPEILTDEFK |
| | | QQLEGKTKTNTSKIFLAKYGIYSADLKGLDRKFQPLKWRVEAL |
| | | QQQVDKEVLAFVISDLKGQIANTSGLLGAISDRSKELYFNKQT |
| | | VGQYLWASLEENPHISIKNKPFYRQDYLDEFEKIWETQAAFHK |
| | | QLTPELKQEIRDIIIFYQRPLKSKKSLISVCELEQRKVKATID |
| | | GKEKEITIGPKVAPKSSPVFQEFRIWQNLNNVLLIDNDTNEKR |
| | | PLDEVERNLLYKELSIKAKLSKTEALKILNKKGKQWDLNYREL |
| | | EGNRTQAILFDCYNRIITLTGHEECDFKKIKASEIRHYVSTIF |
| | | KNLGFSTEILDFDPSLKKHELEKQPMYQLWHLLYSYESDNSRT |
| | | GNESLLRKLETTFGFPEEYATVLCDVVFEEDYGNLSVKAMREI |
| | | LPYLQAGNDYSQACAYAGYNHSRHSLTKEELDQKVYKERLELL |
| | | PKNSLRNPVVEKILNQMINVINAIIDEYGKPDEIRIEMARELK |
| | | SSAADRKKTTHAISQGNAENQRIREILEKEFSLSYISRNDIIK |
| | | YKLYEELEPNYYKTLYSDTYITKDKLFSKDFDIEHIIPKARLF |
| | | DDSFSNKTLEARNINLEKSNKTAFDFIKEKYGEDGAEAYKKKL |
| | | DMLLENDAISRPKYNNLLRAEADIPSDFINRDLRNTQYIAKKA |
| | | CEILGELVKTVTPTTGKITNRLREDWQLVDVMKELNFEKYEKL |
| | | GLTEIVEDRDGRKIKRIKDWTKRNDHRHHAMDALAIAFTKPSF |
| | | IQYLNNLNARSNKGDSIYAIENKELHYEEGKLRFNAPIPVNEF |
| | | RAEAKRHLSAILVSIKAKNKVMTQNVNKIKTKHGIIKKIQLTP |
| | | RGPLHNETIYGTKMRPIIKMVKVGAALDEATINKVSSPAIREA |
| | | LLKRLNEYSGNAKKAFTGKNTLEKNPIYLNAGRTKTVPSLVKT |
| | | VEWESFHPTRKLIDKDLNVDKVVDKGIREILKARLEEFNGDAK |
| | | KAFSNLEENPIYLDEAKKIALKRVSIEGVLSAIPLHTLKNQAG |
| | | KPITGKDGKPVLGNYVQTSNNHHIAFYYDEDGNLQDNAVSFFE |
| | | AAERKSQGIPVIDKDYNRDKGWRFLFTMKQNEYFVFPNEATGF |
| | | IPSEVDLTDEANYGIISPNLYRVQKVSRIDKGTSASRDYWFRH |
| | | HLETILNDDAKLKNLAFKRIRGLLELKDIIKVRINSTGKIVAV |
| | | GEYD |
| 23 | Cas9 from Spiroplasma | MWSRKILKAGSRLFDEANLSDKIASKRREQRGRRRNLRRKITW |
| | taiwanense aa sequence | KQDLINLEVKYNFLQKENDFYELDENFDLLELRKKAINSKIEL |
| | | EQLLIILFNYIKHRGSFNYREDLSELKNISQEELETSSEFKLP |
| | | VDIQFELKEENNKFREINNEKSLINHEWYVKEINLILDAQIEN |
| | | KLINLDFKKDYLKLFNRKREYYDGPGPKDKNLLNPSKYGWKNQ |
| | | EEFFDRFAGKDTYDSKEQRAPKHSLTSYLFNILNDLNNLSING |
| | | DRNQLTYENKKDLINLTLINQKEKAENITLKKIAKYLKINEKN |
| | | ITGYRLKPNSNESIFTVFESANKMRSILVKNNKSIDFICLENI |
| | | DKIDKIVDILTKYQSIEDKSLKLEELNFDFFDKETCEKLAVIS |
| | | LTGTHALSKKTMSKLIEEMFHDNLNHMEALAKLKIKPDYKLKV |
| | | DLTNEKTIPILREKINEMYISPVVKRALIESLKIIKELERHFK |
| | | DFEIKDIVIEMAKKNSAEKKQFISKIQRQNVDLVKKLSNDYSL |
| | | DENKLNFKMKEKFLLLSEQ |
| 24 | Cas9 from Streptococcus | MRKPYSIGLDIGTNSVGWAVITDDYKVPSKKMRIQGTTDRTSI |
| | iniae aa sequence | KKNLIGALLFDNGETAEATRLKRTTRRRYTRRKYRIKELQKIF |
| | | SSEMNELDIAFFPRLSESELVSDDKEFENHPIEGNLKDEITYH |
| | | NDYPTIYHLRQTLADSDQKADLRLIYLALAHIIKFRGHFLIEG |
| | | NLDSENTDVHVLFLNLVNIYNNLFEEDIVETASIDAEKILTSK |
| | | TSKSRRLENLIAEIPNQKRNMLFGNLVSLALGLTPNFKTNFEL |
| | | LEDAKLQISKDSYEEDLDNLLAQIGDQYADLFIAAKKLSDAIL |
| | | LSDIITVKGASTKAPLSASMVQRYEEHQQDLALLKNLVKKQIP |
| | | EKYKEIFDNKEKNGYAGYIDGKTSQEEFYKYIKPILLKLDGTE |
| | | KLISKLEREDFLRKQRTFDNGSIPHQIHLNELKAIIRRQEKFY |
| | | PFLKENQKKIEKLFTFKIPYYVGPLANGQSSFAWLKRQSNESI |
| | | TPWNFEEVVDQEASARAFIERMTNFDTYLPEEKVLPKHSPLYE |
| | | MFMVYNELTKVKYQTEGMKRPVFLSSEDKEEIVNLLFKKERKV |
| | | TVKQLKEEYFSKMKCFHTVTILGVEDRFNASLGTYHDLLKIFK |
| | | DKAFLDDEANQDILEEIVWTLTLFEDQAMIERRLVKYADVFEK |
| | | SVLKKLKKRHYTGWGRLSQKLINGIKDKQTGKTILGFLKDDGV |
| | | ANRNFMQLINDSSLDFAKIIKNEQEKTIKNESLEETIANLAGS |
| | | PAIKKGILQSIKIVDEIVKIMGQNPDNIVIEMARENQSTMQGI |
| | | KNSRQRLRKLEEVHKNTGSKILKEYNVSNTQLQSDRLYLYLLQ |
| | | DGKDMYTGKELDYDNLSQYDIDHIIPQSFIKDNSIDNTVLTTQ |
| | | ASNRGKSDNVPNIETVNKMKSFWYKQLKSGAISQRKFDHLTKA |
| | | ERGALSDFDKAGFIKRQLVETRQITKHVAQILDSRENSNLTED |
| | | SKSNRNVKIITLKSKMVSDERKDEGFYKLREVNDYHHAQDAYL |
| | | NAVVGTALLKKYPKLEAEFVYGDYKHYDLAKLMIQPDSSLGKA |
| | | TTRMFFYSNLMNFFKKEIKLADDTIFTRPQIEVNTETGEIVWD |
| | | KVKDMQTIRKVMSYPQVNIVMKTEVQTGGFSKESIWPKGDSDK |
| | | LIARKKSWDPKKYGGFDSPIIAYSVLVVAKIAKGKTQKLKTIK |
| | | ELVGIKIMEQDEFEKDPIAFLEKKGYQDIQTSSIIKLPKYSLF |
| | | ELENGRKRLLASAKELQKGNELALPNKYVKFLYLASHYTKFTG |
| | | KEEDREKKRSYVESHLYYFDVRLSQVFRVTNVEF |
| 25 | Cas9 from Belliella | MKKILGLDLGTTSIGWAFIKEPEKDVVGSEIVDMGVRIVPLSS |
| | baltica aa sequence | DEENDFAKGNTISINADRTLKRGARRNLQRFKQRRNALLEIFK |
| | | EKKLISTNEKYAEDGPSSTESTLNLRAKAAKEKIELQDLVKVL |
| | | LQINKKRGYKSSRKAKSEEDDGSAIDSMGIAKELYENDLTPGQ |
| | | WVYEALQKGRKNVPDFYRSDLQEEFKKIVNYQSEFFPDIFNAS |
| | | FVEDWMGKASTPTKQYFNKKGVQLAENKGKREERRLQEYKWRA |
| | | EAVNFKIDLSEIALILSQINSQISNSSGYLGAISDRSKELYFK |
| | | NLTVGQYLYQQIKKNPHTRLKGQVFYRQDYLDEFERIWSVQSS |
| | | FYPQLNDALKREVRDITIFFQRRLKSQKHLISNCEFEDHHKVV |
| | | PKSHPVFQEFRIWQNLNNLLLIKKDNLNEKFDLELESKIALAN |
| | | ELAFKRELNVKDALKILGLKPNEWEFNFTKIEGNRTNQAFFDA |
| | | FAKIIELEDGEPIDLGDLKADDILDQFSEAFLRIGIDTELLQV |
| | | NSDIEGAEYEKQSYIQFWHLLYSSEDDQKLKLNLIRKFGFKPE |
| | | HAKILASISLQDDHASLSSRAIKKILPHLQSGLIYDKACTYAG |
| | | YNHSSSFTKDENEKRELRAELELLKKNSLRNPVVEKILNQMIN |
| | | VVNAILKDPELGRPDEIRVEMARELKANAEQRKNMTSNIASAT |
| | | RDHDKYREILKSEFGLKRVTKNDLLRYKLWLETDGISLYTGKP |
| | | IEASKLFSKEYDIEHIIPKARLFDDSFSNKTICERQLNIDKAN |
| | | VTAFSFLQNKLSADEFEQYQSRVKSLYGKLSKAKIQKLLMAND |
| | | KIPEDFIARQLQETRYISKKAKEILFEISRRVSVTTGTITDKL |
| | | REDWGLVEIMKELNWEKYDKLGLTYTIEGKHGERLNKIKDWSK |
| | | RNDHRHHAMDALTVALTKPAYIQYLNNLNAKGLNNKKGTEVFA |
| | | IEQKYLKRENGKLCFIPPIENIRSEAKKHLSRILVSYKAKNKV |
| | | VTINKNKTKSKAGLNEQIALTPRGQLHKETVYGKSFHYSTKFE |
| | | KIGASFNVQKINTVAKKEEREALLKRLAENGNDPKKAFTGKNT |
| | | LNKMPIYLDLGKNIKLSEKVKTVVLEQNYTIRKNIDPDLKVDK |
| | | VIDVGIKRILESRLEEFGGNAKLAFSNLEENPIWLNKEKGISI |
| | | KRVKISGVSNVESLHVKKDHFGEPILDQEGNEIPVDFVSTGNN |
| | | HHVAIYEDENGNLQEEVVSFFEAVVRQNQGLPIIKKNHTLGWK |
| | | FLFTLKQNEYFVFPSDDEVPADVDLMDEQNYHLISPNLERVQK |
| | | IARKNYVENNHLETKAVDNDLLKSKKELSKITYHEYQTPEHLR |
| | | GIIKIRINHLGKIIQIGEY |
| 26 | Cas9 from Psychroflexus | MKRILGLDLGTNSIGWSLIEHDFKNKQGQIEGLGVRIIPMSQE |
| | torquisi aa sequence | ILGKFDAGQSISQTADRTKYRGVRRLYQRDNLRRERLHRVLKI |
| | | LDFLPKHYSESIDFQDKVGQFKPKQEVKLNYRKNEKNKHEFVF |
| | | MNSFIEMVSEFKNAQPELFYNKGNGEETKIPYDWTLYYLRKKA |
| | | LTQQITKEELAWLILNFNQKRGYYQLRGEDIDEDKNKKYMQLK |
| | | VNNLIDSGAKVKGKVLYNVIFDNGWKYEKQIVNKDEWEGRTKE |
| | | FIITTKTLKNGNIKRTYKAVDSEIDWAAIKAKTEQDINKANKT |
| | | VGEYIYESLLDNPSQKIRGKLVKTIERKFYKEEFEKLLSKQIE |
| | | LQPELFNESLYKACIKELYPRNENHQSNNKKQGFEYLFTEDII |
| | | FYQRPLKSQKSNISGCQFEHKIYKQKNKKTGKLELIKEPIKTI |
| | | SRSHPLFQEFRIWQWLQNLKIYNKEKIENGKLEDVTTQLLPNN |
| | | EAYVTLFDFLNTKKELEQKQFIEYFVKKKLIDKKEKEHFRWNF |
| | | VEDKKYPFSETRAQFLSRLAKVKGIKNTEDFLNKNTQVGSKEN |
| | | SPFIKRIEQLWHITYSVSDLKEYEKALEKFAEKHNLEKDSFLK |
| | | NEKKEPPFVSDYASYSKKAISKLLPIMRMGKYWSESAVPTQVK |
| | | ERSLSIMERVKVLPLKEGYSDKDLADLLSRVSDDDIPKQLIKS |
| | | FISFKDKNPLKGLNTYQANYLVYGRHSETGDIQHWKTPEDIDR |
| | | YLNNFKQHSLRNPIVEQVVMETLRVVRDIWEHYGNNEKDFFKE |
| | | IHVELGREMKSPAGKREKLSQRNTENENTNHRIREVLKELMND |
| | | ASVEGGVRDYSPSQQEILKLYEEGIYQNPNTNYLKVDEDEILK |
| | | IRKKNNPTQKEIQRYKLWLEQGYISPYTGKIIPLTKLFTHEYQ |
| | | IEHIIPQSRYYDNSLGNKIICESEVNEDKDNKTAYEYLKVEKG |
| | | SIVFGHKLLNLDEYEAHVNKYFKKNKTKLKNLLSEDIPEGFIN |
| | | RQLNDSRYISKLVKGLLSNIVRENGEQEATSKNLIPVTGVVTS |
| | | KLKQDWGLNDKWNEIIAPRFKRLNKLTNSNDFGFWDNDINAFR |
| | | IQVPDSLIKGFSKKRIDHRHHALDALVVACTSRNHTHYLSALN |
| | | AENKNYSLRDKLVIKNENGDYTKTFQIPWQGFTIEAKNNLEKT |
| | | VVSFKKNLRVINKTNNKFWSYKDENGNLNLGKDGKPKKKLRKQ |
| | | TKGYNWAIRKPLHKETVSGIYNINAPKNKIATSVRTLLTEIKN |
| | | EKHLAKITDLRIRETILPNHLKHYLNNKGEANFSEAFSQGGIE |
| | | DLNKKITTLNEGKKHQPIYRVKIFEVGSKFSISEDENSAKSKK |
| | | YVEAAKGTNLFFAIYLDEENKKRNYETIPLNEVITHQKQVAGF |
| | | PKSERLSVQPDSQKGTFLFTLSPNDLVYVPNNEELENRDLFNL |
| | | GNLNVEQISRIYKFTDSSDKTCNFIPFQVSKLIFNLKKKEQKK |
| | | LDVDFIIQNEFGLGSPQSKNQKSIDDVMIKEKCIKLKIDRLGN |
| | | ISKA |
| 27 | Cas9 from Streptococcus | MTKPYSIGLDIGTNSVGWAVTTDNYKVPSKKMKVLGNTSKKYI |
| | thermophilus aa sequence | KKNLLGVLLFDSGITAEGRRLKRTARRRYTRRRNRILYLQEIF |
| | | STEMATLDDAFFQRLDDSFLVPDDKRDSKYPIEGNLVEEKAYH |
| | | DEFPTIYHLRKYLADSTKKADLRLVYLALAHMIKYRGHFLIEG |
| | | EFNSKNNDIQKNFQDFLDTYNAIFESDLSLENSKQLEEIVKDK |
| | | ISKLEKKDRILKLFPGEKNSGIFSEFLKLIVGNQADFRKCFNL |
| | | DEKASLHFSKESYDEDLETLLGYIGDDYSDVFLKAKKLYDAIL |
| | | LSGFLTVTDNETEAPLSSAMIKRYNEHKEDLALLKEYIRNISL |
| | | KTYNEVFKDDTKNGYAGYIDGKTNQEDFYVYLKKLLAEFEGAD |
| | | YFLEKIDREDFLRKQRTFDNGSIPYQIHLQEMRAILDKQAKFY |
| | | PFLAKNKERIEKILTFRIPYYVGPLARGNSDFAWSIRKRNEKI |
| | | TPWNFEDVIDKESSAEAFINRMTSFDLYLPEEKVLPKHSLLYE |
| | | TFNVYNELTKVRFIAESMRDYQFLDSKQKKDIVRLYFKDKRKV |
| | | TDKDIIEYLHAIYGYDGIELKGIEKQFNSSLSTYHDLLNIIND |
| | | KEFLDDSSNEAIIEEIIHTLTIFEDREMIKQRLSKFENIFDKS |
| | | VLKKLSRRHYTGWGKLSAKLINGIRDEKSGNTILDYLIDDGIS |
| | | NRNFMQLIHDDALSFKKKIQKAQIIGDEDKGNIKEVVKSLPGS |
| | | PAIKKGILQSIKIVDELVKVMGGRKPESIVVEMARENQYTNQG |
| | | KSNSQQRLKRLEKSLKELGSKILKENIPAKLSKIDNNALQNDR |
| | | LYLYYLQNGKDMYTGDDLDIDRLSNYDIDHIIPQAFLKDNSID |
| | | NKVLVSSASNRGKSDDVPSLEVVKKRKTFWYQLLKSKLISQRK |
| | | FDNLTKAERGGLSPEDKAGFIQRQLVETRQITKHVARLLDEKF |
| | | NNKKDENNRAVRTVKIITLKSTLVSQFRKDFELYKVREINDFH |
| | | HAHDAYLNAVVASALLKKYPKLEPEFVYGDYPKYNSFRERKSA |
| | | TEKVYFYSNIMNIFKKSISLADGRVIERPLIEVNEETGESVWN |
| | | KESDLATVRRVLSYPQVNVVKKVEEQNHGLDRGKPKGLFNANL |
| | | SSKPKPNSNENLVGAKEYLDPKKYGGYAGISNSFTVLVKGTIE |
| | | KGAKKKITNVLEFQGISILDRINYRKDKLNFLLEKGYKDIELI |
| | | IELPKYSLFELSDGSRRMLASILSTNNKRGEIHKGNQIFLSQK |
| | | FVKLLYHAKRISNTINENHRKYVENHKKEFEELFYYILEFNEN |
| | | YVGAKKNGKLLNSAFQSWQNHSIDELCSSFIGPTGSERKGLFE |
| | | LTSRGSAADFEFLGVKIPRYRDYTPSSLLKDATLIHQSVTGLY |
| | | ETRIDLAKLGEG |
| 28 | Cas9 from Listeria | MKKPYTIGLDIGTNSVGWAVLTDQYDLVKRKMKIAGDSEKKQI |
| | innocua aa sequence | KKNFWGVRLFDEGQTAADRRMARTARRRIERRRNRISYLQGIF |
| | | AEEMSKTDANFFCRLSDSFYVDNEKRNSRHPFFATIEEEVEYH |
| | | KNYPTIYHLREELVNSSEKADLRLVYLALAHIIKYRGNFLIEG |
| | | ALDTQNTSVDGIYKQFIQTYNQVFASGIEDGSLKKLEDNKDVA |
| | | KILVEKVTRKEKLERILKLYPGEKSAGMFAQFISLIVGSKGNF |
| | | QKPFDLIEKSDIECAKDSYEEDLESLLALIGDEYAELFVAAKN |
| | | AYSAVVLSSIITVAETETNAKLSASMIERFDTHEEDLGELKAF |
| | | IKLHLPKHYEEIFSNTEKHGYAGYIDGKTKQADFYKYMKMTLE |
| | | NIEGADYFIAKIEKENFLRKQRTFDNGAIPHQLHLEELEAILH |
| | | QQAKYYPFLKENYDKIKSLVTFRIPYFVGPLANGQSEFAWLTR |
| | | KADGEIRPWNIEEKVDFGKSAVDFIEKMTNKDTYLPKENVLPK |
| | | HSLCYQKYLVYNELTKVRYINDQGKTSYFSGQEKEQIFNDLFK |
| | | QKRKVKKKDLELFLRNMSHVESPTIEGLEDSFNSSYSTYHDLL |
| | | KVGIKQEILDNPVNTEMLENIVKILTVFEDKRMIKEQLQQFSD |
| | | VLDGVVLKKLERRHYTGWGRLSAKLLMGIRDKQSHLTILDYLM |
| | | NDDGLNRNLMQLINDSNLSFKSIIEKEQVTTADKDIQSIVADL |
| | | AGSPAIKKGILQSLKIVDELVSVMGYPPQTIVVEMARENQTTG |
| | | KGKNNSRPRYKSLEKAIKEFGSQILKEHPTDNQELRNNRLYLY |
| | | YLQNGKDMYTGQDLDIHNLSNYDIDHIVPQSFITDNSIDNLVL |
| | | TSSAGNREKGDDVPPLEIVRKRKVFWEKLYQGNLMSKRKFDYL |
| | | TKAERGGLTEADKARFIHRQLVETRQITKNVANILHQRFNYEK |
| | | DDHGNTMKQVRIVTLKSALVSQFRKQFQLYKVRDVNDYHHAHD |
| | | AYLNGVVANTLLKVYPQLEPEFVYGDYHQFDWFKANKATAKKQ |
| | | FYTNIMLFFAQKDRIIDENGEILWDKKYLDTVKKVMSYRQMNI |
| | | VKKTEIQKGEFSKATIKPKGNSSKLIPRKTNWDPMKYGGLDSP |
| | | NMAYAVVIEYAKGKNKLVFEKKIIRVTIMERKAFEKDEKAFLE |
| | | EQGYRQPKVLAKLPKYTLYECEEGRRRMLASANEAQKGNQQVL |
| | | PNHLVTLLHHAANCEVSDGKSLDYIESNREMFAELLAHVSEFA |
| | | KRYTLAEANLNKINQLFEQNKEGDIKAIAQSFVDLMAFNAMGA |
| | | PASFKFFETTIERKRYNNLKELLNSTIIYQSITGLYESRKRLD |
| | | D |
| 29 | Cas9 from Campylobacter | MARILAFDIGISSIGWAFSENDELKDCGVRIFTKVENPKTGES |
| | jejuni aa sequence | LALPRRLARSARKRLARRKARLNHLKHLIANEFKLNYEDYQSF |
| | | DESLAKAYKGSLISPYELRFRALNELLSKQDFARVILHIAKRR |
| | | GYDDIKNSDDKEKGAILKAIKQNEEKLANYQSVGEYLYKEYFQ |
| | | KFKENSKEFTNVRNKKESYERCIAQSFLKDELKLIFKKQREFG |
| | | FSFSKKFEEEVLSVAFYKRALKDFSHLVGNCSFFTDEKRAPKN |
| | | SPLAFMFVALTRIINLLNNLKNTEGILYTKDDLNALLNEVLKN |
| | | GTLTYKQTKKLLGLSDDYEFKGEKGTYFIEFKKYKEFIKALGE |
| | | HNLSQDDLNEIAKDITLIKDEIKLKKALAKYDLNQNQIDSLSK |
| | | LEFKDHLNISFKALKLVTPLMLEGKKYDEACNELNLKVAINED |
| | | KKDFLPAFNETYYKDEVTNPVVLRAIKEYRKVLNALLKKYGKV |
| | | HKINIELAREVGKNHSQRAKIEKEQNENYKAKKDAELECEKLG |
| | | LKINSKNILKLRLFKEQKEFCAYSGEKIKISDLQDEKMLEIDH |
| | | IYPYSRSFDDSYMNKVLVFTKQNQEKLNQTPFEAFGNDSAKWQ |
| | | KIEVLAKNLPTKKQKRILDKNYKDKEQKNFKDRNLNDTRYIAR |
| | | LVLNYTKDYLDFLPLSDDENTKLNDTQKGSKVHVEAKSGMLTS |
| | | ALRHTWGFSAKDRNNHLHHAIDAVIIAYANNSIVKAFSDFKKE |
| | | QESNSAELYAKKISELDYKNKRKFFEPFSGFRQKVLDKIDEIF |
| | | VSKPERKKPSGALHEETFRKEEEFYQSYGGKEGVLKALELGKI |
| | | RKVNGKIVKNGDMERVDIFKHKKTNKFYAVPIYTMDFALKVLP |
| | | NKAVARSKKGEIKDWILMDENYEFCFSLYKDSLILIQTKDMQE |
| | | PEFVYYNAFTSSTVSLIVSKHDNKFETLSKNQKILFKNANEKE |
| | | VIAKSIGIQNLKVFEKYIVSALGEVTKAEFRQREDFKK |
| 30 | Cas9 from Neisseria | MAAFKPNPINYILGLDIGIASVGWAMVEIDEDENPICLIDLGV |
| | meningitidis aa sequence | RVFERAEVPKTGDSLAMARRLARSVRRLTRRRAHRLLRARRLL |
| | | KREGVLQAADFDENGLIKSLPNTPWQLRAAALDRKLTPLEWSA |
| | | VLLHLIKHRGYLSQRKNEGETADKELGALLKGVADNAHALQTG |
| | | DERTPAELALNKFEKESGHIRNQRGDYSHTFSRKDLQAELILL |
| | | FEKQKEFGNPHVSGGLKEGIETLLMTQRPALSGDAVQKMLGHC |
| | | TFEPAEPKAAKNTYTAERFIWLTKLNNLRILEQGSERPLTDTE |
| | | RATLMDEPYRKSKLTYAQARKLLGLEDTAFFKGLRYGKDNAEA |
| | | STLMEMKAYHAISRALEKEGLKDKKSPLNLSPELQDEIGTAFS |
| | | LEKTDEDITGRLKDRIQPEILEALLKHISFDKFVQISLKALRR |
| | | IVPLMEQGKRYDEACAEIYGDHYGKKNTEEKIYLPPIPADEIR |
| | | NPVVLRALSQARKVINGVVRRYGSPARIHIETAREVGKSFKDR |
| | | KEIEKRQEENRKDREKAAAKFREYFPNFVGEPKSKDILKLRLY |
| | | EQQHGKCLYSGKEINLGRLNEKGYVEIDHALPFSRTWDDSFNN |
| | | KVLVLGSENQNKGNQTPYEYENGKDNSREWQEFKARVETSRFP |
| | | RSKKQRILLQKFDEDGFKERNLNDTRYVNRFLCQFVADRMRLT |
| | | GKGKKRVFASNGQITNLLRGFWGLRKVRAENDRHHALDAVVVA |
| | | CSTVAMQQKITRFVRYKEMNAFDGKTIDKETGEVLHQKTHFPQ |
| | | PWEFFAQEVMIRVFGKPDGKPEFEEADTPEKLRTLLAEKLSSR |
| | | PEAVHEYVTPLFVSRAPNRKMSGQGHMETVKSAKRLDEGVSVL |
| | | RVPLTQLKLKDLEKMVNREREPKLYEALKARLEAHKDDPAKAF |
| | | AEPFYKYDKAGNRTQQVKAVRVEQVQKTGVWVRNHNGIADNAT |
| | | MVRVDVFEKGDKYYLVPIYSWQVAKGILPDRAVVQGKDEEDWQ |
| | | LIDDSFNEKESLHPNDLVEVITKKARMFGYFASCHRGTGNINI |
| | | RIHDLDHKIGKNGILEGIGVKTALSFQKYQIDELGKEIRPCRL |
| | | KKRPPVR |
| 31 | Cas9 from Streptococcus | MDKKYSIGLDIGTNSVGWAVITDDYKVPSKKFKVLGNTDRHSI |
| | pyogenes aa sequence | KKNLIGALLFGSGETAEATRLKRTARRRYTRRKNRICYLQEIF |
| | | SNEMAKVDDSFEHRLEESELVEEDKKHERHPIEGNIVDEVAYH |
| | | EKYPTIYHLRKKLADSTDKADLRLIYLALAHMIKFRGHFLIEG |
| | | DLNPDNSDVDKLFIQLVQIYNQLFEENPINASRVDAKAILSAR |
| | | LSKSRRLENLIAQLPGEKRNGLFGNLIALSLGLTPNFKSNFDL |
| | | AEDAKLQLSKDTYDDDLDNLLAQIGDQYADLFLAAKNLSDAIL |
| | | LSDILRVNSEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFY |
| | | PFLKDNREKIEKILTFRIPYYVGPLARGNSRFAWMTRKSEETI |
| | | TPWNFEEVVDKGASAQSFIERMTNFDKNLPNEKVLPKHSLLYE |
| | | YFTVYNELTKVKYVTEGMRKPAELSGEQKKAIVDLLEKTNRKV |
| | | TVKQLKEDYFKKIECEDSVEISGVEDRFNASLGAYHDLLKIIK |
| | | DKDFLDNEENEDILEDIVLTLTLFEDRGMIEERLKTYAHLFDD |
| | | KVMKQLKRRRYTGWGRLSRKLINGIRDKQSGKTILDFLKSDGF |
| | | ANRNFMQLIHDDSLTEKEDIQKAQVSGQGHSLHEQIANLAGSP |
| | | AIKKGILQTVKIVDELVKVMGHKPENIVIEMARENQTTQKGQK |
| | | NSRERMKRIEEGIKELGSQILKEHPVENTQLQNEKLYLYYLQN |
| | | GRDMYVDQELDINRLSDYDVDHIVPQSFIKDDSIDNKVLTRSD |
| | | KNRGKSDNVPSEEVVKKMKNYWRQLLNAKLITQRKFDNLTKAE |
| | | RGGLSELDKAGFIKRQLVETRQITKHVAQILDSRMNTKYDEND |
| | | KLIREVKVITLKSKLVSDFRKDFQFYKVREINNYHHAHDAYLN |
| | | AVVGTALIKKYPKLESEFVYGDYKVYDVRKMIAKSEQEIGKAT |
| | | AKYFFYSNIMNFFKTEITLANGEIRKRPLIETNGETGEIVWDK |
| | | GRDFATVRKVLSMPQVNIVKKTEVQTGGFSKESILPKRNSDKL |
| | | IARKKDWDPKKYGGFDSPTVAYSVLVVAKVEKGKSKKLKSVKE |
| | | LLGITIMERSSFEKNPIDFLEAKGYKEVKKDLIIKLPKYSLFE |
| | | LENGRKRMLASAGELQKGNELALPSKYVNFLYLASHYEKLKGS |
| | | PEDNEQKQLFVEQHKHYLDEIIEQISEFSKRVILADANLDKVL |
| | | SAYNKHRDKPIREQAENIIHLFTLTNLGAPAAFKYFDTTIDRK |
| | | RYTSTKEVLDATLIHQSITGLYETRIDLSQLGGD |
| 32 | Zinc Finger Protein (ZFP) | atggcccaggctgctcttgagcccggagagaaaccctacaagt |
| | na sequence | gcccggagtgcggaaagtccttctctgagcggagtcacctccg |
| | | agagcaccagcggactcatacgggcgaaaaaccatacaagtgc |
| | | ccagaatgtggtaaatctttttctcgggctgacaacctgactg |
| | | aacatcagcgcacgcacaccggtgaaaaaccttacaagtgtcc |
| | | agagtgtggcaagagcttttctagtagaaggacctgtcgagcg |
| | | catcagcggactcacaccggcgaaaaaccctataagtgtccgg |
| | | aatgtggaaagagctttagccgcaacgacacccttactgaaca |
| | | ccagcgaacacacacgggagaaaaaccatataaatgtccggaa |
| | | tgtggcaaaagttttagtcggagtgataaacttacggagcacc |
| | | aacggacacacaccggagagaagccatataagtgtcctgaatg |
| | | tggaaagtccttctcacagcttgctcatctgcgagcacatcag |
| | | cgcacacacacc |
| 33 | ZFP aa sequence | MAQAALEPGEKPYKCPECGKSFSERSHLREHQRTHTGEKPYKC |
| | | PECGKSFSRADNLTEHQRTHTGEKPYKCPECGKSFSSRRTCRA |
| | | HQRTHTGEKPYKCPECGKSFSRNDTLTEHQRTHTGEKPYKCPE |
| | | CGKSFSRSDKLTEHQRTHTGEKPYKCPECGKSFSQLAHLRAHQ |
| | | RTHT |
| 34 | ZNF-E2C na sequence | atggcgcaggcggcgctggaaccgggcgaaaaaccgtataaat |
| | | gcccggaatgcggcaaaagctttagccgcaaagatagcctggt |
| | | gcgccatcagcgcacccataccggcgaaaaaccgtataaatgc |
| | | ccggaatgcggcaaaagctttagccagagcggcgatctgcgcc |
| | | gccatcagcgcacccataccggcgaaaaaccgtataaatgccc |
| | | ggaatgcggcaaaagctttagcgattgccgcgatctggcgcgc |
| | | catcagcgcacccataccggcgaaaaaccgtataaatgcccgg |
| | | aatgcggcaaaagctttagccagagcagccatctggtgcgcca |
| | | tcagcgcacccataccggcgaaaaaccgtataaatgcccggaa |
| | | tgcggcaaaagctttagcgattgccgcgatctggcgcgccatc |
| | | agcgcacccataccggcgaaaaaccgtataaatgcccggaatg |
| | | cggcaaaagctttagccgcagcgataaactggtgcgccatcag |
| | | cgcacccataccggcaaaaaaaccagcggccaggcgggc |
| 35 | ZNF-E2C aa sequence | MAQAALEPGEKPYKCPECGKSFSRKDSLVRHQRTHTGEKPYKC |
| | | PECGKSFSQSGDLRRHQRTHTGEKPYKCPECGKSFSDCRDLAR |
| | | HQRTHTGEKPYKCPECGKSFSQSSHLVRHQRTHTGEKPYKCPE |
| | | CGKSFSDCRDLARHQRTHTGEKPYKCPECGKSFSRSDKLVRHQ |
| | | RTHTGKKTSGQAG |
| 36 | ZNF-E3 na sequence | atggcgcaggcggcgctggaaccgggcgaaaaaccgtataaat |
| | | gcccggaatgcggcaaaagctttagcgatccgggcgcgctggt |
| | | gcgccatcagcgcacccataccggcgaaaaaccgtataaatgc |
| | | ccggaatgcggcaaaagctttagccagagcagccatctggtgc |
| | | gccatcagcgcacccataccggcgaaaaaccgtataaatgccc |
| | | ggaatgcggcaaaagctttagcgattgccgcgatctggcgcgc |
| | | catcagcgcacccataccggcgaaaaaccgtataaatgcccgg |
| | | aatgcggcaaaagctttagccagagcagccatctggtgcgcca |
| | | tcagcgcacccataccggcgaaaaaccgtataaatgcccggaa |
| | | tgcggcaaaagctttagcgattgccgcgatctggcgcgccatc |
| | | agcgcacccataccggcgaaaaaccgtataaatgcccggaatg |
| | | cggcaaaagctttagccagagcagccatctggtgcgccatcag |
| | | cgcacccataccggcaaaaaaaccagcggccaggcgggc |
| 37 | ZNF-E3 aa sequence | MAQAALEPGEKPYKCPECGKSFSDPGALVRHQRTHTGEKPYKC |
| | | PECGKSFSQSSHLVRHQRTHTGEKPYKCPECGKSFSDCRDLAR |
| | | HQRTHTGEKPYKCPECGKSFSQSSHLVRHQRTHTGEKPYKCPE |
| | | CGKSFSDCRDLARHQRTHTGEKPYKCPECGKSFSQSSHLVRHQ |
| | | RTHTGKKTSGQAG |
| 38 | ZNF-TRCa na sequence | atggcgcaggcggctcttgaacccggggagaaaccctataaat |
| | | gccctgagtgtggcaagagtttttcaaccacaggaaacttgac |
| | | agtccaccaacggacccacaccggcgagaaaccatacaagtgt |
| | | ccggagtgtggtaagtctttctcaagtcctgccgaccttacca |
| | | gacatcaacgcacacatacaggtgaaaaaccttacaagtgccc |
| | | agagtgcggaaaaagtttttcacaatctggcgacctccgcagg |
| | | caccagcgcactcacaccggtgaaaaaccatacaagtgtcctg |
| | | agtgcgggaagagttttagtcaacgagctcatctggagcgaca |
| | | ccaaaggactcatactggggagaaaccgtacaaatgtcccgaa |
| | | tgtgggaagagcttctctaccaagaattcccttacagagcacc |
| | | agcgcacgcatacgggagagaagccgtataagtgtccggaatg |
| | | tggcaagagcttttccagaagtgaccaccttacaacccaccag |
| | | aggacgcacacc |
| 39 | ZNF-TRCa aa sequence | MAQAALEPGEKPYKCPECGKSFSTTGNLTVHQRTHTGEKPYKC |
| | | PECGKSFSSPADLTRHQRTHTGEKPYKCPECGKSFSQSGDLRR |
| | | HQRTHTGEKPYKCPECGKSFSQRAHLERHQRTHTGEKPYKCPE |
| | | CGKSFSTKNSLTEHQRTHTGEKPYKCPECGKSFSRSDHLTTHQ |
| | | RTHT |
| 40 | AAVS1 site | agacggccgcgtcagagc |
| 41 | Zinc Finger 1 domain aa | ERSHLRE |
| | sequence | |
| 42 | Zinc Finger 2 domain aa | RADNLTE |
| | sequence | |
| 43 | Zinc Finger 3 domain aa | SRRTCRA |
| | sequence | |
| 44 | Zinc Finger 4 domain aa | RNDTLTE |
| | sequence | |
| 45 | Zinc Finger 5 domain aa | RSDKLTE |
| | sequence | |
| 46 | Zinc Finger 6 domain aa | QLAHLRA |
| | sequence | |
| 47 | Nuclear Localization | atggctccaaagaaaaagaggaaagtgggaatccacggagtcc |
| | Signal | ccgccgct |
| 48 | GGSx3 linker na sequence | ggtggatctggcggtggatctggtggcggt |
| 49 | GGSx3 linker aa sequence | GGSGGGSGGG |
| 50 | GGS4x Linker na sequence | ggagggagtggtgggtccggtggtagtggcggatcc |
| 51 | GGS4x Linker aa sequence | GGSGGSGGSGGS |
| 52 | GGS5x Linker na sequence | ggaggctccggtgggtctggtgggagcggtggtagtggcggat |
| | | cc |
| 53 | GGS5x Linker aa sequence | GGSGGSGGSGGSGGS |
| 54 | GGS6x Linker na sequence | ggaggcagtggtgggagcggtggttccgggggtagtggtggtt |
| | | ccgggggatcc |
| 55 | GGS6x Linker aa sequence | GGSGGSGGSGGSGGSGGS |
| 56 | GGS7x Linker na sequence | ggaggttctggaggctccggtgggtccgggggaagtggggggt |
| | | caggcggatcaggaggatcc |
| 57 | GGS7x Linker aa sequence | GGSGGSGGSGGSGGSGGSGGS |
| 58 | GGS8x Linker na sequence | ggaggtagcggaggttccggagggagcggcgggagtgggggaa |
| | | gcgggggaagtggaggatccgggggaggatcc |
| 59 | GGS8x Linker aa sequence | GGSGGSGGSGGSGGSGGSGGS |
| 60 | Linker XTEN na sequence | tccggtagcgaaacaccggggacttcagaatcggccaccccgg |
| | | agtct |
| 61 | Linker XTEN aa sequence | SGSETPGTSESATPES |
| 62 | Linker B na sequence | ggaagcgccggtagtgcggctgggtctggcgagttc |
| 63 | Linker B aa sequence | GSAGSAAGSGEF |
| 64 | human Cas9 (hCas9) na | atggacaagaagtactccattgggctcgatatcggcacaaaca |
| | sequence | gcgtcggctgggccgtcattacggacgagtacaaggtgccgag |
| | | caaaaaattcaaagttctgggcaataccgatcgccacagcata |
| | | aagaagaacctcattggcgccctcctgttcgactccggggaga |
| | | cggccgaagccacgcggctcaaaagaacagcacggcgcagata |
| | | tacccgcagaaagaatcggatctgctacctgcaggagatcttt |
| | | agtaatgagatggctaaggtggatgactctttcttccataggc |
| | | tggaggagtcctttttggtggaggaggataaaaagcacgagcg |
| | | ccacccaatctttggcaatatcgtggacgaggtggcgtaccat |
| | | gaaaagtacccaaccatatatcatctgaggaagaagcttgtag |
| | | acagtactgataaggctgacttgcggttgatctatctcgcgct |
| | | ggcgcatatgatcaaatttcggggacacttcctcatcgagggg |
| | | gacctgaacccagacaacagcgatgtcgacaaactctttatcc |
| | | aactggttcagacttacaatcagcttttcgaagagaacccgat |
| | | caacgcatccggagttgacgccaaagcaatcctgagcgctagg |
| | | ctgtccaaatcccggcggctcgaaaacctcatcgcacagctcc |
| | | ctggggagaagaagaacggcctgtttggtaatcttatcgccct |
| | | gtcactcgggctgacccccaactttaaatctaacttcgacctg |
| | | gccgaagatgccaagcttcaactgagcaaagacacctacgatg |
| | | atgatctcgacaatctgctggcccagatcggcgaccagtacgc |
| | | agacctttttttggcggcaaagaacctgtcagacgccattctg |
| | | ctgagtgatattctgcgagtgaacacggagatcaccaaagctc |
| | | cgctgagcgctagtatgatcaagcgctatgatgagcaccacca |
| | | agacttgactttgctgaaggcccttgtcagacagcaactgcct |
| | | gagaagtacaaggaaattttcttcgatcagtctaaaaatggct |
| | | acgccggatacattgacggcggagcaagccaggaggaatttta |
| | | caaatttattaagcccatcttggaaaaaatggacggcaccgag |
| | | gagctgctggtaaagcttaacagagaagatctgttgcgcaaac |
| | | agcgcactttcgacaatggaagcatcccccaccagattcacct |
| | | gggcgaactgcacgctatcctcaggcggcaagaggatttctac |
| | | ccctttttgaaagataacagggaaaagattgagaaaatcctca |
| | | catttcggataccctactatgtaggccccctcgcccggggaaa |
| | | ttccagattcgcgtggatgactcgcaaatcagaagagaccatc |
| | | actccctggaacttcgaggaagtcgtggataagggggcctctg |
| | | cccagtccttcatcgaaaggatgactaactttgataaaaatct |
| | | gcctaacgaaaaggtgcttcctaaacactctctgctgtacgag |
| | | tacttcacagtttataacgagctcaccaaggtcaaatacgtca |
| | | cagaagggatgagaaagccagcattcctgtctggagagcagaa |
| | | gaaagctatcgtggacctcctcttcaagacgaaccggaaagtt |
| | | accgtgaaacagctcaaagaagactatttcaaaaagattgaat |
| | | gtttcgactctgttgaaatcagcggagtggaggatcgcttcaa |
| | | cgcatccctgggaacgtatcacgatctcctgaaaatcattaaa |
| | | gacaaggacttcctggacaatgaggagaacgaggacattcttg |
| | | aggacattgtcctcacccttacgttgtttgaagatagggagat |
| | | gattgaagaacgcttgaaaacttacgctcatctcttcgacgac |
| | | aaagtcatgaaacagctcaagaggcgccgatatacaggatggg |
| | | ggcggctgtcaagaaaactgatcaatgggatccgagacaagca |
| | | gagtggaaagacaatcctggattttcttaagtccgatggattt |
| | | gccaaccggaacttcatgcagttgatccatgatgactctctca |
| | | cctttaaggaggacatccagaaagcacaagtttctggccaggg |
| | | ggacagtcttcacgagcacatcgctaatcttgcaggtagccca |
| | | gctatcaaaaagggaatactgcagaccgttaaggtcgtggatg |
| | | aactcgtcaaagtaatgggaaggcataagcccgagaatatcgt |
| | | tatcgagatggcccgagagaaccaaactacccagaagggacag |
| | | aagaacagtagggaaaggatgaagaggattgaagagggtataa |
| | | aagaactggggtcccaaatccttaaggaacacccagttgaaaa |
| | | cacccagcttcagaatgagaagctctacctgtactacctgcag |
| | | aacggcagggacatgtacgtggatcaggaactggacatcaatc |
| | | ggctctccgactacgacgtggatcatatcgtgccccagtcttt |
| | | tctcaaagatgattctattgataataaagtgttgacaagatcc |
| | | gataaaaatagagggaagagtgataacgtcccctcagaagaag |
| | | ttgtcaagaaaatgaaaaattattggcggcagctgctgaacgc |
| | | caaactgatcacacaacggaagttcgataatctgactaaggct |
| | | gaacgaggtggcctgtctgagttggataaagccggcttcatca |
| | | aaaggcagcttgttgagacacgccagatcaccaagcacgtggc |
| | | ccaaattctcgattcacgcatgaacaccaagtacgatgaaaat |
| | | gacaaactgattcgagaggtgaaagttattactctgaagtcta |
| | | agctggtctcagatttcagaaaggactttcagttttataaggt |
| | | gagagagatcaacaattaccaccatgcgcatgatgcctacctg |
| | | aatgcagtggtaggcactgcacttatcaaaaaatatcccaagc |
| | | ttgaatctgaatttgtttacggagactataaagtgtacgatgt |
| | | taggaaaatgatcgcaaagtctgagcaggaaataggcaaggcc |
| | | accgctaagtacttcttttacagcaatattatgaattttttca |
| | | agaccgagattacactggccaatggagagattcggaagcgacc |
| | | acttatcgaaacaaacggagaaacaggagaaatcgtgtgggac |
| | | aagggtagggatttcgcgacagtccggaaggtcctgtccatgc |
| | | cgcaggtgaacatcgttaaaaagaccgaagtacagaccggagg |
| | | cttctccaaggaaagtatcctcccgaaaaggaacagcgacaag |
| | | ctgatcgcacgcaaaaaagattgggaccccaagaaatacggcg |
| | | gattcgattctcctacagtcgcttacagtgtactggttgtggc |
| | | caaagtggagaaagggaagtctaaaaaactcaaaagcgtcaag |
| | | gaactgctgggcatcacaatcatggagcgatcaagcttcgaaa |
| | | aaaaccccatcgactttctcgaggcgaaaggatataaagaggt |
| | | caaaaaagacctcatcattaagcttcccaagtactctctcttt |
| | | gagcttgaaaacggccggaaacgaatgctcgctagtgcgggcg |
| | | agctgcagaaaggtaacgagctggcactgccctctaaatacgt |
| | | taatttcttgtatctggccagccactatgaaaagctcaaaggg |
| | | tctcccgaagataatgagcagaagcagctgttcgtggaacaac |
| | | acaaacactaccttgatgagatcatcgagcaaataagcgaatt |
| | | ctccaaaagagtgatcctcgccgacgctaacctcgataaggtg |
| | | ctttctgcttacaataagcacagggataagcccatcagggagc |
| | | aggcagaaaacattatccacttgtttactctgaccaacttggg |
| | | cgcgcctgcagccttcaagtacttcgacaccaccatagacaga |
| | | aagcggtacacctctacaaaggaggtcctggacgccacactga |
| | | ttcatcagtcaattacggggctctatgaaacaagaatcgacct |
| | | ctctcagctcggtggagac |
| 65 | nickase Cas9 (nCas9) na | atggacaagaagtactccattgggctcgctatcggcacaaaca |
| | sequence | gcgtcggctgggccgtcattacggacgagtacaaggtgccgag |
| | | caaaaaattcaaagttctgggcaataccgatcgccacagcata |
| | | aagaagaacctcattggcgccctcctgttcgactccggggaga |
| | | cggccgaagccacgcggctcaaaagaacagcacggcgcagata |
| | | tacccgcagaaagaatcggatctgctacctgcaggagatcttt |
| | | agtaatgagatggctaaggtggatgactctttcttccataggc |
| | | tggaggagtcctttttggtggaggaggataaaaagcacgagcg |
| | | ccacccaatctttggcaatatcgtggacgaggtggcgtaccat |
| | | gaaaagtacccaaccatatatcatctgaggaagaagcttgtag |
| | | acagtactgataaggctgacttgcggttgatctatctcgcgct |
| | | ggcgcatatgatcaaatttcggggacacttcctcatcgagggg |
| | | gacctgaacccagacaacagcgatgtcgacaaactctttatcc |
| | | aactggttcagacttacaatcagcttttcgaagagaacccgat |
| | | caacgcatccggagttgacgccaaagcaatcctgagcgctagg |
| | | ctgtccaaatcccggcggctcgaaaacctcatcgcacagctcc |
| | | ctggggagaagaagaacggcctgtttggtaatcttatcgccct |
| | | gtcactcgggctgacccccaactttaaatctaacttcgacctg |
| | | gccgaagatgccaagcttcaactgagcaaagacacctacgatg |
| | | atgatctcgacaatctgctggcccagatcggcgaccagtacgc |
| | | agacctttttttggcggcaaagaacctgtcagacgccattctg |
| | | ctgagtgatattctgcgagtgaacacggagatcaccaaagctc |
| | | cgctgagcgctagtatgatcaagcgctatgatgagcaccacca |
| | | agacttgactttgctgaaggcccttgtcagacagcaactgcct |
| | | gagaagtacaaggaaattttcttcgatcagtctaaaaatggct |
| | | acgccggatacattgacggcggagcaagccaggaggaatttta |
| | | caaatttattaagcccatcttggaaaaaatggacggcaccgag |
| | | gagctgctggtaaagcttaacagagaagatctgttgcgcaaac |
| | | agcgcactttcgacaatggaagcatcccccaccagattcacct |
| | | gggcgaactgcacgctatcctcaggcggcaagaggatttctac |
| | | ccctttttgaaagataacagggaaaagattgagaaaatcctca |
| | | catttcggataccctactatgtaggccccctcgcccggggaaa |
| | | ttccagattcgcgtggatgactcgcaaatcagaagagaccatc |
| | | actccctggaacttcgaggaagtcgtggataagggggcctctg |
| | | cccagtccttcatcgaaaggatgactaactttgataaaaatct |
| | | gcctaacgaaaaggtgcttcctaaacactctctgctgtacgag |
| | | tacttcacagtttataacgagctcaccaaggtcaaatacgtca |
| | | cagaagggatgagaaagccagcattcctgtctggagagcagaa |
| | | gaaagctatcgtggacctcctcttcaagacgaaccggaaagtt |
| | | accgtgaaacagctcaaagaagactatttcaaaaagattgaat |
| | | gtttcgactctgttgaaatcagcggagtggaggatcgcttcaa |
| | | cgcatccctgggaacgtatcacgatctcctgaaaatcattaaa |
| | | gacaaggacttcctggacaatgaggagaacgaggacattcttg |
| | | aggacattgtcctcacccttacgttgtttgaagatagggagat |
| | | gattgaagaacgcttgaaaacttacgctcatctcttcgacgac |
| | | aaagtcatgaaacagctcaagaggcgccgatatacaggatggg |
| | | ggcggctgtcaagaaaactgatcaatgggatccgagacaagca |
| | | gagtggaaagacaatcctggattttcttaagtccgatggattt |
| | | gccaacaggaacttcatgcagttgatccatgatgactctctca |
| | | cctttaaggaggacatccagaaagcacaagtttctggccaggg |
| | | ggacagtcttcacgagcacatcgctaatcttgcaggtagccca |
| | | gctatcaaaaagggaatactgcagaccgttaaggtcgtggatg |
| | | aactcgtcaaagtaatgggaaggcataagcccgagaatatcgt |
| | | tatcgagatggcccgagagaaccaaactacccagaagggacag |
| | | aagaacagtagggaaaggatgaagaggattgaagagggtataa |
| | | aagaactggggtcccaaatccttaaggaacacccagttgaaaa |
| | | cacccagcttcagaatgagaagctctacctgtactacctgcag |
| | | aacggcagggacatgtacgtggatcaggaactggacatcaatc |
| | | ggctctccgactacgacgtggatcatatcgtgccccagtcttt |
| | | tctcaaagatgattctattgataataaagtgttgacaagatcc |
| | | gataaaaatagagggaagagtgataacgtcccctcagaagaag |
| | | ttgtcaagaaaatgaaaaattattggcggcagctgctgaacgc |
| | | caaactgatcacacaacggaagttcgataatctgactaaggct |
| | | gaacgaggtggcctgtctgagttggataaagccggcttcatca |
| | | aaaggcagcttgttgagacacgccagatcaccaagcacgtggc |
| | | ccaaattctcgattcacgcatgaacaccaagtacgatgaaaat |
| | | gacaaactgattcgagaggtgaaagttattactctgaagtcta |
| | | agctggtctcagatttcagaaaggactttcagttttataaggt |
| | | gagagagatcaacaattaccaccatgcgcatgatgcctacctg |
| | | aatgcagtggtaggcactgcacttatcaaaaaatatcccaagc |
| | | ttgaatctgaatttgtttacggagactataaagtgtacgatgt |
| | | taggaaaatgatcgcaaagtctgagcaggaaataggcaaggcc |
| | | accgctaagtacttcttttacagcaatattatgaattttttca |
| | | agaccgagattacactggccaatggagagattcggaagcgacc |
| | | acttatcgaaacaaacggagaaacaggagaaatcgtgtgggac |
| | | aagggtagggatttcgcgacagtccggaaggtcctgtccatgc |
| | | cgcaggtgaacatcgttaaaaagaccgaagtacagaccggagg |
| | | cttctccaaggaaagtatcctcccgaaaaggaacagcgacaag |
| | | ctgatcgcacgcaaaaaagattgggaccccaagaaatacggcg |
| | | gattcgattctcctacagtcgcttacagtgtactggttgtggc |
| | | caaagtggagaaagggaagtctaaaaaactcaaaagcgtcaag |
| | | gaactgctgggcatcacaatcatggagcgatcaagcttcgaaa |
| | | aaaaccccatcgactttctcgaggcgaaaggatataaagaggt |
| | | caaaaaagacctcatcattaagcttcccaagtactctctcttt |
| | | gagcttgaaaacggccggaaacgaatgctcgctagtgcgggcg |
| | | agctgcagaaaggtaacgagctggcactgccctctaaatacgt |
| | | taatttcttgtatctggccagccactatgaaaagctcaaaggg |
| | | tctcccgaagataatgagcagaagcagctgttcgtggaacaac |
| | | acaaacactaccttgatgagatcatcgagcaaataagcgaatt |
| | | ctccaaaagagtgatcctcgccgacgctaacctcgataaggtg |
| | | ctttctgcttacaataagcacagggataagcccatcagggagc |
| | | aggcagaaaacattatccacttgtttactctgaccaacttggg |
| | | cgcgcctgcagccttcaagtacttcgacaccaccatagacaga |
| | | aagcggtacacctctacaaaggaggtcctggacgccacactga |
| | | ttcatcagtcaattacggggctctatgaaacaagaatcgacct |
| | | ctctcagctcggtggagac |
| 66 | dead Cas9 (dCas9) na | atggacaagaagtactccattgggctcgctatcggcacaaaca |
| | sequence | gcgtcggctgggccgtcattacggacgagtacaaggtgccgag |
| | | caaaaaattcaaagttctgggcaataccgatcgccacagcata |
| | | aagaagaacctcattggcgccctcctgttcgactccggggaga |
| | | cggccgaagccacgcggctcaaaagaacagcacggcgcagata |
| | | tacccgcagaaagaatcggatctgctacctgcaggagatcttt |
| | | agtaatgagatggctaaggtggatgactctttcttccataggc |
| | | tggaggagtcctttttggtggaggaggataaaaagcacgagcg |
| | | ccacccaatctttggcaatatcgtggacgaggtggcgtaccat |
| | | gaaaagtacccaaccatatatcatctgaggaagaagcttgtag |
| | | acagtactgataaggctgacttgcggttgatctatctcgcgct |
| | | ggcgcatatgatcaaatttcggggacacttcctcatcgagggg |
| | | gacctgaacccagacaacagcgatgtcgacaaactctttatcc |
| | | aactggttcagacttacaatcagcttttcgaagagaacccgat |
| | | caacgcatccggagttgacgccaaagcaatcctgagcgctagg |
| | | ctgtccaaatcccggcggctcgaaaacctcatcgcacagctcc |
| | | ctggggagaagaagaacggcctgtttggtaatcttatcgccct |
| | | gtcactcgggctgacccccaactttaaatctaacttcgacctg |
| | | gccgaagatgccaagcttcaactgagcaaagacacctacgatg |
| | | atgatctcgacaatctgctggcccagatcggcgaccagtacgc |
| | | agacctttttttggcggcaaagaacctgtcagacgccattctg |
| | | ctgagtgatattctgcgagtgaacacggagatcaccaaagctc |
| | | cgctgagcgctagtatgatcaagcgctatgatgagcaccacca |
| | | agacttgactttgctgaaggcccttgtcagacagcaactgcct |
| | | gagaagtacaaggaaattttcttcgatcagtctaaaaatggct |
| | | acgccggatacattgacggcggagcaagccaggaggaatttta |
| | | caaatttattaagcccatcttggaaaaaatggacggcaccgag |
| | | gagctgctggtaaagcttaacagagaagatctgttgcgcaaac |
| | | agcgcactttcgacaatggaagcatcccccaccagattcacct |
| | | gggcgaactgcacgctatcctcaggcggcaagaggatttctac |
| | | ccctttttgaaagataacagggaaaagattgagaaaatcctca |
| | | catttcggataccctactatgtaggccccctcgcccggggaaa |
| | | ttccagattcgcgtggatgactcgcaaatcagaagagaccatc |
| | | actccctggaacttcgaggaagtcgtggataagggggcctctg |
| | | cccagtccttcatcgaaaggatgactaactttgataaaaatct |
| | | gcctaacgaaaaggtgcttcctaaacactctctgctgtacgag |
| | | tacttcacagtttataacgagctcaccaaggtcaaatacgtca |
| | | cagaagggatgagaaagccagcattcctgtctggagagcagaa |
| | | gaaagctatcgtggacctcctcttcaagacgaaccggaaagtt |
| | | accgtgaaacagctcaaagaagactatttcaaaaagattgaat |
| | | gtttcgactctgttgaaatcagcggagtggaggatcgcttcaa |
| | | cgcatccctgggaacgtatcacgatctcctgaaaatcattaaa |
| | | gacaaggacttcctggacaatgaggagaacgaggacattcttg |
| | | aggacattgtcctcacccttacgttgtttgaagatagggagat |
| | | gattgaagaacgcttgaaaacttacgctcatctcttcgacgac |
| | | aaagtcatgaaacagctcaagaggcgccgatatacaggatggg |
| | | ggcggctgtcaagaaaactgatcaatgggatccgagacaagca |
| | | gagtggaaagacaatcctggattttcttaagtccgatggattt |
| | | gccaaccggaacttcatgcagttgatccatgatgactctctca |
| | | cctttaaggaggacatccagaaagcacaagtttctggccaggg |
| | | ggacagtcttcacgagcacatcgctaatcttgcaggtagccca |
| | | gctatcaaaaagggaatactgcagaccgttaaggtcgtggatg |
| | | aactcgtcaaagtaatgggaaggcataagcccgagaatatcgt |
| | | tatcgagatggcccgagagaaccaaactacccagaagggacag |
| | | aagaacagtagggaaaggatgaagaggattgaagagggtataa |
| | | aagaactggggtcccaaatccttaaggaacacccagttgaaaa |
| | | cacccagcttcagaatgagaagctctacctgtactacctgcag |
| | | aacggcagggacatgtacgtggatcaggaactggacatcaatc |
| | | ggctctccgactacgacgtggctgctatcgtgccccagtcttt |
| | | tctcaaagatgattctattgataataaagtgttgacaagatcc |
| | | gataaagctagagggaagagtgataacgtcccctcagaagaag |
| | | ttgtcaagaaaatgaaaaattattggcggcagctgctgaacgc |
| | | caaactgatcacacaacggaagttcgataatctgactaaggct |
| | | gaacgaggtggcctgtctgagttggataaagccggcttcatca |
| | | aaaggcagcttgttgagacacgccagatcaccaagcacgtggc |
| | | ccaaattctcgattcacgcatgaacaccaagtacgatgaaaat |
| | | gacaaactgattcgagaggtgaaagttattactctgaagtcta |
| | | agctggtctcagatttcagaaaggactttcagttttataaggt |
| | | gagagagatcaacaattaccaccatgcgcatgatgcctacctg |
| | | aatgcagtggtaggcactgcacttatcaaaaaatatcccaagc |
| | | ttgaatctgaatttgtttacggagactataaagtgtacgatgt |
| | | taggaaaatgatcgcaaagtctgagcaggaaataggcaaggcc |
| | | accgctaagtacttcttttacagcaatattatgaattttttca |
| | | agaccgagattacactggccaatggagagattcggaagcgacc |
| | | acttatcgaaacaaacggagaaacaggagaaatcgtgtgggac |
| | | aagggtagggatttcgcgacagtccggaaggtcctgtccatgc |
| | | cgcaggtgaacatcgttaaaaagaccgaagtacagaccggagg |
| | | cttctccaaggaaagtatcctcccgaaaaggaacagcgacaag |
| | | ctgatcgcacgcaaaaaagattgggaccccaagaaatacggcg |
| | | gattcgattctcctacagtcgcttacagtgtactggttgtggc |
| | | caaagtggagaaagggaagtctaaaaaactcaaaagcgtcaag |
| | | gaactgctgggcatcacaatcatggagcgatcaagcttcgaaa |
| | | aaaaccccatcgactttctcgaggcgaaaggatataaagaggt |
| | | caaaaaagacctcatcattaagcttcccaagtactctctcttt |
| | | gagcttgaaaacggccggaaacgaatgctcgctagtgcgggcg |
| | | agctgcagaaaggtaacgagctggcactgccctctaaatacgt |
| | | taatttcttgtatctggccagccactatgaaaagctcaaaggg |
| | | tctcccgaagataatgagcagaagcagctgttcgtggaacaac |
| | | acaaacactaccttgatgagatcatcgagcaaataagcgaatt |
| | | ctccaaaagagtgatcctcgccgacgctaacctcgataaggtg |
| | | ctttctgcttacaataagcacagggataagcccatcagggagc |
| | | aggcagaaaacattatccacttgtttactctgaccaacttggg |
| | | cgcgcctgcagccttcaagtacttcgacaccaccatagacaga |
| | | aagcggtacacctctacaaaggaggtcctggacgccacactga |
| | | ttcatcagtcaattacggggctctatgaaacaagaatcgacct |
| | | ctctcagctcggtggagac |
| 67 | hyperactive PiggyBac (PB) | atgggcagcagcctggacgacgagcacatcctgagcgccctgc |
| | transposase na sequence | tgcagagcgacgacgagctggtcggcgaggacagcgacagcga |
| | | ggtgagcgaccacgtgagcgaggacgacgtgcagtccgacacc |
| | | gaggaggccttcatcgacgaggtgcacgaggtgcagcctacca |
| | | gcagcggctccgagatcctggacgagcagaacgtgatcgagca |
| | | gcccggcagctccctggccagcaacaggatcctgaccctgccc |
| | | cagaggaccatcaggggcaagaacaagcactgctggtccacct |
| | | ccaagcccaccaggcggagcagggtgtccgccctgaacatcgt |
| | | gagaagccagaggggccccaccaggatgtgcaggaacatctac |
| | | gaccccctgctgtgcttcaagctgttcttcaccgacgagatca |
| | | tcagcgagatcgtgaagtggaccaacgccgagatcagcctgaa |
| | | gaggcgggagagcatgacctccgccaccttcagggacaccaac |
| | | gaggacgagatctacgccttcttcggcatcctggtgatgaccg |
| | | ccgtgaggaaggacaaccacatgagcaccgacgacctgttcga |
| | | cagatccctgagcatggtgtacgtgagcgtgatgagcagggac |
| | | agattcgacttcctgatcagatgcctgaggatggacgacaaga |
| | | gcatcaggcccaccctgcgggagaacgacgtgttcacccccgt |
| | | gagaaagatctgggacctgttcatccaccagtgcatccagaac |
| | | tacacccctggcgcccacctgaccatcgacgagcagctgctgg |
| | | gcttcaggggcaggtgccccttcagggtctatatccccaacaa |
| | | gcccagcaagtacggcatcaagatcctgatgatgtgcgacagc |
| | | ggcaccaagtacatgatcaacggcatgccctacctgggcaggg |
| | | gcacccagaccaacggcgtgcccctgggcgagtactacgtgaa |
| | | ggagctgtccaagcccgtccacggcagctgcagaaacatcacc |
| | | tgcgacaactggttcaccagcatccccctggccaagaacctgc |
| | | tgcaggagccctacaagctgaccatcgtgggcaccgtgagaag |
| | | caacaagagagagatccccgaggtcctgaagaacagcaggtcc |
| | | aggcccgtgggcaccagcatgttctgcttcgacggccccctga |
| | | ccctggtgtcctacaagcccaagcccgccaagatggtgtacct |
| | | gctgtccagctgcgacgaggacgccagcatcaacgagagcacc |
| | | ggcaagccccagatggtgatgtactacaaccagaccaagggcg |
| | | gcgtggacaccctggaccagatgtgcagcgtgatgacctgcag |
| | | cagaaagaccaacaggtggcccatggccctgctgtacggcatg |
| | | atcaacatcgcctgcatcaacagcttcatcatctacagccaca |
| | | acgtgagcagcaagggcgagaaggtgcagagccggaaaaagtt |
| | | catgcggaacctgtacatgggcctgacctccagcttcatgagg |
| | | aagaggctggaggcccccaccctgaagagatacctgagggaca |
| | | acatcagcaacatcctgcccaaagaggtgcccggcaccagcga |
| | | cgacagcaccgaggagcccgtgatgaagaagaggacctactgc |
| | | acctactgtcccagcaagatcagaagaaaggccagcgccagct |
| | | gcaagaagtgtaagaaggtcatctgccgggagcacaacatcga |
| | | catgtgccagagctgtttc |
| 68 | hyperactive Sleeping | Atgggaaaatcaaaagaaatcagccaagacctcagaaaaagaa |
| | Beauty (SB100) | ttgtagacctccacaagtctggttcatccttgggagcaatttc |
| | transposase na sequence | caaacgcctggcggtaccacgttcatctgtacaaacaatagta |
| | | cgcaagtataaacaccatgggaccacgcagccgtcataccgct |
| | | caggaaggagacgcgttctgtctcctagagatgaacgtacttt |
| | | ggtgcgaaaagtgcaaatcaatcccagaacaacagcaaaggac |
| | | cttgtgaagatgctggaggaaacaggtacaaaagtatctatat |
| | | ccacagtaaaacgagtcctatatcgacataacctgaaaggcca |
| | | ctcagcaaggaagaagccactgctccaaaaccgacataagaaa |
| | | gccagactacggtttgcaactgcacatggggacaaagatcgta |
| | | ctttttggagaaatgtcctctggtctgatgaaacaaaaataga |
| | | actgtttggccataatgaccatcgttatgtttggaggaagaag |
| | | ggggaggcttgcaagccgaagaacaccatcccaaccgtgaagc |
| | | acgggggtggcagcatcatgttgtgggggtgctttgctgcagg |
| | | agggactggtgcacttcacaaaatagatggcatcatggacgcc |
| | | gtgcagtatgtggatatattgaagcaacatctcaagacatcag |
| | | tcaggaagttaaagcttggtcgcaaatgggtcttccaacacga |
| | | caatgaccccaagcatacttccaaagttgtggcaaaatggctt |
| | | aaggacaacaaagtcaaggtattggagtggccatcacaaagcc |
| | | ctgacctcaatcctatagaaaatttgtgggcagaactgaaaaa |
| | | gcgtgtgcgagcaaggaggcctacaaacctgactcagttacac |
| | | cagctctgtcaggaggaatgggccaaaattcacccaaattatt |
| | | gtgggaagcttgtggaaggctacccgaaacgtttgacccaagt |
| | | taaacaatttaaaggcaatgctaccaaatac |
| 69 | human Cas9 (hCas9) aa | MDKKYSIGLDIGTNSVGWAVITDEYKVPSKKFKVLGNTDRHSI |
| | sequence | KKNLIGALLFDSGETAEATRLKRTARRRYTRRKNRICYLQEIF |
| | | SNEMAKVDDSFEHRLEESELVEEDKKHERHPIEGNIVDEVAYH |
| | | EKYPTIYHLRKKLVDSTDKADLRLIYLALAHMIKFRGHFLIEG |
| | | DLNPDNSDVDKLFIQLVQTYNQLFEENPINASGVDAKAILSAR |
| | | LSKSRRLENLIAQLPGEKKNGLFGNLIALSLGLTPNFKSNFDL |
| | | AEDAKLQLSKDTYDDDLDNLLAQIGDQYADLFLAAKNLSDAIL |
| | | LSDILRVNTEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFY |
| | | PFLKDNREKIEKILTFRIPYYVGPLARGNSRFAWMTRKSEETI |
| | | TPWNFEEVVDKGASAQSFIERMTNFDKNLPNEKVLPKHSLLYE |
| | | YFTVYNELTKVKYVTEGMRKPAELSGEQKKAIVDLLEKTNRKV |
| | | TVKQLKEDYFKKIECEDSVEISGVEDRFNASLGTYHDLLKIIK |
| | | DKDFLDNEENEDILEDIVLTLTLFEDREMIEERLKTYAHLFDD |
| | | KVMKQLKRRRYTGWGRLSRKLINGIRDKQSGKTILDFLKSDGF |
| | | ANRNFMQLIHDDSLTEKEDIQKAQVSGQGDSLHEHIANLAGSP |
| | | AIKKGILQTVKVVDELVKVMGRHKPENIVIEMARENQTTQKGQ |
| | | KNSRERMKRIEEGIKELGSQILKEHPVENTQLQNEKLYLYYLQ |
| | | NGRDMYVDQELDINRLSDYDVDHIVPQSFLKDDSIDNKVLTRS |
| | | DKNRGKSDNVPSEEVVKKMKNYWRQLLNAKLITQRKFDNLTKA |
| | | ERGGLSELDKAGFIKRQLVETRQITKHVAQILDSRMNTKYDEN |
| | | DKLIREVKVITLKSKLVSDFRKDFQFYKVREINNYHHAHDAYL |
| | | NAVVGTALIKKYPKLESEFVYGDYKVYDVRKMIAKSEQEIGKA |
| | | TAKYFFYSNIMNFFKTEITLANGEIRKRPLIETNGETGEIVWD |
| | | KGRDFATVRKVLSMPQVNIVKKTEVQTGGFSKESILPKRNSDK |
| | | LIARKKDWDPKKYGGFDSPTVAYSVLVVAKVEKGKSKKLKSVK |
| | | ELLGITIMERSSFEKNPIDFLEAKGYKEVKKDLIIKLPKYSLF |
| | | ELENGRKRMLASAGELQKGNELALPSKYVNFLYLASHYEKLKG |
| | | SPEDNEQKQLFVEQHKHYLDEIIEQISEFSKRVILADANLDKV |
| | | LSAYNKHRDKPIREQAENIIHLFTLTNLGAPAAFKYFDTTIDR |
| | | KRYTSTKEVLDATLIHQSITGLYETRIDLSQLGGD |
| 70 | nickase Cas9 (nCas9) aa | MDKKYSIGLAIGTNSVGWAVITDEYKVPSKKFKVLGNTDRHSI |
| | sequence | KKNLIGALLFDSGETAEATRLKRTARRRYTRRKNRICYLQEIF |
| | | SNEMAKVDDSFEHRLEESELVEEDKKHERHPIEGNIVDEVAYH |
| | | EKYPTIYHLRKKLVDSTDKADLRLIYLALAHMIKFRGHFLIEG |
| | | DLNPDNSDVDKLFIQLVQTYNQLFEENPINASGVDAKAILSAR |
| | | LSKSRRLENLIAQLPGEKKNGLFGNLIALSLGLTPNFKSNFDL |
| | | AEDAKLQLSKDTYDDDLDNLLAQIGDQYADLFLAAKNLSDAIL |
| | | LSDILRVNTEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFY |
| | | PFLKDNREKIEKILTFRIPYYVGPLARGNSRFAWMTRKSEETI |
| | | TPWNFEEVVDKGASAQSFIERMTNFDKNLPNEKVLPKHSLLYE |
| | | YFTVYNELTKVKYVTEGMRKPAELSGEQKKAIVDLLEKTNRKV |
| | | TVKQLKEDYFKKIECEDSVEISGVEDRFNASLGTYHDLLKIIK |
| | | DKDFLDNEENEDILEDIVLTLTLFEDREMIEERLKTYAHLFDD |
| | | KVMKQLKRRRYTGWGRLSRKLINGIRDKQSGKTILDFLKSDGF |
| | | ANRNFMQLIHDDSLTEKEDIQKAQVSGQGDSLHEHIANLAGSP |
| | | AIKKGILQTVKVVDELVKVMGRHKPENIVIEMARENQTTQKGQ |
| | | KNSRERMKRIEEGIKELGSQILKEHPVENTQLQNEKLYLYYLQ |
| | | NGRDMYVDQELDINRLSDYDVDHIVPQSFLKDDSIDNKVLTRS |
| | | DKNRGKSDNVPSEEVVKKMKNYWRQLLNAKLITQRKFDNLTKA |
| | | ERGGLSELDKAGFIKRQLVETRQITKHVAQILDSRMNTKYDEN |
| | | DKLIREVKVITLKSKLVSDFRKDFQFYKVREINNYHHAHDAYL |
| | | NAVVGTALIKKYPKLESEFVYGDYKVYDVRKMIAKSEQEIGKA |
| | | TAKYFFYSNIMNFFKTEITLANGEIRKRPLIETNGETGEIVWD |
| | | KGRDFATVRKVLSMPQVNIVKKTEVQTGGFSKESILPKRNSDK |
| | | LIARKKDWDPKKYGGFDSPTVAYSVLVVAKVEKGKSKKLKSVK |
| | | ELLGITIMERSSFEKNPIDFLEAKGYKEVKKDLIIKLPKYSLF |
| | | ELENGRKRMLASAGELQKGNELALPSKYVNFLYLASHYEKLKG |
| | | SPEDNEQKQLFVEQHKHYLDEIIEQISEFSKRVILADANLDKV |
| | | LSAYNKHRDKPIREQAENIIHLFTLTNLGAPAAFKYFDTTIDR |
| | | KRYTSTKEVLDATLIHQSITGLYETRIDLSQLGGD |
| 71 | dead Cas9 (dCas9) aa | MDKKYSIGLAIGTNSVGWAVITDEYKVPSKKFKVLGNTDRHSI |
| | sequence | KKNLIGALLFDSGETAEATRLKRTARRRYTRRKNRICYLQEIF |
| | | SNEMAKVDDSFEHRLEESELVEEDKKHERHPIEGNIVDEVAYH |
| | | EKYPTIYHLRKKLVDSTDKADLRLIYLALAHMIKFRGHFLIEG |
| | | DLNPDNSDVDKLFIQLVQTYNQLFEENPINASGVDAKAILSAR |
| | | LSKSRRLENLIAQLPGEKKNGLFGNLIALSLGLTPNFKSNFDL |
| | | AEDAKLQLSKDTYDDDLDNLLAQIGDQYADLFLAAKNLSDAIL |
| | | LSDILRVNTEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFY |
| | | PFLKDNREKIEKILTFRIPYYVGPLARGNSRFAWMTRKSEETI |
| | | TPWNFEEVVDKGASAQSFIERMTNFDKNLPNEKVLPKHSLLYE |
| | | YFTVYNELTKVKYVTEGMRKPAELSGEQKKAIVDLLEKTNRKV |
| | | TVKQLKEDYFKKIECEDSVEISGVEDRFNASLGTYHDLLKIIK |
| | | DKDFLDNEENEDILEDIVLTLTLFEDREMIEERLKTYAHLFDD |
| | | KVMKQLKRRRYTGWGRLSRKLINGIRDKQSGKTILDFLKSDGF |
| | | ANRNFMQLIHDDSLTEKEDIQKAQVSGQGDSLHEHIANLAGSP |
| | | AIKKGILQTVKVVDELVKVMGRHKPENIVIEMARENQTTQKGQ |
| | | KNSRERMKRIEEGIKELGSQILKEHPVENTQLQNEKLYLYYLQ |
| | | NGRDMYVDQELDINRLSDYDVAAIVPQSFLKDDSIDNKVLTRS |
| | | DKARGKSDNVPSEEVVKKMKNYWRQLLNAKLITQRKFDNLTKA |
| | | ERGGLSELDKAGFIKRQLVETRQITKHVAQILDSRMNTKYDEN |
| | | DKLIREVKVITLKSKLVSDFRKDFQFYKVREINNYHHAHDAYL |
| | | NAVVGTALIKKYPKLESEFVYGDYKVYDVRKMIAKSEQEIGKA |
| | | TAKYFFYSNIMNFFKTEITLANGEIRKRPLIETNGETGEIVWD |
| | | KGRDFATVRKVLSMPQVNIVKKTEVQTGGFSKESILPKRNSDK |
| | | LIARKKDWDPKKYGGFDSPTVAYSVLVVAKVEKGKSKKLKSVK |
| | | ELLGITIMERSSFEKNPIDFLEAKGYKEVKKDLIIKLPKYSLF |
| | | ELENGRKRMLASAGELQKGNELALPSKYVNFLYLASHYEKLKG |
| | | SPEDNEQKQLFVEQHKHYLDEIIEQISEFSKRVILADANLDKV |
| | | LSAYNKHRDKPIREQAENIIHLFTLTNLGAPAAFKYFDTTIDR |
| | | KRYTSTKEVLDATLIHQSITGLYETRIDLSQLGGD |
| 72 | Hyperactive PiggyBac (PB) | MGSSLDDEHILSALLQSDDELVGEDSDSEVSDHVSEDDVQSDT |
| | transposase na sequence | EEAFIDEVHEVQPTSSGSEILDEQNVIEQPGSSLASNRILTLP |
| | | QRTIRGKNKHCWSTSKPTRRSRVSALNIVRSQRGPTRMCRNIY |
| | | DPLLCFKLFFTDEIISEIVKWTNAEISLKRRESMTSATFRDTN |
| | | EDEIYAFFGILVMTAVRKDNHMSTDDLFDRSLSMVYVSVMSRD |
| | | REDFLIRCLRMDDKSIRPTLRENDVETPVRKIWDLFIHQCIQN |
| | | YTPGAHLTIDEQLLGERGRCPERVYIPNKPSKYGIKILMMCDS |
| | | GTKYMINGMPYLGRGTQTNGVPLGEYYVKELSKPVHGSCRNIT |
| | | CDNWFTSIPLAKNLLQEPYKLTIVGTVRSNKREIPEVLKNSRS |
| | | RPVGTSMFCFDGPLTLVSYKPKPAKMVYLLSSCDEDASINEST |
| | | GKPQMVMYYNQTKGGVDTLDQMCSVMTCSRKTNRWPMALLYGM |
| | | INIACINSFIIYSHNVSSKGEKVQSRKKFMRNLYMGLTSSFMR |
| | | KRLEAPTLKRYLRDNISNILPKEVPGTSDDSTEEPVMKKRTYC |
| | | TYCPSKIRRKASASCKKCKKVICREHNIDMCQSCF |
| 73 | hyperactive Sleeping | MGKSKEISQDLRKRIVDLHKSGSSLGAISKRLAVPRSSVQTIV |
| | Beauty (SB100) | RKYKHHGTTQPSYRSGRRRVLSPRDERTLVRKVQINPRTTAKD |
| | transposase aa sequence | LVKMLEETGTKVSISTVKRVLYRHNLKGHSARKKPLLQNRHKK |
| | | ARLRFATAHGDKDRTFWRNVLWSDETKIELFGHNDHRYVWRKK |
| | | GEACKPKNTIPTVKHGGGSIMLWGCFAAGGTGALHKIDGIMDA |
| | | VQYVDILKQHLKTSVRKLKLGRKWVFQHDNDPKHTSKVVAKWL |
| | | KDNKVKVLEWPSQSPDLNPIENLWAELKKRVRARRPTNLTQLH |
| | | QLCQEEWAKIHPNYCGKLVEGYPKRLTQVKQFKGNATKY |
| 74 | IN cPPT/CTS domain na | ttttaaaagaaaaggggggattggggggtacagtgcaggggaa |
| | sequence | agaatagtagacataatagcaacagacatacaaactaaagaat |
| | | tacaaaaacaaattacaaaaattcaaaatttt |
| 75 | Primer GG-cPPT-Fw | tcctctcgtctccattattttaaaagaaaaggggggatt |
| 76 | Primer GG-cPPT-STOP-Fw | tcctctcgtctccattaatttaaaagaaaaggggggatt |
| 77 | Primer GG-cPPT-Rv | tcctctcgtctccctgaaaaattttgaatttttgtaatttgtt |
| | | tttg |
| 78 | Primer GG-AAVS1-6d-Fw | tcctctcgtctccattatatggctccaaagaaaaagagg |
| 79 | Primer GG-AAVS1-6d-Rv | tcctctcgtctccctgatcaatcctcatcctgtctacttgcca |
| | | ca |
| 80 | Primer GG-AAVS1-6d | tcctctcgtctccattatatggcccaggctgctct |
| | (-NLS)-Fw | |
| 81 | Primer IN-Fw | ttttagatggaatagataaggccc |
| 82 | Primer XbaI-pSICO_IC- | ctagctctagatggctaactagggaacccact |
| | 5′Fw1 | |
| 83 | Primer SacI-pSICO_IC- | ctagcgagctcccaggctcagatctggtctaac |
| | 5′Rv1 | |
| 84 | Primer XbaI-pSICO_IC- | ctagctctagactaactagggaacccactgc |
| | 5′Fw2 | |
| 85 | Primer SacI-pSICO_IC- | cctctctatgggcagtctagcgagctcctggtctaaccagaga |
| | 5′Rv2 | gaccc |
| 86 | Primer XbaI-pSICO_IC- | ctagctctagatccctcagacccttttagtca |
| | 3′Fw1 | |
| 87 | Primer SacI-pSICO_IC- | ctagcgagctccaacagacgggcacacacta |
| | 3′Rv1 | |
| 88 | Primer XbaI-pSICO_IC- | ctagctctagaaaaatctctagcagcccatcc |
| | 3′Fw2 | |
| 89 | Primer SacI-pSICO_IC- | cctctctatgggcagtctagcgagctcgacgggcacacactac |
| | 3′Rv2 | ttga |
| 90 | Primer CCD1-A128T-F | tcaccagtactacagttaagaccgcctgttggtgg |
| 91 | Primer CCD1-A128T-R | ccaccaacaggcggtcttaactgtagtactggtga |
| 92 | Primer CCD2-E170G-F | acaggtaagagatcaggctggccatcttaagacagcagtac |
| 93 | Primer CCD2-E170G-R | gtactgctgtcttaagatggccagcctgatctcttacctgt |
| 94 | Primer NTD1-E10/13K-F | ggttttttagatggaatagataaggcccaaaaggaacataaga |
| | | aatatcacagtaattggaga |
| 95 | Primer NTD1-E10/13K-R | tctccaattactgtgatatttcttatgttccttttgggcctta |
| | | tctattccatctaaaaaacc |
| 96 | Primer Solubility-F185K-F | aaatggcagtattcatccacaataagaaaagaaaaggggggat |
| | | tggggg |
| 97 | Primer Solubility-F185K-R | cccccaatccccccttttcttttcttattgtggatgaatactg |
| | | ccattt |
| 98 | Primer Primer NGS-aavs fw | acactctttccctacacgacgctcttccgatctaggacagcat |
| | | gtttgctgcct |
| 99 | Primer NGS-aavs rv | gactggagttcagacgtgtgctcttccgatctgctccaggaaa |
| | | tgggggtg |
| 100 | Primer PE R245A | cgtgttcacccccgtggcaaagatctgggacctg |
| 101 | Primer PE R275-277A | agctgctgggcttcgcgggcgcgtgccccttcaggg |
| 102 | Primer PE R388A | gaacagcaggtccgcgcccgtgggcacc |
| 103 | Primer PE S351A | gacaactggttcaccgccatccccctggccaa |
| 104 | Primer PE W465A | gaaagaccaacagggcgcccatggccctgc |
| 105 | Primer PE R372A-K375A | catcgtgggcaccgtggcaagcaacgcgagagagatccccgag |
| 106 | Primer PE D450N | gcgtggacaccctgaaccagatgtgcagc |
| 107 | Primer SYBR-WPRE-3 Fw | acgctatgtggatacgctgct |
| 108 | Primer SYBR-WPRE-3 Rv | agcaaacacagtgcacaccac |
| 109 | Primer SYER-RNaseP Fw | ggagtgaggagggatgtgaa |
| 110 | Primer SYBR-RNaseP Rv | attgagggcactggaaattg |
| 111 | Primer Illumina custom | aatgatacggcgaccaccgagatctacacagctagacactctt |
| | | tccctacacgacgctcttccgatct |
| 112 | Primer NEBNext Index 9 | caagcagaagacggcatacgagatctgatcgtgactggagttc |
| | | agacgtgtgctcttccgatct |
| 113 | Primer NGS cluster 1 fw | acactctttccctacacgacgctcttccgatct |
| | | ctgcgggagaacgacgtgtt |
| 114 | Primer NGS cluster 2 rv | gactggagttcagacgtgtgctcttccgatct |
| | | cctcaccttcctcttcttcttgg |
| 115 | Primer CMV-F | ctgcagcgcggggatctcatgctggagttcttcgcccacccc |
| 116 | Primer ca59 rv | caccttcctcttcttcttggggtca |
| 117 | ZFP TCRa4 na sequence | atggctcctaagaagaagcggaaagtcggcatacacggagtgc |
| | | ctgctgcaatggcagaaaggccattccaatgcagaatatgcat |
| | | gaggaacttctcagatcgcagtaacctctcaaggcatatacgg |
| | | acccatacgggggaaaaaccatttgcctgtgatatatgtggcc |
| | | gcaagttcgctcagaaagtgaccttggcagctcacactaagat |
| | | tcacacacatccaagagcccctatccctaagccgttccaatgt |
| | | aggatatgcatgcgaaacttctctgatcggagtgcactgagta |
| | | ggcacatcagaacacacacgggagaaaagcctttcgcttgcga |
| | | tatctgcgggcggaagttcgcaacatccgggaatctcactcgc |
| | | catacgaaaatacacactggcagccaaaaacctttccaatgcc |
| | | gaatatgtatgagaaattttagctacagaagttcattgaaaga |
| | | acacattagaacccataccggagaaaagccgttcgcgtgcgat |
| | | atctgcggtcggaagttcgctacctcaggcaacctgacacgcc |
| | | acacgaaaatccac |
| 118 | ZFP TCRa4 aa sequence | MAPKKKRKVGIHGVPAAMAERPFQCRICMRNFSDRSNLSRHIR |
| | | THTGEKPFACDICGRKFAQKVTLAAHTKIHTHPRAPIPKPFQC |
| | | RICMRNFSDRSALSRHIRTHTGEKPFACDICGRKFATSGNLTR |
| | | HTKIHTGSQKPFQCRICMRNFSYRSSLKEHIRTHTGEKPFACD |
| | | ICGRKFATSGNLTRHTKIH |
| 119 | Modified hyperactive | SEQ ID NO: 9 |
| | PiggyBac aa sequence | With R245A, R275A, R277A, R275A/R277A, |
| | | G325A, N347A, N347S, S351E, S351P, S351A, |
| | | R372A, K375A, R388A, D450N, W465A, T560A, |
| | | S564P, S573A, M589V, S592G, F594L, or any |
| | | combination thereof |
| 120 | Top1 Modified hyperactive | SEQ ID NO: 9 |
| | PiggyBac aa sequence | With A at position 245, |
| | | R or A at position 275, |
| | | R or A at position 277, |
| | | A or G at position 325, |
| | | N or A at position 347, |
| | | E, P or A at position 351, |
| | | R at position 372, |
| | | A at position 375, |
| | | D or N at position 450 |
| | | W or A at position 465 |
| | | T or A at position 560, |
| | | P or S at position 564, |
| | | S or A at position 573, |
| | | G or S at position 592, |
| | | L or F at position 594, or any combination |
| | | thereof. |
| 121 | Top1.1 Modified | MGSSLDDEHILSALLQSDDELVGEDSDSEVSDHVSEDDVQSDT |
| | hyperactive PiggyBac aa | EEAFIDEVHEVQPTSSGSEILDEQNVIEQPGSSLASNRILTLP |
| | sequence | QRTIRGKNKHCWSTSKPTRRSRVSALNIVRSQRGPTRMCRNIY |
| | | DPLLCFKLFFTDEIISEIVKWTNAEISLKRRESMTSATFRDTN |
| | | EDEIYAFFGILVMTAVRKDNHMSTDDLFDRSLSMVYVSVMSRD |
| | | REDFLIRCLRMDDKSIRPTLRENDVETPVAKIWDLFIHQCIQN |
| | | YTPGAHLTIDEQLLGERGRCPERVYIPNKPSKYGIKILMMCDS |
| | | GTKYMINGMPYLGRGTQTNGVPLAEYYVKELSKPVHGSCRNIT |
| | | CDNWFTEIPLAKNLLQEPYKLTIVGTVRSNAREIPEVLKNSRS |
| | | RPVGTSMFCFDGPLTLVSYKPKPAKMVYLLSSCDEDASINEST |
| | | GKPQMVMYYNQTKGGVDTL(D/N)QMCSVMTCSRKTNR(W/A) |
| | | PMALLYGMINIACINSFIIYSHNVSSKGEKVQSRKKFMRNLYM |
| | | GLTSSFMRKRLEAPTLKRYLRDNISNILPKEVPGTSDDSTEEP |
| | | VMKKRTYCTYCPPKIRRKASASCKKCKKVICREHNIDMCQGCL |
| | | position 450 can be D or N |
| | | position 465 can be W or A |
| 122 | Top1.2 Modified | MGSSLDDEHILSALLQSDDELVGEDSDSEVSDHVSEDDVQSDT |
| | hyperactive PiggyBac aa | EEAFIDEVHEVQPTSSGSEILDEQNVIEQPGSSLASNRILTLP |
| | sequence | QRTIRGKNKHCWSTSKPTRRSRVSALNIVRSQRGPTRMCRNIY |
| | | DPLLCFKLFFTDEIISEIVKWTNAEISLKRRESMTSATFRDTN |
| | | EDEIYAFFGILVMTAVRKDNHMSTDDLFDRSLSMVYVSVMSRD |
| | | REDFLIRCLRMDDKSIRPTLRENDVETPVAKIWDLFIHQCIQN |
| | | YTPGAHLTIDEQLLGFAGACPFRVYIPNKPSKYGIKILMMCDS |
| | | GTKYMINGMPYLGRGTQTNGVPLGEYYVKELSKPVHGSCRNIT |
| | | CDAWFTPIPLAKNLLQEPYKLTIVGTVRSNAREIPEVLKNSRS |
| | | RPVGTSMFCFDGPLTLVSYKPKPAKMVYLLSSCDEDASINEST |
| | | GKPQMVMYYNQTKGGVDTL(D/N)QMCSVMTCSRKTNR(W/A) |
| | | PMALLYGMINIACINSFIIYSHNVSSKGEKVQSRKKFMRNLYM |
| | | GLTSSFMRKRLEAPTLKRYLRDNISNILPKEVPGTSDDSTEEP |
| | | VMKKRTYCAYCPSKIRRKASASCKKCKKVICREHNIDMCQSCF |
| | | position 450 can be D or N |
| | | position 465 can be W or A |
| 123 | Top1.3 Modified | MGSSLDDEHILSALLQSDDELVGEDSDSEVSDHVSEDDVQSDT |
| | hyperactive PiggyBac aa | EEAFIDEVHEVQPTSSGSEILDEQNVIEQPGSSLASNRILTLP |
| | sequence | QRTIRGKNKHCWSTSKPTRRSRVSALNIVRSQRGPTRMCRNIY |
| | | DPLLCFKLFFTDEIISEIVKWTNAEISLKRRESMTSATFRDTN |
| | | EDEIYAFFGILVMTAVRKDNHMSTDDLFDRSLSMVYVSVMSRD |
| | | REDFLIRCLRMDDKSIRPTLRENDVETPVAKIWDLFIHQCIQN |
| | | YTPGAHLTIDEQLLGERGRCPERVYIPNKPSKYGIKILMMCDS |
| | | GTKYMINGMPYLGRGTQTNGVPLAEYYVKELSKPVHGSCRNIT |
| | | CDAWFTAIPLAKNLLQEPYKLTIVGTVRSNAREIPEVLKNSRS |
| | | RPVGTSMFCFDGPLTLVSYKPKPAKMVYLLSSCDEDASINEST |
| | | GKPQMVMYYNQTKGGVDTL(D/N)QMCSVMTCSRKTNR(W/A) |
| | | PMALLYGMINIACINSFIIYSHNVSSKGEKVQSRKKFMRNLYM |
| | | GLTSSFMRKRLEAPTLKRYLRDNISNILPKEVPGTSDDSTEEP |
| | | VMKKRTYCAYCPSKIRRKASAACKKCKKVICREHNIDMCQSCF |
| | | position 450 can be D or N |
| | | position 465 can be W or A |
| 124 | Regular modified 1 | MGSSLDDEHILSALLQSDDELVGEDSDSEVSDHVSEDDVQSDT |
| | hyperactive PiggyBac aa | EEAFIDEVHEVQPTSSGSEILDEQNVIEQPGSSLASNRILTLP |
| | sequence | QRTIRGKNKHCWSTSKPTRRSRVSALNIVRSQRGPTRMCRNIY |
| | | DPLLCFKLFFTDEIISEIVKWTNAEISLKRRESMTSATFRDTN |
| | | EDEIYAFFGILVMTAVRKDNHMSTDDLFDRSLSMVYVSVMSRD |
| | | RFDFLIRCLRMDDKSIRPTLRENDVFTPVRKIWDLFIHQCIQN |
| | | YTPGAHLTIDEQLLGERGRCPERVYIPNKPSKYGIKILMMCDS |
| | | GTKYMINGMPYLGRGTQTNGVPLGEYYVKELSKPVHGSCRNIT |
| | | CDAWFTSIPLAKNLLQEPYKLTIVGTVASNKREIPEVLKNSRS |
| | | RPVGTSMFCFDGPLTLVSYKPKPAKMVYLLSSCDEDASINEST |
| | | GKPQMVMYYNQTKGGVDTL(D/N)QMCSVMTCSRKTNR(W/A) |
| | | PMALLYGMINIACINSFIIYSHNVSSKGEKVQSRKKFMRNLYM |
| | | GLTSSFMRKRLEAPTLKRYLRDNISNILPKEVPGTSDDSTEEP |
| | | VMKKRTYCTYCPSKIRRKASASCKKCKKVICREHNIDMCQSCF |
| | | position 450 can be D or N |
| | | position 465 can be W or A |
| 125 | Regular modified 2 | MGSSLDDEHILSALLQSDDELVGEDSDSEVSDHVSEDDVQSD |
| | hyperactive PiggyBac aa | TEEAFIDEVHEVQPTSSGSEILDEQNVIEQPGSSLASNRILT |
| | sequence | LPQRTIRGKNKHCWSTSKPTRRSRVSALNIVRSQRGPTRMCR |
| | | NIYDPLLCFKLFFTDEIISEIVKWTNAEISLKRRESMTSATF |
| | | RDTNEDEIYAFFGILVMTAVRKDNHMSTDDLFDRSLSMVYVS |
| | | VMSRDRFDFLIRCLRMDDKSIRPTLRENDVFTPVRKIWDLFI |
| | | HQCIQNYTPGAHLTIDEQLLGERGRCPERVYIPNKPSKYGIK |
| | | ILMMCDSGTKYMINGMPYLGRGTQTNGVPLGEYYVKELSKPV |
| | | HGSCRNITCDNWFTSIPLAKNLLQEPYKLTIVGTVRSNKREI |
| | | PEVLKNSRSRPVGTSMFCFDGPLTLVSYKPKPAKMVYLLSSC |
| | | DEDASINESTGKPQMVMYYNQTKGGVDTL(D/N)QMCSVMTC |
| | | SRKTNR(W/A)PMALLYGMINIACINSFITYSHNVSSKGEKV |
| | | QSRKKFMRNLYMGLTSSFMRKRLEAPTLKRYLRDNISNILPK |
| | | EVPGTSDDSTEEPVMKKRTYCTYCPSKIRRKASASCKKCKKV |
| | | ICREHNIDMCQGCF |
| | | position 450 can be D or N |
| | | position 465 can be W or A |
| 126 | Regular modified 3 | MGSSLDDEHILSALLQSDDELVGEDSDSEVSDHVSEDDVQSD |
| | hyperactive PiggyBac aa | TEEAFIDEVHEVQPTSSGSEILDEQNVIEQPGSSLASNRILT |
| | sequence | LPQRTIRGKNKHCWSTSKPTRRSRVSALNIVRSQRGPTRMCR |
| | | NIYDPLLCFKLFFTDEIISEIVKWTNAEISLKRRESMTSATF |
| | | RDTNEDEIYAFFGILVMTAVRKDNHMSTDDLFDRSLSMVYVS |
| | | VMSRDRFDFLIRCLRMDDKSIRPTLRENDVFTPVRKIWDLFI |
| | | HQCIQNYTPGAHLTIDEQLLGERGRCPERVYIPNKPSKYGIK |
| | | ILMMCDSGTKYMINGMPYLGRGTQTNGVPLGEYYVKELSKPV |
| | | HGSCRNITCDNWFTSIPLAKNLLQEPYKLTIVGTVRSNKREI |
| | | PEVLKNSRSRPVGTSMFCFDGPLTLVSYKPKPAKMVYLLSSC |
| | | DEDASINESTGKPQMVMYYNQTKGGVDTL(D/N)QMCSVMTC |
| | | SRKTNR(W/A)PMALLYGMINIACINSFITYSHNVSSKGEKV |
| | | QSRKKFMRNLYMGLTSSFMRKRLEAPTLKRYLRDNISNILPK |
| | | EVPGTSDDSTEEPVMKKRTYCTYCPSKIRRKASASCKKCKKV |
| | | ICREHNIDMCQSCF |
| | | position 450 can be D or N |
| | | position 465 can be W or A |
| 127 | Regular modified 4 | MGSSLDDEHILSALLQSDDELVGEDSDSEVSDHVSEDDVQSD |
| | hyperactive PiggyBac aa | TEEAFIDEVHEVQPTSSGSEILDEQNVIEQPGSSLASNRILT |
| | sequence | LPQRTIRGKNKHCWSTSKPTRRSRVSALNIVRSQRGPTRMCR |
| | | NIYDPLLCFKLFFTDEIISEIVKWTNAEISLKRRESMTSATF |
| | | RDTNEDEIYAFFGILVMTAVRKDNHMSTDDLFDRSLSMVYVS |
| | | VMSRDRFDFLIRCLRMDDKSIRPTLRENDVFTPVAKIWDLFI |
| | | HQCIQNYTPGAHLTIDEQLLGFAGACPFRVYIPNKPSKYGIK |
| | | ILMMCDSGTKYMINGMPYLGRGTQTNGVPLGEYYVKELSKPV |
| | | HGSCRNITCDNWFTSIPLAKNLLQEPYKLTIVGTVASNAREI |
| | | PEVLKNSRSRPVGTSMFCFDGPLTLVSYKPKPAKMVYLLSSC |
| | | DEDASINESTGKPQMVMYYNQTKGGVDTL(D/N)QMCSVMTC |
| | | SRKTNR(W/A)PMALLYGMINIACINSFITYSHNVSSKGEKV |
| | | QSRKKFMRNLYMGLTSSFMRKRLEAPTLKRYLRDNISNILPK |
| | | EVPGTSDDSTEEPVMKKRTYCTYCPSKIRRKASASCKKCKKV |
| | | ICREHNIDMCQSCL |
| | | position 450 can be D or N |
| | | position 465 can be W or A |
| 128 | Regular modified 5 | MGSSLDDEHILSALLQSDDELVGEDSDSEVSDHVSEDDVQSD |
| | hyperactive PiggyBac aa | TEEAFIDEVHEVQPTSSGSEILDEQNVIEQPGSSLASNRILT |
| | sequence | LPQRTIRGKNKHCWSTSKPTRRSRVSALNIVRSQRGPTRMCR |
| | | NIYDPLLCFKLFFTDEIISEIVKWTNAEISLKRRESMTSATF |
| | | RDTNEDEIYAFFGILVMTAVRKDNHMSTDDLFDRSLSMVYVS |
| | | VMSRDRFDFLIRCLRMDDKSIRPTLRENDVFTPVRKIWDLFI |
| | | HQCIQNYTPGAHLTIDEQLLGFAGRCPFRVYIPNKPSKYGIK |
| | | ILMMCDSGTKYMINGMPYLGRGTQTNGVPLAEYYVKELSKPV |
| | | HGSCRNITCDSWFTAIPLAKNLLQEPYKLTIVGTVASNKREI |
| | | PEVLKNSRSRPVGTSMFCFDGPLTLVSYKPKPAKMVYLLSSC |
| | | DEDASINESTGKPQMVMYYNQTKGGVDTL(D/N)QMCSVMTC |
| | | SRKTNR(W/A)PMALLYGMINIACINSFITYSHNVSSKGEKV |
| | | QSRKKFMRNLYMGLTSSFMRKRLEAPTLKRYLRDNISNILPK |
| | | EVPGTSDDSTEEPVMKKRTYCAYCPSKIRRKASASCKKCKKV |
| | | ICREHNIDMCQGCF |
| | | With position 450 can be D or N |
| | | position 465 can be W or A |
| 129 | Regular modified 6 | MGSSLDDEHILSALLQSDDELVGEDSDSEVSDHVSEDDVQSD |
| | hyperactive PiggyBac aa | TEEAFIDEVHEVQPTSSGSEILDEQNVIEQPGSSLASNRILT |
| | sequence | LPQRTIRGKNKHCWSTSKPTRRSRVSALNIVRSQRGPTRMCR |
| | | NIYDPLLCFKLFFTDEIISEIVKWTNAEISLKRRESMTSATF |
| | | RDTNEDEIYAFFGILVMTAVRKDNHMSTDDLFDRSLSMVYVS |
| | | VMSRDRFDFLIRCLRMDDKSIRPTLRENDVFTPVRKIWDLFI |
| | | HQCIQNYTPGAHLTIDEQLLGFAGRCPFRVYIPNKPSKYGIK |
| | | ILMMCDSGTKYMINGMPYLGRGTQTNGVPLGEYYVKELSKPV |
| | | HGSCRNITCDNWFTAIPLAKNLLQEPYKLTIVGTVASNAREI |
| | | PEVLKNSRSRPVGTSMFCFDGPLTLVSYKPKPAKMVYLLSSC |
| | | DEDASINESTGKPQMVMYYNQTKGGVDTL(D/N)QMCSVMTC |
| | | SRKTNR(W/A)PMALLYGMINIACINSFITYSHNVSSKGEKV |
| | | QSRKKFMRNLYMGLTSSFMRKRLEAPTLKRYLRDNISNILPK |
| | | EVPGTSDDSTEEPVMKKRTYCAYCPSKIRRKASASCKKCKKV |
| | | ICREHNIDMCQGCF |
| | | With position 450 can be D or N |
| | | position 465 can be W or A |
| 130 | Linker aa sequence | KLAGGAPAVGGGPK |
| 131 | Linker aa sequence | EFGGGGSGGGGSGGGGSQF |
| 132 | Primer SV40pA-R | Gaaatttgtgatgctattgc |
| 133 | Linker | (GGGGS)n |
| | | n is an integer between 1 and 50 |
| 134 | Linker | (EAAAK)n |
| | | n is an integer between 1 and 50 |

1-26. (canceled)

27. A nucleic acid construct comprising:
a) a first polynucleotide sequence comprising a nucleic acid encoding a first DNA binding protein engineered to bind to a specific genomic DNA sequence in a genome; wherein the first DNA binding protein is a zinc finger protein or a Cas9 protein;
b) a second polynucleotide sequence comprising a nucleic acid encoding a second DNA binding protein which enables insertion of an exogenous nucleic acid into a genome, wherein the second DNA binding protein is
(i) a hyperactive PiggyBac transposase, or a modified hyperactive PiggyBac with improved specificity of inserting the exogenous nucleic acid into the genome compared to the hyperactive PiggyBac, or
(ii) a human immunodeficiency virus (HIV) integrase, or a modified HIV integrase with improved specificity of inserting the exogenous nucleic acid into the genome compared to the HIV integrase; and
c) an optional polynucleotide sequence comprising a nucleic acid encoding a linker;
wherein the nucleic acid construct encodes a fusion protein comprising the first DNA binding protein, the second DNA binding protein, and the optional linker between the first DNA binding protein and the second DNA binding protein; and
wherein the fusion protein enables insertion of the exogenous nucleic acid into a specific site of the genome.

28. The nucleic acid construct of claim 27, wherein the Cas9 protein is a protein comprising an active DNA cleavage domain of Cas9 and the gRNA binding domain of Cas9. [p. 34, par. 167].

29. The nucleic acid construct of claim 27 wherein the linker comprises a XTEN sequence or a GGS sequence.

30. The nucleic acid construct of claim 27, wherein the 3′ end of the first polynucleotide sequence is connected to the 5′ end of the second polynucleotide.

31. The nucleic acid construct of claim 27, wherein the modified hyperactive PiggyBac transposase comprises a mutation of one or more of amino acids 245, 268, 275, 277, 287, 290, 315, 325, 341, 346, 347, 350, 351, 356, 357, 372, 375, 388, 409, 412, 432, 447, 450, 460, 461, 465, 517, 560, 564, 571, 573, 576, 586, 587, 589, 592, and 594 corresponding to the amino acid sequence SEQ ID NO: 9 of the hyperactive PiggyBac.

32. The nucleic acid construct of claim 31, wherein the modified hyperactive PiggyBac transposase mutation comprises one or more of the amino acid modifications selected from: R245A, D268N, R275A/R277A, K287A, K290A, K287A/K290A, R315A, G325A, R341A, D346N, N347A, N347S, T350A, S351E, S351P, S351A, K356E, N357A, R372A, K375A, R372A/K375A, R388A, K409A, K412A, K409A/K412A, K432A, D447A, D447N, D450N, R460A, K461A, R460A/K461A, W465A, S517A, T560A, S564P, S571N, S573A, K576A, H586A, I587A, M589V, S592G, or F594L corresponding to the amino acid sequence SEQ ID NO: 9 of the hyperactive PiggyBac.

33. The nucleic acid construct of claim 27, wherein the modified hyperactive PiggyBac transposase comprises a mutation of one or more of amino acids 245, 275, 277, 325, 347, 351, 372, 375, 388, 450, 465, 560, 564, 573, 589, 592, 594 corresponding to the amino acid sequence SEQ ID NO: 9 of the hyperactive PiggyBac.

34. The nucleic acid construct of claim 33, wherein the modified hyperactive PiggyBac transposase mutation comprises one or more of the amino acid modifications selected from: R245A, R275A, R277A, R275A/R277A, G325A, N347A, N347S, S351E, S351P, S351A, R372A, K375A, R388A, D450N, W465A, T560A, S564P, S573A, M589V, S592G, or F594L corresponding to the amino acid sequence SEQ ID NO: 9 of the hyperactive PiggyBac.

35. The nucleic acid construct of claim 27, wherein the modified hyperactive PiggyBac transposase comprises the amino acid sequence SEQ ID NO: 9, wherein:
i. amino acid at position 245 is A,
ii. amino acid at position 275 is R or A,
iii. amino acid at position 277 is R or A,
iv. amino acid at position 325 is A or G,
v. amino acid at position 347 is N or A,
vi. amino acid at position 351 is E, P or A,
vii. amino acid at position 372 is R,
viii. amino acid at position 375 is A,
ix. amino acid at position 450 is D or N,
x. amino acid at position 465 is W or A,
xi. amino acid at position 560 is T or A,
xii. amino acid at position 564 is P or S,
xiii. amino acid at position 573 is S or A,
xiv. amino acid at position 592 is G or S, and
xv. amino acid at position 594 is L or F.

36. The nucleic acid construct of claim 27, wherein the modified hyperactive PiggyBac transposase comprises an amino acid sequence selected from the group consisting of SEQ ID NO: 120, 121, 122, 123, 124, 125, 126, 127, 128, and 129.

37. The nucleic acid construct of claim 27, wherein the modified hyperactive PiggyBac transposase comprises an amino acid sequence having at least 80% identical to a sequence selected from the group consisting of SEQ ID NO: 119, 120, 121, 122, 123, 124, 125, 126, 127, 128 and 129, wherein the modified hyperactive PiggyBac shows higher specificity of DNA integration into a genome compared to hyperactive PiggyBac.

38. A vector comprising the nucleic acid construct of claim 27, wherein the vector is suitable for expression in mammalian cells, yeast cells, insect cells, plant cells, fungal cells, or algal cells.

39. A host cell comprising the nucleic acid construct of claim 27.

40. A fusion protein obtained from the expression of the nucleic acid construct of claim 27.

41. A composition comprising the nucleic acid construct of claim 27, and a polynucleotide sequence encoding an exogenous nucleic acid for insertion in a genome, the composition contained in or bound to a packaging vector.

42. The composition of claim 41, wherein the nucleic acid construct is in form of RNA, DNA or protein, and the polynucleotide sequence encoding the exogenous nucleic acid is in form of RNA or DNA.

43. The composition of claim 41, wherein the packaging vector is a nanoparticle or a lentiviral particle.

44. A method for controlled, site-specific integration of a single copy or multiple copies of an exogenous nucleic acid sequence into a cell, the method comprising:
a) delivering the nucleic acid construct of claim 27 to the cell, and
b) delivering the exogenous nucleic acid to the cell;
wherein binding of the fusion protein to the specific genomic DNA sequence in the genome of the cell, results in cleavage of the genome and integration of one or more copies of the exogenous nucleic acid into the genome of the cell.

45. A modified hyperactive PiggyBac transposase comprising the amino acid sequence SEQ ID NO: 9, wherein:
i. amino acid at position 245 is A,
ii. amino acid at position 275 is R or A,
iii. amino acid at position 277 is R or A,
iv. amino acid at position 325 is A or G,
v. amino acid at position 347 is N or A,
vi. amino acid at position 351 is E, P or A,
vii. amino acid at position 372 is R,
viii. amino acid at position 375 is A,
ix. amino acid at position 450 is D or N,
x. amino acid at position 465 is W or A,
xi. amino acid at position 560 is T or A,
xii. amino acid at position 564 is P or S,
xiii. amino acid at position 573 is S or A,
xiv. amino acid at position 592 is G or S, and
xv. amino acid at position 594 is L or F.

46. The modified hyperactive PiggyBac transposase of claim 45, which comprises an amino acid sequence selected from the group consisting of SEQ ID NO: 120, 121, 122, 123, 124, 125, 126, 127, 128, and 129.

47. The modified hyperactive PiggyBac transposase of claim 45, which comprises an amino acid sequence having at least 80% identical to a sequence selected from the group consisting of SEQ ID NO: 119, 120, 121, 122, 123, 124, 125, 126, 127, 128 and 129, wherein the modified hyperactive PiggyBac shows higher specificity of DNA integration into a genome compared to hyperactive PiggyBac.


SEQUENCE LISTING (134 sequences)

SEQ ID NO: 1 - protein, 288 aa, Artificial Sequence
  Wildtype HIV-1 integrase amino acid sequence
FLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 2 - DNA, 867 bp, Artificial Sequence
  Wildtype HIV-1 integrase nucleic acid sequence
tttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattag

SEQ ID NO: 3 - protein, 288 aa, Artificial Sequence
  Modified HIV-1 integrase
  misc_feature (10)..(10): Xaa can be Lys, Asp
  misc_feature (11)..(11): Xaa can be any naturally occurring amino acid
  misc_feature (13)..(13): Xaa can be Glu, Lys
  misc_feature (64)..(64): Xaa can be Ala, Glu, Asp
  misc_feature (94)..(94): Xaa can be Asp, Glu, Arg, Lys, Gly
  misc_feature (116)..(116): Xaa can be Asp, Ala, Glu
  misc_feature (117)..(117): Xaa can be Asp, Glu, Arg, Lys, Asn
  misc_feature (119)..(119): Xaa can be Ala, Pro, Thr, Gly, Asp, Glu, Arg, Lys, Ser
  misc_feature (120)..(120): Xaa can be Asp, Glu, Arg, Lys, Asn
  misc_feature (122)..(122): Xaa can be Lys, Ile, Val, Ala, Arg, Thr
  misc_feature (124)..(124): Xaa can be Asp, Glu, Arg, Lys, Ala
  misc_feature (125)..(125): Xaa can be Asp, Glu, Arg, Lys, Thr
  misc_feature (128)..(128): Xaa can be Ala, Thr
  misc_feature (152)..(152): Xaa can be Ala, Asp, Glu
  misc_feature (168)..(168): Xaa can be Leu, Ala, Gln
  misc_feature (170)..(170): Xaa can be Glu, Gly
  misc_feature (185)..(185): Xaa can be Phe, Lys
  misc_feature (231)..(231): Xaa can be Arg, Gly, Lys, Asp, Glu, Ser
  misc_feature (264)..(264): Xaa can be Lys, Arg
  misc_feature (266)..(266): Xaa can be Lys, Arg
  misc_feature (273)..(273): Xaa can be Lys, Arg
FLDGIDKAQXXHXKYHSNWRAMASDFNLPPVVAKEIVASCDKCQLKGEAMHGQVDCSPGIWQLXCTHLEGKVILVAVHVASGYIEAEVIPAETXQETAYFLLKLAGRWPVKTIHTXXGXXFXGXXVRXACWWAGIKQEFGIPYNPQSQGVVXSMNKELKKIIGQVRDXAEHLKTAVQMAVFIHNXKRKGGIGGYSAGERIVDIIATDIQTKELQKQITKIQNFRVYYRDSXNPLWKGPAKLLWKGEGAVVIQDNSDIKVVPRRXAXIIRDYGXQMAGDDCVASRQDED

SEQ ID NO: 4 - protein, 288 aa, Artificial Sequence
  Modified integrase with impaired DNA binding
  misc_feature (94)..(94): Xaa can be Gly, Asp, Glu, Arg, Lys
  misc_feature (117)..(117): Xaa can be Asn, Asp, Glu, Arg, Lys
  misc_feature (119)..(119): Xaa can be Ser, Ala, Pro, Thr, Gly, Asp, Glu, Arg, Lys
  misc_feature (120)..(120): Xaa can be Asn, Asp, Glu, Arg, Lys
  misc_feature (124)..(124): Xaa can be Asp, Glu, Arg, Lys
  misc_feature (125)..(125): Xaa can be Asp, Glu, Arg, Lys, Thr
  misc_feature (231)..(231): Xaa can be Arg, Gly, Lys, Asp, Glu, Ser
FLDGIDKAQDEHEKYHSNWRAMASDFNLPPVVAKEIVASCDKCQLKGEAMHGQVDCSPGIWQLDCTHLEGKVILVAVHVASGYIEAEVIPAETXQETAYFLLKLAGRWPVKTIHTDXGXXFTGXXVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSXNPLWKGPAKLLWKGEGAVVIQDNSDIKVVPRRKAKIIRDYGKQMAGDDCVASRQDED

SEQ ID NO: 5 - protein, 288 aa, Artificial Sequence
  Modified integrase with enhanced DNA binding
  Misc_feature (94)..(94): Xaa can be Gly, Asp, Glu, Arg, Lys
  Misc_feature (117)..(117): Xaa can be Asn, Asp, Glu, Arg, Lys
  Misc_feature (119)..(119): Xaa can be Ala, Pro, Thr, Gly, Asp, Glu, Arg, Lys, Ser
  Misc_feature (120)..(120): Xaa can be  Asn, Asp, Glu, Arg, Lys
  Misc_feature (122)..(122): Xaa can be  Thr, Lys, Ile, Val, Ala, Arg
  Misc_feature (124)..(124): Xaa can be  Asp, Glu, Arg, Lys, Ala
  Misc_feature (125)..(125): Xaa can be  Asp, Glu, Arg, Lys, Thr
  Misc_feature (168)..(168): Xaa can be  Leu, Ala, Gln
  Misc_feature (231)..(231): Xaa can be Arg, Gly, Lys, Asp, Glu, Ser
FLDGIDKAQDEHEKYHSNWRAMASDFNLPPVVAKEIVASCDKCQLKGEAMHGQVDCSPGIWQLDCTHLEGKVILVAVHVASGYIEAEVIPAETXQETAYFLLKLAGRWPVKTIHTDXGXXFXGXXVRAACWWAGIKQEFGIPYNPQSQGVVESMNKELKKIIGQVRDXAEHLKTAVQMAVFIHNFKRKGGIGGYSAGERIVDIIATDIQTKELQKQITKIQNFRVYYRDSXNPLWKGPAKLLWKGEGAVVIQDNSDIKVVPRRKAKIIRDYGKQMAGDDCVASRQDED

SEQ ID NO: 6 - protein, 288 aa, Artificial Sequence
  Modified integrase with acetylation mutations
  misc_feature (264)..(264): Xaa can be Lys, Arg
  misc_feature (266)..(266): Xaa can be Lys, Arg
  misc_feature (273)..(273): Xaa can be Lys, Arg
FLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRXAXIIRDYGXQMAGDDCVASRQDED

SEQ ID NO: 7 - protein, 288 aa, Artificial Sequence
  Modified integrase with mutations in retroviral integrative recombination
  misc_feature (10)..(10): Xaa can be  Asp, Lys
  misc_feature (11)..(11): Xaa can be any naturally occurring amino acid
  misc_feature (13)..(13): Xaa can be  Glu, Lys
  misc_feature (64)..(64): Xaa can be Asp, Ala, Glu
  misc_feature (116)..(116): Xaa can be Ala, Glu, Asp
  misc_feature (128)..(128): Xaa can be Ala, Thr
  misc_feature (152)..(152): Xaa can be Glu, Ala, Asp
  misc_feature (168)..(168): Xaa can be Gln, Leu, Ala
  misc_feature (170)..(170): Xaa can be Asp, Gly
FLDGIDKAQXXHXKYHSNWRAMASDFNLPPVVAKEIVASCDKCQLKGEAMHGQVDCSPGIWQLXCTHLEGKVILVAVHVASGYIEAEVIPAETGQETAYFLLKLAGRWPVKTIHTXNGSNFTGATVRXACWWAGIKQEFGIPYNPQSQGVVXSMNKELKKIIGQVRDXAXHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 8 - protein, 288 aa, Artificial Sequence
  Modified integrase with mutations in HIV-1 replication
  misc_feature (168)..(168): Xaa can be Gln, Leu, Ala
FLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDXAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 9 - protein, 594 aa, Artificial Sequence
  Hyperactive PiggyBac aa sequence
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 10 - protein, 594 aa, Artificial Sequence; identical to SEQ ID NO: 9
  Modified hyperactive PiggyBac
  misc_feature (245)..(245): Wherein amino acid can be Arg, Ala
  misc_feature (268)..(268): Wherein amino acid can be Asp, Asn
  misc_feature (275)..(275): Wherein amino acid can be Arg, Ala
  misc_feature (277)..(277): Wherein amino acid can be Arg, Ala
  misc_feature (287)..(287): Wherein amino acid can be Ala, Lys
  misc_feature (290)..(290): Wherein amino acid can be Ala, Lys
  misc_feature (315)..(315): Wherein amino acid can be Arg, Ala
  misc_feature (325)..(325): Wherein amino acid can be Gly, Ala
  misc_feature (341)..(341): Wherein amino acid can be Arg, Ala
  misc_feature (346)..(346): Wherein amino acid can be Asp, Asn
  misc_feature (347)..(347): Wherein amino acid can be Asn, Ala, Ser
  misc_feature (350)..(350): Wherein amino acid can be Thr, Ala
  misc_feature (351)..(351): Xaa can be Ser, Glu, Pro, Ala
  misc_feature (356)..(356): Wherein amino acid can be Lys, Glu
  misc_feature (357)..(357): Wherein amino acid can be Asn, Ala
  misc_feature (372)..(372): Wherein amino acid can be Arg, Ala
  misc_feature (375)..(375): Wherein amino acid can be Lys, Ala
  misc_feature (388)..(388): Wherein amino acid can be Arg, Ala
  misc_feature (409)..(409): Wherein amino acid can be Lys, Ala
  misc_feature (412)..(412): Wherein amino acid can be Lys, Ala
  misc_feature (432)..(432): Wherein amino acid can be Lys, Ala
  misc_feature (460)..(460): Wherein amino acid can be Arg, Ala
  misc_feature (461)..(461): Wherein amino acid can be Ala, Lys
  misc_feature (465)..(465): Wherein amino acid can be Trp, Ala
  misc_feature (560)..(560): Wherein amino acid can be Thr, Ala
  misc_feature (564)..(564): Wherein amino acid can be Ser, Pro
  misc_feature (571)..(571): Wherein amino acid can be Asn, Ser
  misc_feature (573)..(573): Wherein amino acid can be Ser, Ala
  misc_feature (576)..(576): Wherein amino acid can be Lys, Ala
  misc_feature (586)..(586): Wherein amino acid can be His, any naturally occuring amino acid
  misc_feature (587)..(587): Wherein amino acid can be Ile, Ala
  misc_feature (589)..(589): Wherein amino acid can be Met, Val
  misc_feature (592)..(592): Wherein amino acid can be Ser, Gly
  misc_feature (594)..(594): Wherein amino acid can be Phe, Leu

SEQ ID NO: 11 - protein, 594 aa, Artificial Sequence
  Modified hyperactive PiggyBac with mutations in the catalytic triad
  misc_feature (268)..(268): Xaa can be Asp, Asn
  misc_feature (346)..(346): Xaa can be Asp, Asn
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIXEQLLGFRGRCPFRVYIPNKPSKYGIKILMMCDSGTKYMINGMPYLGRGTQTNGVPLGEYYVKELSKPVHGSCRNITCXNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 12 - protein, 594 aa, Artificial Sequence
  Modified hyperactive PiggyBac with mutations in amino acids that are critical for excision
  misc_feature (287)..(287): Xaa can be Lys, Ala
  misc_feature (290)..(290): Xaa can be Lys, Ala
  misc_feature (460)..(460): Xaa can be Arg, Ala
  misc_feature (461)..(461): Xaa can be Lys, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNXPSXYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSXXTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 13 - protein, 594 aa, Artificial Sequence
  Modified hyperactive PiggyBac with mutation that are involved in target joining
  misc_feature (351)..(351): Xaa can be Ser, Glu, Pro, Ala
  misc_feature (356)..(356): Xaa can be Lys, Glu
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTXIPLAXNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 14 - protein, 594 aa, Artificial Sequence; identical to SEQ ID NO: 9
  Modified hyperactive PiggyBac with mutations that are critical for integration
  misc_feature (560)..(560): Wherein amino acid can be Thr, Ala,
  misc_feature (564)..(564): Wherein amino acid can be Ser, Pro
  misc_feature (571)..(571): Wherein amino acid can be Asn, Ser
  misc_feature (573)..(573): Wherein amino acid can be Ser, Ala
  misc_feature (589)..(589): Wherein amino acid can be Met, Val
  misc_feature (592)..(592): Wherein amino acid can be Ser, Gly
  misc_feature (594)..(594): Wherein amino acid can be Phe, Leu

SEQ ID NO: 15 - protein, 594 aa, Artificial Sequence
  Modified hyperactive PiggyBac with mutations that are involved in alignment
  misc_feature (325)..(325): Xaa can be Gly, Ala
  misc_feature (347)..(347): Xaa can be Asn, Ala, Ser
  misc_feature (350)..(350): Xaa can be Thr, Ala
  misc_feature (465)..(465): Xaa can be Trp, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLXEYYVKELSKPVHGSCRNITCDXWFXSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRXPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 16 - protein, 594 aa, Artificial Sequence
  Modified hyperactive PiggyBac with mutations at well conserved amino acids
  misc_feature (576)..(576): Xaa can be lys, Ala
  misc_feature (587)..(587): Xaa can be Ile, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKXCKKVICREHNXDMCQSCF

SEQ ID NO: 17 - protein, 594 aa, Artificial Sequence; identical to SEQ ID NO: 9
  Modified hyperactive PiggyBac with mutations involved in Zn2+ binding
  misc_feature (586)..(586): Wherein amino acid can be His, any naturally occuring amino acid

SEQ ID NO: 18 - protein, 594 aa, Artificial Sequence; identical to SEQ ID NO: 9
  Modified hyperactive PiggyBac with mutations that are involved in integration
  MISC_FEATURE (315)..(315): Wherein amino acid can be Arg, Ala
  MISC_FEATURE (341)..(341): Wherein amino acid can be Arg, Ala
  MISC_FEATURE (372)..(372): Wherein amino acid can be Arg, Ala
  MISC_FEATURE (375)..(372): Wherein amino acid can be Lys, Ala

SEQ ID NO: 19 - protein, 897 aa, Artificial Sequence
  Cas9 from Corynebacterium ulcerans
MTNAVANHHVLWAKFDNVSEPYPLLAHLLDTATAATCLFNHWLRKGLRDRLSTELGPDAEKILGFVAGIHDLGKANPYFQAQRRNKKEEWITLRDAIQKAGFPLSNGTSALFEETKEKRRHENITLSILGWEITKFLQVKDVWPQLAIIGHHGNFSAPGFLSDEDDLEDIEDIFDDNGWSPTHELLVSSLLQAVGLEKQPEIKHISPASAILISGLVVLADRIASQSEMASDGLQALQKEELFFHQPEKWIANRKAFCREIIENTVGTYHPWESEAAGIRAVLGDYEPRFTQKAALNAGDGLFNVMETTGAGKTEAALLRHVKRKERLLFFLPTQATTNAIMDRIGKIFDGTPNVASLAHGLAVTEDFYAHPILPVQGSSDDANYKDNGGLYPTEFVRSAGTPRLLAPVCVGTIDQALMGALPSKFNHLRLLALANAHVVVDEVHTMDQYQSELMSGLLEWWSATDTPVTLLTATMPAWQREKFHLSYTGKDPHFKGVFPSLEDWSTPSKNTETSQENIPTEAFTIPINIDKIAHNEIVDSHVQWVIEQRKLFPQARIGIICNTVGRAQSIAEALAHESPIVLHSRMTAGHRKEAATKLEQAIGKKGTANATLVIGTQAIEASLDIDLDLLRTELCPAPSLIQRAGRLWRRLDPQREVRVPGMVGKKLTIAVVDSPSTGQTLPYLRSQLYRVESWLKQRDRIEFPADIQDFIDATTPGLQELFQKVSLPEDCGSAEEREALADDYLNEVASWVTKQRQAGTSRIDFAKHGKPRQVLASDCVVEDFLQITSANNLEESATRLIDYPSISAILCDPTGTIPGAWTDSVEKLIAISAKDSESLRRALRASISIPHSKKFLPITSREIPLSEAKTLLSGYSAVHIQPDEYDLQSGLKGPQK

SEQ ID NO: 20 - protein, 876 aa, Artificial Sequence
  Cas9 from Corynebacterium diphtheria
MNPHEELWAKQKGLAKPYPLLAHLLDSAAVAGALWDHWLRQDLRQMFIEELGSNAREIIQFVVGSHDIGKATPLFQYQKAQKGEVWDSIRYAIDRTGRYQKPLPSSYLVKKTSGGPNRHEQWSSFASKNEYLKPSAAAKENWIGLAIGGHHGRFEPVGYGRHQRKAAEDLAKSGWSAAQQDLLRALEKASGITRASLPSELSPELTLVLSGLTILADRISSTESFVITGARMIDDGTLHLATPIDWLKTRKLDSEKHVAKTVGIYHGWNNHESAIHSILKGYDPRPLQTIALQNQVGLLNLMAPTGNGKTEAAILRHSLKENDRLIFLLPTQATSNAIMRRVQGIYSDTPNAAALAHSLASVEDFYQTPLSVFDDHYDPSKEQFESSMSGGLYPSSFVCSGAARLLAPICIGTVDQALATALPGKWIHLRILALANAHIVIDEVHTLDHYQTALLENILPILAKLKTKITFLTATMPSWQRTKLLTAYGGEDLQIPPTVFPAAETVLPGQFNRTLIDSDSTTIDFTMEETSYDHLVESHVKWHQTTRLNAPHARIGLICNTVKRAQEIAAALEKTNDRIVLLHSRMTTEHRRRSAELLESLLGPNGNRKTITVVGTQAIEASLDIDLDILRTELCPAPSLVQRAGRVWRRNDPYRSSRITADHKPISVVFIAEAKDWQVLPYLRAETSRTQRWLEKHNQMFLPQMAQEFIDAATVDLDTATSEMDLDALALMGIHLMKADGAKARIQDVLNSDSKVSDFALLTSKNEIDEAQTRLIEEGTHLRIILGDENESIPGGWKHGLSSLLKLKASDRESLRTALLASIPLLVSEKQKQLLYQHNLVPLSSSKTVLAGFYFLPKAQNFYSKNLGFIWPEEKD

SEQ ID NO: 21 - protein, 773 aa, Artificial Sequence
  Cas9 from Spiroplasma syrphidicola
MNYKKLILGLDLGIASCGWAVTGQMEDGNWVLDDFGVRLFQTPENSKDGTTNAAARRLKRGARRLIKRRKNRIKDLKNLFEKINFINKASLDKYINEHSATNLVEDFNRHELYNPYFLRSIGITEKLTREELVWSLIHIANRRGYKNKFAFDIEGDGKKRETKLDEAISNALISSNLTISQEIVRNKKFRDAKNKKALLVRNKGGKEGENNFQFLFARDDYKKEVDLLLAKQAKFYPELTEEIRAKAADIIFRQRDFEDGPGPKKQELREIYKKENKQFSKNFTQLEGRCTFLRELSVGYKSSILFDLFHIISEVSKISKYIEENDQLAQDIISSFLYNEAGKKGKTLLKEILKKHHINDDIFDTNAYKNIDFKTNYLNLLKEVFGNDVLKNLSLNRLEDNIYHQLGFIIHTNITPERKEKAINQWLLENNIILAKEKLNILLKPNSSISTTVKTSFKWMSIAISNFLKGIPYGKFQAQFIKEDNFKLPESYAKQYQKYLTGEKTFEMFAPIIDPDLWRNPIVFRAINQARKVIKKLFEKYTFIDQINIELTREMGLSFSDRKKVKERQDDSLKENAKAKEFLMANGIIVNDTNVLKYKLWIQQNKKSLYSGKEITIADLGASNVLQIDHIIPYSKLADDSFNNKVLVFSKENQEKGNQFADQYVKSLGTENYNNYKKRVNYLLFQNQINQKKAEYLLCSNQNEEILNDFVSRNLNDTRYITRYVTNWLKAEFELQSRFGLAKPKIMTLNGAITSRFRRTWLRNSPWGLEKKS

SEQ ID NO: 22 - protein, 1380 aa, Artificial Sequence
  Cas9 from Prevotella intermedia
MKRILGLDLGTTSIGWALVNEAENNNEASSIVRLGVRVNPLTVDEKSNFEKGKAITTNADRQLRHGARINLQRYKLRRQNLHDCLQKQGWLGTEAMYEEGKASTFETYKLRAKAAEEEISLHEFARVLFMLNKKRGYKSNRKANNKEDGQLFDGMTIAKKLYEEHLTPAEYSLQLLNKGKKFTQGYYRSDLNAELERIWDEQKKYYPEILTDEFKQQLEGKTKTNTSKIFLAKYGIYSADLKGLDRKFQPLKWRVEALQQQVDKEVLAFVISDLKGQIANTSGLLGAISDRSKELYFNKQTVGQYLWASLEENPHISIKNKPFYRQDYLDEFEKIWETQAAFHKQLTPELKQEIRDIIIFYQRPLKSKKSLISVCELEQRKVKATIDGKEKEITIGPKVAPKSSPVFQEFRIWQNLNNVLLIDNDTNEKRPLDEVERNLLYKELSIKAKLSKTEALKILNKKGKQWDLNYRELEGNRTQAILFDCYNRIITLTGHEECDFKKIKASEIRHYVSTIFKNLGFSTEILDFDPSLKKHELEKQPMYQLWHLLYSYESDNSRTGNESLLRKLETTFGFPEEYATVLCDVVFEEDYGNLSVKAMREILPYLQAGNDYSQACAYAGYNHSRHSLTKEELDQKVYKERLELLPKNSLRNPVVEKILNQMINVINAIIDEYGKPDEIRIEMARELKSSAADRKKTTHAISQGNAENQRIREILEKEFSLSYISRNDIIKYKLYEELEPNYYKTLYSDTYITKDKLFSKDFDIEHIIPKARLFDDSFSNKTLEARNINLEKSNKTAFDFIKEKYGEDGAEAYKKKLDMLLENDAISRPKYNNLLRAEADIPSDFINRDLRNTQYIAKKACEILGELVKTVTPTTGKITNRLREDWQLVDVMKELNFEKYEKLGLTEIVEDRDGRKIKRIKDWTKRNDHRHHAMDALAIAFTKPSFIQYLNNLNARSNKGDSIYAIENKELHYEEGKLRFNAPIPVNEFRAEAKRHLSAILVSIKAKNKVMTQNVNKIKTKHGIIKKIQLTPRGPLHNETIYGTKMRPIIKMVKVGAALDEATINKVSSPAIREALLKRLNEYSGNAKKAFTGKNTLEKNPIYLNAGRTKTVPSLVKTVEWESFHPTRKLIDKDLNVDKVVDKGIREILKARLEEFNGDAKKAFSNLEENPIYLDEAKKIALKRVSIEGVLSAIPLHTLKNQAGKPITGKDGKPVLGNYVQTSNNHHIAFYYDEDGNLQDNAVSFFEAAERKSQGIPVIDKDYNRDKGWRFLFTMKQNEYFVFPNEATGFIPSEVDLTDEANYGIISPNLYRVQKVSRIDKGTSASRDYWFRHHLETILNDDAKLKNLAFKRIRGLLELKDIIKVRINSTGKIVAVGEYD

SEQ ID NO: 23 - protein, 535 aa, Artificial Sequence
  Cas9 from Spiroplasma taiwanense
MWSRKILKAGSRLFDEANLSDKIASKRREQRGRRRNLRRKITWKQDLINLFVKYNFLQKENDFYELDFNFDLLELRKKAINSKIELEQLLIILFNYIKHRGSFNYREDLSELKNISQEELETSSEFKLPVDIQFELKEENNKFREINNEKSLINHEWYVKEINLILDAQIENKLINLDFKKDYLKLFNRKREYYDGPGPKDKNLLNPSKYGWKNQEEFFDRFAGKDTYDSKEQRAPKHSLTSYLFNILNDLNNLSINGDRNQLTYENKKDLINLTLINQKEKAENITLKKIAKYLKINEKNITGYRLKPNSNESIFTVFESANKMRSILVKNNKSIDFICLENIDKIDKIVDILTKYQSIEDKSLKLEELNFDFFDKETCEKLAVISLTGTHALSKKTMSKLIEEMFHDNLNHMEALAKLKIKPDYKLKVDLTNFKTIPILREKINEMYISPVVKRALIESLKIIKELERHFKDFEIKDIVIEMAKKNSAEKKQFISKIQRQNVDLVKKLSNDYSLDENKLNFKMKEKFLLLSEQ

SEQ ID NO: 24 - protein, 1281 aa, Artificial Sequence
  Cas9 from Streptococcus iniae
MRKPYSIGLDIGTNSVGWAVITDDYKVPSKKMRIQGTTDRTSIKKNLIGALLFDNGETAEATRLKRTTRRRYTRRKYRIKELQKIFSSEMNELDIAFFPRLSESFLVSDDKEFENHPIFGNLKDEITYHNDYPTIYHLRQTLADSDQKADLRLIYLALAHIIKFRGHFLIEGNLDSENTDVHVLFLNLVNIYNNLFEEDIVETASIDAEKILTSKTSKSRRLENLIAEIPNQKRNMLFGNLVSLALGLTPNFKTNFELLEDAKLQISKDSYEEDLDNLLAQIGDQYADLFIAAKKLSDAILLSDIITVKGASTKAPLSASMVQRYEEHQQDLALLKNLVKKQIPEKYKEIFDNKEKNGYAGYIDGKTSQEEFYKYIKPILLKLDGTEKLISKLEREDFLRKQRTFDNGSIPHQIHLNELKAIIRRQEKFYPFLKENQKKIEKLFTFKIPYYVGPLANGQSSFAWLKRQSNESITPWNFEEVVDQEASARAFIERMTNFDTYLPEEKVLPKHSPLYEMFMVYNELTKVKYQTEGMKRPVFLSSEDKEEIVNLLFKKERKVTVKQLKEEYFSKMKCFHTVTILGVEDRFNASLGTYHDLLKIFKDKAFLDDEANQDILEEIVWTLTLFEDQAMIERRLVKYADVFEKSVLKKLKKRHYTGWGRLSQKLINGIKDKQTGKTILGFLKDDGVANRNFMQLINDSSLDFAKIIKNEQEKTIKNESLEETIANLAGSPAIKKGILQSIKIVDEIVKIMGQNPDNIVIEMARENQSTMQGIKNSRQRLRKLEEVHKNTGSKILKEYNVSNTQLQSDRLYLYLLQDGKDMYTGKELDYDNLSQYDIDHIIPQSFIKDNSIDNTVLTTQASNRGKSDNVPNIETVNKMKSFWYKQLKSGAISQRKFDHLTKAERGALSDFDKAGFIKRQLVETRQITKHVAQILDSRFNSNLTEDSKSNRNVKIITLKSKMVSDFRKDFGFYKLREVNDYHHAQDAYLNAVVGTALLKKYPKLEAEFVYGDYKHYDLAKLMIQPDSSLGKATTRMFFYSNLMNFFKKEIKLADDTIFTRPQIEVNTETGEIVWDKVKDMQTIRKVMSYPQVNIVMKTEVQTGGFSKESIWPKGDSDKLIARKKSWDPKKYGGFDSPIIAYSVLVVAKIAKGKTQKLKTIKELVGIKIMEQDEFEKDPIAFLEKKGYQDIQTSSIIKLPKYSLFELENGRKRLLASAKELQKGNELALPNKYVKFLYLASHYTKFTGKEEDREKKRSYVESHLYYFDVRLSQVFRVTNVEF

SEQ ID NO: 25 - protein, 1352 aa, Artificial Sequence
  Cas9 from Belliella baltica
MKKILGLDLGTTSIGWAFIKEPEKDVVGSEIVDMGVRIVPLSSDEENDFAKGNTISINADRTLKRGARRNLQRFKQRRNALLEIFKEKKLISTNFKYAEDGPSSTFSTLNLRAKAAKEKIELQDLVKVLLQINKKRGYKSSRKAKSEEDDGSAIDSMGIAKELYENDLTPGQWVYEALQKGRKNVPDFYRSDLQEEFKKIVNYQSEFFPDIFNASFVEDWMGKASTPTKQYFNKKGVQLAENKGKREERRLQEYKWRAEAVNFKIDLSEIALILSQINSQISNSSGYLGAISDRSKELYFKNLTVGQYLYQQIKKNPHTRLKGQVFYRQDYLDEFERIWSVQSSFYPQLNDALKREVRDITIFFQRRLKSQKHLISNCEFEDHHKVVPKSHPVFQEFRIWQNLNNLLLIKKDNLNEKFDLELESKIALANELAFKRELNVKDALKILGLKPNEWEFNFTKIEGNRTNQAFFDAFAKIIELEDGEPIDLGDLKADDILDQFSEAFLRIGIDTELLQVNSDIEGAEYEKQSYIQFWHLLYSSEDDQKLKLNLIRKFGFKPEHAKILASISLQDDHASLSSRAIKKILPHLQSGLIYDKACTYAGYNHSSSFTKDENEKRELRAELELLKKNSLRNPVVEKILNQMINVVNAILKDPELGRPDEIRVEMARELKANAEQRKNMTSNIASATRDHDKYREILKSEFGLKRVTKNDLLRYKLWLETDGISLYTGKPIEASKLFSKEYDIEHIIPKARLFDDSFSNKTICERQLNIDKANVTAFSFLQNKLSADEFEQYQSRVKSLYGKLSKAKIQKLLMANDKIPEDFIARQLQETRYISKKAKEILFEISRRVSVTTGTITDKLREDWGLVEIMKELNWEKYDKLGLTYTIEGKHGERLNKIKDWSKRNDHRHHAMDALTVALTKPAYIQYLNNLNAKGLNNKKGTEVFAIEQKYLKRENGKLCFIPPIENIRSEAKKHLSRILVSYKAKNKVVTINKNKTKSKAGLNEQIALTPRGQLHKETVYGKSFHYSTKFEKIGASFNVQKINTVAKKEEREALLKRLAENGNDPKKAFTGKNTLNKMPIYLDLGKNIKLSEKVKTVVLEQNYTIRKNIDPDLKVDKVIDVGIKRILESRLEEFGGNAKLAFSNLEENPIWLNKEKGISIKRVKISGVSNVESLHVKKDHFGEPILDQEGNEIPVDFVSTGNNHHVAIYEDENGNLQEEVVSFFEAVVRQNQGLPIIKKNHTLGWKFLFTLKQNEYFVFPSDDFVPADVDLMDEQNYHLISPNLFRVQKIARKNYVFNNHLETKAVDNDLLKSKKELSKITYHFYQTPEHLRGIIKIRINHLGKIIQIGEY

SEQ ID NO: 26 - protein, 1509 aa, Artificial Sequence
  Cas9 from Psychroflexus torquisI
MKRILGLDLGTNSIGWSLIEHDFKNKQGQIEGLGVRIIPMSQEILGKFDAGQSISQTADRTKYRGVRRLYQRDNLRRERLHRVLKILDFLPKHYSESIDFQDKVGQFKPKQEVKLNYRKNEKNKHEFVFMNSFIEMVSEFKNAQPELFYNKGNGEETKIPYDWTLYYLRKKALTQQITKEELAWLILNFNQKRGYYQLRGEDIDEDKNKKYMQLKVNNLIDSGAKVKGKVLYNVIFDNGWKYEKQIVNKDEWEGRTKEFIITTKTLKNGNIKRTYKAVDSEIDWAAIKAKTEQDINKANKTVGEYIYESLLDNPSQKIRGKLVKTIERKFYKEEFEKLLSKQIELQPELFNESLYKACIKELYPRNENHQSNNKKQGFEYLFTEDIIFYQRPLKSQKSNISGCQFEHKIYKQKNKKTGKLELIKEPIKTISRSHPLFQEFRIWQWLQNLKIYNKEKIENGKLEDVTTQLLPNNEAYVTLFDFLNTKKELEQKQFIEYFVKKKLIDKKEKEHFRWNFVEDKKYPFSETRAQFLSRLAKVKGIKNTEDFLNKNTQVGSKENSPFIKRIEQLWHIIYSVSDLKEYEKALEKFAEKHNLEKDSFLKNFKKFPPFVSDYASYSKKAISKLLPIMRMGKYWSESAVPTQVKERSLSIMERVKVLPLKEGYSDKDLADLLSRVSDDDIPKQLIKSFISFKDKNPLKGLNTYQANYLVYGRHSETGDIQHWKTPEDIDRYLNNFKQHSLRNPIVEQVVMETLRVVRDIWEHYGNNEKDFFKEIHVELGREMKSPAGKREKLSQRNTENENTNHRIREVLKELMNDASVEGGVRDYSPSQQEILKLYEEGIYQNPNTNYLKVDEDEILKIRKKNNPTQKEIQRYKLWLEQGYISPYTGKIIPLTKLFTHEYQIEHIIPQSRYYDNSLGNKIICESEVNEDKDNKTAYEYLKVEKGSIVFGHKLLNLDEYEAHVNKYFKKNKTKLKNLLSEDIPEGFINRQLNDSRYISKLVKGLLSNIVRENGEQEATSKNLIPVTGVVTSKLKQDWGLNDKWNEIIAPRFKRLNKLTNSNDFGFWDNDINAFRIQVPDSLIKGFSKKRIDHRHHALDALVVACTSRNHTHYLSALNAENKNYSLRDKLVIKNENGDYTKTFQIPWQGFTIEAKNNLEKTVVSFKKNLRVINKTNNKFWSYKDENGNLNLGKDGKPKKKLRKQTKGYNWAIRKPLHKETVSGIYNINAPKNKIATSVRTLLTEIKNEKHLAKITDLRIRETILPNHLKHYLNNKGEANFSEAFSQGGIEDLNKKITTLNEGKKHQPIYRVKIFEVGSKFSISEDENSAKSKKYVEAAKGTNLFFAIYLDEENKKRNYETIPLNEVITHQKQVAGFPKSERLSVQPDSQKGTFLFTLSPNDLVYVPNNEELENRDLFNLGNLNVEQISRIYKFTDSSDKTCNFIPFQVSKLIFNLKKKEQKKLDVDFIIQNEFGLGSPQSKNQKSIDDVMIKEKCIKLKIDRLGNISKA

SEQ ID NO: 27 - protein, 1388 aa, Artificial Sequence
  Cas9 from Streptococcus thermophilus
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 28 - protein, 1334 aa, Artificial Sequence
  Cas9 from Listeria innocua
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 29 - protein, 984 aa, Artificial Sequence
  Cas9 from Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 30 - protein, 1082 aa, Artificial Sequence
  Cas9 from Neisseria. meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 31 - protein, 1367 aa, Artificial Sequence
  Cas9 from Streptococcus pyogenes
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 32 - protein, 528 aa, Artificial Sequence
  ZFP nucleic acid sequence
ATGGCCCAGGCTGCTCTTGAGCCCGGAGAGAAACCCTACAAGTGCCCGGAGTGCGGAAAGTCCTTCTCTGAGCGGAGTCACCTCCGAGAGCACCAGCGGACTCATACGGGCGAAAAACCATACAAGTGCCCAGAATGTGGTAAATCTTTTTCTCGGGCTGACAACCTGACTGAACATCAGCGCACGCACACCGGTGAAAAACCTTACAAGTGTCCAGAGTGTGGCAAGAGCTTTTCTAGTAGAAGGACCTGTCGAGCGCATCAGCGGACTCACACCGGCGAAAAACCCTATAAGTGTCCGGAATGTGGAAAGAGCTTTAGCCGCAACGACACCCTTACTGAACACCAGCGAACACACACGGGAGAAAAACCATATAAATGTCCGGAATGTGGCAAAAGTTTTAGTCGGAGTGATAAACTTACGGAGCACCAACGGACACACACCGGAGAGAAGCCATATAAGTGTCCTGAATGTGGAAAGTCCTTCTCACAGCTTGCTCATCTGCGAGCACATCAGCGCACACACACC

SEQ ID NO: 33 - protein, 176 aa, Artificial Sequence
  ZFP amino acid sequence
MAQAALEPGEKPYKCPECGKSFSERSHLREHQRTHTGEKPYKCPECGKSFSRADNLTEHQRTHTGEKPYKCPECGKSFSSRRTCRAHQRTHTGEKPYKCPECGKSFSRNDTLTEHQRTHTGEKPYKCPECGKSFSRSDKLTEHQRTHTGEKPYKCPECGKSFSQLAHLRAHQRTHT

SEQ ID NO: 34 - DNA, 555 bp, Artificial Sequence
  ZNF-E2C Nucleotide
atggcgcaggcggcgctggaaccgggcgaaaaaccgtataaatgcccggaatgcggcaaaagctttagccgcaaagatagcctggtgcgccatcagcgcacccataccggcgaaaaaccgtataaatgcccggaatgcggcaaaagctttagccagagcggcgatctgcgccgccatcagcgcacccataccggcgaaaaaccgtataaatgcccggaatgcggcaaaagctttagcgattgccgcgatctggcgcgccatcagcgcacccataccggcgaaaaaccgtataaatgcccggaatgcggcaaaagctttagccagagcagccatctggtgcgccatcagcgcacccataccggcgaaaaaccgtataaatgcccggaatgcggcaaaagctttagcgattgccgcgatctggcgcgccatcagcgcacccataccggcgaaaaaccgtataaatgcccggaatgcggcaaaagctttagccgcagcgataaactggtgcgccatcagcgcacccataccggcaaaaaaaccagcggccaggcgggc

SEQ ID NO: 35 - protein, 185 aa, Artificial Sequence
  ZNF-E2C Aminoacid
MAQAALEPGEKPYKCPECGKSFSRKDSLVRHQRTHTGEKPYKCPECGKSFSQSGDLRRHQRTHTGEKPYKCPECGKSFSDCRDLARHQRTHTGEKPYKCPECGKSFSQSSHLVRHQRTHTGEKPYKCPECGKSFSDCRDLARHQRTHTGEKPYKCPECGKSFSRSDKLVRHQRTHTGKKTSGQAG

SEQ ID NO: 36 - DNA, 555 bp, Artificial Sequence
  ZNF-E3 Nucleotide
atggcgcaggcggcgctggaaccgggcgaaaaaccgtataaatgcccggaatgcggcaaaagctttagcgatccgggcgcgctggtgcgccatcagcgcacccataccggcgaaaaaccgtataaatgcccggaatgcggcaaaagctttagccagagcagccatctggtgcgccatcagcgcacccataccggcgaaaaaccgtataaatgcccggaatgcggcaaaagctttagcgattgccgcgatctggcgcgccatcagcgcacccataccggcgaaaaaccgtataaatgcccggaatgcggcaaaagctttagccagagcagccatctggtgcgccatcagcgcacccataccggcgaaaaaccgtataaatgcccggaatgcggcaaaagctttagcgattgccgcgatctggcgcgccatcagcgcacccataccggcgaaaaaccgtataaatgcccggaatgcggcaaaagctttagccagagcagccatctggtgcgccatcagcgcacccataccggcaaaaaaaccagcggccaggcgggc

SEQ ID NO: 37 - protein, 185 aa, Artificial Sequence
  ZNF-E3 Amino acid
MAQAALEPGEKPYKCPECGKSFSDPGALVRHQRTHTGEKPYKCPECGKSFSQSSHLVRHQRTHTGEKPYKCPECGKSFSDCRDLARHQRTHTGEKPYKCPECGKSFSQSSHLVRHQRTHTGEKPYKCPECGKSFSDCRDLARHQRTHTGEKPYKCPECGKSFSQSSHLVRHQRTHTGKKTSGQAG

SEQ ID NO: 38 - protein, 528 aa, Artificial Sequence
  ZNF-TRCa Nucleotide
ATGGCGCAGGCGGCTCTTGAACCCGGGGAGAAACCCTATAAATGCCCTGAGTGTGGCAAGAGTTTTTCAACCACAGGAAACTTGACAGTCCACCAACGGACCCACACCGGCGAGAAACCATACAAGTGTCCGGAGTGTGGTAAGTCTTTCTCAAGTCCTGCCGACCTTACCAGACATCAACGCACACATACAGGTGAAAAACCTTACAAGTGCCCAGAGTGCGGAAAAAGTTTTTCACAATCTGGCGACCTCCGCAGGCACCAGCGCACTCACACCGGTGAAAAACCATACAAGTGTCCTGAGTGCGGGAAGAGTTTTAGTCAACGAGCTCATCTGGAGCGACACCAAAGGACTCATACTGGGGAGAAACCGTACAAATGTCCCGAATGTGGGAAGAGCTTCTCTACCAAGAATTCCCTTACAGAGCACCAGCGCACGCATACGGGAGAGAAGCCGTATAAGTGTCCGGAATGTGGCAAGAGCTTTTCCAGAAGTGACCACCTTACAACCCACCAGAGGACGCACACC

SEQ ID NO: 39 - protein, 176 aa, Artificial Sequence
  ZNF-TRCa Aminoacid
MAQAALEPGEKPYKCPECGKSFSTTGNLTVHQRTHTGEKPYKCPECGKSFSSPADLTRHQRTHTGEKPYKCPECGKSFSQSGDLRRHQRTHTGEKPYKCPECGKSFSQRAHLERHQRTHTGEKPYKCPECGKSFSTKNSLTEHQRTHTGEKPYKCPECGKSFSRSDHLTTHQRTHT

SEQ ID NO: 40 - protein, 18 aa, Artificial Sequence
  AAVS1 site
AGACGGCCGCGTCAGAGC

SEQ ID NO: 41 - protein, 7 aa, Artificial Sequence
  Zinc Finger 1
ERSHLRE

SEQ ID NO: 42 - protein, 7 aa, Artificial Sequence
  Zinc Finger 2
RADNLTE

SEQ ID NO: 43 - protein, 7 aa, Artificial Sequence
  Zinc Finger 3
SRRTCRA

SEQ ID NO: 44 - protein, 7 aa, Artificial Sequence
  Zinc Finger 4
RNDTLTE

SEQ ID NO: 45 - protein, 7 aa, Artificial Sequence
  Zinc Finger 5
RSDKLTE

SEQ ID NO: 46 - protein, 7 aa, Artificial Sequence
  Zinc Finger 6
QLAHLRA

SEQ ID NO: 47 - DNA, 51 bp, Artificial Sequence
  Nuclear Localization Signal
atggctccaaagaaaaagaggaaagtgggaatccacggagtccccgccgct

SEQ ID NO: 48 - DNA, 30 bp, Artificial Sequence
  GGS linker Nucleic Acid
ggtggatctggcggtggatctggtggcggt

SEQ ID NO: 49 - protein, 10 aa, Artificial Sequence
  GGS linker Amino Acid
GGSGGGSGGG

SEQ ID NO: 50 - DNA, 36 bp, Artificial Sequence
  GGS4x Linker Nucleic Acid
ggagggagtggtgggtccggtggtagtggcggatcc

SEQ ID NO: 51 - protein, 12 aa, Artificial Sequence
  GGS4x Linker Amino Acid
GGSGGSGGSGGS

SEQ ID NO: 52 - DNA, 45 bp, Artificial Sequence
  GGS5x Linker Nucleic Acid
ggaggctccggtgggtctggtgggagcggtggtagtggcggatcc

SEQ ID NO: 53 - protein, 15 aa, Artificial Sequence
  GGS5x Linker Amino Acid
GGSGGSGGSGGSGGS

SEQ ID NO: 54 - DNA, 54 bp, Artificial Sequence
  GGS6x Linker Nucleic Acid
ggaggcagtggtgggagcggtggttccgggggtagtggtggttccgggggatcc

SEQ ID NO: 55 - protein, 18 aa, Artificial Sequence
  GGS6x Linker Amino Acid
GGSGGSGGSGGSGGSGGS

SEQ ID NO: 56 - DNA, 63 bp, Artificial Sequence
  GGS7x Linker Nucleic Acid
ggaggttctggaggctccggtgggtccgggggaagtggggggtcaggcggatcaggaggatcc

SEQ ID NO: 57 - protein, 21 aa, Artificial Sequence
  GGS7x Linker Amino Acid
GGSGGSGGSGGSGGSGGSGGS

SEQ ID NO: 58 - DNA, 75 bp, Artificial Sequence
  GGS8x Linker Nucleic Acid
ggaggtagcggaggttccggagggagcggcgggagtgggggaagcgggggaagtggaggatccgggggaggatcc

SEQ ID NO: 59 - protein, 21 aa, Artificial Sequence
  GGS8x Linker Amino Acid
GGSGGSGGSGGSGGSGGSGGS

SEQ ID NO: 60 - DNA, 48 bp, Artificial Sequence
  Linker XTEN Nucleic Acid
tccggtagcgaaacaccggggacttcagaatcggccaccccggagtct

SEQ ID NO: 61 - protein, 16 aa, Artificial Sequence
  Linker XTEN Amino Acid
SGSETPGTSESATPES

SEQ ID NO: 62 - DNA, 36 bp, Artificial Sequence
  Linker B Nucleic Acid
ggaagcgccggtagtgcggctgggtctggcgagttc

SEQ ID NO: 63 - protein, 12 aa, Artificial Sequence
  Linker B Amino Acid
GSAGSAAGSGEF

SEQ ID NO: 64 - DNA, 4104 bp, Artificial Sequence
  human Cas9 (hCas9) nucleic acid
atggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 65 - DNA, 4104 bp, Artificial Sequence
  nickase Cas9 (nCas9)  nucleic acid
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaacaggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 66 - DNA, 4104 bp, Artificial Sequence
  dead Cas9 (dCas9)  nucleic acid
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 67 - DNA, 1782 bp, Artificial Sequence
  hyperactive PiggyBac (PB) transposase nucleic acid
atgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttc

SEQ ID NO: 68 - DNA, 1020 bp, Artificial Sequence
  hyperactive Sleeping Beauty (SB100) transposase nucleic acid
atgggaaaatcaaaagaaatcagccaagacctcagaaaaagaattgtagacctccacaagtctggttcatccttgggagcaatttccaaacgcctggcggtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccactcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatggacgccgtgcagtatgtggatatattgaagcaacatctcaagacatcagtcaggaagttaaagcttggtcgcaaatgggtcttccaacacgacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaaattattgtgggaagcttgtggaaggctacccgaaacgtttgacccaagttaaacaatttaaaggcaatgctaccaaatac

SEQ ID NO: 69 - protein, 1368 aa, Artificial Sequence [sequence omitted]

SEQ ID NO: 70 - protein, 1368 aa, Artificial Sequence
  nickase Cas9 (nCas9)  amino acid
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 71 - protein, 1368 aa, Artificial Sequence
  dead Cas9 (dCas9)  amino acid
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 72 - protein, 594 aa, Artificial Sequence; identical to SEQ ID NO: 9
  hyperactive PiggyBac (PB) transposase amino acid

SEQ ID NO: 73 - protein, 340 aa, Artificial Sequence
  hyperactive Sleeping Beauty (SB100) transposase amino acid
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 74 - DNA, 118 bp, Artificial Sequence
  IN cPPT/CTS domain Nucleic Acid
ttttaaaagaaaaggggggattggggggtacagtgcaggggaaagaatagtagacataatagcaacagacatacaaactaaagaattacaaaaacaaattacaaaaattcaaaatttt

SEQ ID NO: 75 - protein, 39 aa, Artificial Sequence
  Primer GG-cPPT-Fw
TCCTCTCGTCTCCATTATTTTAAAAGAAAAGGGGGGATT

SEQ ID NO: 76 - protein, 39 aa, Artificial Sequence
  Primer GG-cPPT-STOP-Fw
TCCTCTCGTCTCCATTAATTTAAAAGAAAAGGGGGGATT

SEQ ID NO: 77 - protein, 47 aa, Artificial Sequence
  Primer GG-cPPT-Rv
TCCTCTCGTCTCCCTGAAAAATTTTGAATTTTTGTAATTTGTTTTTG

SEQ ID NO: 78 - protein, 39 aa, Artificial Sequence
  Primer GG-AAVS1-6d-Fw
TCCTCTCGTCTCCATTATATGGCTCCAAAGAAAAAGAGG

SEQ ID NO: 79 - protein, 45 aa, Artificial Sequence
  Primer GG-AAVS1-6d-Rv
TCCTCTCGTCTCCCTGATCAATCCTCATCCTGTCTACTTGCCACA

SEQ ID NO: 80 - protein, 35 aa, Artificial Sequence
  Primer GG-AAVS1-6d (-NLS)-Fw
TCCTCTCGTCTCCATTATATGGCCCAGGCTGCTCT

SEQ ID NO: 81 - protein, 24 aa, Artificial Sequence
  Primer IN-Fw
TTTTAGATGGAATAGATAAGGCCC

SEQ ID NO: 82 - protein, 32 aa, Artificial Sequence
  Primer XbaI-pSICO_IC-5′Fw1
CTAGCTCTAGATGGCTAACTAGGGAACCCACT

SEQ ID NO: 83 - protein, 33 aa, Artificial Sequence
  Primer SacI-pSICO_IC-5′Rv1
CTAGCGAGCTCCCAGGCTCAGATCTGGTCTAAC

SEQ ID NO: 84 - protein, 31 aa, Artificial Sequence
  Primer XbaI-pSICO_IC-5′Fw2
CTAGCTCTAGACTAACTAGGGAACCCACTGC

SEQ ID NO: 85 - protein, 48 aa, Artificial Sequence
  Primer SacI-pSICO_IC-5′Rv2
CCTCTCTATGGGCAGTCTAGCGAGCTCCTGGTCTAACCAGAGAGACCC

SEQ ID NO: 86 - protein, 32 aa, Artificial Sequence
  Primer XbaI-pSICO_IC-3′Fw1
CTAGCTCTAGATCCCTCAGACCCTTTTAGTCA

SEQ ID NO: 87 - protein, 31 aa, Artificial Sequence
  Primer SacI-pSICO_IC-3′Rv1
CTAGCGAGCTCCAACAGACGGGCACACACTA

SEQ ID NO: 88 - protein, 32 aa, Artificial Sequence
  Primer XbaI-pSICO_IC-3′Fw2
CTAGCTCTAGAAAAATCTCTAGCAGCCCATCC

SEQ ID NO: 89 - protein, 47 aa, Artificial Sequence
  Primer SacI-pSICO_IC-3′Rv2
CCTCTCTATGGGCAGTCTAGCGAGCTCGACGGGCACACACTACTTGA

SEQ ID NO: 90 - protein, 35 aa, Artificial Sequence
  Primer CCD1-A128T-F
TCACCAGTACTACAGTTAAGACCGCCTGTTGGTGG

SEQ ID NO: 91 - protein, 35 aa, Artificial Sequence
  Primer CCD1-A128T-R
CCACCAACAGGCGGTCTTAACTGTAGTACTGGTGA

SEQ ID NO: 92 - protein, 41 aa, Artificial Sequence
  Primer CCD2-E170G-F
ACAGGTAAGAGATCAGGCTGGCCATCTTAAGACAGCAGTAC

SEQ ID NO: 93 - protein, 41 aa, Artificial Sequence
  Primer CCD2-E170G-R
GTACTGCTGTCTTAAGATGGCCAGCCTGATCTCTTACCTGT

SEQ ID NO: 94 - protein, 63 aa, Artificial Sequence
  Primer NTD1-E10/13K-F
GGTTTTTTAGATGGAATAGATAAGGCCCAAAAGGAACATAAGAAATATCACAGTAATTGGAGA

SEQ ID NO: 95 - protein, 63 aa, Artificial Sequence
  Primer NTD1-E10/13K-R
TCTCCAATTACTGTGATATTTCTTATGTTCCTTTTGGGCCTTATCTATTCCATCTAAAAAACC

SEQ ID NO: 96 - protein, 49 aa, Artificial Sequence
  Primer Solubility-F185K-F
AAATGGCAGTATTCATCCACAATAAGAAAAGAAAAGGGGGGATTGGGGG

SEQ ID NO: 97 - protein, 49 aa, Artificial Sequence
  Primer Solubility-F185K-R
CCCCCAATCCCCCCTTTTCTTTTCTTATTGTGGATGAATACTGCCATTT

SEQ ID NO: 98 - protein, 54 aa, Artificial Sequence
  Primer Primer NGS-aavs fw
ACACTCTTTCCCTACACGACGCTCTTCCGATCTAGGACAGCATGTTTGCTGCCT

SEQ ID NO: 99 - protein, 51 aa, Artificial Sequence
  Primer NGS-aavs rv
GACTGGAGTTCAGACGTGTGCTCTTCCGATCTGCTCCAGGAAATGGGGGTG

SEQ ID NO: 100 - protein, 34 aa, Artificial Sequence
  Primer PB R245A
CGTGTTCACCCCCGTGGCAAAGATCTGGGACCTG

SEQ ID NO: 101 - protein, 36 aa, Artificial Sequence
  Primer PB R275-277A
AGCTGCTGGGCTTCGCGGGCGCGTGCCCCTTCAGGG

SEQ ID NO: 102 - protein, 28 aa, Artificial Sequence
  Primer PB R388A
GAACAGCAGGTCCGCGCCCGTGGGCACC

SEQ ID NO: 103 - protein, 32 aa, Artificial Sequence
  Primer PB S351A
GACAACTGGTTCACCGCCATCCCCCTGGCCAA

SEQ ID NO: 104 - protein, 30 aa, Artificial Sequence
  Primer PB W465A
GAAAGACCAACAGGGCGCCCATGGCCCTGC

SEQ ID NO: 105 - protein, 43 aa, Artificial Sequence
  Primer PB R372A-K375A
CATCGTGGGCACCGTGGCAAGCAACGCGAGAGAGATCCCCGAG

SEQ ID NO: 106 - protein, 29 aa, Artificial Sequence
  Primer PB D450N
GCGTGGACACCCTGAACCAGATGTGCAGC

SEQ ID NO: 107 - protein, 21 aa, Artificial Sequence
  Primer SYBR-WPRE-3_Fw
ACGCTATGTGGATACGCTGCT

SEQ ID NO: 108 - protein, 21 aa, Artificial Sequence
  Primer SYBR-WPRE-3_Rv
AGCAAACACAGTGCACACCAC

SEQ ID NO: 109 - protein, 20 aa, Artificial Sequence
  Primer SYBR-RNaseP_Fw
GGAGTGAGGAGGGATGTGAA

SEQ ID NO: 110 - protein, 20 aa, Artificial Sequence
  Primer SYBR-RNaseP_Rv
ATTGAGGGCACTGGAAATTG

SEQ ID NO: 111 - protein, 68 aa, Artificial Sequence
  Primer Illumina custom
AATGATACGGCGACCACCGAGATCTACACAGCTAGACACTCTTTCCCTACACGACGCTCTTCCGATCT

SEQ ID NO: 112 - protein, 64 aa, Artificial Sequence
  Primer NEBNext Index 9
CAAGCAGAAGACGGCATACGAGATCTGATCGTGACTGGAGTTCAGACGTGTGCTCTTCCGATCT

SEQ ID NO: 113 - protein, 53 aa, Artificial Sequence
  Primer NGS cluster 1 fw
ACACTCTTTCCCTACACGACGCTCTTCCGATCTCTGCGGGAGAACGACGTGTT

SEQ ID NO: 114 - protein, 55 aa, Artificial Sequence
  Primer NGS cluster 2 rv
GACTGGAGTTCAGACGTGTGCTCTTCCGATCTCCTCACCTTCCTCTTCTTCTTGG

SEQ ID NO: 115 - protein, 42 aa, Artificial Sequence
  Primer CMV-F
CTGCAGCGCGGGGATCTCATGCTGGAGTTCTTCGCCCACCCC

SEQ ID NO: 116 - protein, 25 aa, Artificial Sequence
  Primer cas9 rv
CACCTTCCTCTTCTTCTTGGGGTCA

SEQ ID NO: 117 - protein, 573 aa, Artificial Sequence
  ZFP_TCRa4 na sequence
ATGGCTCCTAAGAAGAAGCGGAAAGTCGGCATACACGGAGTGCCTGCTGCAATGGCAGAAAGGCCATTCCAATGCAGAATATGCATGAGGAACTTCTCAGATCGCAGTAACCTCTCAAGGCATATACGGACCCATACGGGGGAAAAACCATTTGCCTGTGATATATGTGGCCGCAAGTTCGCTCAGAAAGTGACCTTGGCAGCTCACACTAAGATTCACACACATCCAAGAGCCCCTATCCCTAAGCCGTTCCAATGTAGGATATGCATGCGAAACTTCTCTGATCGGAGTGCACTGAGTAGGCACATCAGAACACACACGGGAGAAAAGCCTTTCGCTTGCGATATCTGCGGGCGGAAGTTCGCAACATCCGGGAATCTCACTCGCCATACGAAAATACACACTGGCAGCCAAAAACCTTTCCAATGCCGAATATGTATGAGAAATTTTAGCTACAGAAGTTCATTGAAAGAACACATTAGAACCCATACCGGAGAAAAGCCGTTCGCGTGCGATATCTGCGGTCGGAAGTTCGCTACCTCAGGCAACCTGACACGCCACACGAAAATCCAC

SEQ ID NO: 118 - protein, 191 aa, Artificial Sequence
  ZFP_TCRa4 aa sequence
MAPKKKRKVGIHGVPAAMAERPFQCRICMRNFSDRSNLSRHIRTHTGEKPFACDICGRKFAQKVTLAAHTKIHTHPRAPIPKPFQCRICMRNFSDRSALSRHIRTHTGEKPFACDICGRKFATSGNLTRHTKIHTGSQKPFQCRICMRNFSYRSSLKEHIRTHTGEKPFACDICGRKFATSGNLTRHTKIH

SEQ ID NO: 119 - protein, 594 aa, Artificial Sequence; identical to SEQ ID NO: 9
  Modified hyperactive PiggyBac aa sequence
  MISC_FEATURE (245)..(245): Wherein amino acid could be Ala
  MISC_FEATURE (275)..(275): Wherein amino acid could be Ala
  MISC_FEATURE (277)..(277): Wherein amino acid could be Ala
  MISC_FEATURE (325)..(325): Wherein amino acid could be Ala
  MISC_FEATURE (347)..(347): Wherein amino acid could be Ala, Ser
  MISC_FEATURE (351)..(351): Wherein amino acid could be Glu, Pro, Ala
  MISC_FEATURE (372)..(372): Wherein amino acid could be Ala
  MISC_FEATURE (375)..(375): Wherein amino acid could be Ala
  MISC_FEATURE (388)..(388): Wherein amino acid could be Ala
  MISC_FEATURE (450)..(450): Wherein amino acid could be Asn
  MISC_FEATURE (465)..(465): Wherein amino acid could be Ala
  MISC_FEATURE (560)..(560): Wherein amino acid could be Ala
  MISC_FEATURE (564)..(564): Wherein amino acid could be Pro
  MISC_FEATURE (573)..(573): Wherein amino acid could be Ala
  MISC_FEATURE (589)..(589): Wherein amino acid could be Val
  MISC_FEATURE (592)..(592): Wherein amino acid could be Gly
  MISC_FEATURE (594)..(594): Wherein amino acid could be Leu

SEQ ID NO: 120 - protein, 594 aa, Artificial Sequence; identical to SEQ ID NO: 9
  Top1 Modified hyperactive PiggyBac aa sequence
  MISC_FEATURE (245)..(245): Wherein amino acid could be Ala
  MISC_FEATURE (275)..(275): Wherein amino acid could be Arg, Ala
  MISC_FEATURE (277)..(277): Wherein amino acid could be Arg, Ala
  MISC_FEATURE (325)..(325): Wherein amino acid could be Ala, Gly
  MISC_FEATURE (347)..(347): Wherein amino acid could be Asn, Ala
  MISC_FEATURE (351)..(351): Wherein amino acid could be Glu, Pro, Ala
  MISC_FEATURE (372)..(372): Wherein amino acid could be Arg

SEQ ID NO: 121 - protein, 594 aa, Artificial Sequence
  Top1.1 Modified hyperactive PiggyBac aa sequence
  MISC_FEATURE (450)..(450): Wherein the amino acid can be Asp, Asn
  MISC_FEATURE (456)..(456): Wherein the amino acid can be Trp, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVAKIWDLFIHQCIQNYTPGAHLTIDEQLLGFRGRCPFRVYIPNKPSKYGIKILMMCDSGTKYMINGMPYLGRGTQTNGVPLAEYYVKELSKPVHGSCRNITCDNWFTEIPLAKNLLQEPYKLTIVGTVRSNAREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPPKIRRKASASCKKCKKVICREHNIDMCQGCL

SEQ ID NO: 122 - protein, 594 aa, Artificial Sequence
  Top1.2 Modified hyperactive PiggyBac aa sequence
  MISC_FEATURE (450)..(450): Wherein the amino acid can be Asp, Asn
  MISC_FEATURE (456)..(456): Wherein the amino acid can be Trp, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVAKIWDLFIHQCIQNYTPGAHLTIDEQLLGFAGACPFRVYIPNKPSKYGIKILMMCDSGTKYMINGMPYLGRGTQTNGVPLGEYYVKELSKPVHGSCRNITCDAWFTPIPLAKNLLQEPYKLTIVGTVRSNAREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCAYCPSKIRRKASASCKKCKKVICREHNIDMCQSCF

SEQ ID NO: 123 - protein, 594 aa, Artificial Sequence
  Top1.3 Modified hyperactive PiggyBac aa sequence
  MISC_FEATURE (450)..(450): Wherein the amino acid can be Asp, Asn
  MISC_FEATURE (456)..(456): Wherein the amino acid can be Trp, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVAKIWDLFIHQCIQNYTPGAHLTIDEQLLGFRGRCPFRVYIPNKPSKYGIKILMMCDSGTKYMINGMPYLGRGTQTNGVPLAEYYVKELSKPVHGSCRNITCDAWFTAIPLAKNLLQEPYKLTIVGTVRSNAREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCAYCPSKIRRKASAACKKCKKVICREHNIDMCQSCF

SEQ ID NO: 124 - protein, 594 aa, Artificial Sequence
  Regular modified 1 hyperactive PiggyBac aa sequence
  MISC_FEATURE (450)..(450): Wherein the amino acid can be Asp, Asn
  MISC_FEATURE (456)..(456): Wherein the amino acid can be Trp, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDAWFTSIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 125 - protein, 594 aa, Artificial Sequence
  Regular modified 2 hyperactive PiggyBac aa sequence
  MISC_FEATURE (450)..(450): Wherein the amino acid can be Asp, Asn
  MISC_FEATURE (456)..(456): Wherein the amino acid can be Trp, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQGCF

SEQ ID NO: 126 - protein, 594 aa, Artificial Sequence; identical to SEQ ID NO: 9
  Regular modified 3 hyperactive PiggyBac aa sequence
  MISC_FEATURE (450)..(450): Wherein the amino acid can be Asp, Asn
  MISC_FEATURE (456)..(456): Wherein the amino acid can be Trp, Ala

SEQ ID NO: 127 - protein, 594 aa, Artificial Sequence
  Regular modified 4 hyperactive PiggyBac aa sequence
  MISC_FEATURE (450)..(450): Wherein the amino acid can be Asp, Asn
  MISC_FEATURE (456)..(456): Wherein the amino acid can be Trp, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVAKIWDLFIHQCIQNYTPGAHLTIDEQLLGFAGACPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCL

SEQ ID NO: 128 - protein, 594 aa, Artificial Sequence
  Regular modified 5 hyperactive PiggyBac aa sequence
  MISC_FEATURE (450)..(450): Wherein the amino acid can be Asp, Asn
  MISC_FEATURE (456)..(456): Wherein the amino acid can be Trp, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFAGRCPFRVYIPNKPSKYGIKILMMCDSGTKYMINGMPYLGRGTQTNGVPLAEYYVKELSKPVHGSCRNITCDSWFTAIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCAYCPSKIRRKASASCKKCKKVICREHNIDMCQGCF

SEQ ID NO: 129 - protein, 594 aa, Artificial Sequence
  Regular modified 6 hyperactive PiggyBac aa sequence
  MISC_FEATURE (450)..(450): Wherein the amino acid can be Asp, Asn
  MISC_FEATURE (456)..(456): Wherein the amino acid can be Trp, Ala
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFAGRCPFRVYIPNKPSKYGIKILMMCDSGTKYMINGMPYLGRGTQTNGVPLGEYYVKELSKPVHGSCRNITCDNWFTAIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCAYCPSKIRRKASASCKKCKKVICREHNIDMCQGCF

SEQ ID NO: 130 - protein, 14 aa, Artificial Sequence
  Linker aa sequence
KLAGGAPAVGGGPK

SEQ ID NO: 131 - protein, 19 aa, Artificial Sequence
  Linker aa sequence
EFGGGGSGGGGSGGGGSQF

SEQ ID NO: 132 - protein, 20 aa, Artificial Sequence
  Primer SV40pA-R
GAAATTTGTGATGCTATTGC

SEQ ID NO: 133 - protein, 5 aa, Artificial Sequence
  Linker
  MISC_FEATURE (1)..(5): Wherein the sequence can be repeated 1 to 50 times
GGGGS

SEQ ID NO: 134 - protein, 5 aa, Artificial Sequence
  Linker
  MISC_FEATURE (1)..(5): Wherein the sequence can be repeated 1 to 50 times
EAAAK